(19) United States
(12) Patent Application Publication
(10) Pub. No.: US 2008/0292560 A1
(43) Pub. Date: Nov. 27, 2008

(54) SILICONE IN GLYCOL PHARMACEUTICAL AND COSMETIC COMPOSITIONS WITH ACCOMMODATING AGENT

(76) Inventors: Dov TAMARKIN, Maccabim (IL); Doron FRIEDMAN, Karmei Yosef (IL); Ella ZLATKIS, Rehovot (IL); Tal BERMAN, Rishon LeZiyyon (IL); David SCHUZ, Moshav Gimzu (IL)

Correspondence Address: WILMERHALE/BOSTON, 60 STATE STREET, BOSTON, MA, 02109, US

(21) Appl. No.: 12/049,203

(22) Filed: Mar. 14, 2008

Related U.S. Application Data
(63) Continuation-in-part of application No. 12/014,088, filed on Jan. 14, 2008.
(60) Provisional application No. 60/918,025, filed on Mar. 14, 2007.
(60) Provisional application No. 60/880,434, filed on Jan. 12, 2007.
(60) Provisional application No. 60/919,303, filed on Mar. 21, 2007.

(51) Int. Cl. A61K 9/12; A61K 8/04

(52) U.S. Cl. 424/45

(57) ABSTRACT

A carrier, composition or foam formulation comprising; a silicone; about 25% to about 98% of a solvent selected from the group consisting of (1) a propylene glycol or derivative and (2) a polyethylene glycol (PEG) or derivative or mixtures thereof; 0% to about 48% of at least one secondary solvent; and an Accommodating Agent or Complex; and methods of treatment. A hygroscopic silicone in glycol containing composition includes at least one hygroscopic substance at a concentration sufficient to provide an Aw value of at least 0.9 and a therapeutic agent.

### CROSS REFERENCE TO RELATED APPLICATIONS

This application claims the benefit under 35 U.S.C. §119(e) of U.S. Provisional Patent Application No. 60/918,025, filed Mar. 14, 2007, entitled “Silicone in Glycol Pharmaceutical and Cosmetic Compositions with Accommodating Agent,” which is herein incorporated by reference in its entirety.

This application is a continuation-in-part of U.S. patent application Ser. No. 12/014,088, filed on Jan. 14, 2008, entitled “Hydrophilic Non-Aqueous Pharmaceutical Carriers and Compositions and Uses” which claims the benefit under 35 U.S.C. §119(e) to U.S. Provisional Application No. 60/880,434 filed Jan. 12, 2007, and entitled “Hydrophilic or Waterless Vehicle and Foamable Pharmaceutical Compositions,” and to 60/919,303 filed Mar. 21, 2007, and entitled “Hydrophilic and Non-Aqueous Pharmaceutical Carriers and Compositions and Uses,” all of which are incorporated in their entirety by reference.

### BACKGROUND

The invention relates to waterless or substantially waterless carriers, compositions and foams comprising a silicone and the use of them.

External topical administration is an important route for the administration of drugs in disease treatment. Many groups of drugs, including, for example, antibiotic, anti-fungal, anti-inflammatory, anesthetic, analgesic, anti-allergic, corticosteroid, retinoid and anti-proliferative medications are preferably administered in hydrophobic media, namely ointment. However, ointments often form an impermeable barrier, so that metabolic products and excreta from the wounds to which they are applied are not easily removed or drained away. Furthermore, it is difficult for the active drug dissolved in the carrier to pass through the white petrolatum barrier layer into the wound tissue, so the efficacy of the drug is reduced. In addition, ointments and creams often do not create an environment for promoting respiration of the wound tissue and it is not favorable to the normal respiration of the skin. An additional disadvantage of petroleum jelly-based products relates to the greasy feeling left following their topical application onto the skin, mucosal membranes and wounds.

Foams and, in particular, foams that are substantially based on non-aqueous solvents are complicated systems which do not form under all circumstances.

There remains an unmet need for improved, easy to use, stable and non-irritating foam formulations, intended for treatment of dermal and mucosal tissues. Particularly, there remains an unmet need for improved, easy to use, stable and non-irritating foam formulations, with unique therapeutic properties. There is more particularly a need to develop hygroscopic carriers and compositions, foamable carriers and foamable compositions and foams with active agents, which are stable, are non irritating, that facilitate penetration at a target, that are presentable in an easily applicable stable form, that can be handled with ease thereby facilitating compliance and that are adaptable where there is a need to minimize the amount of free water and in consequence, the potential breakdown of ingredients/agents by oxidation/hydrolysis.

Some active agents are known to be generally unstable or susceptible to isomerization or to breakdown, resulting in loss of activity and the use of stabilizers, anti oxidants antimicrobials and buffers and the like in aqueous compositions to protect active or cosmetic agents is known. The problems of protecting active pharmaceutical and cosmetic agents in waterless environments, such as polar compositions are multifold and can vary according to the type of waterless environment and the nature of the agent being used. It has been surprisingly found that factors like small levels of acid residues in the raw materials can be significant in influencing agent stability. Similarly, the presence of low levels of metal ions can act to catalyze reactions or breakdown. There is therefore a need for simple and elegant solutions to stabilize active ingredients in a waterless or substantially waterless environment. On one level it is far from simple or obvious to produce waterless foamable compositions that, when released, produce foams of quality suitable for pharmaceutical or cosmetic application. On a further level having realized a carrier that will produce a waterless foam of quality there is an additional difficulty to be overcome, namely how to adapt the formula and achieve a formulation, which can accept a range of various active pharmaceutical and cosmetic agents such that the composition and active agent are stable and the foam produced remains of quality. Specifically, one of the challenges in preparing such waterless or substantially waterless foamable compositions is ensuring that the active pharmaceutical or therapeutic agent does not react, isomerizes or otherwise break down to any significant extent during its storage and use.

Polyethylene glycol or derivatives or mixtures thereof and propylene glycol or derivatives are believed, in addition to their function as a solvent, to support, facilitate, improve or optimize the function and effect of active agents and may themselves have a therapeutic effect. There is thus, also an unmet need for compositions especially foamable compositions comprising combinations of polyethylene glycols or derivatives or mixtures thereof and polyethylene glycol or derivatives with an active agent, especially synergistic compositions.

Silicones are hydrophobic oily substances that offer some anti friction anti tangle properties and are used in some hair and skin preparations. Unmodified silicones, for example, are known to stay on or near the surface of the skin and can act to protect the skin both as a water proof barrier and as a lubricant. But silicones are known to be defoamers and are contraindicated for producing good quality breakable foam especially in substantial quantities. Nevertheless because of the properties silicones offer and in particular unmodified silicones there is an unmet need to develop foamable formulations that can produce easy to use good quality silicone containing foam especially in substantial quantities.

Oil in water emulsions have long been considered a good vehicle for pharmaceutical and cosmetic compositions. There is, however, an unmet need for waterless emulsions particularly foamable waterless emulsions that can produce easy to use, good quality and non initiating foam having a satisfactory skin or body cavity feeling with unique therapeutic or beneficial properties containing a stable or stabilized active pharmaceutical or cosmetic agent.

Compositions formulated using a base comprising polyethylene glycol or derivatives or mixtures thereof and propylene glycol or derivatives combined with different silicones to produce waterless emulsions are investigated and developed herein as pharmaceutical and cosmetic waterless carriers suitable for delivery of a wide range of active agents despite the defoaming properties of silicones.

### SUMMARY

The invention relates to waterless or substantially waterless carriers, compositions and foams comprising a silicone and the use of them. More particularly it relates to waterless emulsions formulated using a base comprising polyethylene glycol or derivatives or mixtures thereof and propylene glycol or derivatives combined with different silicones as pharmaceutical and cosmetic waterless carriers, compositions and foams suitable for delivery of a wide range of active agents.

Foam formation and stability is a very sensitive process. Silicones can be used as efficient foam control agents and can prevent foam formation or cause foam to collapse rapidly. Silicone fluids can, for example, enter into the foam lamella and displace the foam stabilizing surfactants from the interphase. The foam lamellas are therefore destabilized and burst resulting in foam collapse. Thus silicones are essentially contra-indicated for the preparation of foamable carriers and compositions.

In general terms foam formed from waterless or substantially waterless compositions may by their inherent nature be less firm or inherently weaker than water based emulsion compositions. Thus, not only are silicones inherently unsuitable for forming foamable compositions but it may additionally go against the grain to try and use them in waterless compositions.

Water based carriers and foam formulations by virtue of the unique and anomalous properties and qualities of water have a good skin feeling when compared to waterless or substantially waterless carriers and foam formulations.

There is an unmet need for and there is provided easy to use, stable and non-irritating waterless and substantially waterless carriers and foam formulations, which have a good or special skin feeling and or are not so readily distinguishable from water based emulsion compositions and foams.

There also is an unmet need for and there is provided easy to use, stable and non-irritating waterless and substantially waterless carriers, compositions and foams, intended for application on or treatment of dermal and mucosal tissues. Particularly, there remains an unmet need for and there are provided easy to use, stable and non-irritating waterless and substantially waterless carriers, compositions and foams, with unique physical and or therapeutic properties.

There is a particular need to develop and there is provided waterless and substantially waterless emulsion carriers, compositions, foamable compositions and foams, comprising silicone.

There is also a need to develop and there is provided hygroscopic waterless and substantially waterless emulsion carriers, compositions, foamable carriers, foamable compositions and foams comprising silicone.

Such carriers, compositions, and foams as mentioned in the preceding paragraphs may comprise one or more active agents. There is there is a need for and there are described pharmaceutical and cosmetic carriers, compositions, and foams comprising one or more silicones and one or more active agents, that ought to facilitate penetration at a target, that can be handled with ease thereby facilitating compliance and that are adaptable and where there is a need to minimize or effectively eliminate the amount of free water and in consequence, potential breakdown of ingredients/agents by oxidation/hydrolysis.

Some active agents are known to be generally unstable or susceptible to isomerization or to breakdown, resulting in loss of activity and the use of stabilizers, anti oxidants antimicrobials and buffers and the like in aqueous compositions to protect active or cosmetic agents is known. The problems of protecting active pharmaceutical and cosmetic agents in waterless environments, such as polar compositions are multifold and can vary according to the type of waterless environment and the nature of the agent being used. It has been surprisingly observed that factors like small levels of acid residues in the raw materials can be significant in influencing agent stability. Similarly, the presence of low levels of metal ions can act to catalyze reactions or breakdown. There is therefore a need for simple and elegant solutions to stabilize active ingredients in a waterless or substantially waterless environment. On one level it is far from simple or obvious to produce waterless foamable compositions that when released produce foams of quality suitable for pharmaceutical or cosmetic application. On a further level having realized a carrier that will produce a waterless foam of quality there is an additional difficulty to be overcome, namely how to adapt the formula and achieve a formulation, which can accept a range of various active pharmaceutical and cosmetic agents such that the composition and active agent are stable and the foam produced remains of quality. Specifically, one of the challenges in preparing such waterless or substantially waterless foamable compositions is ensuring that the active pharmaceutical or therapeutic agent does not react, isomerizes or otherwise break down to any significant extent during is storage and use. Particularly, there remains an unmet need for improved, easy to use, stable and non-irritating foam formulations, with unique physical, therapeutic or beneficial properties containing a stable or stabilized active pharmaceutical or cosmetic agent. It is postulated that silicone comprising carriers, compositions and foams further comprising one or more active agents and further where appropriate comprising a modulating or protective agent to shield an active agent from reacting, isomerizing or otherwise breaking down, will satisfy that need.

Polyethylene glycol or derivatives or mixtures thereof and propylene glycol or derivatives are believed, in addition to their function as a solvent, to support, facilitate, improve or optimize the function and effect of active agents and may themselves have a therapeutic effect. There is thus, also an unmet need for and there are described non aqueous or substantially non aqueous emulsion carriers, compositions and foams with silicone comprising polyethylene glycol or derivatives or mixtures thereof; or propylene glycol or derivatives or mixtures thereof; or combinations of one or more polyethylene glycol or derivatives or mixtures thereof and propylene glycol or derivatives. Such carriers, compositions and foams may, with one or more active agents be synergistic.

This invention, thus, relates to pharmaceutical and cosmetic hygroscopic carriers, compositions and foams comprising silicone and a waterless or substantially waterless solvent, wherein the solvent includes a polyethylene glycol or derivative or mixtures thereof or includes a propylene glycol or derivative or mixtures thereof or combinations of one or more polyethylene glycols with one or more propylene glycols. The invention further relates to such carriers compositions and foams comprising one or more active agents. More particularly it relates to the incorporation and selection or use of an Accommodating Agent or Complex in such carriers, compositions and foams.

An Accommodating Agent or Complex is a substance, which when present in a carrier, composition or foam is capable of facilitating the presence of a silicone having anti foaming or foam destabilizing properties in a waterless or substantially waterless carrier, composition or foam such that the silicone comprising carrier, composition or foam is effectively or substantially stabilized or such that the defoaming or destabilizing effect of the presence of silicone in the carrier, composition of foam is effectively or substantially neutralized, reduced or ameliorated or such that the effect of the presence of silicone in the carrier or composition when placed in a canister, filled with propellant and subsequently released expanded to form a foam is ineffective or substantially ineffective to prevent foam formation of quality or to cause foam to collapse rapidly. The Accommodating Agent or Complex can be one or more of an emulsifier, a surfactant, a polymer, a stabilizer, a co-emulsifier, and a foam adjuvant, or combinations thereof, wherein the emulsifier or surfactant can also be a combination of emulsifiers or surfactants or a complex emulgator. In each case respectively, the selected Accommodating Agent or Complex functions in its usual role as a surfactant, polymer, stabilizer etc and in parallel acts to counteract the defoaming effect. In order to achieve good quality breakable foam the formulation should comprise a solid excipient or agent. Thus, in a preferred embodiment the Accommodating Agent or complex comprises a solid. The accommodating agent is preferably at least one surfactant or emulsifier and more preferably a combination of emulsifiers or surfactants or a complex emulgator. Where, for example, the accommodating agent is a surfactant or emulsifier it ability to achieve its task is a function of several factors including the nature (e.g. preferably non ionic) and type (e.g. preferably solid and low HLB) and amount of surfactant (e.g. adding extra due to the defoaming nature of the silicones or using the surfactant in combination with a foam adjuvant) and how it interacts both with the polyethylene or propylene glycol solvent base and silicone. In this connection whilst the HLB system is used for predicting suitable surfactants for oil in water emulsions its relevance to waterless emulsions is questionable and to a large extent unknown. Nevertheless, it has been observed herein that in the silicone glycol waterless emulsions described low HLB surfactants are preferred. This may be, without being bound by any theory that the surfactants show greater miscibility in the silicone component when the HLB is lower.

In one or more embodiments, there is provided a carrier, composition or foam formulation comprising:
(a) a silicone;
(b) about 25% to about 98% of a solvent selected from the group consisting of (1) a propylene glycol or derivative and (2) a polyethylene glycol (PEG) or derivative and mixtures thereof;
(c) 0% to about 48% of at least one secondary solvent;
(d) an Accommodating Agent or Complex;
(e) optionally about 0.01% to about 5% by weight of at least one polymeric agent.

In some limited embodiments it may be possible to produce a foamable composition and foam in the absence of an emulsifier provided the composition contains a polymeric agent. Preferably the polymeric agent is one which has some surfactant like properties and is supported by the presense of one or more stabilizing agents.

In one or more embodiments there is provided a waterless carrier, composition or foam formulation comprising:
(a) a silicone;
(b) about 25% to about 98% of a solvent, selected from the group consisting of (1) a propylene glycol or derivative and (2) a polyethylene glycol (PEG) or derivative and mixtures thereof;
(c) 0% to about 48% of at least one secondary solvent;
(d) about 0.05% to about 20% of an Accommodating Agent or Complex;
(e) optionally about 0.01% to about 5% by weight of at least one polymeric agent; and
wherein the formulation is a silicone in glycol emulsion; and
wherein the Accomodating Agent or Complex is selected from one or more of the group consisting of
   a. at least one surface-active agent at a concentration of about 0.1% to less than about 15% by weight;
   b. at least one polymeric agent at a concentration of about 0.1% to about 5% by weight, wherein the at least one polymeric agent is selected from the group consisting of a bioadhesive agent, a gelling agent, a film forming agent and a phase change agent;
   c. at least one foam adjuvant at a concentration of about 0.1% to about 5% by weight selected from the group consisting of a fatty alcohol, a fatty acid and a hydroxyl fatty acid; and
   d. at least one Stabilizing agent at a concentration of about 0.1% to about 5% by weight; and
wherein at least one of the formulation components is solid, semi-solid or waxy.

In one or more embodiments, there is provided a carrier, composition or foam formulation comprising:
(a) a silicone;
(b) about 25% to about 98% of a solvent selected from the group consisting of (1) a propylene glycol or derivative and (2) a polyethylene glycol (PEG) or derivative or mixtures thereof;
(c) 0% to about 48% of at least one secondary solvent;
(d) at least one polymeric agent;
(e) a second agent selected from the group consisting of a foam adjuvant, a co-emulsifier, a stabilizer and a Stabilizing Agent.

In one or more embodiments the carrier, composition or foam formulation further comprises a liquefied or compressed gas propellant at a concentration of about 3% to about 25% by weight of the total composition. In some embodiments, the ratio of the liquefied or compressed gas propellant to the other components of the formulation ranges from about 3:100 to about 25:100 by weight, from about 3:100 to about 35:100, or from about 3:100 to about 45:100. In some embodiments, the ratio of the liquefied or compressed gas propellant to the other components of the formulation is at least about 3:100, at least about 10:100, at least about 15:100, at least about 20:100, or at least about 25:100.

In one or more embodiments the carrier, composition or foam formulation further comprises a therapeutically effective amount of an active agent.

### DETAILED DESCRIPTION

There is provided waterless or substantially waterless carriers, compositions and foams comprising a silicone and the use of them. More particularly waterless emulsions formulated using a base comprising polyethylene glycol or derivatives or mixtures thereof and propylene glycol or derivatives combined with different silicones as pharmaceutical and cosmetic waterless carriers, compositions and foams suitable for delivery of a wide range of active agents provided.

In order to generate a suitable silicone in glycol waterless emulsions that are sufficiently stable and are either reversible on separation or do not separate for prolonged time period and that can produce foam of good quality the formulation also comprises an Accomodating Agent or Complex in an effective amount.

In the absence of a solid agent in a formulation the Accomodating agent should also be a solid

In one or more embodiments, the Accommodating Agent or Complex comprises at least one emulsifier or surfactant.

In one or more embodiments, the Accommodating Agent or Complex comprises at least one solid emulsifier or surfactant.

In one or more embodiments, the Accommodating Agent or Complex comprises at least one emulsifier or surfactant, which is steareth 2.

In one or more embodiments, the Accommodating Agent or Complex comprises at least one emulsifier or surfactant, which is steareth 21.

In one or more embodiments, the Accommodating Agent or Complex comprises at least one emulsifier or surfactant, which is methyl glucose sesqui stearate.

In one or more embodiments, the Accommodating Agent or Complex comprises an emulsifier or surfactant, which is cetearyl alcohol and cetearyl glucoside.

In one or more embodiments, the Accommodating Agent or Complex comprises at least one emulsifier or surfactant, which is a polysorbate.

In one or more embodiments, the Accommodating Agent or Complex further comprises one or more of a polymeric agent or complex, a stabilizing agent or complex, a foam adjuvant and a co-emulsifying agent or complex.

In one or more embodiments, the Accommodating Agent or Complex comprises at least one emulsifier or surfactant and a polymeric agent.

In one or more embodiments, the carrier, composition or foam comprises as its prime solvent a polyethylene glycol or derivatives or mixtures thereof and the Accommodating Agent or Complex comprises an emulsifier or surfactant.

In one or more embodiments, the carrier, composition or foam comprises as its prime solvent a polyethylene glycol or derivatives or mixtures thereof and the Accommodating Agent or Complex comprises two or more emulsifiers or surfactants.

In one or more embodiments, the carrier, composition or foam comprises as its prime solvent a polyethylene glycol or derivatives or mixtures thereof and the Accommodating Agent or Complex comprises an emulsifier or surfactant and further comprises a polymeric agent. In a preferred embodiment, the surfactant comprises a solid surfactant and the polymeric agent is klucel. In a more preferred embodiment the solid surfactant has a HLB of about 2 to about 9 and more preferably about 4 to about 8. In another preferred embodiment the surfactant is steareth 2. In this connection for example the required HLB for cyclomethicone 7.75 and dimethicone 5. In one or more other embodiments the surfactant is a combination of surfactants or complex emulgator where the weighted average of the combination produces a HLB in the range of about 4 to about 8.

In one or more embodiments, the carrier, composition or foam comprises as its prime solvent a polyethylene glycol or derivatives or mixtures thereof and the Accommodating Agent or Complex comprises two or more emulsifiers or surfactants and further comprises a polymeric agent.

In one or more embodiments, the carrier, composition or foam comprises as its prime solvent a polyethylene glycol or derivatives or mixtures thereof and the Accommodating Agent or Complex comprises an emulsifier or surfactant and further comprises a foam adjuvant, or stabilizer or Stabilizing Agent.

In one or more embodiments, the carrier, composition or foam comprises as its prime solvent a polyethylene glycol or derivatives or mixtures thereof and the Accommodating Agent or Complex comprises an emulsifier or surfactant, and further comprises a polymeric agent and a foam adjuvant or stabilizer or Stabilizing Agent.

In one or more embodiments, the carrier, composition or foam comprises as its prime solvent a propylene glycol or derivatives or mixtures thereof and the Accommodating Agent or Complex comprises an emulsifier or surfactant and a polymeric agent. In one embodiment the surfactant comprises a solid surfactant and the polymeric agent is klucel. In another embodiment the solid surfactant has a HLB of about 2 to about 9 or of about 4 to about 8. In yet another embodiment the surfactant is steareth 2.

In one or more embodiments, the carrier, composition or foam comprises as its prime solvent a propylene glycol or derivatives or mixtures thereof and the Accommodating Agent or Complex comprises at least two emulsifiers or surfactants.

In one or more embodiments, the carrier, composition or foam comprises as its prime solvent a propylene glycol or derivatives or mixtures thereof and the Accommodating Agent or Complex comprises at least two emulsifiers or surfactants and further comprises a polymeric agent.

In one or more embodiments, the carrier, composition or foam comprises as its prime solvent a propylene glycol or derivatives or mixtures thereof and the Accommodating Agent or Complex comprises at least two emulsifiers or surfactants and further comprises a foam adjuvant or stabilizer or Stabilizing Agent.

In one or more embodiments, the carrier, composition or foam comprises as its prime solvent a propylene glycol or derivatives or mixtures thereof and the Accommodating Agent or Complex comprises at least two emulsifiers or surfactants and further comprises a polymeric agent and a foam adjuvant or stabilizer or Stabilizing Agent.

In one or more embodiments, the carrier, composition or foam comprises as its prime solvent a propylene glycol or derivatives or mixtures thereof and the Accommodating Agent or Complex comprises at least two emulsifiers or surfactants, wherein the at least two emulsifiers are steareth 2 and methyl glucose sesqui stearate.

In one or more embodiments, the carrier, composition or foam comprises as its prime solvent a propylene glycol or derivatives or mixtures thereof and the Accommodating Agent or Complex comprises at least two emulsifiers or surfactants, wherein the emulsifiers are steareth 2 and cetearyl alcohol and cetearyl glucoside.

In one or more embodiments, the stabilizer is a stabilizing complex of stearic acid and trolamine.

In one or more embodiments, the polymeric agent is selected from a bioadhesive agent, a gelling agent, a film forming agent and a phase change agent; and a surface-active agent.

In one or more embodiments, the polymeric agent is selected from the group consisting of hydroxypropyl cellulose, aluminum starch octyl succinate (ASOS), klucel and carbopol

In one or more embodiments, the polymeric agent is about 0.01% to about 5% by weight of the composition

In one or more embodiments, the pharmaceutical and cosmetic hygroscopic carriers, compositions and foams comprise silicone, an anti defoaming agent or complex and a waterless or substantially waterless solvent, wherein the solvent includes the one or more hygroscopic substance selected from the group consisting of polyethylene glycols (PEGs), surfactants comprising PEG, polyols, monosaccharides, disaccharides, oligosaccharides and sugar alcohols in an amount to provide hygroscopic properties, and honey.

In another aspect, the invention provides a foamable carrier including a silicone; about 25% to about 98% by weight of a solvent selected from the group consisting of (1) a propylene glycol or derivative and (2) a polyethylene glycol (PEG) or derivative or mixtures thereof; 0% to about 48% by weight of a secondary solvent; one or more emulsifiers or surface-active agents; optionally about 0.01% to about 5% by weight of at least one polymeric agent; and a liquefied or compressed gas propellant at a concentration of about 3% to about 25% by weight of the total composition. In some embodiments, the ratio of propellant to the sum of the remaining components ranges from about 100:3 to about 100:25.

In another aspect, the invention provides a foamable carrier including a silicone; about 50% to about 98% by weight of a solvent selected from the group consisting of (1) a propylene glycol or derivative and (2) a polyethylene glycol (PEG) or derivative or mixtures thereof, 0% to about 48% by weight of a secondary solvent; about 0.01% to about 10% by weight of one or more emulsifiers or surface-active agents; optionally about 0.01% to about 5% by weight of at least one polymeric agent; and a liquefied or compressed gas propellant at a concentration of about 3% to about 25% by weight of the total composition. In some embodiments, the ratio of propellant to the sum of the remaining components ranges from about 100:3 to about 100:25.

It was discovered that in certain embodiments it is possible to create hydrophilic foam with silicone.

In one or embodiments the at least one polymeric agent is selected from a bioadhesive agent, a gelling agent, a film forming agent and a phase change agent, which preferably has some surfactant properties.

In one or embodiments there is provided a foamable hydrophilic carrier or composition, comprising:
(a) a silicone;
(b) a waterless solvent comprising about 25% to about 95% by weight of at least a polar solvent selected from the group consisting of (1) a propylene glycol or derivative and (2) a polyethylene glycol (PEG) or derivative or mixtures thereof;
(c) 0% to about 48% of a secondary waterless solvent
(d) an emulsifier or surface-active agent at a concentration of about 0.1% to about 10% by weight; and or
(e) optionally at least one polymeric agent at a concentration of about 0.1% to about 5% by weight, wherein the at least one polymeric agent is selected from a bioadhesive agent, a gelling agent, a film forming agent and a phase change agent which preferably has some surfactant;
(f) a liquefied or compressed gas propellant at a concentration of about 3% to about 25% by weight of the total composition; and
(g) a therapeutically effective amount of an active agent; and
   wherein the composition is stored in an aerosol container and upon release expands to form breakable foam. In some embodiments, the ratio of propellant to the sum of the remaining components ranges from about 100:3 to about 100:25.

In one or more embodiments, the carrier, composition or foam further comprises a foam adjuvant.

In one or more embodiments, the carrier, composition or foam further comprises a non substantial amount of water.

In one or more embodiments, the carrier, composition or foam further comprises a hydrophobic solvent.

In one or more embodiments, the composition is substantially non-aqueous and/or substantially alcohol-free.

In one or more embodiments, the composition is non-aqueous.

In one or more embodiments, the composition ingredients are pretreated to reduce, remove or eliminate any residual or associated or absorbed water.

In one or more embodiments, the composition further comprises a therapeutically effective concentration of one or more active, therapeutic, pharmaceutical or cosmetic agents.

In one or more embodiments, the composition further comprises one or more modulating agents.

In one or more embodiments, the secondary solvent is a polyol selected from the group consisting of a diol, a triol and a saccharide, and the triol may be selected from the group consisting of glycerin, butane-1,2,3-triol, butane-1,2,4-triol and hexane-1,2,6-triol, or the diol is selected from the group consisting of propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol and dibutylene glycol.

In one or more embodiments, the polyol consists of at least one diol and at least one triol, and wherein the ratio between the diol and triol is between 9:1 and 1:1.

In one or more embodiments, the composition includes a mixture of PEGs, and the PEG may be selected from the group consisting of PEG 200, PEG 300, PEG 400, PEG 600. PEG 1000, PEG 4000, PEG 6000 and PEG 8000. In one or more embodiments, the composition contains one or more PEGs in a concentration to provide viscosity of less than 60,000 CPs.

In one or more embodiments, the composition includes a mixture of at least one polyol and at least one PEG, and the PEG may be selected from the group consisting of PEG 200, PEG 300, PEG 400, PEG 600, PEG 1000, PEG 4000, PEG 6000 and PEG 8000.

In one or more embodiments, the composition includes a secondary solvent selected from the group consisting of dimethyl isosorbide, tetrahydrofuryl alcohol polyethyleneglycol, ether, DMSO, a pyrrolidone, N-Methyl-2-pyrrolidone, 1-Methyl-2-pyrrolidinone, ethyl proxitol, dimethylacetamide, a PEG-type surfactant, an alpha hydroxy acid, lactic acid and glycolic acid. In one or more embodiments, the secondary solvent is dimethyl isosorbide.

In one or more embodiments, the composition includes (1) at least one solvent selected from a propylene glycol and a PEG, and (2) at least one secondary solvent, and for example, the solvent comprises a mixture of at least one polyol and at least one PEG, and for example, the polyol comprises a mixture of at least two polyols.

In one or more embodiments, the ratio between the propylene glycol and/or PEG and the secondary solvent is between 9:1 and 1:1.

In another aspect there is provided a foamable therapeutic composition including a silicone; about 25% to about 98% by weight of a solvent selected from the group consisting of (1) a propylene glycol or derivative and (2) a polyethylene glycol (PEG) or derivative or mixtures thereof; 0% to about 48% by weight of a secondary solvent; about 0.01% to about 10% by weight of an emulsifier or surface-active agent; about 0.01% to about 5% by weight of at least one polymeric agent; a therapeutic agent at a therapeutically effective concentration; and a liquefied or compressed gas propellant at a concentration of about 3% to about 25% by weight of the total composition. In some embodiments, the ratio of propellant to the sum of the remaining components ranges from about 100:3 to about 100:25.

In another aspect there is provided a foamable therapeutic composition including a silicone; about 50% to about 98% by weight of a solvent selected from the group consisting of (1) a propylene glycol or derivative and (2) a polyethylene glycol (PEG) or derivative or mixtures thereof; 0% to about 48% by weight of a secondary solvent; about 0.01% to about 10% by weight of an emulsifier or surface-active agent; about 0.01% to about 5% by weight of at least one polymeric agent; a therapeutic agent at a therapeutically effective concentration; and a liquefied or compressed gas propellant at a concentration of about 3% to about 25% by weight of the total composition. In a further aspect the composition additionally comprises a modulating agent.

In yet an additional embodiment, the foamable therapeutic composition further contains an additional therapeutic agent.

In another aspect, a method of treating, ameliorating or preventing a disorder of mammalian subject includes administering a foamable therapeutic composition to a target area, the composition comprising a therapeutically effective concentration of an active agent, a silicone; about 50% to about 98% of a solvent selected from the group consisting of (1) a propylene glycol or derivative and (2) a polyethylene glycol (PEG) or derivative or mixtures thereof; 0% to about 48% of a secondary solvent; about 0.01% to about 5% by weight of optionally at least one polymeric agent; an Accommodating Agent or Complex; optionally a modulating agent; and a liquefied or compressed gas propellant at a concentration of about 3% to about 25% by weight of the total composition. In some embodiments, the ratio of propellant to the sum of the remaining components ranges from about 100:3 to about 100:25.

In one or more embodiments, the target site is selected from the group consisting of the skin, a body cavity, a mucosal surface, the nose, the mouth, the eye, the ear canal, the respiratory system, the vagina and the rectum.

In one or more embodiments the silicone is a liquid.

In one or more embodiments the silicone is linear.

In one or more embodiments the silicone is branched.

In one or more embodiments the silicone is cyclic.

In one or more embodiments the silicone is unmodified.

In one or more embodiments the silicone is selected from the group consisting of a volatile, a non volatile and a partially volatile silicone or combinations of two or more thereof.

In one or more embodiments the silicone is selected from the group consisting of a volatile, a non volatile and a partially volatile silicone or combinations of two or more thereof wherein on application onto a site the volatile silicones evaporate and leaving the other silicone at the site of application.

In one or more embodiments the Accommodating Agent or Complex can be increased from about 10% up to about 15% or up to about 20% by weight of composition depending on the silicone and amount selected.

In one or more embodiments the one or more emulsifiers or surface active agents can be increased from about 10% up to about 15% or up to about 20% by weight of composition depending on the silicone and amount selected.

Compositions including a polyethylene glycol solvent or derivative or mixtures thereof or includes a propylene glycol derivative or combinations of polyethylene glycols with or without propylene glycol containing an effective amount of one or more active, therapeutic, pharmaceutical or cosmetic agents can be applied to the skin, a body cavity, a mucosal surface, the nose, the mouth, the eye, the ear canal, the respiratory system, the vagina and the rectum. Such carriers and compositions are adaptable to deliver active, therapeutic, pharmaceutical or cosmetic agents without water. This can be convenient where agents are susceptible to oxidation and breakdown in solution. Some vitamins and some antibiotics may for example breakdown in the presence of water and may not be stable in compositions for sufficiently long periods of time to facilitate satisfactorily cosmetic and pharmaceutical uses.

In one or more embodiments the present invention of waterless or substantially waterless silicone carriers, compositions and foams is for use as a vehicle in which an active pharmaceutical or cosmetic agent, when added is stable or stabilized. Active pharmaceutical and cosmetic agents are more generally referred to as a therapeutic agent.

In one or more embodiments the present invention silicone is itself an active agent.

In one or more embodiments the present invention silicone is an active agent in combination with one or more active agents.

In one or more embodiments the Accommodating Agent or Complex ranges from about less than 0.01% up to about 5% up to about 10% up to about 15% or up to about 20% by weight of composition depending on the silicone(s) selected and their amounts and preferably ranges from about 0.2% to about 10% by weight of composition. To the extent it is desirable to increase the amount of silicone it may be necessary to increase the amount of Accommodating Agent or Complex or one of the elements thereof alone or in combination with other elements as will be appreciated by a man in the art. For example, it may be sufficient to increase an emulsifier, a foam adjuvant or a polymeric agent or a stabilizer or a Stabilizing agent alone or in other situations it will be appropriate to increase two or more of them or in other cases it will be helpful to increase all the elements in proportion. In general terms the increase will usually be at the expense of the prime and or secondary solvent, which will be reduced accordingly.

Due to the hygroscopic nature of the solvents described herein, in particular the waterless solvents, water is readily or rapidly absorbed into the composition and accordingly is associated with them, albeit at low levels. Thus, in certain embodiments, the composition is substantially non-aqueous or substantially waterless. The term “substantially non-aqueous” or “substantially waterless” is intended to indicate that the compositions contain at most incidental and trace amounts of water. Thus in some embodiments, the compositions described herein have a free or non-associated water content less than about 5%, less than about 2%, or less than about 1.5%. In certain other embodiments the composition is non-aqueous or waterless.

In certain cases, the composition contains two active agents that require different pH environments in order to remain stable. For example, corticosteroids are typically stable at acidic pH (they have a maximum stability at a pH of about 4-6) and vitamin D analogues are typically stable at basic pH (they have a maximum stability at pH values above about 8). In other cases, the active agent degrades in the presence of water, and therefore, in such cases the present of water in the composition is not desirable.

According to one or more embodiments, the foamable carrier, includes a silicone, a waterless solvent, a stabilizing surfactant, a polymeric agent, a modulating agent and a propellant.

According to one or more embodiments, the foamable pharmaceutical or cosmetic foamable composition, includes a silicone, a waterless solvent, a stabilizing surfactant, a polymeric agent, a modulating agent, a propellant and an active pharmaceutical or cosmetic agents.

According to one or more embodiments, the foamable carrier, includes:
(a) a silicone
(b) a waterless or substantially waterless solvent comprising about 25% to about 98% of at least polar solvent selected from the group consisting of (1) a propylene glycol or derivative and (2) a polyethylene glycol (PEG) or derivative or mixtures thereof,
(c) an Accommodating Agent or Complex;
(d) optionally about 0.01% to about 5% by weight of at least one polymeric agent; and
(e) a liquefied or compressed gas propellant at a concentration of about 3% to about 25% by weight of the total composition;
   wherein the composition is shakable; and
   wherein the composition is stored in an aerosol container and upon release expands to form a breakable foam.

According to one or more embodiments, the foamable therapeutic composition, includes:
(a) a silicone,
(b) waterless solvent comprising about 25% to about 98% of at least polar solvent selected from the group consisting of (1) a propylene glycol (PG) or derivative and (2) a polyethylene glycol (PEG) or derivative or mixtures thereof;
(c) a modulating agent;
(d) a surface-active agent;
(e) about 0.01% to about 5% by weight of at least one polymeric agent;
(f) a liquefied or compressed gas propellant at a concentration of about 3% to about 25% by weight of the total composition; and
(g) an effective amount of an active pharmaceutical or cosmetic agent;
   wherein the carrier is shakable or flowable; and
   wherein the composition is stored in an aerosol container and upon release expands to form a breakable foam.

‘Shakability’ means that the composition contains some or sufficient flow to allow the composition to be mixed or remixed on shaking. That is, it has fluid or semi fluid properties. In some very limited cases possibly aided by the presence of silicone it may exceptionally be possible to have a foamable composition which is flowable but not apparently shakable. By ‘shakable’ it indicates that some motion or movement of the formulation can be sensed when the canister containing the formulation is shaken or is firmly shaken. Accordingly, as explained further herein, ‘shakability’ represents the degree to which the user is able to feel and/or hear the presence of the liquid contents when the filled pressurized canister is shaken.

A breakable foam is one that is thermally stable, yet breaks under sheer force.

A breakable foam is not “quick breaking”, i.e., it does not readily collapse upon exposure to body temperature environment. Sheer-force breakability of the foam is clearly advantageous over thermally induced breakability, since it allows comfortable application and well directed administration to the target area.

In one or more embodiments the ratio of polymeric agent to surfactant is about 1:10 to about 10:1; about 1:5 to about 5:1; about 3:7 to about 7:3; and about 2:1 to about 1:2.

The provision and selection of polymeric agent is however not straightforward. The polymers should be miscible or swell in the waterless solvent. It has been found that in the case of modified cellulose, the lower molecular weight cellulose polymer derivatives are preferable.

In one embodiment the polymeric agent is hydroxypropyl cellulose.

In one embodiment the polymeric agent is klucel.

In another embodiment the polymeric agent is or Carbomer such as Carbopol 9340®.

In another embodiment the polymeric agent is aluminum starch octenylsuccinate.

In another embodiment the polymeric agent is a combination of two or more polymeric agents.

According to one or more embodiments the pre-foamable carrier; the pre-foamable pharmaceutical or cosmetic composition; the foamable carrier, or the foamable pharmaceutical or cosmetic composition further includes 0.1% to about 45% of a secondary solvent.

In one or more embodiments there is provided a foamable vehicle that is suitable for use as a base for delivery of not merely one type of active pharmaceutical ingredient (“API”) but is adaptable for use with one or more API's from a wide range of different types of API's with appropriate and usually relatively minimal or minor adjustment to the vehicle. For example, by altering the amount of a component or by the addition of a stabilizer or an antioxidant, as would be appreciated by a person skilled in the art.

In a further embodiment the surfactant and polymeric agent and their amounts are selected so that the composition is sufficiently shakable or flowable so that foam extrusion and substantially uniform foam formation is not hampered. To this extent, the maximum effective amount of surfactant and polymeric agent that may be used for a foam may be limited by the need for shakability. For example as the level of waxy surfactants and or polymeric agents the composition will become thicker until it reaches a point where it will no longer be shakable or flowable. As mentioned elsewhere some limited exceptions may occur due to the presence of silicone where the composition is still flowable but not apparently shakable. For an ointment or gel the levels may be further increased albeit a solid non flowable composition is not suitable for foams. On the other hand for PEG and/or PG silicone emulsion compositions the presence of a solid excipient or agent is desirable so that the pre foam composition has sufficient body as to produce foam of good quality.

In one or more embodiments the carrier or composition with or without an active agent my be formulated as an ointment, gel, lotion or spray for pharmaceutical or cosmetic use.

In a further embodiment the propellant is preferably between about 5% to about 12% by weight of the composition.

In one or more embodiments of the pharmaceutical or cosmetic foamable product is non-flammable.

By waterless is meant that the composition contains no or substantially no, free or unassociated or absorbed water. It will be understood by a person of the art that the waterless solvents and substances miscible with them can be hydrophilic and can contain water in an associated or unfree or absorbed form and may absorb water from the atmosphere and the ability to do so is its hygroscopic water capacity. In some embodiments the composition ingredients are pretreated to reduce, remove or eliminate any residual or associated or absorbed water.

Upon release from an aerosol container, the foamable carrier or composition forms an expanded foam suitable for the treatment of an infected surface and for topical administration to the skin, a body surface, a body cavity or a mucosal surface.

In one or more embodiments there is provided a waterless carrier, composition or foam formulation comprising:
(f) a silicone;
(g) about 25% to about 98% of a primary solvent, the solvent including (1) a propylene glycol or derivative, (2) a polyethylene glycol (PEG) or derivative, or mixtures thereof;
(h) 0% to about 48% of at least one secondary solvent;
(i) about 0.05% to about 20% of an Accommodating Agent or Complex;
(j) optionally about 0.01% to about 5% by weight of at least one polymeric agent; and
wherein the formulation is a silicone in glycol emulsion; and
wherein the Accomodating Agent or Complex is selected from one or more of the group consisting of
   e. at least one surface-active agent at a concentration of about 0.1% to less than about 15% by weight;
   f. at least one polymeric agent at a concentration of about 0.1% to about 5% by weight, wherein the at least one polymeric agent is a bioadhesive agent, a gelling agent, a film forming agent or a phase change agent;
   g. at least one foam adjuvant at a concentration of about 0.1% to about 5% by weight selected from the group consisting of a fatty alcohol, a fatty acid and a hydroxyl fatty acid; and
   h. at least one Stabilizing agent at a concentration of about 0.1% to about 5% by weight; and
wherein at least one of the formulation components is a solid, semi-solid or waxy

In one or more embodiments the formulation has some or partial resistance to creaming when subjected to centrifugation at 3000 rpm for 10 min or to FTC for at least one cycle.

In one or more embodiments there is provided a waterless foamable hydrophilic carrier formulation, comprising:
(a) a silicone;
(b) a primary waterless solvent comprising about 25% to about 98% of at least one solvent selected from the group consisting of (1) a propylene glycol or derivative and (2) a polyethylene glycol (PEG) or derivative or mixtures thereof;
(c) 0% to about 48% of at least one secondary waterless solvent;
(d) about 0.05% to about 20% of an Accommodating Agent or Complex; and
(e) a liquefied or compressed gas propellant at a concentration of about 3% to about 25% by weight of the total composition;
wherein the formulation is a hygroscopic emulsion;
wherein the Accomodating Agent or Complex is selected from one or more of the group consisting of
   a. at least one surface-active agent at a concentration of about 0.1% to less than about 15% by weight;
   b. at least one polymeric agent at a concentration of about 0.1% to about 5% by weight, wherein the at least one polymeric agent is a bioadhesive agent, a gelling agent, a film forming agent or a phase change agent;
   c. at least one foam adjuvant at a concentration of about 0.1% to about 5% by weight; selected from the group consisting of a fatty alcohol, a fatty acid and a hydroxyl fatty acid. and
   d. at least one Stabilizing agent at a concentration of about 0.1% to about 5% by weight;
wherein at least one of the formulation components is a solid semi solid or waxy.
wherein the composition is shakable or flowable; and
wherein the composition is stored in an aerosol container and upon release expands to form a breakable foam.

In some embodiments, the primary waterless solvent includes about 25% to about 98% of at least one solvent selected from the group consisting of (1) a propylene glycol or derivative and (2) a polyethylene glycol (PEG) or derivative or mixtures thereof. In some embodiments, the primary waterless solvent is about 25% to about 98% of the foamable carrier formulation. In some embodiments, when the primary waterless solvent is a propylene glycol, the formulation further includes about 0.01% to about 5% by weight of at least one polymeric agent.

In some embodiments, the waterless foamable hydrophilic carrier formulations described herein further include an effective amount of an active agent and optionally a modulating agent.

In one or more embodiments the surface active agent is a solid, semi solid or waxy surfactant; a combination of two or more surface active agents; or a surfactant capable of forming liquid crystals. When there is more than one surface active agents in the formulation, they need not all be solid, semi-solid, or waxy. In some embodiments, when the surface active agent is a combination of two or more surface active agents, al least one of the two or more surface active agents is solid, semi solid or waxy.

In one or more embodiments the surface active agent is selected from one of the groups consisting of
a. a solid, semi solid or waxy surfactant;
b. a combination of two or more surface active agents and at least one is solid, semi solid or waxy; and
c. a surfactant capable of forming liquid crystals.

In one or more embodiments the surface active agent is one or more of the following:
a. a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkylyl ether, polyoxyethylene cetyl ether, polyoxyethylene palmityl ether, polyethylene oxide hexadecyl ether, polyethylene glycol cetyl ether, brij 38, brij 52, brij 56 and brij W1, a sucrose ester, a partial ester of sorbitol, sorbitan monolaurate, sorbitan monolaurate a monoglyceride, a diglyceride, isoceteth-20, staereth 2 or a mono, di or tri fatty acid sucrose ester;
b. laureth-4, glyceryl stearate, PEG-100 stearate, ceteareth-6, stearyl alcohol, myrj 52, steareth-2, steareth 21, poyglyceryl 10 laurate, POE (2) cetyl ether, cetearyl glucoside, cetyryl alcohol, methyl glucose sesquistearate, span 60, sucrose stearic acid esters, sorbitan stearate, sucrose cocoate, Peg 40 stearate or isostearath 20;
c. a polymeric emulsifier, particularly Pennulen (TR1 or TR2); liquid crystal systems, particularly Arlatone (2121), Stepan (Mild RM1), Nikomulese (41) or Montanov (68);
d. a combination of at least two of the surfactants, such as for example: steareth-2 and steareth-21; glyceryl stearate and PEG-100 stearate; ceteareth-6 and stearyl alcohol; cetearyl glucoside and cetyryl alcohol; sorbitan stearate and sucrose cocoate; and polysorbate 80 and PEG-40 stearate; seareth 2 and methyl glucose sesqui stearate; or steareth 2 and cetearyl glucoside and cetearyl alcohol;
e. a combination of at least two surfactants, such as for example: combinations of polyoxyethylene alkyl ethers, particularly Brij 59/Brij 10; Brij 52/Brij 10; Steareth 2/Steareth 20; Steareth 2/Steareth 21 (Brij 72/BRIJ 721); Myrj 52/Myrj 59; combinations of sucrose esters, particularly Surphope 1816/Surphope 1807; combinations of sorbitan esters, particularly Span 20/Span 80; Span 20/Span 60; combinations of sucrose esters and sorbitan esters, particularly Surphope 1811 and Span 60; or combinations of liquid polysorbate detergents and PEG compounds, particularly Twin 80/PEG-40 stearate/methyl glucose sequistearate.

In one or more embodiments the polymeric agent is one of the following:
a. locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, an amine-bearing polymer, chitosan, alginic acid, hyaluronic acid, a chemically modified starch, a carboxyvinyl polymer, polyvinylpyrrolidone, polyvinyl alcohol, a polyacrylic acid polymer, a polymethacrylic acid polymer, polyvinyl acetate, a polyvinyl chloride polymer, a polyvinylidene chloride polymer, methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, a cationic cellulose, PEG 1000, PEG 4000, PEG 6000 or PEG 8000;
b. hydroxypropylcellulose, aluminum starch octenylsuccinate or a carbomer;
c. Carbopol® 934, Carbopol® 940, Carbopo® 941, Carbopol® 980 pr Carbopol® 981.

Examples of the silicone used in the foamable formulations described herein include, without limitation, unmodified silicones, liquid silicones, cyclic silicones, linear silicones, branched silicones, low molecular weight silicones, high molecular weight silicones, volatile silicones, non-volatile silicones, partially non-volatile silicones, or combinations of volatile, non-volatile, or partially non-volatile silicones. In some embodiments, the silicone component(s) is selected for particular volatility properties. For example, in some embodiments, a combination of silicones having different volatility properties is selected. In some embodiments, the foamable formulation includes at least a volatile phase including at least one volatile or partially volatile silicone and a non-volatile phase including at least one non-volatile silicone. Upon application to a surface, one or all of the silicones of the volatile phase evaporates and the non-volatile phase remains at the site of application.

In one or more embodiments the silicone is selected from one or more of the groups consisting of
a) wherein the silicone is unmodified;
b) wherein the silicone is a liquid;
c) wherein the silicone is cyclic;
d) wherein the silicone is linear;
e) wherein the silicone is branched;
f) wherein the silicone is a low molecular weight silicone;
g) wherein the silicone is a high molecular weight silicone;
h) a volatile, a non volatile and a partially volatile silicone or combinations of two or more thereof;
i) a volatile, a non volatile and a partially volatile silicone or combinations of two or more thereof wherein on application to a site the volatile silicones evaporate and leaving the other silicone at the site of application;

In one or more embodiments the silicone is dimethicone, cetyl dimethicone, cyclomethicone, cyclodimethicone, simethicone, polydimethylsiloxane polymer, cyclopentasiloxane DC245, Dow Corning® 345 Fluid, and bis-PEG-18 methyl ether dimethyl silane or mixtures thereof.

In one or more embodiments, where PEG or a derivative is the primary or secondary solvent then less than 15% of the PEG or derivative is a solid.

In one or more embodiments, where PEG or a derivative is the primary or secondary solvent then less than 5% of the PEG or derivative is a solid.

In one or more embodiments, where PEG or a derivative is the primary or secondary solvent, the surface active agent further comprises an ionic surfactant, selected from the group consisting of a cationic surfactant, a zwitterionic surfactant, an amphoteric surfactant and an ampholytic surfactant.

In one or more embodiments, the formulation further comprises a second hygroscopic substance. The second hygroscopic substance includes, without limitation, one or more of the following:
a) one or more polyethylene glycols (PEGs);
b) one or more surfactants comprising PEG;
c) one or more polyols;
d) one or more monosaccharides, disaccharides, oligosaccharides and sugar alcohols in an amount to provide hygroscopic properties; and
e) one or more honey.

In one or more embodiments, the formulation further comprises at least one co solvent or secondary solvent. Exemplary co-solvents or secondary solvents include, without limitation, one or more of the following:
(a) a polyol, such as a diol, a triol or a saccharide; exemplary triols include, without limitation, glycerin, butane-1,2,3-triol, butane-1,2,4-triol and hexane-1,2,6-triol; exemplary diols include, without limitation, propylene glycol, butanediol, butenediol, butynediol, pentanediol, hexanediol, octanediol, neopentyl glycol, 2-methyl-1,3-propanediol diethylene glycol, triethylene glycol, tetraethylene glycol, dipropylene glycol and dibutylene glycol; or (ii) at least one diol and at least one triol; or
(b) dimethyl isosorbide, tetrahydrofurfuryl alcohol polyethyleneglycol, ether, DMSO, a pyrrolidone, N-Methyl-2-pyrrolidone, 1-Methyl-2-pyrrolidinone, ethyl proxitol, dimethylacetamide, a PEG-type surfactant, an alpha hydroxy acid, lactic acid and glycolic acid or an alkyl alcohol.
   In some embodiments, when the co-solvent includes at least one diol and at least one triol, the ratio between the diol and triol is between 9:1 and 1:1, between 8:1 and 1:1, between 7:1 and 1:1, between 6:1 and 1:1, between 5:1 and 1:1, between 4:1 and 1:1, between 3:1 and 1:1, and between 2:1 and 1:1.

In one or more embodiments the ratio between the primary waterless solvent (e.g., polyethylene glycol and/or propylene glycol) and the co-solvent or secondary solvent is between about 9:1 and about 1:1, between 8:1 and 1:1, between 7:1 and 1:1, between 6:1 and 1:1, between 5:1 and 1:1, between 4:1 and 1:1, between 3:1 and 1:1, and between 2:1 and 1:1.

In one or more embodiments when the primary solvent is propylene glycol then the Accomodating Agent or Complex comprises a combination of a surface active agent and a polymeric agent.

In one or more embodiments, the formulation further comprises a hydrophobic solvent In one or more embodiments, the formulation further comprises a small amount of water, which can be up to about 5%.

In one or more embodiments, the formulation further an anti-perspirant, an anti-static agent, a buffering agent, a bulking agent, a chelating agent, a colorant, a conditioner, a deodorant, a diluent, a dye, an emollient, fragrance, a humectant, an occlusive agent, a penetration enhancer, a perfuming agent, a permeation enhancer, a pH-adjusting agent, a preservative, a skin penetration enhancer, a sunscreen, a sun blocking agent, or a sunless tanning agent.

In one or more embodiments, the formulation ingredients are pretreated to reduce, remove or eliminate any residual or associated or absorbed water.

In one or more embodiments the active agent includes, without limitation, one or more of the following: anti-infective agents, antifungal agents, antiviral agents, anesthesic analgesics, corticosterois, non steroid anti inflammatory agents, retinoids, lubricating agents anti warts, antiproliferative agents, vasoactive agents, keratolytic agents, insectisides and repellants, dicarboxylic acids and esters; calcium channel blockers, cholinergic agents, N-oxide doners, photodynamic agents, anti acne agents, anti wrinkle agents, antioxidants, self tanning active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers.

In one or more embodiments the active agent is acyclovir, azelaic acid, benzoyl peroxide, betamethasone 17 valerate micronized, caffeine, calcipotriol, calcipotriol hydrate, calcitriol, ciclopiroxolamine, diclofenac sodium, ketoconazole, miconazole nitrate, minoxidil, mupirocin, nifedipine regular, permethrin bpc (cis:trans 25:75), piroxicam, salicylic acid or terbinafine HCl, or combinations thereof

In one or more embodiments, the active agent is unstable in the presence of water or is susceptible to oxidation.

In one or more embodiments, there is provided a waterless foamable hydrophilic carrier formulation, comprising:
(a) a silicone, wherein the silicone is selected from the group consisting of dimethicone, cetyl dimethicone, cyclomethicone, cyclodimethicone, simethicone, polydimethylsiloxane polymer, cyclopentasiloxane DC245, Dow Corning® 345 Fluid, and bis-PEG-18 methyl ether dimethyl silane and mixtures thereof
(b) a waterless solvent comprising about 25% to about 98% of at least one solvent selected from the group consisting of (1) a propylene glycol or derivative and (2) a polyethylene glycol (PEG) or derivative or mixtures thereof;
(c) 0% to about 48% of at least one secondary waterless solvent
(d) about 0.05% to about 20% of an Accommodating Agent or Complex;
(e) about 0.01% to about 5% by weight of at least one polymeric agent when the primary solvent is a propylene glycol;
(f) a therapeutically effective amount of an active agent;
(g) 0% to about 1% of a modulating agent; and
(h) a liquefied or compressed gas propellant at a concentration of about 3% to about 25% by weight of the total composition;
wherein the formulation is a hygroscopic emulsion;
   e. wherein the Accomodating Agent or Complex comprises at least one surface-active agent at a concentration of about 0.1% to less than about 15% by weight; at least one polymeric agent at a concentration of about 0.1% to about 5% by weight, wherein the at least one polymeric agent is selected from a bioadhesive agent, a gelling agent, a film forming agent and a phase change agent; and at least one foam adjuvant at a concentration of about 0% to about 5% by weight; selected from the group consisting of a fatty alcohol, a fatty acid and a hydroxyl fatty acid.
wherein at least one of the formulation components is a solid semi solid or waxy;
wherein the formulation has some or partial resistance to creaming when subjected to centrifugation at 300 rpm for 10 mm or to FTC for at least one cycle;
wherein the composition is shakable or flowable; and
wherein the composition is stored in an aerosol container and upon release expands to form a breakable foam.

In one or more embodiments, there is provided a waterless foamable hydrophilic carrier formulation including a precursor composition and a liquefied or compressed gas propellant. The precursor includes:
(i) a silicone, examples of which include without limitation dimethicone, cetyl dimethicone, cyclomethicone, cyclodimethicone, simethicone, polydimethylsiloxane polymer, cyclopentasiloxane DC245, Dow Corning® 345 Fluid, and bis-PEG-18 methyl ether dimethyl silane and mixtures thereof.
(j) a waterless solvent, examples of which include without limitation (1) a propylene glycol or derivative, (2) a polyethylene glycol (PEG) or derivative and mixtures thereof;
(k) 0% to about 48% of at least one secondary waterless solvent
(l) about 0.05% to about 20% of an Accommodating Agent or Complex;
(m) a therapeutically effective amount of an active agent; and
(n) 0% to about 1% of a modulating agent;
   In some embodiments, when the primary solvent is a propylene glycol, the precursor composition also includes at least one polymeric agent. The polymeric agent is present in the precursor composition at a concentration of about 0.01% to about 5%, about 0.01% to about 4% about 0.01% to about 3%, about 0.01% to about 2%, or about 0.01% to about 1%. In some embodiments, the primary waterless solvent is present in the precursor composition at a concentration of about 25% to about 98%, about 25% to about 90%, about 25% to about 80%, about 25% to about 70%, about 25% to about 60%, about 25% to about 50%, about 25% to about 40%, about 25% to about 30%, or about 70% to about 96.5%. In some embodiments, the primary waterless solvent includes about 25% to about 98%, about 25% to about 90%, about 25% to about 80%, about 25% to about 70%, about 25% to about 60%, about 25% to about 50%, about 25% to about 40%, about 25% to about 30%, or about 70% to about 96.5% of at least one of the following: (1) a propylene glycol or derivative, (2) a polyethylene glycol or derivative, or mixtures thereof. In some embodiments, the formulation is a hygroscopic emulsion. In some embodiments, the Accomodating Agent or Complex is at least one of the following: at least one surface-active agent, at least one polylmeric agent, or at least one foam adjuvant. In some embodiments, the surface-active agent(s) is present in the precursor composition a concentration of about 0.1% to less than about 15% by weight. In some embodiments, the polymeric agent(s) is present in the precursor composition at a concentration of about 0.11% to about 5% by weigh. In some embodiments, the foam adjuvant(s) is present in the precursor composition at a concentration of about 0% to about 5% by weight. Exemplary polymeric agents include, without limitation, a bioadhesive agent, a gelling agent, a film forming agent or a phase change agent. Exemplary foam adjuvants include, without limitation, fatty alcohols, fatty acids and hydroxyl fatty acids. In some embodiments, at least one of the components of the precursor composition is solid, semi solid or waxy. In some embodiments, the formulation including the precursor composition and the propellant has some or partial resistance to creaming when subjected to centrifugation at 3000 rpm for 10 minutes or is subjected to at least one freeze-thaw cycle. In some embodiments, the foamable formulation the composition is stored in an aerosol container and upon release expands to form a breakable foam.

In one or more embodiments the waterless solvent comprises about 70% to about 96.5% of a polyethylene glycol (PEG) or derivative or mixtures thereof,

In one or more embodiments the waterless solvent comprising about 70% to about 96.5% of a propylene glycol or derivative.

In one or more embodiments, the formulation has some or partial resistance to creaming when subjected to centrifugation at 3000 rpm for 10 min or to FTC for at least one cycle (i.e., following one or more freeze-thaw cycles, for example, one FTC, two FTCs, three FTCs, four FTCs or five FTCs).

In one or more embodiments there is provided a method of treating, alleviating or preventing a dermatological, cosmetic or mucosal disorder, comprising administering topically to a subject having said disorder a therapeutically effective amount of any of the formulations described herein

In one or more embodiments there is provided a hygroscopic silicone in glycol emulsion composition comprising a polyethylene glycol or derivatives and mixtures thereof or comprising a propylene glycol or derivatives at a sufficient concentration alone as a component in the composition or with one or more other hygroscopic substances to provide
(a) at least one hygroscopic substance at a sufficient concentration to provide an Aw value of the hygroscopic therapeutic containing composition of less than 0.9; and
(b) a therapeutic agent thereof or combinations thereof.

In one or more embodiments the Aw value is selected from the group consisting of a) in the range of about 0.8 and about 0.9; b) in the range of about 0.7 and about 0.8; and c) in the range of about less than about 0.7. In one or more embodiments the emulsions have an average droplet size of less than about 30 microns, less than about 15 microns or less than about 5 microns.

In one or more embodiments the foams have an average bubble size of less than about 200 microns, or less than about 100 microns.

In one or more embodiments the emulsions have a reduced sensation of dryness when applied topically to the skin in comparison to a similar formulation without silicone.

In one or more embodiments the formulations further comprise about 1% to about 10% microsponges containing an effective amount of at least one active agent.

In one or more embodiments the carrier is essentially solid or gel like.

In one or more embodiments there is provided a method of liquefying a carrier, composition or formulation as described herein, including
a) preparing and adding the carrier, composition or formulation to a container capable of being sealed and withstanding pressure of a liquefied or compressed gas propellant at a concentration of about 3% to about 25% by weight of the total composition;
b) allowing the composition to form a wax solid or gel;
c) adding a liquefied or compressed gas propellant at a concentration of about 3% to about 25% by weight of the total composition to the sealed container;
d) shaking or agitating the container.
   As described herein, the carriers, compositions or formulations amenable to liquefication are silicone-comprising, waterless or substantially waterless, waxy, solid, or gel carriers.

In one or more embodiments the ratio of propellant to the solid or gel in the emulsions is in the range of about or less than about 1:4 to less than about 1:15. In some embodiments, the ratio of propellant to the remaining components of the carrier, composition or formulation is from about 1:4 to about 1:15 or less than about 1:15.

In one or more embodiments there is provided a kit comprising a dual chamber device or dual dispenser head, a first canister and a second canister, wherein each canister is connectable to the said device or head. The first canister includes a first foamable composition prepared as described herein having a first active pharmaceutical ingredient. The second canister includes a second foamable composition prepared as described herein having a second active pharmaceutical ingredient.

In one or more embodiments the first active pharmaceutical ingredient is a steroid and the second active pharmaceutical ingredient is a vitamin D derivative.

All % values are provided on a weight (w/w) basis.

### Silicone

A “Silicone” is a largely inert compound with a wide variety of forms and uses. Silicones (more accurately called polymerized siloxanes or polysiloxanes) are inorganic-organic polymers with the chemical formula [R2SiO]n, where R=organic groups such as (C₁-C₆)-alkyl or (C₁-C₈)-aryl groups (e.g., methyl, ethyl, propyl, and phenyl). These materials consist of an inorganic silicon-oxygen backbone ( . . . —Si—O—Si—O—Si—O— . . . ) with organic side groups attached to the silicon atoms, which are four-coordinate. In some cases organic side groups can be used to link two or more of these —Si—O— backbones together. By varying the —Si—O— chain lengths, side groups, and crosslinking, silicones can be synthesized with a wide variety of properties and compositions. They can vary in consistency from liquid to gel to rubber to hard plastic. The most common type is linear polydimethylsiloxane or PDMS (a silicone oil). The second largest group of silicone materials is based on silicone resins, which are formed by branched and cage-like oligosiloxanes.

The name silicone encompasses a large number of compounds based on polydialkylsiloxanes; amongst them, the most common are the trimethylsilyloxyterminated polydimethylsiloxanes. These are linear polymers, liquid even at very high molecular weights. Numerous other structures can easily be obtained, either by substitution of methyl groups by other groups like —CH═CH2, —H, —CH2—CH2-CF3 or by replacing some of the Me2SiO2/2 chain units with MeSiO3/2 or SiO4/2 units where the silicon is substituted with 3 or 4 oxygen atoms to give non-linear branched structures. The preferred polymers for pharmaceutical applications are the ones essentially substituted by methyl groups.

Silicones have found many uses in healthcare applications, in the construction of medical devices, as non-metabolized active in gastro-enterology (antifoam) or as excipient in pharmaceutical formulations. As excipients, many of the unique properties of the polydimethylsiloxanes can be associated in controlled release drug delivery systems: chemical stability, high level of purity (absence of organic solvent or heavy metal contamination; low level of residual polydimethylcyclosiloxanes), very high permeability to many active drugs. As a result, polydimethylsiloxanes are used in many anti-acid/anti-gas drugs, in transdermal systems (patches), in inserts or implants or in topical drugs. None of these applications would be possible without the outstanding biocompatibility shown by the polydimethylsiloxanes.

As a result of their efficient anti foam properties, silicones (Dimethicone, Simethicone or Simethicone emulsion) are used as additives in the manufacture of pharmaceuticals e.g. in fermentation processes, in mixing or filling operations: they are efficacious at very low levels, 1 to 50 ppm. This makes them contraindicated for use in foamable carriers and compositions and especially in waterless or substantially waterless carriers, compositions and foams.

Dimethicones and simethicones are used as antifoams in anti-flatulent or anti-acid formulations. Silicones in these products help to suppress the formation of foam in the stomach without modifying the gastric pH. This is not surprising as silicones, with their low Surface tension (and in particular when compounded with silicon dioxide) are known to destroy foams in many applications, e.g. in petrol, paper pulp or food processing. In pharmaceutical formulations, the mode of action is physical as the polydimethylsiloxanes are not metabolized but excreted as such. Silicones are often compounded with other anti-acid actives such as Al or Mg hydroxides, Mg or Ca carbonates. Silicones appear in many liquid formulations like syrups as well as in effervescent tablets formulations, most likely either to control foaming during processing and filling operations or during use. Silicones are also used in diagnostic formulations to eliminate foam in the stomach during endoscopy or, in conjunction with barium sulphate, during X-Ray examination.

While most silicones are hydrophobic and often used to formulate lipophilic drugs, low molecular weight OH end blocked fluids, HO—(SiMe2O)n—H (n˜12) [3], have been shown to dissolve hydrophilic actives. Thus the combination of a hydrophilic solvent system and a hydrophobic material can be potentially very useful in carrying and delivering a very wide range of active agents.

Whilst silicones have been used because of their biocompatibility and probably because of their aesthetic benefits, which is well known in personal care formulations, silicones may also affect the bio-availability of active agents.

Apart from their use as antifoaming agents (see above), they are used in topical formulations as excipients in topical products particularly for the skin and hair and in some cases as active agents against acne and for the prevention of skin ulceration around stoma, for skin diseases, mainly as creams followed by gels and lotions for the treatment of acne, fungal diseases or psoriasis, in contact with fragile mucosa in the treatment of hemorrhoids, anal dermatoses or itch relief as well as for the delivery of antibiotics in gynecological capsules or creams.

Unmodified silicones stay on or near the surface of the skin. Not only are the molecules too big to physically enter past the upper living cells—they associate with the upper layer of drying skin—but they also cannot penetrate cell membranes due to their large size. They also dislike both the water and proteins inside cells. Silicones may be used in the foam compositions for topical and body cavity compositions. Cyclomethicones are unmodified silicones. They evaporate quickly after helping to carry oils into the top layer of epidermis. From there, they may be absorbed by the skin. Cyclomethicones a similar function in hair care products by helping nutrients enter the hair shaft. Dimethicones are also unmodified silicones. They form a barrier layer on the skin which must be renewed as the skin sloughs off. Dimethicones coat the surface of the hair and lubricate it, improving combing providing detangling, and thus, hair loss and breakage. Silicones form a protective layer which helps prevent transdermal water loss—a very useful characteristic for many products. Silicone gums provide instant shine to hair. Silicones act to help seal moisture into the hair, which helps prevent many kinds of damage. Silicones have varying properties which affects how they are used. Their solubility in a variety of ingredients is a most important consideration. Silicones are usually blend readily with each other to provide desired properties. The solubility of silicones in other ingredients varies and must be observed when formulating. Generally, unmodified silicones are insoluble in water, and other polar compounds. All-silicone emulsions are possible. Silicones can be modified or changed to improve water solubility. Silicone oils dissolve well in and will dissolve non-polar materials. These include essential oils, mineral oil, fixed oils, light esters, and sunscreen agents. Solubility decreases, however, as the size and viscosity of the silicone increases. Silicone gums are not soluble in fixed oils, but the carrier is. Thus the carrier dissolves, leaving the gum as a troublesome blob. Silicone oils are somewhat soluble in waxes, lanolin, castor oil and similar materials. The viscosity limitation is higher in these materials than it is for the fixed oils.

Silicone compounds generally have fairly low usage rates in most applications. Their usual applications include lotions, salves, conditioners and bath products that use 1 to 5% silicone as an additive to modify “feel” and provide skin protection. Cyclomethicones are most commonly used, with the low- to medium-viscosity dimethicones. Different types of silicone may be used alone or together. Many formulas use 2 parts cyclomethicone to 1 part dimethicone. Silicone gums are generally not usable for lotions and other emulsified products. This is due to the insolubility of the gum in fixed oils (as discussed above). It is possible to make emulsions from silicone oils only, or with mineral oil. These types of emulsions allow for the incorporation of the gums into products such as hair dressings. Cyclomethicone may be used alone to carry essential oils in a dry oil spray—referred to commonly as “dry perfume oil sprays”. It may also be used to dilute gums to produce spray detanglers. The gum base content in a hair serum spray is usually between 5 and 10%.

Dimethicones and simethicones are mostly used. (See Table 1 below) Other specific silicones used as excipients could be cyclomethicones (cyclomethicones are widely used in personal care because of their volatility, “aesthetic” and safety profile); decamethylpentacyclosiloxane (Me2SiO)5; hexamethyldisiloxane, Me3SiOSiMe3 (Bp.=100° C.), can be used as a volatile excipient for topical applications, e.g. in combination with fungicides. The low surface tension of this disiloxane improves the coverage of the skin and possibly increases the bio-availability of the active drug. The advantage of this disiloxane, despite its flammability, is its very low heat of vaporization, which, despite its rather high boiling point, allows the film to dry quickly; stearyloxytrimethylsilane, CH3(CH2)17OSiMe3, a wax with occlusive properties but still with a pleasant silky feel as normally associated with silicones; dimethicone copolyol which may be used in conjunction with cyclomethicones.

TABLE 1

| INCI | | SCIENTIFIC | |
|---|---|---|---|
| NAME | CAS No. | NAME | VISCOSITY |
| Dimethicone | 9006-65-9 | polydimethylsiloxane | 350 CPS |
| | | | med. weight |
| Cyclomethicone | 541-02-06 | decamethylcyclo- | 4 CPS |
| | | pentasiloxane | (v. light) |
| Botanisil | 541-02-6 | cyclomethicone, | 4000 to 8000 CPS |
| | and | dimethicone | (v. thick) |
| | 9006-65-9 | | |

* to figure CPS by comparison: H₂O (water) has a viscosity of 1-5 CPS.

A substantial number of registered products contain silicones that are not described in Compendia, e.g. methylpolysiloxane, silicone for powder treatment, silicone or fluoro silicone for polyester film coating, silicone copolyol, HMDS, Silastic®, silicone wax, and the like.

Silicones can come in many different forms. Silicones can be linear, substituted, crosslinked or cyclic polymers. They range from low to high molecular weight polymers. The can take the form of liquids, solids, elastomers, gums, and resins. They can be modified to produce silicone emulsifiers.

The most widely used silicones are the polydimethylsiloxanes, Me3SiO(SiMe2O)rSiMe3, with viscosities between 10 to 100,000 mPa·s. These have not shown toxicity during administration via typical exposure routes. Due to their high molecular weight, they are not absorbed in the gastrointestinal tract and are excreted without modification, nor are they absorbed through the skin. Repeated oral or cutaneous dosages have not indicated effects on different species. Inhalation of aerosols of oily or fatty-type materials, including silicones, into alveolar regions of the lung may result in physical disturbances of the lining of the lung with associated effects. This makes the selection of foams as a method of application of silicone containing substances desirable since it should avoid the risk of inhalation when compared to aerosol sprays.

Lower molecular weight siloxanes are frequently used due their volatility and generally dry skin feel. The can be important in trying to overcome the particularly dry skin feeling associated with waterless foam compositions. These can include linear as well as cyclic siloxanes. The lowest molecular weight linear material is hexamethyldisiloxane, (Me)3SiOSi(Me)3 (HMDS), which has a viscosity of 0.48 mPa·s. HMDS has generally shown little effects toxicologically. Other linear molecules of three, four, or five siloxane units are not known for toxic effects. The materials have very limited absorption via typical exposure routes. Like the higher molecular weight polymers, the low molecular weight linear polymers are not mutagenic, irritating, or acutely toxic. Cyclic siloxanes, (SiMe2O)n are widely used in skin care products, in particular the four (n=4) and five (n=5) members cyclic silicones. The innocuous nature of silicones explains their numerous applications where a prolonged contact with the human body is involved: on textile fabrics, in cosmetics, in contact with food and in medical applications. Silicone elastomers are used in many class II or III medical devices regulated by the European Medical Devices Directive. Their excellent biocompatibility is partly due to the low chemical reactivity displayed by silicones, their low surface energy and their hydrophobicity.

Silicone oils possess skin protective properties and readily facilitate regulating residence of an active ingredient in the skin regulating residence of an active ingredient in the skin regulating residence of an active ingredient in the skin. Silicone oil may be either a volatile silicon oil or a non-volatile silicone oil. Some silicones are water-soluble silicones, such as dimethicone copolyol.

Polysiloxanes improve the spreading of oils on the surfaces of, for example, human skin. A disadvantage of the polysiloxanes, used as agents for improving the spreading ability, is their generally poor compatibility with oils, such that the compatibility (solubility) of the polysiloxanes with the oils decreases as the number of dimethylsiloxy units increases. Thus, it is far from straight forward to discover waterless and substantially waterless silicone carriers, compositions and foams which also contain hydrophobic solvents like oils or which also comprise other fatty or greasy substances.

In one or more embodiments the silicone comprising carriers further comprise one or more of an oil, an emollient, a petrolatum, a humectants or a moisturizer.

The amount of silicone or combinations of silicone that may be included in the composition can be increased by increasing the amount of Accommodating Agent or Complex.

Detailed information on dimethicone, cyclomethicone, and simethicone can be found in Pharmaceutical Excipients London, Pharmaceutical Press, Electronic Version 2007 and is incorporated herein by reference. Detailed information on Silicones can be obtained from Dow Corning http://www.dowcorning.com/ and which is also incorporated by reference.

Silicones can be dimethicone 350, dimethicone 360, dimethicone copolyol, cyclomethicone, silicon dioxide, silicone, simethicone, colloidal silicone, poly(dimethylsiloxane/methylvinylsiloxane/methylhydrogensiloxane) dimethylvinyl or dimethylhydroxy or trimethyl end blocked, polydimethylsiloxanes, polydimethylsiloxane oils or polydimethylsiloxane oils modified with ionic or nonionic organic groups, a linear functional polydiorganosiloxane, a linear nonfunctional polydiorganosiloxane, a cyclic polydiorganosiloxane, an alkoxydimethicone, an alkyldimethicone, a phenyldimethicone, a silicone resin and mixtures thereof. More examples are dimethylpolysiloxane, methylphenylpolysiloxanes, cyclic silicones, and amino-, fatty acid-, alcohol-, polyether-, epoxy-fluoro- and/or alkyl-modified silicone compounds, and also polyalkylsiloxanes, polyalkylarylsiloxanes, polyethersiloxanes, as described in U.S. Pat. No. 5,104,645 and the documents cited therein and incorporated herein by reference.

U.S. Pat. No. 5,645,842 discusses silicone waxes, which represent a further class of spreading agents, which have one or more long-chain alkyl groups linked to the backbone. It mentions that the melting point of the silicone waxes increases as the content of long-chain alkyl groups increases and as the chain length of the alkyl group increases and makes reference is to the paper “Silicone Surfactants”, D. Schaefer, Tenside 1990, pages 154 to 158. Silicone waxes are said to lower the surface tension of organic systems, such as the surface tension of mineral oils, and improve the spreading ability of cosmetic oils and waxes. It is noted in the patent that as a rule, however, the spreading ability of mixtures of oils and silicone waxes is only slightly better than that of pure oils.

Bis PEG 18 Methyl Ether Dimethyl Silane is not a standard silicone with foam destroying properties. It is a derivative with a low melting point and melts in contact with skin. It is said to be easy to use with water based compositions, having stabilizing and moisturizing properties and may improve water based foam quality.

Polydimethylsiloxane polymer, Dow Corning® 345 Fluid, cetyl dimethicone and bis-PEG-18 methyl ether dimethyl silane are all used in the Examples. Thus, in one or more particular embodiments the silicone is selected from the group consisting of Polydimethylsiloxane polymer, Dow Corning® 345 Fluid, cetyl dimethicone and his-PEG-18 methyl ether dimethyl silane.

### Stabilizing Agent

A stabilizing agent is an agent that may have to some extent one or more of the properties of foam adjuvant, friction ameliorator, gelling agent, look and feel ameliorator, lubricant, stabilizer, anti-destabilizer, surfactant, thickener and viscosity modifier or enhancer.

In one embodiment the stabilizing agent may help to ameliorate, counteract, or overcome undesirable effects and drawbacks of using a silicone.

In one or more embodiments, the stabilizing agent can be, a polymer or a polymeric agent; more specifically it can be an alkyl lactate for example a C12-15 alkyl lactate, a metal starch; a hydrophobic starch; a microcrystalline cellulose; a cellulose ether and or long chain polysaccharide; a (alpha-tocopheryl polyethylene glycol succinate); polyoxyethylene alkyl ethers and crosslinked polyacrylic acid polymers and the like.

### Polyol

In one or more embodiments, the solvent or secondary solvent is a polyol. A polyol is an organic substance that contains at least two hydroxy groups in its molecular structure. In one or more embodiments, the foamable carrier contains at least one diol (a compound that contains two hydroxy groups in its molecular structure). Examples of diols include propylene glycol (e.g., 1,2-propylene glycol and 1,3-propylene glycol), butanediol (e.g., 1,2-butanediol, 1,3-butanediol, 2,3-butanediol and 1,4-butanediol), butanediol (e.g., 1,3-butanediol and 1,4-butenediol), butynediol, pentanediol (e.g., pentane-1,2-diol, pentane-1,3-diol, pentane-1,4-diol, pentane-1,5-diol, pentane-2,3-diol and pentane-2,4-diol), hexanediol (e.g., hexane-1,6-diol hexane-2,3-diol and hexane-2,56-diol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

In one or more embodiments, the foamable carrier contains at least one triol (a compound that contains three hydroxy groups in its molecular structure), such as glycerin, butane-1,2,3-triol, butane-1,2,4-triol and hexane-1,2,6-triol.

In one or more embodiments, the polyol is a mixture of polyols. In one or more embodiments, the mixture of polyols contains at least one diol and at least one triol. According to certain embodiments the ratio between the diol and triol is between 9:1 and 1:1.

In one or more embodiments, part of mixture of polyols is a saccharide. Exemplary saccharides include, but are not limited to monosaccharide, disaccharides, oligosaccharides and sugar alcohols.

A monosaccharide is a simple sugar that cannot be hydrolyzed to smaller units. Empirical formula is (CH2O)n and range in size from trioses (n=3) to heptoses (n=7). Exemplary monosaccharide compounds are ribose, glucose, fructose and galactose.

Disaccharides are made up of two monosaccharides joined together, such as sucrose, maltose and lactose.

A sugar alcohol (also known as a polyol, polyhydric alcohol, or polyalcohol) is a hydrogenated form of saccharide, whose carbonyl group (aldehyde or ketone, reducing sugar) has been reduced to a primary or secondary hydroxyl group. They are commonly used for replacing sucrose in foodstuffs, often in combination with high intensity artificial sweeteners to counter the low sweetness. Some exemplary sugar alcohols, which are suitable for use are mannitol, sorbitol, xylitol, maltitol, lactitol. (Maltitol and lactitol are not completely hydrogenated compounds—they are a monosaccharide combined with a polyhydric alcohol.) Mixtures of polyols, including (1) at least one polyol selected from a diol and a triol; and (2) a saccharide are contemplated within the scope.

### Polyethylene Glycol

In an embodiment, the solvent consists of a polymerized ethylene glycol, namely polyethylene glycol, which is also termed “PEG”. Exemplary PEGs are provided in Table 2.

TABLE 2

| Composition | Av. Molecular weight | Appearance | Melting point (° C.) |
|---|---|---|---|
| PEG 200 | 190~210 | Oily liquid | |
| PEG 300 | 285~315 | Oily liquid | |
| PEG 400 | 380~420 | Oily liquid | |
| PEG 600 | 570~630 | Oily liquid | 17~22 |
| PEG 1000 | 950~1050 | Solid | 35~40 |
| PEG 4000 | 3800~4400 | Solid | 53~58 |
| PEG 6000 | 5600~6400 | Solid | 55~60 |
| PEG 8000 | 7500~8500 | Solid | 58~65 |

Thus, in an embodiment, the PEG is selected from the group consisting of PEG 200, PEG 300, PEG 400, PEG 600, PEG 1000, PEG 4000, PEG 6000 and PEG 8000. The foamable carrier can contain a single PEG or a mixture of two or more PEGs. PEGs having molecular weight of more that about 1000 possess gelling properties; i.e., they increase the viscosity of a composition. Therefore, by combining PEGs with different molecular weights/melting points, one can attain varying levels of flowability as desirable for the treatment of a given target site.

### Secondary Solvent

Optionally, a secondary solvent is added to the foamable composition. The secondary solvent is selected from a variety of organic solvents that are typically miscible on both water and oil. Non limiting examples of solvent that can be contained in the foamable carrier include dimethyl isosorbide, tetrahydrofurfuryl alcohol polyethyleneglycol ether (glycofurol), DMSO, pyrrolidones, (such as N-Methyl-2-pyrrolidone and 1-Methyl-2-pyrrolidinone), ethyl proxitol, dimethylacetamide (DMAc), PEG-type surfactants and alpha hydroxy acids, such as lactic acid and glycolic acid as well as polar and potent and hydrophobic solvents discussed elsewhere herein.

In one or more embodiments non limiting examples of a secondary non aqueous solvent are solvents such as isosorbide derivatives, such as dimethyl isosorbide, hexylene glycol and glycerin, diethylene glycol monoethyl ether, a liquid polyethylene glycol, glycofurol, tetrahydrofurfuryl alcohol, polyethyleneglycol, ether, DMSO, a pyrrolidone, N-methyl pyrrolidones, N-Methyl-2-pyrrolidone, 1-Methyl-2-pyrrolidinone, ethyl proxitol, dimethylacetamide, a PEG-type surfactant, an alpha hydroxy acid, lactic acid and glycolic acid, hexylene glycol, benzyl alcohol, DMSO, and ethoxydiglycol (transcutol), butylene glycols, glycerol, pentaerythritol, sorbitol, mannitol, oligosaccharides, monooleate of ethoxylated glycerides having about 8 to 10 ethylene oxide units, and cyclodextrins, esters, such as ethyl propionate, tributylcitrate, acetyl triethylcitrate, acetyl tributyl citrate, triethylcitrate, ethyl butyrate, propylene glycol monoacetate, propylene glycol diacetate, .epsilon.-caprolactone and isomers thereof, delta.-valerolactone and isomers thereof, beta.-butyrolactone and isomers thereof, and other solubilizers known in the art, such as dimethyl acetamide.

In an embodiment the secondary non aqueous solvent is monooctanoin.

In one or more embodiments the carrier or composition can comprises a unique silicon in hydrophillic solvent with petrolatum emulsion, wherein the hydrophilic solvent is selected from a liquid polyethylene glycol, a propylene glycol or dimethyl isosorbide.

Appropriate use of a secondary solvent in a waterless foam composition can help improve delivery of active agents to a target area. Foam compositions, for which the solvent includes a secondary solvent, can increase the levels of the active agent in the waterless composition and thus, provide high delivery and improved therapy.

### Solubilization and Penetration Enhancement

In many cases, polyols, PEGs and polar solvents possess a high solubilizing power and thus, they can enable increased concentrations of an active agent. Polyols, PEGs and polar solvents are also known for their skin penetration enhancement properties. These properties enable high drug bioavailability in the target area of treatment, resulting in an enhanced therapeutic effect. Occasionally, combinations of a polyol, PEGs and a secondary polar solvent, exhibit an increased permeability across the skin, as suggested, for example, in Eur J Pharm Biopharm. 1998 November; 46(3):265-71.

Thus, in one or more embodiments, the foamable carrier contains (1) at least one polar solvent, selected from a polyol (selected from a diol and a triol) and PEG; and (2) at least one secondary polar solvent.

In one or more embodiments, the foamable carrier contains (1) a mixture of at least two polyols; and (2) at least one secondary polar solvent. In additional embodiments, the foamable carrier contains a mixture of at least one polyol and at least one PEG; yet in other embodiments the foamable carrier contains (1) a mixture of at least one polyol and at least one PEG and (2) at least one secondary polar solvent.

According to certain embodiments the ratio between the polyol and/or PEG and the secondary polar solvent is between 9:1 and 1:1.

In certain embodiments, the polyol is selected from the group consisting of propylene glycol, hexylene glycol and glycerin (and mixtures thereof); and the secondary polar solvent is selected from the group consisting of dimethyl isosorbide, diethylene glycol monoethyl ether, a liquid polyethylene glycol and glycofurol.

In certain embodiments, the foamable carrier contains (1) at least one polyol; and (2) dimethyl isosorbide.

Short chain alcohols, such as ethanol and propanol are known as polar solvents, however, according to one or more embodiments, the composition is substantially alcohol-free, i.e., free of short chain alcohols. Short chain alcohols, having up to 5 carbon atoms in their carbon chain skeleton and one hydroxyl group, such as ethanol, propanol, isopropanol, butanol, iso-butanol, t-butanol and pentanol, are considered less desirable polar solvents due to their skin-irritating effect.

Thus, in certain embodiments, the composition is substantially alcohol-free and includes less than about 5% final concentration of lower alcohols, preferably less than about 2%, more preferably less than about 1%. However, in other embodiments, a short chain alcohol can be included in the composition, and preferably the ratio between the short chain alcohol and the polyol is less than 1:4 by weight.

### Modulating Agent

The term modulating agent is used to describe an agent which can improve the stability of or stabilize a foamable carrier or composition and or an active agent by modulating the effect of a substance or residue present in the carrier or composition. In one or more embodiments the substance or residue may for example be acidic or basic and potentially alter an artificial pH in a waterless or substantially non aqueous emulsion environment or it may be one or more metal ions which may act as a potential catalyst in a waterless or substantially non aqueous environment.

In one or more embodiments the modulating agent is used in a silicone in PEG or PG emulsion, a unique waterless or substantially emulsion.

In one or more embodiments the modulating agent is used to describe an agent which can affect pH in an aqueous solution. The agent can be any of the known buffering systems used in pharmaceutical or cosmetic formulations as would be appreciated by a man of the art. It can also be an organic acid, a carboxylic acid, a fatty acid an amino acid, an aromatic acid, an alpha or beta hydroxyl acid an organic base or a nitrogen containing compound. In certain embodiments the active agent may function a modulating agent.

In one or more further embodiments the modulating agent is used to describe an agent, which is a chelating or sequestering or complexing agent that is sufficiently soluble or functional in the waterless solvent to enable it to “mop up” or “lock” metal ions.

In the embodiment modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basesity balance of a waterless or substantially non aqueous carrier, composition, foamable carrier or foamable composition or resultant foam.

The substance or residue can be introduced into the formulation from any one or more of the ingredients, some of which themselves may have acidic or basic properties. For example the polymer or solvent may contain basic residues in which case it may be desirable or beneficial to add an acid. Alternatively the surfactant may contain some acid residues in which case the addition of a base may be desirable and beneficial. In some cases more than one ingredient may contain residues which may ameliorate or compound their significance. For example if one ingredient provided weak acid residues and another stronger acid residues the artificial pH in a waterless environment should be lower. In contrast if one residue was acid and the other basic the net effect in the formulation maybe significantly reduced. In an embodiment sufficient modulating agent is added to achieve an artificial pH in which the active agent is preferably stable.

The terms pH, pKa, and pKb, buffers and the like are used in classical measurements of an aqueous solution. Such measurements are artificial in a waterless environment. Nevertheless, reference to and description below of such terms are made for convenience and clarity, since such terms are well defined and understood with reference to aqueous solutions and further due to the lack of an appropriate uniform way of describing and identifying the artificial or virtual pH, pK etc. in a waterless environment in relation to the present invention. Although predictions of artificial pH can be made using dilution techniques of measurements of waterless formulations diluted in water they are formulation sensitive and specific and have to be carefully calibrated with complex formulas.

Waterless medium can be polar and protic yet it does not conform to classical ionic behavior.

A buffer, as defined by Van Slyke [Van Slyke, J. Biol. Chem., 52, 525 (1922)], is “a substance which by its presence in solution increases the amount of acid or alkali that must be added to cause unit change in pH”.

A buffer solution is a solution of a definite pH made up in such a way that this pH alters only gradually with the addition of alkali or acid. Such a solution consists of a solution of a salt of the week acid in the presence of the three acid itself. The pH of the solution is determined by the dissociation equilibrium of the free acid.

An acid can be a strong acid or a weak acid. A strong acid is an acid, which is a virtually 100% ionized in solution. In contrast, a week acid is one which does not ionize fully when it is dissolved in water. The lower the value for pKa, the stronger is the acid and likewise, the higher the value for pKa the weaker is the acid.

A base can be a strong base or a weak base. A strong base is something, which is fully ionic with 100% hydroxide ions. In contrast, a weak base is one which does not convert fully into hydroxide ions in solution. The lower the value for pKb, the stronger is the base and likewise, the higher the value for pKb the weaker is the base.

In one or more embodiments of the present invention the modulating agent comprises an organic compound.

In one or more embodiments of the present invention the modulating agent includes trolamine or stearic acid.

In one or more preferred embodiments the chelating agent is selected from the group consisting of ethylenediaminetetraacetic acid (“EDTA”) and salts thereof such as disodium EDTA, tetrasodium EDTA and calcium disodium EDTA; diethylenetriaminepentaacetic acid (“DTPA”) and salts thereof, hydroxyethlethylenediaminctriacetic acid (“HEDTA”) and salts thereof and nitrilotriacetic acid (“NTA”); more preferably EDTA, HEDTA and their salts; most preferably EDTA and its salts.

In one or more embodiments a preferred non limiting example of the chelating agent is EDTA. Typically, the chelating and sequestering agent is present in the composition at a level of up to about 5.0%, preferably 1.0 percent, by weight, of the composition.

In one or more embodiments the modulating agent may also be a preservative or an antioxidant or an ionization agent. Any preservative, antioxidant or ionization agents suitable for pharmaceutical or cosmetic application may be used. Non limiting examples of antioxidants are tocopherol succinate, propyl galate, butylated hydroxy toluene and butyl hydroxy anisol as well as a whole range of flavanoids such as quercitin and rutin. Ionization agents may be positive or may be negative depending on the environment and the active agent or composition that is to be protected. Ionization agents may for example act to protect or reduce sensitivity of active agents. Non limiting examples of positive ionization agents are benzyl conium chloride, and cetyl pyridium chloride. Non limiting examples of negative ionization agents are sodium lauryl sulphate, sodium lauryl lactylate and phospholipids.

### Polymeric Agent

In one or more embodiments the composition contains a polymeric agent. The presence of a polymeric agent is helpful for the creation of foam, having fine bubble structure, which does not readily collapse upon release from the pressurized aerosol can. The polymeric agent serves to stabilize the foam composition and to control drug residence in the target organ. Preferably, the polymeric agent is soluble or readily dispersible in the polyol; or in the mixture of a polyol and an additional polar solvent. Solid polymeric agents also play an important role in providing body to the formulation especially where all or virtually all of the components are liquid, since watery like formulations do not produce good foams.

Non-limiting examples of polymeric agents that are soluble or readily dispersible in propylene glycol are Hydroxypropylcellulose (klucel) and carbomer (homopolymer of acrylic acid is crosslinked with an allyl ether pentaerythritol, an allyl ether of sucrose, or an allyl ether of propylene, such as Carbopol® 934, Carbopol® 940, Carbopo® 941, Carbopol® 980 and Carbopol® 981.

In some embodiments the polymer is selected from one or more of the following (Table 3) and in some embodiments the polymer is hydroxypropyl cellulose.

TABLE 3
gelling agents
mame

Hydroxypropyl cellulose
Aluminum starch octenylsuccinate (ASOS)
carbopol

Other polymeric agents are suitable for use provided that they are soluble or readily dispersible in the polyol; or in the mixture of a polyol and an additional polar solvent, on a case by case basis.

Exemplary polymeric agents include, in a non-limiting manner, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, amine-bearing polymers such as chitosan; acidic polymers obtainable from natural sources, such as alginic acid and hyaluronic acid; chemically modified starches and the like, carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like.

Additional exemplary polymeric agents include semi-synthetic polymeric materials such as cellulose ethers, such as methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxynethylhydroxyethylcellulose, and cationic celluloses. Polyethylene glycol, having molecular weight of 1000 or more (e.g., PEG 1,000, PEG1500 PEG 4,000, PEG 6,000 and PEG 10,000) also have gelling capacity and while they are considered herein as “polar solvents”, as detailed herein, they are also considered polymeric agents.

Mixtures of the above polymeric agents are contemplated.

The concentration of the polymeric agent should be selected so that the composition, after filling into aerosol canisters, is flowable, and can be shaken in the canister. In one or more embodiments, the concentration of the polymeric agent is selected such that the viscosity of the composition, prior to filling of the composition into aerosol canisters, is less than 60,000 CPs, and more preferably, less than 53,000 CPs.

### Emulsifier or Surface-Active Agent

The composition further contains an emulsifier a surface-active agent or surfactant and such terms can be used interchangeably. Surface-active agents (also termed “surfactants”) include any agent linking oil and water in the composition, in the form of emulsion. A surfactant's hydrophilic/lipophilic balance (HLB) describes the emulsifier's affinity toward water or oil. HLB is defined for non-ionic surfactants. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average). In many cases a single surfactant may suffice. In other cases a combination of two or more surfactants is desired. Reference to surfactant in the specification can also apply to a combination or to a surfactant system. As will be appreciated by a person skilled in the art which surfactant or surfactant system is more appropriate is related to the vehicle and intended purpose. In general terms a combination of surfactants is usually preferable where the vehicle is an emulsion. In a waterless or substantially waterless environment it has been discovered that the presence of a surfactant or combination of surfactants can be significant in producing breakable forms of good quality. It has been further discovered that the generally thought considerations for HLB values for selecting a surfactant or surfactant combination are not always binding for emulsions particularly in a waterless environment and that good quality foams can be produced with a surfactant or surfactant combination both where the HLB values are in or towards the lipophilic side of the scale and where the HLB values are in or towards the hydrophilic side of the scale. Nevertheless, it has been observed herein—even though the usual considerations for HLB may not apply to a waterless environment—that using solid non ionic surfactants with a HLB reasonably close to that of the required HLB of the silicone appears to result in both an improved foam quality and emulsion stability when compared to the same formulation with a solid non ionic surfactant with a high HLB. In a similar comparative experiment using liquid surfactants no significant difference was observed between high or low HLB. Similar results were also seen when the experiments were repeated with the addition of a polymeric agent in the formulation save that since the presence of the polymeric agent also acted to improve emulsion stability any possible significant effect of changing HLB on emulsion stability was not seen. As will be appreciated the chemical nature of the surfactant may be significant in determining whether a molecule is partly miscible with silicone and partly miscible with a PEG or PG to form a stable emulsion. Without being bound by any theory it may simply be the case that the low HLB surfactant is more miscible in silicone than its high HLB counterpart.

It has been further discovered that the physical nature of the surfactant or combination thereof can affect the quality of foam produced. For example and in very general oversimplified terms the presence of a solid or waxy surfactant may help where the composition is more liquid or less viscous in nature and similarly where a formulation is less liquid and more viscous the presence of a liquid surfactant may help. More particularly a combination of a solid or waxy surfactant with a liquid surfactant may be of significance and the ratio between them may be adjusted to take into account to an extent whether the composition is otherwise more liquid or otherwise more viscous in nature. The position is more complex than this since the presence and interaction of other agents such as foam adjuvants, polymeric agents as well as unctuous additives and hydrophobic agents all have an influence on achieving a breakable foam of quality.

According to one or more embodiments the composition contains a single surface active agent having an HLB value between about 2 and 9, or more than one surface active agent and the weighted average of their HLB values is between about 2 and about 9. Lower HLB values may in certain embodiments be more applicable.

According to one or more embodiments the composition contains a single surface active agent having an HLB value between about 7 and 14, or more than one surface active agent and the weighted average of their HLB values is between about 7 and about 14. Mid range HLB values may in certain embodiments be more suitable.

According to one or more other embodiments the composition contains a single surface active agent having an HLB value between about 9 and about 19, or more than one surface active agent and the weighted average of their HLB values is between about 9 and about 19. In a waterless or substantially waterless environment a wide range of HLB values may be suitable.

According to one or more embodiments a wide range of HLB values giving about an average mid range can be achieved with combinations of two, three or more surfactants.

Preferably, the composition contains a non-ionic surfactant. Non-limiting examples of possible non-ionic surfactants include a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkyl ether, polyoxyethylene cetyl ether, polyoxyethylene palmityl ether, polyethylene oxide hexadecyl ether, polyethylene glycol cetyl ether, brij 38, brij 52, brij 56 and brij W1, a sucrose ester, a partial ester of sorbitol and its anhydrides, sorbitan monolaurate, sorbitan monolaurate a monoglyceride, a diglyceride, isoceteth-20 and mono-, di- and tri-esters of sucrose with fatty acids.

In certain embodiments, surfactants are selected which can provide a close packed surfactant layer. To achieve such objectives combinations of at least two surfactants are selected. Preferably, they should be complex emulgators and more preferably they should both be of a similar molecular type; for example, a pair of ethers, like steareth 2 and steareth 21, or a pair of esters, for example, PEG-40 stearate and polysorbate 80. Ideally, the surfactants can be ethers. In certain circumstances POE esters cannot be used and a combination of sorbitan laurate and sorbitan stearate or a combination of sucrose stearic acid ester mixtures and sodium laurate may be used. All these combinations due to their versatility and strength may also be used satisfactorily and effectively with ether formulations, although the amounts and proportion may be varied according to the formulation and its objectives as will be appreciated by a man of the art.

It has been discovered also that by using a derivatized hydrophilic polymer with hydrophobic alkyl moieties as a polymeric emulsifier such as pemulen it is possible to stabilize the emulsion better about or at the region of phase reversal tension. Other types of derivatized polymers like silicone copolymers, derivatized starch [Aluminum Starch Octenylsuccinate (ASOS)]/[DRY-FLO AF Starch], and derivatized dexrin may also a similar stabilizing effect.

A series of dextrin derivative surfactants prepared by the reaction of the propylene glycol polyglucosides with a hydrophobic oxirane-containing material of the glycidyl ether are highly biodegradable. [Hong-Rong Wang and Keng-Ming Chen, Colloids and Surfaces A: Physicochemical and Engineering Aspects Volume 281, Issues 1-3, 15 Jun. 2006, Pages 190-193].

In one embodiment, the surfactant is selected from one or more of the following (Table 4) and in one embodiment at least one surfactant is a solid with a low HLB.

TABLE 4
surfactants

| mame | solid/liquid | HLB |
|---|---|---|
| Methyl glucose sesqui stearate | solid | 6.6 |
| Steareth 2 | solid | 4.9 |
| cetearyl glucoside* | solid | 11 |
| Steareth 21 | solid | 15.5 |
| Sorbitan monooleate | liquid | 4.3 |
| Polysorbate 80 | liquid | 15 |

*Cetearyl alcohol and cetearyl glucoside are usually combined and available as - montanov 68

In one or more embodiments the stability of the composition can be improved when a combination of at least one non-ionic surfactant having HLB of less than 9 and at least one non-ionic surfactant having HLB of equal or more than 9 is employed. Thus, in an exemplary embodiment, a combination of at least one non-ionic surfactant having HLB of less than 7 and at least one non-ionic surfactant having HLB of equal or more than 7 is employed, at a ratio of between 1:8 and 8:1, or at a ratio of 4:1 to 1:4, wherein the HLB of the combination of emulsifiers is preferably between about 4 and about 15.

Non-limiting examples of non-ionic surfactants that have HLB of about 7 to about 12 include steareth 2 (HLB˜4.9); glyceryl monostearate/PEG 100 stearate (Av HLB˜11.2); stearate Laureth 4 (HLB˜9.7) and cetomacrogol ether (e.g., polyethylene glycol 1000 monocetyl ether). Exemplary stabilizing surfactants which may be suitable for use in the present invention are found below (Tables 5-12).

TABLE 5
PEG-Fatty Acid Monoester Surfactants

| Chemical name | Product example name | HLB |
|---|---|---|
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-45 stearate | Nikkol MYS-45 (Nikko) | 18 |
| PEG-50 stearate | Myrj 53 | >10 |
| PEG-100 stearate | Myrj 59, Arlacel 165 (ICI) | 19 |

TABLE 6
PEG-Fatty Acid Diester Surfactants:

| Chemical name | Product example name | HLB |
|---|---|---|
| PEG-4 dilaurate | Mapeg 200 DL (PPG), Kessco | 7 |
| | PEG 200 DL (Stepan), LIPOPEG | |
| | 2-DL (Lipo Chem.) | |
| PEG-4 | distearate Kessco 200 DS | 5 |
| | (Stepan) | |
| PEG-32 dioleate | Kessco PEG 1540 DO (Stepan) | 15 |
| PEG-400 dioleate) | Cithrol 4DO series (Croda | >10 |
| PEG-400 disterate | Cithrol 4DS series (Croda) | >10 |
| PEG-20 glyceryl oleate | Tagat O (Goldschmidt) | >10 |

TABLE 7
Transesterification Products of Oils and Alcohols

| Chemical name | Product example name | HLB |
|---|---|---|
| PEG-30 castor oil | Emalex C-30 (Nihon Emulsion) | 11 |
| PEG-40 hydrogenated castor | Cremophor RH 40 (BASF), | 13 |
| oil) | Croduret (Croda), Emulgin HRE | |
| | 40 (Henkel) | |

TABLE 8
Polyglycerized Fatty Acids

| Chemical name | Product example name | LB |
|---|---|---|
| Polyglyceryl-6 dioleate | Caprol 6G20 (ABITEC); PGO-62 | 8.5 |
| | (Calgene), PLUROL | |
| | OLEIQUE CC 497 | |
| | (Gattefosse) Hodag | |

TABLE 9
PEG-Sorbitan Fatty Acid Esters

| Chemical name | Product example name | HLB |
|---|---|---|
| PEG-20 sorbitan | Tween 40 (Atlas/ICI), Crillet 2 | 16 |
| monopalmitate | (Croda) | |
| PEG-20 sorbitan monostearate | Tween-60 (Atlas/ICI), Crillet 3 | 15 |
| | (Croda) | |
| PEG-20 sorbitan | Tween-80 (Atlas/ICI), Crillet 4 | 15 |
| | (Croda) | |
| PEG-20 sorbitan | Tween-80 (Atlas/ICI), Crillet 4 | 15 |
| | (Croda) | |

TABLE 10
Polyethylene Glycol Alkyl Ethers

| Chemical name | Product example name | HLB |
|---|---|---|
| PEG-2 oleyl ether | oleth-2 Brij 92/93 (Atlas/ICI) | 4.9 |
| PEG-3 oleyl ether | oleth-3 Volpo 3 (Croda) | <10 |
| PEG-5 oleyl ether | oleth-5 Volpo 5 (Croda) | <10 |
| PEG-10 oleyl ether | oleth-10 Volpo 10 (Croda), Brij | 12 |
| | 96/97 (Atlas/ICI) | |
| PEG-20 oleyl ether | oleth-20 Volpo 20 (Croda), Brij | 15 |
| | 98/99 (Atlas/ICI) | |
| PEG-4 lauryl ether | laureth-4Brij 30 (Atlas/ICI) | 9.7 |
| PEG-23 lauryl ether | laureth-23Brij 35 (Atlas/ICI) | 17 |
| PEG-10 stearyl ether | Brij 76 (ICI) | 12 |
| PEG-2 cetyl ether | Brij 52 (ICI) | 5.3 |

TABLE 11
Sugar Ester Surfactants

| Chemical name | Product example name | HLB |
|---|---|---|
| Sucrose distearate | Sisterna SP50, Surfope 1811 | 11 |

TABLE 12
Sorbitan Fatty Acid Ester Surfactants

| Chemical name | Product example name | HLB |
|---|---|---|
| Sorbitan monolaurate | Span-20 (Atlas/ICI), Crill 1 | 8.6 |
| | (Croda), Arlacel 20 (ICI) | |
| Sorbitan monopalmitate | Span-40 (Atlas/ICI), Crill 2 | 6.7 |
| | (Croda), Nikkol SP-10 (Nikko) | |
| Sorbitan monooleate | Span-80 (Atlas/ICI), Crill 4 | 4.3 |
| | (Croda), Crill 50 (Croda) | |
| Sorbitan monostearate | Span-60 (Atlas/ICI), Crill 3 | 4.7 |
| | (Croda), Nikkol SS-10 (Nikko) | |

In one or more embodiments the surface active agent is a complex emulgator in which the combination of two or more surface active agents can be more effective than a single surfactant and provides a more stable emulsion or improved foam quality than a single surfactant. For example and by way of non-limiting explanation it has been found that by choosing say two surfactants, one hydrophobic and the other hydrophilic the combination can produce a more stable emulsion than a single surfactant. Preferably, the complex emulgator comprises a combination of surfactants wherein there is a difference of about 4 or more units between the HLB values of the two surfactants or there is a significant difference in the chemical nature or structure of the two or more surfactants.

Specific non limiting examples of surfactant systems are, combinations of polyoxyethylene alkyl ethers, such as Brij 59/Brij 10; Brij 52/Brij 10; Steareth 2/Steareth 20; Steareth 2/Steareth 21 (Brij 72/Brij 721); combinations of polyoxyethylene stearates such as Myrj 52/Myrj 59; combinations of sucrose esters, such as Surphope 1816/Surphope 1807; combinations of sorbitan esters, such as Span 20/Span 80; Span 20/Span 60; combinations of sucrose esters and sorbitan esters, such as Surphope 1811 and Span 60; combinations of liquid polysorbate detergents and PEG compounds, such as Tween 80/PEG-40 stearate; methyl glucose sesquistearate; polymeric emulsifiers, such as Permulen (TR1 or TR2); liquid crystal systems, such as Arlatone (2121), Stepan (Mild RM1), Nikomulese (41) and Montanov (68) and the like.

In certain embodiments the surfactant is preferably a combination of steareth-2 and steareth-21; in certain other embodiments the surfactant is a combination of polysorbate 80 and PEG-40 stearate. In certain other embodiments the surfactant is a combination of glyceryl monostearate/PEG 100 stearate. In certain other embodiments the surfactants is a combination of steareth 2 and methyl glucose sesquistearate. In certain other embodiments the surfactants is a combination of steareth 2 and cetearyl alcohol and cetearyl glucoside.

In certain cases, the surface active agent is selected from the group of cationic, zwitterionic, amphoteric and ampholytic surfactants, such as sodium methyl cocoyl taurate, sodium methyl oleoyl taurate, sodium lauryl sulfate, triethanolamine lauryl sulfate and betaines.

Many amphiphilic molecules can show lyotropic liquid-crystalline phase sequences depending on the volume balances between the hydrophilic part and hydrophobic part. These structures are formed through the micro-phase segregation of two incompatible components on a nanometer scale. Soap is an everyday example of a lyotropic liquid crystal. Certain types of surfactants tend to form lyotropic liquid crystals in emulsions interface (oil-in-water) and exert a stabilizing effect. Non limiting examples of surfactants with postulated tendency to form interfacial liquid crystals are: phospholipids, alkyl glucosides, sucrose esters, sorbitan esters. In certain embodiments surfactants which tend to form liquid crystals may improve the quality of foams produced from compositions.

In one or more embodiments the carrier or composition is capable of forming or tends to form liquid crystals.

In one or more embodiments the carrier or composition, comprises liquid crystals.

In one or more embodiments the carrier or composition, comprises liquid crystals wherein the liquid crystals are relatively few.

In one or more embodiments the at least one surface active agent is solid, semi solid or waxy.

In one or more embodiments the surfactant can be, a surfactant system comprising of a surfactant and a co surfactant, a waxy emulsifier, a liquid crystal emulsifier, an emulsifier which is solid or semi solid at room temperature and pressure, or combinations of two or more agents in an appropriate proportion as will be appreciated a person skilled in the art. Where a solid or semi solid emulsifier combination is used it can also comprise a solid or semi solid emulsifier and a liquid emulsifier.

In one or more embodiments, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. We have surprisingly found that non-ionic surfactants alone provide formulations and foams of good or excellent quality in the waterless and substantially waterless carriers and compositions.

Thus, in a preferred embodiment, the surface active agent, the composition contains a non-ionic surfactant. In another preferred embodiment the composition includes a mixture of non-ionic surfactants as the sole surface active agent. Yet, in additional embodiments, the foamable composition includes a mixture of at least one non-ionic surfactant and at least one ionic surfactant in a ratio in the range of about 100:1 to 6:1. In further embodiments, surface active agent comprises a combination of a non-ionic surfactant and an ionic surfactant, at a ratio of between 1:1 and 20:1. In one or more embodiments, a combination of a non-ionic surfactant and an ionic surfactant (such as sodium lauryl sulphate and cocamidopropylbetaine) is employed.

In selecting a suitable surfactant or combination thereof it should be borne in mind that the upper amount of surfactant that may be used may be limited by the shakability of the composition. In general terms, as the amount of non liquid surfactant is increased the shakability of the formulation reduces until a limitation point is reached where the formulation becomes non shakable. Thus in an embodiment any effective amount of surfactant may be used provided the formulation remains shakable or at least flowable. In the present invention where it is desirable to use a high molecular weight solvent and more particularly significant amounts it may be helpful to include a liquid surfactant in addition to or if a solid foam adjuvant and/or polymeric agent are also present then in place of a more waxy surfactant and or to increase the level of the surfactant.

If the composition as formulated is a substantially non shakable composition it is nevertheless possible as an exception in the scope for the formulation to be flowable to a sufficient degree to be able to flow through an actuator valve and be released and still expand to form a good quality foam. This surprising and unusual exception may be due one or more of a number of factors such as the high viscosity, the softness, the lack of crystals, the pseudoplastic or semi pseudo plastic nature of the composition and the dissolution of the propellant into the petrolatum. Thus in an embodiment any effective amount of surfactant may be used provided the formulation remains flowable.

In one or more embodiments, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content, which have higher HLB values.

In certain embodiments the amount of surfactant or combination of surfactants is between about 0.05% to about 20%; between about 0.05% to about 15%. or between about 0.05% to about 10%. In a preferred embodiment the concentration of surface active agent is between about 0.2% and about 8%. In a more preferred embodiment the concentration of surface active agent is between about 1% and about 6%.

If the composition is formulated as a substantially non flowing composition for use as a gel, ointment or cream, then the above limitation of shakability does not apply. Suitable formulations include polyethylene glycol or derivatives or mixtures thereof or propylene glycol comprising higher levels of waxy or semi solid or solid surfactants and/or comprising higher molecular weight polymers or polymer combinations which are usually waxy or solid at room temperature such as PEG 4000 or Peg 4000/400 combination with significant amounts of say PEG 4000 of 5% of 10% of 15% of 20% of 25% of 30% of as a basis for gels, ointments or creams. Other similar combinations may be envisaged of say PEG6000/PEG200; PEG4000/PEG200; PEG4000/PEG400/PEG200; and the like.

In one or more embodiments there is provided a composition, comprising:
(a) a silicone
(b) about more than 3% of high molecular weight solid PEG;
(c) a waterless solvent comprising about 25% to about 95% of at least a polar solvent selected from the group consisting of (1) a propylene glycol or derivative and (2) a polyethylene glycol (PEG) or derivative or mixtures thereof;
(d) 0% to about 48% of a secondary waterless solvent;
(e) an Accommodating Agent or Complex;
(f) optionally at least one polymeric agent at a concentration of about 0.1% to about 5% by weight of the total composition, wherein the at least one polymeric agent is selected from a bioadhesive agent, a gelling agent, a film forming agent and a phase change agent which is selected such that it has some surfactant properties if it is used in the absence of surface active agent; and
(g) a therapeutically effective amount of an active agent.

In one or more further embodiments there is provided a composition, comprising:
(a) a silicone
(b) a mixture of polyethylene glycol (PEG) or PEG derivatives, wherein the PEG mixtures is present at a concentration of about 70% to about 96.5% by weight of the total composition;
(c) 0% to about 28% of a secondary solvent
(d) an Accommodating Agent or Complex; and
(e) optionally at least one polymeric agent at a concentration of about 0.11% to about 5% by weight of the total composition, wherein the at least one polymeric agent is selected from a bioadhesive agent, a gelling agent, a film forming agent and a phase change agent which is selected such that it has some surfactant properties if it is used in the absence of surface active agent; and
(f) a therapeutically effective amount of an active agent
   wherein at least one PEG comprises about more than 5% of high molecular weight solid PEG.

As will be appreciated by someone skilled in the art to the extent higher molecular weight PEGs are introduced or the amount thereof are increased the composition will become more suitable for a gel or ointment pharmaceutical or cosmetic composition.

### Hydrophobic Solvent

Optionally, the foamable carrier further contains at least one hydrophobic solvent. The identification of a “hydrophobic solvent”, as used herein, is not intended to characterize the solubilization capabilities of the solvent for any specific active agent or any other component of the foamable composition. Rather, such information is provided to aid in the identification of materials suitable for use as a part in the foamable compositions described herein.

A “hydrophobic solvent” as used herein refers to a material having solubility in distilled water at ambient temperature of less than about 1 gm per 100 mL, more preferable less than about 0.5 gm per 100 mL, and most preferably less than about 0.1 gm per 100 mL.

In one or more embodiments, the hydrophobic organic carrier is an oil, such as mineral oil, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers.

### Humectant

A humectant, is a substance that helps retain moisture and also prevents rapid evaporation. Non limiting examples are propylene glycol, propylene glycol derivatives, glycerin, hydrogenated starch hydrosylate, hydrogenated lanolin, lanolin wax, D manitol, sorbitol, sodium 2-pyrrolidone-5-carboxylate, sodium lactate, sodium PCA, soluble collagen, dibutyl phthalate, and gelatin. Non limiting preferred examples of suitable humectants are propylene glycol, propylene glycol derivatives, and glycerin. Further examples are provided elsewhere in the description. Other examples of humectants and moisturizers may be found in the Handbook of Pharmaceutical Additives published by Gower. Suitable ones for use with and soluble in the waterless and substantially waterless compositions may be selected as will be appreciated by a person skilled in the art.

### Moisturizers

A moisturizer, is a substance that helps retain moisture or add back moisture to the skin. Examples are allantoin, petrolatum, urea, lactic acid, sodium PCV, glycerin, shea butter, caprylic/capric/stearic triglyceride, candelilla wax, propylene glycol, lanolin, hydrogenated oils, squalene, sodium hyaluronate and lysine PCA. Other examples may be found in the Handbook of Pharmaceutical Additives published by Gower.

Pharmaceutical compositions may in one or more embodiments usefully comprise in addition a humectant or a moisturizer or combinations thereof.

### Foam Adjuvant

Optionally, a foam adjuvant is included in the foamable carriers to increase the foaming capacity of surfactants and/or to stabilize the foam. In one or more embodiments, the foam adjuvant agent includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols, derived from beeswax and including a mixture of alcohols, a majority of which has at least 20 carbon atoms in their carbon chain, are especially well suited as foam adjuvant agents. The amount of the fatty alcohol required to support the foam system is inversely related to the length of its carbon chains. Foam adjuvants, as defined herein are also useful in facilitating improved spreadability and absorption of the composition.

In one or more embodiments, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. As for fatty alcohols, the amount of fatty acids required to support the foam system is inversely related to the length of its carbon chain.

Optionally, the carbon atom chain of the fatty alcohol or the fatty acid may have at least one double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

In one or more embodiments of the present invention the foam adjuvant is one or more of the following (Table 13):

TABLE 13
foam adjuvants

| mame | RHLB |
|---|---|
| Stearyl alcohol | 15.5 |
| Cetearyl alcohol* | 15.5 |
| Stearic acid | 15 |
| Myristyl alcohol | ~16** |

*Cetearyl alcohol and cetearyl glucoside are usually combined together and available as - montanov 68
**no information (estimated)

### Additional Components

The composition may further optionally include a variety of formulation excipients, which are added in order to fine-tune the consistency of the formulation, protect the formulation components from degradation and oxidation and modify their consistency. In an embodiment, a composition includes one or more additional components. Such additional components include but are not limited to anti perspirants, anti-static agents, buffering agents, bulking agents, chelating agents, cleansers, colorants, conditioners, deodorants, diluents, dyes, emollients, fragrances, hair conditioners, humectants, pearlescent aids, perfuming agents, permeation enhancers, pH-adjusting agents, preservatives, protectants, skin penetration enhancers, softeners, solubilizers, sunscreens, sun blocking agents, sunless tanning agents, and viscosity modifiers. As is known to one skilled in the art, in some instances a specific additional component may have more than one activity, function or effect.

In an embodiment, the additional component is a pH adjusting agent or a buffering agent. Suitable buffering agents include but are not limited to acetic acid, adipic acid, calcium hydroxide, citric acid, glycine, hydrochloric acid, lactic acid, magnesium aluminometasilicates, phosphoric acid, sodium carbonate, sodium citrate, sodium hydroxide, sorbic acid, succinic acid, tartaric acid, and derivatives, salts and mixtures thereof.

In an embodiment, the additional component is an emollient. Suitable emollients include but are not limited to mineral oil, lanolin oil, coconut oil, cocoa butter, olive oil, aloe vera extract, jojoba oil, castor oil, fatty acids, fatty alcohols, diisopropyl adipate, hydroxybenzoate esters, benzoic acid esters of C9 to C15 alcohols, isononyl iso-nonanoate, silicone oils, polyethers, C12 to C15 alkyl benzoates, oleic acid, stearic fatty acid, cetyl alcohols, hexadecyl alcohol, dimethyl polysiloxane, polyoxypropylene cetyl ether, polyoxypropylene butyl ether, and derivatives, esters, salts and mixtures thereof.

In an embodiment, the additional component is a humectant. Suitable humectants include but are not limited to guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, alkoxylated glucose, hyaluronic acid, lactamide monoethanolamine, acetamide monoethanolaine and derivatives, esters, salts and mixtures thereof.

In an embodiment, the additional component is a preservative. Suitable preservatives include but are not limited to C12 to C15 alkyl benzoates, alkyl p-hydroxybenzoates, aloe vera extract, ascorbic acid, benzalkonium chloride, benzoic acid, benzoic acid esters of C9 to C15 alcohols, butylated hydroxytoluene, castor oil, cetyl alcohols, chlorocresol, citric acid, cocoa butter, coconut oil, diazolidinyl urea, diisopropyl adipate, dimethyl polysiloxane, DMDM hydantoin, ethanol, fatty acids, fatty alcohols, hexadecyl alcohol, hydroxybenzoate esters, iodopropynyl butylcarbamate, isononyl iso-nonanoate, jojoba oil, lanolin oil, methylparaben, mineral oil, oleic acid, olive oil, polyethers, polyoxypropylene butyl ether, polyoxypropylene cetyl ether, potassium sorbate, silicone oils, sodium propionate, sodium benzoate, sodium bisulfite, sodium metabisulfite (disodium metabisulfite), sorbic acid, stearic fatty acid, vitamin E, vitamin E acetate and derivatives, esters, salts and mixtures thereof.

In an embodiment, the additional component is a skin penetration enhancer. Suitable skin penetration enhancers include but are not limited to acetone, acyl lactylates, acyl peptides, acylsarcosinates, alkanolamine salts of fatty acids, alkyl benzene sulphonates, alkyl ether sulphates, alkyl sulphates, anionic surface-active agents, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl)ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dimethyl sulphoxide, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiallylammonium chloride), polyglycerol esters, polyoxyethylene lauryl ether, polyoxy:polyoxyethylene stearate, polyoxypropylene 15 stearyl ether, poly(vinyl pyridinium chloride), propan-1-ol, propan-2-ol, propylene glycol dipelargonate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quatenised poly (dimethylaminoethylmethacryl-ate), quaternised poly (vinyl alcohol), sapamin hydrochloride, sodium cocaminopropionate, sodium dioctyl sulphonsuccinate, sodium laurate, sodium lauryl ether sulphate, sodium lauryl sulphate, sugar esters, sulphosuccinate, tetrahydrofuran, tetrahydrofurfural alcohol, transcutol, triethanolamine dodecyl benzene sulphonate, triethanolamine oleate, urea, water and derivatives, esters, salts and mixtures thereof.

### Propellants

Aerosol propellants are used to generate and administer the foamable composition as a foam. The total composition including propellant, foamable compositions and optional ingredients is referred to as the foamable carrier. The propellant makes up about 3% to about 25% (w/w) of the foamable carrier or composition. Examples of suitable propellants include volatile hydrocarbons such as butane, propane, isobutane, and fluorocarbon gases or mixtures thereof. In an embodiment the propellant is 1681, which is a mixture of three gas propellants propane, isobutene and butane. In another embodiment it is AP 70, which is a mixture of propane, isobutene and butane with a higher pressure. In some circumstances the propellant may be up to 35%. The total composition including propellant, foamable compositions and optional ingredients is referred to as the foamable composition. However, for the purposes of calculating the percentage of each component and thereby the amount present in the resultant foam the propellant is not included in the 100% but is instead added to the 100% since the propellant is essentially discharged into the atmosphere upon expulsion of the formulation. Accordingly, when referring to the percentage of propellant in the total composition, this percentage is in addition to the remaining components of the composition (i.e., >100%). Alternatively, the amount of propellant in the total composition is expressed as a ratio of the propellant to the remaining formulation or composition components. In some embodiments, the remaining formulation or composition components are referred to as a precursor composition or carrier. When the precursor composition or carrier is combined with the propellant, the amount of propellant is expressed as a ratio of precursor to propellant.

Alcohol and organic solvents render foams inflammable. It has been surprisingly discovered that fluorohydrocarbon propellants, other than chloro-fluoro carbons (CMCs), which are non-ozone-depleting propellants, are particularly useful in the production of a non-flammable foamable composition. A test according to European Standard prEN 14851, titled “Aerosol containers—Aerosol foam flammability test” revealed that compositions containing an organic carrier that contains a hydrophobic organic carrier and/or a polar solvent, which are detected as inflammable when a hydrocarbon propellant is used, become non-flammable, while the propellant is an HFC propellant.

Such propellants include, but are not limited to, hydro uorocarbon (HFC) propellants, which contain no chlorine atoms, and as such, fall completely outside concerns about stratospheric ozone destruction by chlorofluorocarbons or other chlorinated hydrocarbons. Exemplary non-flammable propellants according to this aspect include propellants made by DuPont under the registered trademark Dymel, such as 1,1,1,2 tetrafluorethane (Dymel 134), and 1,1,1,2,3,3,3 heptafluoropropane (Dymel 227). HFCs possess Ozone Depletion Potential of 0.00 and thus, they are allowed for use as propellant in aerosol products.

Notably, the stability of foamable emulsions including HFC as the propellant can be improved in comparison with the same composition made with a hydrocarbon propellant.

In one or more embodiments foamable compositions comprise a combination of a HFC and a hydrocarbon propellant such as n-butane or mixtures of hydrocarbon propellants such as propane, isobutane and butane.

### Hygroscopic Property of the Composition

A hydroscopic substance is a substance that absorbs water readily from its surroundings. Microorganisms require water to grow and reproduce, and such water requirements are best defined in terms of water activity of the substrate. The water activity of a solution is expressed as Aw=P/Po, where P is the water vapor pressure of the solution and Po is the vapor pressure of pure water at the same temperature. Addition of a hygroscopic substance to an aqueous solution in which a microorganism is growing will have the effect of lowering the Aw, with a consequent effect upon cell growth. Every microorganism has a limiting Aw, below which it will not grow, e.g., for streptococci, klebsiella spp., escherichia coli, clostridium perfringens, and pseudomonas spp., the Aw value is 0.95. Staphylococcus aureus is most resistant and can proliferate with an Aw as low as 0.86.

The water activity of a product can be determined from the relative humidity of the air surrounding the sample when the air and the sample are at equilibrium. Measurement is performed by placing a sample in an enclosed space where this equilibrium can take place. Once this occurs, the water activity of the sample and the relative humidity of the air are equal. The measurement taken at equilibrium is called an equilibrium relative humidity or ERH. The relationship between the water activity and ERH is in accordance with the following formula:

Aw=ERH/100

Various types of water activity instruments are commercially available. One exemplary instrument uses chilled-mirror dew point technology while other instruments measure relative humidity with sensors that change electrical resistance or capacitance.

Polyols, PEG's propylene glycols and other polar solvents have a great affinity for water, and as such, they exhibit hygroscopic properties. The concentration of the polyol, the PEG and/or other polar solvents determines the Aw of the carrier. In one or more embodiments, the polyols, the PEG and/or the secondary polar solvent is contained in the composition at a sufficient concentration to provide an Aw value of the hygroscopic carrier of less than 0.9. In other embodiments, the concentration of the polyol, the PEG and/or secondary polar solvent in the composition is selected to provide a Aw value selected from the ranges of (1) about 0.8 and about 0.9; (2) about 0.7 and about 0.8; and (3) less than about 0.7.

As such, a composition containing a polyol, a PEG with or without a secondary polar solvent can be used as topical treatment of superficial infectious conditions.

The advantage of providing a hygroscopic composition in a pressurized packaging presentation is readily perceived. The usage of all other presentations, such as solutions, creams, lotions, ointments and the like involves repeated opening of the package closure, resulting in absorption of water from the surrounding environment and a subsequent elevation of the Aw (thus lowering the hygroscopicity of the product, and therefore decreasing its anti-infective potential. By contrast, a pressurized packaging does not allow for any humidity to be absorbed by the preparation, and therefore, the hygroscopic character of the composition cannot be damaged.

In one or more embodiments, the hygroscopic composition further contains an anti-infective agent, selected from the group of an antibiotic agent, an antibacterial agent, an antifungal agent, an agent that controls yeast, an antiviral agent and an antiparasitic agent. Combining the anti-infective effect of a hygroscopic composition that acts through a dehydration mechanism, with an anti-infective agent that acts through alternate mechanisms may result in a synergistic effect and consequently higher success rate of the treatment.

### Composition and Foam Physical Characteristics and Advantages

A pharmaceutical or cosmetic composition manufactured using the foamable carrier is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foamable composition is stable, having an acceptable shelf-life of at least one year, or preferably, at least two years at ambient temperature, as revealed in accelerated stability tests. Organic carriers and propellants tend to impair the stability of emulsions and to interfere with the formation of stable foam upon release from a pressurized container. It has been observed, however, that the foamable compositions are surprisingly stable for waterless or substantially waterless compositions. Following accelerated stability studies, they demonstrate desirable texture; they form fine bubble structures that do not break immediately upon contact with a surface, spread easily on the treated area and absorb quickly.

Foamable carriers and compositions should be free flowing, or at least sufficiently flowable to allow them to flow through the aperture of the container, e.g., and aerosol container, and create an acceptable foam.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

As further aspect of the foam is breakability. The breakable foam is thermally stable yet breaks under sheer force. Sheer-force breakability of the foam is clearly advantageous over thermally induced breakability. Thermally sensitive foams immediately collapse upon exposure to skin temperature and, therefore, cannot be applied on the hand and afterwards delivered to the afflicted area.

The foam has several advantages, when compared with hydroalcoholic foam compositions, such as described in WO 2004/071479:
1. Breakability. The foam is thermally stable. Unlike hydroalcoholic foam compositions of the prior art, the foam is not “quick breaking”, i.e., it does not readily collapse upon exposure to body temperature environment. Sheer-force breakability of the foam is clearly advantageous over thermally induced breakability, since it allows comfortable application and well directed administration to the target area.
2. Skin drying and skin barrier function. Short chain alcohols are known to dry the skin and impair the integrity of the skin barrier. By contrast, including a film forming agent in the composition foes not cause unwanted skin barrier damage.
3. Irritability. Due to the lack or relatively low levels of alcohol, the use of ionic surfactants, the presence of silicone and improvement in skin barrier function, skin irritability is eliminated.
4. Dry feeling. The presense of silicone can significantly overcome the dry feeling of waterless formulations possibly due to its lubricating property.

Another property of the foam is specific gravity, as measured upon release from the aerosol can. Typically, foams have specific gravity of less than 0.12 g/mL; or less than 0.10 g/mL; or less than 0.08 g/mL, depending on their composition and on the propellant concentration.

### Pharmaceutical Composition

The foamable compositions herein are an ideal vehicle for active pharmaceutical ingredients (API's) and active cosmetic ingredients, vitamins, flavanoids and the like. In the context herein, active pharmaceutical ingredients and active cosmetic ingredients are collectively termed “active agent(s)” or “therapeutic agent(s)”. Thus, these vehicles compositions and foams are especially suitable for storage and delivery of sensitive active agents that are not long term stable in an aqueous environment. The silicone waterless emulsions coupled with the use of modulating agents can uniquely be adapted to protect and preserve active agents when stored in compatible sealed canisters with propellant A foamable composition, comprising an active agent has the following advantages:
1. The foamable composition provides a preferred solvent for active agents, particularly water-insoluble agents.
2. The inclusion of a propylene glycol and/or a PEG and a secondary polar solvent in the foamable composition facilitates a co-solvent effect, resulting increased concentrations of soluble active agent in the dosage form, thus facilitating enhanced skin penetration of the active agent. In many cases, increased penetration is positively correlated with improved clinical outcome. In certain case, attaining an increased drug penetration into the target site of action enables a decrease of treatment frequency, for example, from twice or three times daily to once daily.
3. Polyols and PEGs; and combinations of a polyol and/or PEG with a secondary polar solvent are known as skin penetration enhancers, thus, increasing drug residence in the target area and increasing clinical efficacy, as detailed above.
4. The fact that in certain embodiments the carrier, composition or foam contains no water or substantially no water and is hydrophilic minimizes the probability of degradation of water-sensitive active agents. Furthermore, as exemplified herein, a foam containing a polyol and/or PEG with no water at all can be formed in accordance with the composition and process. Such compositions ensure high stability of water sensitive active agents.
5. Combining the anti-infective effect of a hygroscopic composition, which acts through a dehydration mechanism, with an additional anti-infective agent, selected from the group of an antibiotic agent, an antibacterial agent, an antifungal agent, an agent that controls yeast, an antiviral agent and an antiparasitic agent, that acts through alternate mechanisms results in a synergistic effect and consequently higher success rate of the treatment.
6. The foamable polyol composition in contained in an impermeable pressurized packaging presentation is impermeable and thus, the active agent is not exposed to environmental degradation factors, such as light and oxidating agent during storage.

Thus, in a preferred embodiment, the composition includes at least one active agent being

a therapeutically effective concentration of an active agent; and
(a) a silicone,
(b) about 50% to about 98% of a solvent, selected from the group consisting of a propylene glycol or derivative and a polyethylene glycol or derivative and mixtures thereof;
(c) 0% to about 48% of a secondary polar solvent;
(d) about 0.2% to about 10% by weight of an Accommodating Agent or Complex;
(e) optionally about 0.01% to about 5% by weight of at least one polymeric agent;
(f) optionally a modifying agent; and
(g) a liquefied or compressed gas propellant at a concentration of about 3% to about 25% by weight of the total composition;
wherein the composition has some or partial resistance to creaming when subjected to centrifugation at 300 rpm for ten minutes. In a preferred embodiment it the rpm is 11000 and in a more preferred embodiment it is 3000.

In the context of combining a hygroscopic carrier and an anti-infective active agent, a pharmaceutical composition is provided, including:
(a) a silicone
(b) one or more hygroscopic substances at a sufficient concentration to provide an Aw value of the hygroscopic carrier of less than 0.9. The concentration of the hygroscopic substance in the composition can be designed to provide a Aw value selected from the ranges of (1) about 0.8 and about 0.9; (2) about 0.7 and about 0.8; and (3) less than about 0.7. One of the hygroscopic substances is preferably a propylene glycol or derivative or preferably a polyethylene glycol or derivative and mixtures thereof;
(c) about 0.2% to about 10% by weight of an Accommodating Agent or Complex;
(d) optionally, about 0.01% to about 5% by weight of at least one polymeric agent selected from a bioadhesive agent, a gelling agent, a film forming agent and a phase change agent;
(e) a therapeutically effective concentration of an anti-infective agent; and
(f) a liquefied or compressed gas propellant at a concentration of about 3% to about 25% by weight of the total composition.

An exemplary case for the inclusion of an anti-infective agent in a hygroscopic composition is provided herewith. Combining an antifungal agent in a hygroscopic composition results in an anti-infective effect on strains that are not supposed to be affected by the said antifungal agent. For example, terbinafine is know to be highly effective against dermatophite pathogens, but not against candida. In-vitro studies have revealed, however that terbinafine, dissolved in a hygroscopic carrier, effectively inhibited the spreading of candida albicans, while a control preparation, comprising the same concentration of terbinafine in an emulsion base was not effective. Thus, combining an antifungal agent in a hygroscopic composition results in an expansion of the spectrum of infective strains that can benefit form the therapy, and furthermore, it can render an improved effect of such a composition on mixed infections or in infections that are not accurately diagnosed.

Consequently, in another aspect, a silicone comprising pharmaceutical composition, which possesses an improved antifungal activity or that possesses an antifungal activity on an expanded spectrum of pathogens, is provided, including:
(a) a hygroscopic composition, comprising a hygroscopic substance at a sufficient concentration to provide an Aw value of the hygroscopic carrier of less than 0.9. The concentration of the hygroscopic substance in the composition can be designed to provide a Aw value selected from the ranges of (1) about 0.8 and about 0.9; (2) about 0.7 and about 0.8; and (3) less than about 0.7; and
(b) an anti-infective agent, selected from the group of an antibiotic agent, an antibacterial agent, an antifungal agent, an agent that controls yeast, an antiviral agent and an antiparasitic agent. Preferably, the anti-infective agent is an antifungal agent, and more preferably the anti-infective agent is terbinafine.

### Active Agents

Active agents can be used on their own or in combination with other agents. Suitable therapeutic agents include but are not limited to active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers. As is known to one skilled in the art, in some instances a specific active agent may have more than one activity, function or effect.

In an embodiment, the therapeutic agent is an active herbal extract. Suitable active herbal extracts include but are not limited to angelica, anise oil, astragali radix, azalea, benzyl acetate, birch tar oil, bornyl acetate, cacumen biotae, camphor, cantharidin, capsicum, cineole, cinnamon bark, cinnamon leaf, citronella, citroneliol, citronellyl acetate, citronellyl formate, eucalyptus, eugenyl acetate, flos carthami, fructus mori, garlic, geraniol, geranium, geranyl acetate, habanera, isobutyl angelicate, lavender, ledum latifolium, ledum palustre, lemongrass, limonene, linalool, linalyl acetate, methyl anthranilate, methyl cinnamate, mezereum, neem, nerol, neryl acetate, nettle root extract, oleum ricini, oregano, pinenes, .alpha.-pinene, .beta.-pinene, radix angelicae sinesis, radix paenoiae rubra, radix polygoni multiflori, radix rehmanniae, rhizoma pinelliae, rhizoma zingiberis recens, sabadilla, sage, sandalwood oil, saw palmetto extract, semen sesami nigrum, staphysagria, tea tree oil, terpene alcohols, terpene hydrocarbons, terpene esters, terpinene, terpineol, terpinyl acetate and derivatives, esters, salts and mixtures thereof. In an embodiment, the active agent is an acaricide. Suitable acaricides include but are not limited to amitraz, flumethrin, fluvalinate and derivatives, esters, salts and mixtures thereof.

In one or more embodiments, the active agent is encapsulated in particles, microparticles, nanoparticles, microcapsules, spheres, microsphres, nanocapsules, nanospheres, liposomes, niosomes, polymer matrix, nanocrystals or microsponges.

### Microsponges

By using specialized delivery systems like microsponges it may be possible to incorporate active ingredients within the microsponges, which active ingredients are released when the microsponges come into mechanical contact with the skin. This may be useful where the active agent would otherwise be unstable in the formulation but for its entrapment. Thus, in one or more embodiments, topical and body cavity foam formulations containing microsponges comprising one or more active ingredients are provided.

A “Microsponge” is an insoluble body that is insoluble in water and in the final formulations described herein that is capable of entrapping, incorporating or otherwise suspending an active agent within its structure or confines and releasing it upon a certain signal or stimulus, which can be a physical or a chemical or other signal.

Microsponges are macroporous beads, typically 10-25 microns in diameter, loaded with active agent. They consist of a copolymer, such as methyl methacrylate/glycol dimethacrylate crosspolymer. Depending on their chemical composition, microsponges may be biodegradable. Microsponges have the capacity to entrap a wide range of active ingredients such as emollients, fragrances, essential oils, sunscreens and anti-infective, etc. Examples of drugs that have been incorporated in microsponges include ibuprofen ketoprofen (non-steroidal anti-inflammatory agent), benzyl peroxide (an anti-acne agent), retinoids, such as retinoic acid and retinol, fluconazole (an antifungal agent)

Microsponges are used as a topical carrier system. When applied to the skin, the microsponges release the active ingredient on a time mode and also in response to other stimuli (rubbing, temperature, pH, etc). By delivering the active gradually to the skin, Microsponge-active agent formulations, for example, have excellent efficacy with minimal irritation.

In addition to their drug entrapping properties, microsponges are capable of absorbing skin secretions, therefore reducing oiliness and shine from the skin. Clinical studies demonstrated that the use of microsponges in a lotion reduced perceived oiliness on the skin by 50%, shine was reduced by 20% microspheres.

“Microsponges” are rigid, porous and spongelike round microscopic particles of cross-linked polymer beads (e.g., polystyrene or copolymers thereof), each defining a substantially non-collapsible pore network. The Microsponges can be loaded with an active ingredient and can provide a controlled time release of the active ingredient to skin or to a mucosal membrane upon application of the formulation. The slow release is intended to reduce irritation by the active. Microsponge® delivery technology was developed by Advanced Polymer Systems.

Microsponges have a size range in between 5 to 300 μm depending upon the degree of smoothness or after feel required for the end formulations and can reduce perceived oiliness.

Wide ranges of uses for microsponges incorporated in foamable compositions are suggested aiming to provide increased efficacy for delivery of active agents topically and in a body cavity with enhanced control, spreadability, safety, stability and improved aesthetic properties.

Microspheres can store an active agent until its release is triggered by application to the skin surface such as through rubbing and or higher than-ambient skin temperature. Microsponge do not pass through the skin but collect on the skin surface and slowly release the entrapped agent. The empty spheres are washed away with cleansing.

Microsponges may be incorporated in wide ranges of foam formulations. In one or more embodiments microsponges may be incorporated into the formulations exemplified and described herein. In an embodiment the amount of microsponges may be varied from about 1% to about 25% of the formulation, preferably about 5% to 15%.

In an embodiment any active agent suitable for loading in microsponges may be used, such as benzyl peroxide (BPO), tretinoin, hydroquinone, kotoprofen, retinol, fluconazole, ibuprofen, trolamine and the like.

In an embodiment the microsponges are loaded with one or more vitamins or with one or more flavonoids or combinations thereof. In another embodiment the vitamins and or flavonoids are fat soluble. In another embodiment they are water soluble. In an embodiment the vitamin is a retinoid, preferably a vitamin A, more preferably, retinoic acid or isoretinoic acid. In an embodiment the vitamin is preferably a vitamin D, a derivative or analogue thereof, more preferably calcipotriol or calcitriol or tacalcitol with or without a corticosteroid such as betmethasone or its esters (eg bmv), flucinonide, hydrocortisone or clobetasol proprionate.

In an embodiment, since retinoids and BPO can dry the skin, water soluble humectants, e.g., urea, sodium PCA, alpha-hydroxy acids, glycerin and other polyols may be added.

As can be noted from above and herein different types of active agents may be loaded into the microsponges. Accordingly the foam formulation selected in which to disperse the microsponges should be adapted so that the active agent remains substantially entrapped in the microsponges. In another embodiment the active agent is present both in the foam formulation and in the microsponges so that some of the active agent is available for immediate penetration on application of the foam and that other amounts of active agent are provided by slow or controlled release from the microsponges now sitting on the topical surface.

The methodology of loading microsponges with active agent and amounts that can be loaded are described in WO 01/85102, which is incorporated herein by way of reference. Where Drug Microsponge X % w/w is provided it refers to the microsponges including the trapped drug and any other ingredients incorporated when loading the microsponges.

In an embodiment, the therapeutic agent is an age spot and keratoses removing agent. Suitable age spot and keratoses removing agent include but are not limited to hydroxy acids, azelaic acid and other related dicarboxylic acids, retinoids, kojic acid, arbutin, nicotinic, ascorbic acid, hydroquinone and derivatives, esters, salts and mixtures thereof. Certain nonsteroidal anti-inflammatory agents, such as diclofenac are also useful for the treatment of keratoses.

In an embodiment, the therapeutic agent is an analgesic. Suitable analgesics include but are not limited to benzocaine, butamben picrate, dibucaine, dimethisoquin, dyclonine, lidocaine, pramoxine, tetracaine, salicylates and derivatives, esters, salts and mixtures thereof.

In an embodiment, the therapeutic agent is a local anesthetic. Suitable local anesthetics include but are not limited to benzocaine, benzyl alcohol, bupivacaine, butamben picrate, chlorprocaine, cocaine, dibucaine, dimethisoquin, dyclonine, etidocaine, hexylcaine, ketamine, lidocaine, mepivacaine, phenol, pramoxine, procaine, tetracaine, salicylates and derivatives, esters, salts and mixtures thereof.

In an embodiment, the therapeutic agent is an antiacne agent. Suitable antiacne agents include but are not limited to N-acetylcysteine, adapalene, azelaic acid, benzoyl peroxide, cholate, clindamycin, deoxycholate, erythromycin, flavinoids, glycolic acid, meclocycline, metronidazol, mupirocin, octopirox, phenoxy ethanol, phenoxy proponol, pyruvic acid, resorcinol, retinoic acid, salicylic acid, scymnol sulfate, sulfacetamide-sulfur, sulfur, tazarotene, tetracycline, tretinoin triclosan and derivatives, esters, salts and mixtures thereof.

In an embodiment, the therapeutic agent is an antiaging agent. Suitable antiaging agents include but are not limited to sulfur-containing D and L amino acids, alpha-hydroxy acids s, beta-hydroxy acids (e.g. salicylic acid), urea, hyaluronic acid, phytic acid, lipoic acid; lysophosphatidic acid, skin peel agents (e.g., phenol, resorcinol and the like), vitamin B3 compounds (e.g., niacinamide, nicotinic acid and nicotinic acid salts and esters, including non-vasodilating esters of nicotinic acid (such as tocopheryl nicotinate), nicotinyl amino acids, nicotinyl alcohol esters of carboxylic acids, nicotinic acid N-oxide and niacinamide N-oxide), vitamin B5 and retinoids (e.g., retinol, retinal, retinoic acid, retinyl acetate, retinyl palmitate, retinyl ascorbate) skin barrier forming agents, melatonin and derivatives, esters, salts and mixtures thereof.

### Dicarboxylic Acid and Esters Thereof

In an embodiment, the organic carrier comprises an ester of a dicarboxylic acid. In the context, a dicarboxylic acid is an organic material, having two carboxylic acid moieties on its carbon atom skeleton. They have the general molecular formula HOOC—(CH₂)ₙ—COOH.

In an embodiment, the dicarboxylic acid is a short-chain dicarboxylic acid. The simplest Short-chain dicarboxylic acid are oxalic acid (n=0), malonic acid (n=1) succinic acid (n=2) and glutaric acid (n=3).

Additional members of dicarboxylic acid group are derived from natural products or from synthesis, having “n” value from 4 up to 21. In one or more embodiments, the dicarboxylic acid is selected from the group consisting of adipic acid (hexanedioic acid; n 4), pimelic acid (heptanedioic acid; n=5), suberic acid (octanedioic acid; n=6), azelaic acid (nonanedioic acid; n=7), sebacic acid (decanedioic acid; n=8) and dodecanedioic acid (n=10).

In an additional embodiment, the dicarboxylic acid contains 10 to 32 carbon atoms in their carbon atom skeleton, such as brassylic acid (n=11), thapsic acid (n=14), 14-methylnonacosanedioic acid (C29) and 14,15-dimethyltriacontanedioic acid (C30).

The carbon atom skeleton of the dicarboxylic acid can be saturated or unsaturated, such as in the case of maleic acid and fumaric acid.

An ester of a dicarboxylic acid is a chemical compound produced by the reaction between a dicarboxylic acid and at least one alcohol, with the elimination of a molecule of water. The reaction of a dicarboxylic acid with one alcohol molecule results in a mono ester of said dicarboxylic acid, and the reaction of a dicarboxylic acid with two alcohol molecules results in a diester of the dicarboxylic acid.

The alcohol molecule, to be linked to the dicarboxylic acid, can be selected from the group of an alkyl an aryl alcohol. Exemplary alcohol, suitable include methyl alcohol, ethyl alcohol, propyl alcohol, isopropyl alcohol, butyl alcohol, isobutyl alcohol, t-butyl alcohol, pentyl alcohol, hexyl alcohol, octyl alcohol, decyl alcohol, capryl alcohol, phenol, benzyl alcohol and the like.

In one or more embodiments, the alcohol is a biologically active alcohol. In an embodiment, biologically active alcohol possesses keratolytic activities. Examples of keratolytically active alcohol suitable include oitho-, meta- and para-hydroxyalkylbenzoate, salicylic acid, ortho-, meta-, and para-dihydroxybenzene, ortho-, meta-, and para-hydroxytoluene, alpha-hydroxy acid, retinol, and derivatives thereof such as provided in U.S. Pat. No. 6,180,669. 22. In an embodiment, the biologically active alcohol is selected from the group consisting of steroidal hormones, steroidal anti-inflammatory agents, vitamin E and vitamin D, such as provided in U.S. Pat. Appl. 20040191196.

### Antibiotics

In an embodiment, the therapeutic agent is an antibiotic. The terms “antibiotic” as used herein shall include, but is not limited to, any substance being destructive to or inhibiting the growth of bacteria or any substance having the capacity to inhibit the growth of or to destroy bacteria.

In one or more embodiments, the antibiotic agent is selected from the group consisting of a beta-lactam antibiotic, an aminoglycoside, an ansa-type antibiotic, an anthraquinone, an azole, an antibiotic glycopeptide, a macrolide, an antibiotic nucleoside, an antibiotic peptide, an antibiotic polyene, an antibiotic polyether, an antibiotic quinolone, an antibiotic steroid, a sulfonamide, an antibiotic metal, an oxidizing agent, a periodate, a hypochlorite, a permanganate, a substance that release free radicals and/or active oxygen, a cationic antimicrobial agent, a quaternary ammonium compound, a biguanide, a triguanide, a bisbiguanide, a polymeric biguanide, and analogs, derivatives, salts, ions and complexes thereof.

Suitable antibiotics include but are not limited to amanfadine hydrochloride, amanfadine sulfate, amikacin, arnikacin sulfate, aminoglycosides, amoxicillin, ampicillin, ansamycins, bacitracin, beta-lactams, candicidin, capreomycin, carbenicillin, cephalexin, cephaloridine, cephalothin, cefazolin, cephapirin, cephradine, cephaloglycin, chloramphenicols chlorhexidine, chlorhexidine gluconate, chlorhexidine hydrochloride, chloroxine, chlorquinaldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erythromycin stearate, farnesol, floxacillin, gentamicin, gentamicin sulfate, gramicidin, griseofulvin, haloprogin, haloquinol, hexachlorophene, iminocyldline, iodate, iodine, iodochlorhydroxyquin, kanamycin, kanamycin sulfate, lincomycin, lineomycin, lineomycin hydrochloride, macrolides, meclocycline, methacycline, methacycline hydrochloride, methenamine, methenamine hippurate, methenamine mandelate, methicillin, metronidazole, miconazole, miconazole hydrochloride, microcrystalline and nanocrystalline particles of silver, copper, zinc, mercury, tin, lead, bismuth, cadmium and chromium, minocycline, minocycline hydrochloride, mupirocin, nafcillin, neomycin, neomycin sulfate, netilmicin, netilmicin sulfate, nitrofurazone, norfloxacin, nystatin, octopirox, oleandomycin, orcephalosporins, oxacillin, oxytetracycline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, spectinomycin, spiramycin, streptomycin, sulfonamide, tetracyclines, tetracycline, tobramycin, tobramycin sulfate, triclocarbon, triclosan, trimethoprim-sulfamethoxazole, tylosin, vancomycin, yrothricin and derivatives, esters, salts and mixtures thereof.

In one or more embodiments, the antibiotic agent is selected from the classes consisting of beta-lactam antibiotics, aminoglycosides, ansa-type antibiotics, anthraquinones, antibiotic azoles, antibiotic glycopeptides, macrolides, antibiotic nucleosides, antibiotic peptides, antibiotic polyenes, antibiotic polyethers, quinolones, antibiotic steroides, sulfonamides, tetracycline, dicarboxylic acids, antibiotic metals, oxidizing agents, substances that release free radicals and/or active oxygen, cationic antimicrobial agents, quaternary ammonium compounds, biguanides, triguanides, bisbiguanides and analogs and polymers thereof and naturally occurring antibiotic compounds.

Beta-lactam antibiotics include, but are not limited to, 2-(3-alanyl)clavam, 2-hydroxymethylclavam, 8-epi-thienamycin, acetyl-thienamycin, amoxicillin, amoxicillin sodium, amoxicillin trihydrate, amoxicillin-potassium clavulanate combination, ampicillin, ampicillin sodium, ampicillin trihydrate, ampicillin-sulbactam, apalcillin, aspoxicillin, azidocillin, azlocillin, aztreonam, bacampicillin, biapenem, carbenicillin, carbenicillin disodium, carfecillin, carindacillin, carpetimycin, cefacetril, cefaclor, cefadroxil, cefalexin, cefaloridine, cefalotin, cefamandole, cefamandole, cefapirin, cefatrizine, cefatrizine propylene glycol, cefazedone, cefazolin, cefbuperazone, cefcapene, cefcapene pivoxil hydrochloride, cefdinir, cefditoren, cefditoren pivoxil, cefepime, cefetamet, cefetamet pivoxil, cefixime, cefmenoxime, cefmetazole, cefminox, cefminox, cefmolexin, cefodizime, cefonicid, cefoperazone, ceforanide, cefoselis, cefotaxime, cefotetan, cefotiam, cefoxitin, cefozopran, cefpiramide, cefpirome, cefpodoxime, cefpodoxime proxetil, cefprozil, cefquinome, cefradine, cefroxadine, cefsulodin, ceftazidime, cefteram, cefteram pivoxil, ceftezole, ceftibuten, ceftizoxime, ceftriaxone, cefuroxime, cefuroxime axetil, cephalosporin, cephamycin, chitinovorin, ciclacillin, clavulanic acid, clometocillin, cloxacillin, cycloserine, deoxy pluracidomycin, dicloxacillin, dihydro pluracidomycin, epicillin, epithienamycin, ertapenem, faropenem, flomoxef, flucloxacillin, hetacillin, imipenem, lenampicillin, loracarbef, mecillinam, meropenem, metampicillin, meticillin, mezlocillin, moxalactam, nafcillin, northienamycin, oxacillin, panipenem, penamecillin, penicillin, phenethicillin, piperacillin, tazobactam, pivampicillin, pivcefalexin, pivmecillinam, pivmecillinam hydrochloride, pluracidomycin, propicillin, sarmoxicillin, sulbactam, sulbenicillin, talampicillin, temocillin, terconazole, thienamycin, ticarcillin and analogs, salts and derivatives thereof.

Aminoglycosides include, but are not limited to, 1,2′-N-DL-isoseryl-3′,4′-dideoxykanamycin B, 1,2′-N-DL-isoseryl-kanamycin B, 1,2′-N-[(S)-4-amino-2-hydroxybutyryl]-3′,4′-dideoxykanamycin B, 1,2′-N-[(S)-4-amino-2-hydroxybutyryl]-kanamycin B, 1-N-(2-Aminobutanesulfonyl) kanamycin A, 1-N-(2-aminoethanesulfonyl)3′,4′-dideoxyribostamycin, 1-N-(2-Aminoethanesulfonyl)3′-deoxyribostamycin, 1-N-(2-aminoethanesulfonyl)3′4′-dideoxykanamycin B, 1-N-(2-aminoethanesulfonyl)kanamycin A, 1-N-(2-aminoethanesulfonyl)kanamycin B, 1-N-(2-aminoethanesulfonyl)ribostamycin, 1-N-(2-aminopropanesulfonyl)3′-deoxykanamycin B, 1-N-(2-aminopropanesulfonyl)3′4′-dideoxykanamycin B, 1-N-(2-aminopropanesulfonyl)kanamyein A, 1-N-(2-aminopropanesulfonyl)kanamycin B, 1-N-(L-4-amino-2-hydroxy-butyryl)2,′3-dideoxy-2′-fluorokanamycin A, 1-N-(L-4-amino-2-hydroxy-propionyl)2,′3′-dideoxy-2′-fluorokanamycin A, 1-N-DL-3′,4′-dideoxy-isoserylkanamycin B, 1-N-DL-isoserylkanamycin, 1-N-DL-isoserylkanamycin B, 1-N-[L-(−)-(alpha-hydroxy-gamma-aminobutyryl)]-XK-62-2,2′,3′-dideoxy-2′-fluorokanamycin A,2-hydroxygentamycin A3,2-hydroxygentamycin B, 2-hydroxygentamycin B1, 2-hydroxygentamycin JI-20A, 2-hydroxygentamycin JI-20B, 3″-N-methyl-4″-C-methyl-3′,4′-dodeoxy kanamycin A, 3″-N-methyl-4″-C-methyl-3′,4′-dodeoxy kanamycin B, 3″-N-methyl-4-C-methyl-3′,4′-dodeoxy-6′-methyl kanamycin B, 3′,4′-Dideoxy-3′-eno-ribostamycin,3′),4′-dideoxyneamine,3′,4′-dideoxyribostamycin, 3′)-deoxy-6′-N-methyl-kanamycin B, 3′-deoxyneamine, 3′-deoxyribostamycin, 3′-oxysaccharocin, 3,3′-nepotrehalosadiamine, 3-demethoxy-2″-N-formimidoylistamycin B disulfate tetrahydrate, 3-demethoxyistamycin B,3-O-demethyl-2-N-formimidoylistamycin B, 3-O-demethylistamycin B, 3-trehalosamine,4″,6″-dideoxydibekacin, 4-N-glycyl-KA-6606VI, 5″-Amino-3′,4′,5″-trideoxy-butirosin A, 6″-deoxydibekacin,6′-epifortimicin A, 6-deoxy-neomycin (structure 6-deoxy-neomycin B),6-deoxy-neomycin B, 6-deoxy-neomycin C, 6-deoxy-paromomycin, acmimycin, AHB-3′,4′-dideoxyribostamycin, AHB-3′-deoxykanamycin B, AHB-3′-deoxyneamine,AHB-3′-deoxyribostamycin, AHB-4″-6″-dideoxydibekacin, AHB-6″-deoxydibekacin, AHB-dideoxyneamine, AHB-kanamycin B, AHB-methyl-3′-deoxykanamycin B, amikacin, amikacin sulfate, apramycin, arbekacin, astromicin, astromicin sulfate, bekanamycin, bluensomycin, boholmycin, butirosin, butirosin B, catenulin, coumamidine gamma1, coumarnmidine gamma2,D,L-1-N-(alpha-hydroxy-beta-aminopropionyl)-XK-62-2, dactimicin,de-O-methyl-4-N-glycyl-KA-6606VI, de-O-methyl-KA-66061, de-O-methyl-KA-7038I, destomycin A, destomycin B, di-N6′,O3-demethylistamycin A, dibekacin, dibekacin sulfate, dihydrostreptomycin, dihydrostreptomycin sulfate, epi-formamidoylglycidylfortimicin B, epihygromycin, formimidoyl-istamycin A, formimidoyl-istamycin B, fortimicin B, fortimicin C, fortimicin D, fortimicin KE, fortimicin KF, fortimicin KG, fortimicin KG1 (stereoisomer KG1/KG2), fortimicin KG2 (stereoisomer KG1/KG2), fortimicin KG3, framycetin, framycetin sulphate, gentamicin, gentamycin sulfate, globeomycin, hybrimycin A1, hybrimycin A2, hybrimycin B1, hybrimycin B2, hybrimycin C1, hybrimycin C2, hydroxystreptomycin, hygromycin, hygromycin B, isepamicin, isepamicin sulfate, istamycin, kanamycin, kanamycin sulphate, kasugamycin, lividomycin, marcomycin, micronomicin, micronomicin sulfate, mutamicin, myomycin, N-demethyl-7-O-demethylcelesticetin, demethylcelesticetin, methanesulfonic acid derivative of istamycin, nebramycin, nebramycin, neomycin, netilmicin, oligostatin, paromomycin, quintomycin, ribostamycin, saccharocin, seldomycin, sisomicin, sorbistin, spectinomycin, streptomycin, tobramnycin, trehalosmaine, trestatin, validamycin, verdamycin, xylostasin, zygomycin and analogs, salts and derivatives thereof.

Ansa-type antibiotics include, but are not limited to, 21-hydroxy-25-demethyl-25-methylthioprotostreptovaricin, 3-methylthiorifamycin, ansamitocin, atropisostreptovaricin, awamycin, halomicin, maytansine, naphthomycin, rifabutin, rifamide, rifampicin, rifamycin, rifapentine, rifaximin, rubradirin, streptovaricin, tolypomycin and analogs, salts and derivatives thereof.

Antibiotic anthraquinones include, but are not limited to, auramycin, cinerubin, ditrisarubicin, ditrisarubicin C, figaroic acid fragilomycin, minomycin, rabelomycin, rudolfomycin, sulfurmycin and analogs, salts and derivatives thereof.

Antibiotic azoles include, but are not limited to, azanidazole, bifonazole, butoconazol, chlormidazole, chlormidazole hydrochloride, cloconazole, cloconazole monohydrochloride, clotrimazol, dimetridazole, econazole, econazole nitrate, enilconazole, fenticonazole, fenticonazole nitrate, fezatione, fluconazole, flutrimazole, isoconazole, isoconazole nitrate, itraconazole, ketoconazole, lanoconazole, metronidazole, metronidazole benzoate, miconazole, miconazole nitrate, neticonazole, nimorazole, niridazole, omoconazol, ornidazole, oxiconazole, oxiconazole nitrate, propenidazole, secnidazol, sertaconazole, sertaconazole nitrate, sulconazole, sulconazole nitrate, timidazole, tioconazole, voriconazol and analogs, salts and derivatives thereof.

Antibiotic glycopeptides include, but are not limited to, acanthomycin, actaplanin, avoparcin, balhimycin, bleomycin B (copper bleomycin), chloroorienticin, chloropolysporin, demethylvancomnycin, enduracidin, galacardin, guanidylfungin, hachimycin, demethylvancomycin, N-nonanoyl-teicoplanin, phlenomycin, platomycin, ristocetin, staphylocidin, talisomycin, teicoplanin, vancomycin, victomycin, xylocandin, zorbamycin and analogs, salts and derivatives thereof.

Macrolides include, but are not limited to, acetylleucomycin, acetylkitasamycin, angolamycin, azithromycin, bafilomycin, brefeldin, carbomycin, chalcomycin, cirramycin, clarithromycin, concanamycin, deisovaleryl-niddamycin, demycinosyl-mycinamycin, Di-O-methyltiacumicidin, dirithromycin, erythromycin, erythromycin estolate, erythromycin ethyl succinate, erythromycin lactobionate, erythromycin stearate, flurithromycin, focusin, foromacidin, haterumalide, haterumalide, josamycin, josamycin ropionate, juvenimycin, juvenimycin, kitasamycin, ketotiacumicin, lankavacidin, lankavamycin, leucomycin, machecin, maridomycin, megalomicin, methylleucomycin, methymycin, midecamycin, miocamycin, mycaminosyltylactone, mycinomycin, neutramycin, niddamycin, nonactin, oleandomycin, phenylacetyldeltamycin, pamamycin, picromycin, rokitainycin, rosaramicin, roxithromycin, sedecamycin, shincomycin, spiramycin, swalpamycin, tacrolimus, telithromycin, tiacumicin, tilmicosin, treponemycin, troleandomycin, tylosin, venturicidin and analogs, salts and derivatives thereof

Antibiotic nucleosides include, but are not limited to, amicetin, angustmycin, azathymidine, blasticidin S, epiroprim, flucytosine, gougerotin, mildiomycin, nikkomycin, nucleocidin, oxanosine, oxanosine, puromycin, pyrazomycin, showdomycin, sineftingin, sparsogenin, spicamycin, tunicamycin, uracil polyoxin, vengicide and analogs, salts and derivatives thereof.

Antibiotic peptides include, but are not limited to, actinomycin, aculeacin, alazopeptin, amfomycin, amythiamycin, antifungal from Zalerion arboricola, antrimycin, apid, apidaecin, aspaltocin, auromomycin, bacileucin, bacillomycin, bacillopeptin, bacitracin, bagacidin, berninamycin, beta-alanyl-L-tyrosine, bottromycin, capreomycin, caspofingine, cepacidine, cerexin, cilofungin, circulin, colistin, cyclodepsipeptide, cytophagin, dactinomycin, daptomycin, decapeptide, desoxymulundocandin, echanomycin, echinocandin B, echinomycin, ecomycin, enniatin, etamycin, fabatin, ferrimycin, ferrimycin, ficellomycin, fluoronocathiacin, fusaricidin, gardimycin, gatavalin, globopeptin, glyphomycin, gramicidin, herbicolin, iomycin, iturin, iyomycin, izupeptin, janiemycin, janthinocin, jolipeptin, katanosin, killertoxin, lipopeptide antibiotic, lipopeptide from Zalerion sp., lysobactin, lysozyme, macromomycin, magainin, melittin, mersacidin, mikamycin, mureidomycin, mycoplanecin, mycosubtilin, neopeptifluorin, neoviridogrisein, netropsin, nisin, nocathiacin, nocathiacin 6-deoxyglycoside, nosiheptide, octapeptin, pacidamycin, pentadecapeptide, peptifluorin, permetin, phytoactin, phytostreptin, planothiocin, plusbacin, polcillin, polymyxin antibiotic complex, polymyxin B, polymyxin B1, polymyxin F, preneocarzinostatin, quinomycin, quinupristin-dalfopristin, safracin, salmycin, salmycin, salmycin, sandramycin, saramycetin, siomycin, sperabillin, sporamycin, a streptomyces compound, subtilin, teicoplanin aglycone, telomycin, thermothiocin, thiopeptin, thiostrepton, tridecaptin, tsushimycin, tuberactinomycin, tuberactinomycin, tyrothricin, valinomycin, viomycin, virginiamycin, zervacin and analogs, salts and derivatives thereof.

In one or more embodiments, the antibiotic peptide is a naturally-occurring peptide that possesses an antibacterial and/or an antifungal activity. Such peptide can be obtained from a herbal or a vertebrate source.

Polyenes include, but are not limited to, amphotericin, amphotericin, aureofungin, ayfactin, azalomycin, blasticidin, candicidin, candicidin methyl ester, candimycin, candimvcin methyl ester, chinopricin, filipin, flavofungin, fradicin, hamycin, hydropricin, levorin, lucensomycin, lucknomycin, mediocidin, mediocidin methyl ester, mepartricin, methylamphotericin, natamycin, niphimycin, nystatin, nystatin methyl ester, oxypricin, partricin, pentamycin, perimycin, pimaricin, primycin, proticin, rimocidin, sistomycosin, sorangicin, trichomycin and analogs, salts and derivatives thereof.

Polyethers include, but are not limited to, 20-deoxy-epi-narasin, 20-deoxysalinomycin, carriomycin, dianemycin, dihydrolonomycin, etheromycin, ionomycin, iso-lasalocid, lasalocid, lenoremycin, lonomycin, lysocellin, monensin, narasin, oxolonomycin, a polycyclic ether antibiotic, salinomycin and analogs, salts and derivatives thereof.

Quinolones include, but are not limited to, an alkyl-methylendioxy-4(1H)-oxocinnoline-3-carboxylic acid, alatrofloxacin, cinoxacin, ciprofloxacin, ciprofloxacin hydrochloride, danofloxacin, dennofongin A, enoxacin, enrofloxacin, fleroxacin, flumequine, gatifloxacin, gemifloxacin, grepafloxacin, levofloxacin, lomefloxacin, lomefloxacin, hydrochloride, miloxacin, moxifloxacin, nadifloxacin, nalidixic acid, nifuroquine, norfloxacin, ofloxacin, orbifloxacin, oxolinic acid, pazufloxacine, pefloxacin, pefloxacin mesylate, pipemidic acid, piromidic acid, premafloxacin, rosoxacin, rufloxacin, sparfloxacin, temafloxacin, tosufloxacin, trovafloxacin and analogs, salts and derivatives thereof.

Antibiotic steroids include, but are not limited to, aminosterol, ascosteroside, cladosporide A, dihydrofusidic acid, dehydro-dihydrofusidic acid, dehydrofusidic acid, fusidic acid, squalamine and analogs, salts and derivatives thereof.

Sulfonamides include, but are not limited to, chloramine, dapsone, mafenide, phthalylsulfathiazole, succinylsulfathiazole, sulfabenzamide, sulfacetamide, sulfachlorpyridazine, sulfadiazine, sulfadiazine silver, sulfadicramide, sulfadimethoxine, sulfadoxine, sulfaguanidine, sulfalene, sulfamazone, sulfamerazine, sulfamethazine, sulfamethizole, sulfamethoxazole, sulfamethoxypyridazine, sulfamonomethoxine, sulfamoxol, sulfanilamide, sulfaperine, sulfaphenazol, sulfapyridine, sulfaquinoxaline, sulfasuccinamide, sulfathiazole, sulfathiourea, sulfatolamide, sulfatriazin, sulfisomidine, sulfisoxazole, sulfisoxazole acetyl, sulfacarbamide and analogs, salts and derivatives thereof.

Tetracyclines include, but are not limited to, dihydrosteffimycin, demethyltetracycline, aclacinomycin, akrobomycin, baumycin, bromotetracycline, cetocyclin, chlortetracycline, clomocycline, daunorubicin, demeclocycline, doxorubicin, doxorubicin hydrochloride, doxycycline, lymecyclin, marcellomycin, meclocycline, meclocycline suliosalicylate, methacycline, minocycline, minocycline hydrochloride, musettamycin, oxytetracycline, rhodirubin, rolitetracycline, rubomycin, serirubicin, steffimycin, tetracycline and analogs, salts and derivatives thereof.

Dicarboxylic acids, having between about 6 and about 14 carbon atoms in their carbon atom skeleton are particularly useful in the treatment of disorders of the skin and mucosal membranes that involve microbial. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid. Thus, in one or more embodiments, dicarboxylic acids, having between about 6 and about 14 carbon atoms in their carbon atom skeleton, as well as their salts and derivatives (e.g., esters, amides, mercapto-derivatives, anhydraides), are useful immunomodulators in the treatment of disorders of the skin and mucosal membranes that involve inflammation. Azelaic acid and its salts and derivatives are preferred. It has antibacterial effects on both aerobic and anaerobic organisms, particularly propionibacterium acnes and staphylococcus epidermidis, normalizes keratinization, and has a cytotoxic effect on malignant or hyperactive melanocytes. In a preferred embodiment, the dicarboxylic acid is azelaic acid in a concentration greater than 10%. Preferably, the concentration of azelaic acid is between about 10% and about 25%. In such concentrates, azelaic acid is suitable for the treatment of a variety of skin disorders, such as acne, rosacea and hyperpigmentation.

In one or more embodiments, the antibiotic agent is an antibiotic metal. A number of metals ions been shown to possess antibiotic activity, including silver, copper, zinc, mercury, tin, lead, bismutin, cadmium, chromium and ions thereof. It has been theorized that these antibiotic metal ions exert their effects by disrupting respiration and electron transport systems upon absorption into bacterial or fungal cells. Anti-microbial metal ions of silver, copper, zinc, and gold, in particular, are considered safe for in vivo use. Anti-microbial silver and silver ions are particularly useful due to the fact that they are not substantially absorbed into the body.

Thus, in one or more embodiment, the antibiotic metal consists of an elemental metal, selected from the group consisting of silver, copper, zinc, mercury, tin, lead, bismutin, cadmium, chromium and gold, which is suspended in the composition as particles, microparticles, nanoparticles or colloidal particles. The antibiotic metal can further be intercalated in a chelating substrate.

In further embodiments, the antibiotic metal is ionic. The ionic antibiotic metal can be presented as an inorganic or organic salt (coupled with a counterion), an organometallic complex or an intercalate. Non binding examples of counter inorganic and organic ions are sulfadiazine, acetate, benzoate, carbonate, iodate, iodide, lactate, laurate, nitrate, oxide, palmitate, a negatively charged protein. In preferred embodiments, the antibiotic metal salt is a silver salt, such as silver acetate, silver benzoate, silver carbonate, silver iodate, silver iodide, silver lactate, silver laurate, silver nitrate, silver oxide, silver palmitate, silver protein, and silver sulfadiazine.

In one or more embodiments, the antibiotic metal or metal ion is embedded into a substrate, such as a polymer, a mineral (such as zeolite, clay and silica).

Oxidizing agents and substances that release free radicals and/or active oxygen. In one or more embodiments, the antibiotic agent comprises strong oxidants and free radical liberating compounds, such as oxygen, hydrogen peroxide, benzoyl peroxide, elemental halogen species, as well as oxygenated halogen species, bleaching agents (e.g., sodium, calcium or magnesium hypochloride and the like), perchlorite species, iodine, iodate, and benzoyl peroxide. Organic oxidizing agents are also included in the definition of “oxidizing agent”, such as quinones. Such agents possess a potent broad spectrum activity

In one or more embodiments the antibiotic agent is a cationic antimicrobial agent. The outermost surface of bacterial cells universally carries a net negative charge, making them sensitive to cationic substances. Examples of cationic antibiotic agents include: quaternary ammonium compounds (QAC's)—QAC's are surfactants, generally containing one quaternary nitrogen associated with at least one major hydrophobic moiety; alkyltrimethyl ammonium bromides are mixtures of where the alkyl group is between 8 and 18 carbons long, such as cetrimide (tetradecyltrimethylammonium bromide); benzalkonium chloride, which is a mixture of n-alkyldimethylbenzyl ammonium chloride where the alkyl groups (the hydrophobic moiety) can be of variable length; dialkylmethyl ammonium halides; dialkylbenzyl ammonium halides; and QAC dimmers, which bear bi-polar positive charges in conjunction with interstitial hydrophobic regions.

In one or more embodiments, the antibiotic agent is selected from the group of biguanides, triguanides, bisbiguanides and analogs thereof.

Guanides, biguanides, biguanidines and triguanides are unsaturated nitrogen containing molecules that readily obtain one or more positive charges, which make them effective antimicrobial agents. The basic structures a guanide, a biguanide, a biguanidine and a triguanide are provided below.

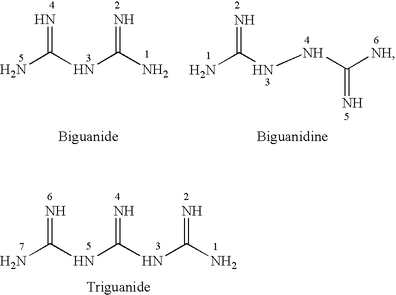

In one or more preferred embodiments, the guanide, biguanide, biguanidine or triguanide, provide bi-polar configurations of cationic and hydrophobic domains within a single molecule.

Examples of guanides, biguanides, biguanidines and triguanides that are currently been used as antibacterial agents include chlorhexidine and chlorohexidine salts, analogs and derivatives, such as chlorhexidine acetate, chlorhexidine gluconate and chlorhexidine hydrochloride, picloxydine, alexidine and polihexanide. Other examples of guanides, biguanides, biguanidines and triguanides that can conceivably be used are chlorproguanil hydrochloride, proguanil hydrochloride (currently used as antimalarial agents), metformin hydrochloride, phenformin and buformin hydrochloride (currently used as antidiabetic agents).

In one or more embodiments, the cationic antimicrobial agent is a polymer.

Cationic antimicrobial polymers include, for example, guanide polymers, biguanide polymers, or polymers having side chains containing biguanide moieties or other cationic functional groups, such as benzalkonium groups or quarternium groups (e.g., quaternary amine groups). It is understood that the term “polymer” as used herein includes any organic material comprising three or more repeating units, and includes oligomers, polymers, copolymers, block copolymers, terpolymers, etc. The polymer backbone may be, for example a polyethylene, ploypropylene or polysilane polymer.

In one or more embodiments, the cationic antimicrobial polymer is a polymeric biguanide compound. When applied to a substrate, such a polymer is known to fomm a barrier film that can engage and disrupt a microorganism. An exemplary polymeric biguanide compound is polyhexamethylene biguanide (PHMB) salts. Other exemplary biguanide polymers include, but are not limited to poly(hexamethylenebiguanide), poly(hexamethylenebiguanide) hydrochloride, poly(hexamethylenebiguanide) gluconate, poly(hexamethylenebiguanide) stearate, or a derivative thereof. In one or more embodiments, the antimicrobial material is substantially water-insoluble.

Yet, in one or more embodiment, the antibiotic is a non-classified antibiotic agent, including, without limitation, aabomycin, acetomycin, acetoxycycloheximide, acetylnanaomycin, an actinoplanes sp. Compound, actinopyrone, aflastatin, albacarcin, albacarcin, albofungin, albofungin, alisamycin, alpha-R,S-methoxycarbonylbenzylmonate, altromycin, amicetin, amycin, amycin demanoyl compound, amycine, amycomycin, anandimycin, anisomycin, anthramycin, anti-syphilis imune substance, anti-tuberculosis imune substance, antibiotic from Eschericia coli, antibiotics from Streptomyces refuineus, anticapsin, antimycin, aplasmomycin, aranorosin, aranorosinol, arugomycin, ascofuranone, ascomycin, ascosin, Aspergillus flavus antibiotic, asukamycin, aurantinin, an Aureolic acid antibiotic substance, aurodox, avilamycin, azidamfenicol, azidimycin, bacillaene, a Bacillus larvae antibiotic, bactobolin, benanomycin, benzanthrin, benzylmonate, bicozamycin, bravomicin, brodimoprim, butalactin, calcimycin, calvatic acid, candiplanecin, carumonam, carzinophilin, celesticetin, cepacin, cerulenin, cervinomycin, chartreusin, chloramphenicol, chloramphenicol palmitate, chloramphenicol succinate sodium, chlorflavonin, chlorobiocin, chlorocarcin, chromomycin, ciclopirox, ciclopirox olamine, citreamicin, cladospoiin, clazamycin, clecannycin, clindamycin, coliformin, collinomycin, copiamycin, corallopyronin, corynecandin, coumennycin, culpin, cuprimyxin, cyclamidomycin, cycloheximide, dactylomycin, danomycin, danubomycin, delaminomycin, demethoxyrapamycin, demethylscytophycin, dennadin, desdamethine, dexylosyl-benanomycin, pseudoaglycone, dihydromocimycin, dihydronancimycin, diumycin, dnacin, dorrigocin, dynemycin, dynemycin triacetate, ecteinascidin, efrotomycin, endomycin, ensanchomycin, equisetin, ericamycin, esperamicin, ethylmonate, everninomicin, feldamycin, flambamycin, flavensomycin, florfenicol, fluvomycin, fosfomycin, fosfonochliorin, fredericamycin, frenolicin, fumagillin, fumifungin, funginon, fusacandin, fusafungin, gelbecidine, glidobactin, gtahamimycin, granaticin, griseofulvin, griseoviridin, grisonomycin, hayumicin, hayumicin, hazymicin, hedamycin, heneicomycin, heptelicid acid, holomycin, humidin, isohematinic acid, karnatakin, kazusamycin, kristenin, L-dihydrophenylalanine, a L-isoleucyl-L-2-amino-4-(4′-amino-2′,5′-cyclohexadienyl) derivative, lanomycin, leinamycin, leptomycin, libanomycin, lincomycin, lomofungin, lysolipin, magnesidin, manumycin, melanomycin, methoxycarbonylmethylmonate, methoxycarbonylethylmonate, methoxycarbonylphenylmonate, methyl pseudomonate, methylmonate, microcin, mitomalcin, mocimycin, moenomycin, monoacetyl cladosporin, monomethyl cladosporin, mupirocin, mupirocin calcium, mycobacidin, myriocin, myxopyronin, pseudoaglycone, nanaomycin, nancimycin, nargenicin, neocarcinostatin, neoenactin, neothramycin, nifurtoinol, nocardicin, nogalamycin, novobiocin, octylmonate, olivomycin, orthosomycin, oudemansin, oxirapentyn, oxoglaucine methiodide, pactacin, pactamycin, papulacandin, paulomycin, phaeoramularia fungicide, phenelfamycin, phenyl, cerulenin, phenylmonate, pholipomycin, pirlimycin, pleuromutilin, a polylactone derivative, polynitroxin, polyoxin, porfiromycin, pradimicin, prenomycin, prop-2-enylmonate, protomycin, pseudomonas antibiotic, pseudomonic acid, purpuromycin, pyrinodemin, pyrrolnitrin, pyrrolomycin, amino, chloro pentenedioic acid, rapamycin, rebeccamycin, resistomycin, reuterin, reveromycin, rhizocticin, roridin, rubiflavin, naphthyridinomycin, saframycin, saphenamycin, sarkomycin, sarkomycin, sclopularin, selenomycin, siccanin, spartanamicin, spectinomycin, spongistatin, stravidin, streptolydigin, streptomyces arenae antibiotic complex, streptonigrin, streptothricins, streptovitacin, streptozotocine, a strobilurin derivative, stubomycin, sulfamethoxazol-trimethoprim, sakamycin, tejeramycin, terpentecin, tetrocarcin, thermorubin, thermozymocidin, thiamphenicol, thioaurin, thiolutin, thiomarinol, thiomarinol, tirandamycin, tolytoxin, trichodermin, trienomycin, trimethoprim, trioxacarcin, tyrissamycin, umbrinomycin, unphenelfamycin, urauchimycin, usnic acid, uredolysin, variotin, vermisporin, verrucarin and analogs, salts and derivatives thereof

In one or more embodiments, the antibiotic agent is a naturally occurring antibiotic compound. As used herein, the term “naturally-occurring antibiotic agent” includes all antibiotic that are obtained, derived or extracted from plant or vertebrate sources. Non-limiting examples of families of naturally-occurring antibiotic agents include phenol, resorcinol, antibiotic aminoglycosides, anamycin, quinines, anthraquinones, antibiotic glycopeptides, azoles, macrolides, avilamycin, agropyrene, cnicin, aucubin antibioticsaponin fractions, berberine (isoquinoline alkaloid), arctiopicrin (sesquiterpene lactone), lupulone, humulone (bitter acids), allicin, hyperforin, echinacoside, coniosetin, tetramic acid, imanine and novoimanine.

Ciclopirox and ciclopiroxolamine possess fungicidal, fungistatic and sporicidal activity. They are active against a broad spectrum of dermatophytes, yeasts, moulds and other fungi, such as trichoplilyton species, microsporum species, epidermoplyton species and yeasts (candida albicans, candida glabrata, other candida species and cryptococcus neoformans). Some aspergillus species are sensitive to ciclopirox as are some penicillium. Likewise, ciclopirox is effective against many gram-positive and gram-negative bacteria (e.g., escherichia coli, proteus mirabilis, pseudomonas aeruginosa, staphylococcus and streptococcus species), as well as mycoplasma species, trichomonas vaginalis and actinomyces.

Plant oils and extracts which contain antibiotic agents are also useful. Non limiting examples of plants that contain agents include thyme, perilla, lavender, tea tree, terfezia clayeryi, Micromonospora, putterlickia verrucosa, putterlickia pyracantha, putterlickia retrospinosa, Maytenus ilicifolia, maytenus evonymoides, maytenus aquifolia, faenia interjecta, cordyceps sinensis, couchgrass, holy thistle, plantain, burdock, hops, echinacea, buchu, chaparral, myrrh, red clover and yellow dock, garlic and St. John's wort.

Mixtures of these antibiotic agents may also be employed.

In an embodiment, the therapeutic agent is an antidandruff agent. Suitable antidandruff agents include but are not limited to aminexil, benzalkonium chloride, benzethonium chloride, 3-bromo-1-chloro-5,5-dimethyl-hydantoin, chloramine B, chloramine T, chlorhexidine, N-chlorosuccinimide, climbazole-,1,3-dibromo-5,5-dimethylhydantoin, 1,3-dichloro-5,5-dimethyl-hydantoin, betulinic acid, betulonic acid, celastrol, crataegolic acid, cromakalin, cyproterone acetate, dutasteride, finesteride, ibuprofen, ketoconozole, oleanolic acid, phenyloin, picrotone olamine, salicylic acid, selenium sulphides, triclosan, triodothyronine, ursolic acid, zinc gluconate, zinc omadine, zinc pyrithione and derivatives, esters, salts and mixtures thereof.

In an embodiment, the therapeutic agent is an antihistamine. Suitable antihistamines include but are not limited to chlorcyclizine, diphenhydramine, mepyramine, methapyrilene, tripelennamine and derivatives, esters, salts and mixtures thereof.

### Antifungal

In an embodiment, the therapeutic agent is an antimycotic, also termed antifungal agent. The terms “antimycotic” and “antifungal” as used herein include, but is not limited to, any substance being destructive to or inhibiting the growth of fungi and yeast or any substance having the capacity to inhibit the growth of or to destroy fungi and/or yeast.

In one or more embodiments, the antifungal agent is an agent that is useful in the treatment of a superficial fungal infection of the skin, dermatophytosis, microsporum, trichophyton and epidennophyton infections, candidiasis, oral candidiasis (thrush), candidiasis of the skin and genital mucous membrane, candida paronychia, which inflicts the nail and nail bed and genital and vaginal candida, which inflict genitalia and the vagina. Thus, in one or more embodiments, the antifungal agent is selected from the group including but not limited to, azoles, diazoles, triazoles, miconazole, fluconazole, ketoconazole, clotrimazole, itraconazole, Climbazole, griseofulvin, ciclopirox, ciclopirox-olamine, amorolfine, terbinafine, Amphotericin B, potassium iodide and flucytosine (5FC) at a therapeutically effective concentration.

Azoles are pharmaceutically active compounds that are unsaturated five member ring heterocyclic compound, wherein one, two or three members of the ring are nitrogen atoms, as exemplified in a non-limiting way and illustrated in the following schemes:

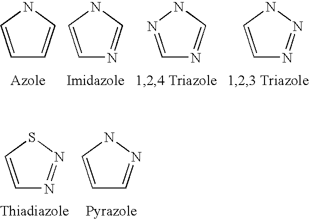

The azole is a compound including an unsaturated five member ring heterocyclic compound, wherein one, two or three members of the ring are nitrogen atoms.

Examples of therapeutic azoles include, but are not limited to, azanidazole, bifonazole, butoconazol, chlormidazole, climbazole, cloconazole, clotrimazole, dimetridazole, econazole, enilconazole, fenticonazole, fezatione, fluconazole, flutrimazole, isoconazole, itraconazole, ketoconazole, lanoconazole, metronidazole, metronidazole benzoate, miconazole, neticonazole, nimorazole, niridazole, omoconazol, omidazole, oxiconazole, posaconazole, propenidazole, ravuconazole, secnidazol, sertaconazole, sulconazole, thiabendazole, timidazole, tioconazole, voriconazol and salts and derivatives thereof. Such azoles are mainly used as antifungal agents, yet several of them also possess other therapeutic benefits, such as anti-inflammatory, antibacterial and antiviral effects.

Additional non-limiting exemplary classes of azoles include oxazoles, thiazoles, thiadiazoles and thiatriazoles, benzimidazoles, and salts and derivatives thereof.

In an embodiment, the azole is metronidazole.

In one or more embodiments, the antifungal agent is a peptide.

In certain embodiments, antifungal agent is a naturally-occurring peptide that possesses an antibacterial and/or an antifungal activity. Such peptide can be obtained from a herbal or a vertebrate source.

In an embodiment, the antifungal agent is a polyene. Polyene compounds are so named because of the alternating conjugated double bonds that constitute a part of their macrolide ring structure. Polyenes include, but are not limited to, amphotericin, aureofungin, ayfactin, azalomycin, blasticidin, candicidin, candicidin methyl ester, candimycin, candimycin methyl ester, chinopricin, filipin, flavofungin, fradicin, hamycin, hydropricin, levorin, lucensomycin, lucknomycin, mediocidin, mediocidin methyl ester, mepartricin, methylamphotericin, natamycin, niphimycin, nystatin, oxypricin, partricin, pentamycin, perimycin, pimaricin, primycin, proticin, rimocidin, sistomycosin, sorangicin, trichomycin and analogs, salts and derivatives thereof.

In an embodiment, the antifungal agent is a pyrimidine, such as Flucytosine.

In an embodiment, the antifungal agent is an allylamine, such as terbinafine and naftifine.

In an embodiment, the antifungal agent is a morpholine derivative, such as amorolfine.

In an embodiment, the antifungal agent is selected from the group consisting of Ciclopirox, ciclopiroxolmine, griseofulvin.

In an embodiment, the antifungal agent is a Thiocarbamate, such as tolnaftate. In an embodiment, the antifungal agent is a Sulfonamide, such as Mafenide and Dapsone.

In an embodiment, the antifungal agent consists of a plant oil or a plant extract possessing antifungal activity; or a plant oil or extract which contains antifungal agents. Non-limiting examples of plants containing agents include, but are not limited to, anise, basil, bergemont, burdock, buchu, chaparral, camphor, cardamom, carrot, canola, cassia, catnip, cedarwood, citronella, clove, couchgrass, cypress, echinacea, eucalyptus, faenia interjecta, garlic, ginger, grapefruit, holy thistle, hops, hyssop, jasmine, jojova, lavender, lavandin, lemon, lime, mandarin, marigold, marjoram, maytenus ilicifolia, maytenus evonymoides, maytenus aquifolia, micromonospora, myrrh, neroli, nutmeg, orange, ordyceps sinensis, peppermint, perilla, petitgrain, plantain, putterlickia verrucosa, putterlickia pyracantha, putterlickia retrospinosa, rosemary, sage, spearmint, star anise, St. John's wort, red clover, tangerine, tea tree, terfezia clayeryi, thyme vanilla, verbena, white clover and yellow dock.

In an embodiment, the antifungal agent is an anti-microbial metal. A number of metals ions been shown to possess antibiotic activity, including silver, copper, zinc, mercury, tin, lead, bismutin, cadmium, chromium and ions thereof. It has been theorized that these anti-microbial metal ions exert their effects by disrupting respiration and electron transport systems upon absorption into bacterial or fungal cells. Anti-microbial metal ions of silver, copper, zinc, and gold, in particular, are considered safe for in vivo use. Anti-microbial silver and silver ions are particularly useful due to the fact that they are not substantially absorbed into the body.

Thus, in one or more embodiment, the anti-microbial metal consists of an elemental metal, selected from the group consisting of silver, copper, zinc, mercury, tin, lead, bismutin, cadmium, chromium and gold, which is suspended in the composition as particles, microparticles, nanoparticles or colloidal particles. The anti-microbial metal can further be intercalated in a chelating substrate.

In further embodiments, the anti-microbial metal is ionic. The ionic antibiotic metal can be presented as an inorganic or organic salt (coupled with a counterion), an organometallic complex or an intercalate. Non binding examples of counter inorganic and organic ions are sulfadiazine, acetate, benzoate, carbonate, iodate, iodide, lactate, laurate, nitrate, oxide, palmitate, a negatively charged protein. In preferred embodiments, the antibiotic metal salt is a silver salt, such as silver acetate, silver benzoate, silver carbonate, silver iodate, silver iodide, silver lactate, silver laurate, silver nitrate, silver oxide, silver palmitate, silver protein, and silver sulfadiazine.

Yet, in another embodiment, the antifungal agent is an oxidizing agent or a substance that releases free radicals and/or active oxygen. Exemplary oxidizing agents are hydrogen peroxide, benzoyl peroxide, elemental halogen species (compounds), as well as oxygenated halogen species (compounds), bleaching agents (e.g., sodium, calcium or magnesium hypochloride and the like), perchlorite species (compounds), iodine and iodate compounds. Organic oxidizing agents are also included in the definition of “oxidizing agent”, such as quinones. Such agents possess a potent broad spectrum activity

In further embodiments the antibiotic agent is a cationic antimicrobial agent. The outermost surface of bacterial and fungal cells universally carries a net negative charge, making them sensitive to cationic substances. Examples of cationic antibiotic agents include: quaternary ammonium compounds, such as alkyltrimethyl ammonium bromides, benzalkonium chloride, dialkylbenzyl ammonium halides, and dimmers thereof, which bear bi-polar positive charges in conjunction with interstitial hydrophobic regions.

In one or more embodiments, the antifungal agent is an agent that is useful in the treatment of a superficial fungal infection of the skin, dermatophytosis, microsporum, trichophyton and epidermophyton infections, candidiasis, oral candidiasis (thrush), candidiasis of the skin and genital mucous membrane, candida paronychia, which inflicts the nail and nail bed and genital and vaginal candida, which inflict genitalia and the vagina.

Suitable antimycotics include but are not limited to allylamines, amorolfine, amphotericin B, azole compounds, bifonazole, butoconazole, chloroxine, clotrimazole, ciclopirox olamine, clotrimazole, econazole, elubiol, fenticonazole, fluconazole, flucytosine (5FC), griseofulvin, itraconazole, ketoconazole, mafenide acetate, miconazole, naftifine, natamycin, tolnaftate, nystatin, polyenes, oxiconazole, sulbentine, sulconazole, terbinafine, terconazole, tioconazole, undecylenic acid and derivatives, esters, salts and mixtures thereof.

### Vasoactive, Calcium Channel Blocker and Cholinergic Agent

### Vasoactive

In the context, a vasoactive agent is a substance that changes the diameter of a blood vessel.

In one or more embodiments, the vasoactive agent is a vasodilator. A vasodilator is any of various agents that relax or widen blood vessels and thereby maintain or lower blood pressure.

Alteration in the release and action of endothelium-derived vasoactive factors is responsible for changes in vascular reactivity early in the course of vascular disease. These factors include nitric oxide, eicosanoids, endothelium-derived hyperpolarizing factor, endothelin, and angiotensin II.

Nitric oxide (NO) has been recognized as an important messenger molecule having a broad spectrum of functions in many biological systems ranging from physiological control to pathological cytotoxic effectl-3. Along with prostacyclin, NO is responsible for endothelium derived tonic relaxation of all types of blood vessels. NO is formed from L-arginine through the action of a family of isoenzymes, the nitric oxide synthases (NOS). Thus, in one or more embodiments, the vasoactive agent is selected from the group of therapeutic agents that modulate the production of nitric oxide or otherwise modulate or activate the effect of nitric oxide. In one or more embodiments, the vasoactive agent is selected from the group of therapeutic agents that modulate the activity of the enzyme nitric oxide synthase. In one or more embodiments, the vasoactive agent is selected from the group of therapeutic agents that enhance the effect of NO by inhibiting enzymes from the phosphodiesterase group, such as phosphodiesterase type 5 (PDE5).

In one or more embodiments, the vasoactive agent is selected from the group including nitrites, nitrates and their analogs, esters and salts. In one or more embodiments the vasoactive agent possesses a moiety selected from the group consisting of ONO, and ONO2.

Exemplary vasodilators include, but are not limited to, amyl nitrite, amyl nitrate, ethyl nitrite, butyl nitrite, isobutyl nitrite, glyceryl trinitrate, also known as nitroglycerin, octyl nitrite, sodium nitrite, sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate (triethanolamine trinitrate diphosphate), propatylnitrate, nitrite esters of sugars, nitrite esters of polyols, nitrate esters of sugars, nitrate esters of polyols, nicorandil, apresoline, diazoxide, hydralazine, hydrochlorothiazide, minoxidil, pentaerythritol, tolazoline, scoparone (6,7-dimethoxycoumarin) and salts, isomers, analogs and derivatives thereof.

In one or more embodiments, the vasoactive agent belongs to a class of drugs that are known of possess vasodilator properties. Non limiting examples of drug classes that possess vasodilator properties include, but are not limited to, beta-adrenergic blockers, alpha-adrenoceptor blockers, prostaglandin and prostaglandin-like compounds, inhibitors of type 5 phosphodiesterase (PDE-5), angiotensin converting enzyme inhibitors, calcium antagonists, angiotensin II receptor antagonists, direct acting smooth muscle vasodilators, adrenergic inhibitors, endothelin antagonists, mineralocorticoid receptor antagonists, vasopeptidase inhibitors and renin inhibitors. Active agents belonging to such drug classes, as well as active agents belonging to other classes, which cause a vasodilator effect are also included in the scope of vasoactive agents.

Non-nitrate vasodilators from different classes include, but are not limited to sildenafil, dipyridamole, catecholamine, isoproternol, furosemide, prostaglandin, prostacyclin, enalaprilat (ACE-inhibitor), morphine (opiate), acepromazine (α-blocker), prazosin (α-blocker), enalapril(ACE-inhibitor), captopril (ACE-inhibitor), amlodipine (Ca channel blocker), minoxidil, tadalafil, vardenafil, phenylephrin, etilefein, caffeine, capsaicin and salts, isomers, analogs and derivatives thereof.

In one or more embodiments, the vasoactive agent is selected from the group of vasodilator peptides and proteins. Non-limiting examples of vasodilator paprides include, but are not limited to bradykinin, bradykinin-like peptide I, bradykinin-like peptide III Phyllokinin (bradykinyl-isoleucyl-tyrosine O-sulfate), megascoliakinin ([Thr6]bradykinin-Lys-Ala), lysyl-bradykinin-like waspkinin, lysyl-bradykinin, maximakinin (Bombinakinin M), bombinakinin-GAP, kininogen-1 associated peptides, kininogen-2 associated peptides, T-kinin, thiostatin, prolixin-S, vespulakinin 2, vespakinin X, relaxin, adrenomedullin, ghrelin, maxadilan, substance P, calcitonin gene-related peptide (CGRP), Natriuretic peptides (NPs), e.g., atrial natriuretic peptide (ANP), C-type natriuretic peptide (CNP), and adrenomedullin (ADM), adrenomedullin, ovine corticotropin-releasing factor, sauvagine, urotensin and salts, isomers, analogs and derivatives thereof.

In one or more embodiments, the vasoactive agent is selected from the group of therapeutic agents that induce the production of a vasodilator peptide or otherwise enhance or activate the effect of a vasodilator peptide.

In one or more embodiments, the vasoactive agent is a substance derived or extracted from herbs having a vasodilator effect. Non limiting examples of herbs that contain vasoactive agents include achillea millefolium (Yarrow), allium sativum (garlic), amoracia rusticana (horseradish), berberis vulgaris (barberry), cimicifuga racemosa (black cohosh), coleus forskholii (coleus), coptis (Goldentiread), crataegus (hawthorn), eleutherococcus senticosus (siberian ginseng), ginkgo biloba(ginkgo), melissa offiicnalis (lemon balm), olea europaea (olive leat), panax ginseng (Chinese ginseng), petroselinum crispum (parsley), scutellaria baicalensis (baical skullcap), tilia curopaea (linden flower), trigonella foenum-graecum (fenugreek), urtica dioica (nettles), valeriana officinalis (valerian), viburnum (cramp, bark, black haw), veratrum viride (American hellebore), verbena officinalis (vervain), xanthoxylum americanum (prickly ash), zingiber officinale (ginger), rauwolfia serpentina (Indian snakeroot), viscum album, wild yam, sasparilla, licorice, damiana, yucca, saw palmetto, gotu kola (centella asiatica), yohimbine and salts, hazel nut, brazil nut, walnut and analogs and derivatives thereof.

According to one or more embodiments, the foamable composition includes a vasodilator and a vasoactive agent such that the vasodilator can have a synergistic effect by readily facilitating facile penetration of the vasoactive agent.

In one or more embodiments, the vasoactive agent is a vasoconstrictor. A vasoconstrictor is any of various agents that narrow blood vessels and thereby maintain or increase blood pressure, and/or decrease blood flow. There are many disorders that can benefit from treatment using a vasoconstrictor. For example, redness of the skin (e.g., erythema or cuperose), which typically involves dilated blood vessels, benefit from treatment with a vasoconstrictor, which shrinks the capillaries thereby decreasing the untoward redness.

Other descriptive names of the vasoconstrictor group include vasoactive agonists, vasopressor agents and vasoconstrictor drugs. Certain vasoconstrictors act on specific receptors, such as vasopressin receptors or adrenoreceptors.

In one or more embodiments, the vasoconstrictor is a calcium channel agonist. Calcium channel agonists are agents that increase calcium influx into calcium channels of excitable tissues, thereby causing vasoconstriction.

Non limiting examples of vasoconstrictors include ephedrine, epinephrine, phenylephrine, angiotensin, vasopressin, and analogs and derivatives thereof.

In one or more embodiments, the vasoactive agent is a substance derived or extracted from herbs, having a vasoconstrictor effect.

Thus, in one or more embodiments, the vasoactive agent is a substance derived or extracted from a herbal source, selected from the group including ephedra sinica (ma huang), polygonum bistorta (bistort root), hamamelis virginiana (witch hazel), hydrastis canadensis (goldenseal), lycopus virginicus (bugleweed), aspidosperma quebracho (quebracho blanco), cytisus scoparius (scotch broom), cypress and salts, isomers, analogs and derivatives thereof.

Yet, in additional embodiments, the vasoactive agent is a metal oxide or a mineral, such as zinc oxide and bismuth subgallate.

The McKenzie vasoconstrictor assay, as described, for example, in the British Journal of Dermatology 1975; 93:563-71 and versions thereof, has been the primary method used for classifying the strength of a vasoconstrictor clinical efficacy. Thus, in one or more embodiments, the vasoactive agent is an agent that positively affects the vasoconstrictor assay.

Mixtures of these vasoactive agents may also be employed.

Solubility of the vasoactive agent is an important factor in the development of a stable foamable composition.

### Calcium Channel Blockers

Calcium channel blockers are a chemically and pharmacologically heterogeneous group of drugs, but physiologically they all share the ability to selectively antagonize the calcium ion movements that are responsible for the excitation-contraction coupling in the cardiovascular system. Beyond their cardiovascular effects, calcium channel blockers are known to possess other effects, such as inhibition of the growth and proliferation of vascular smooth muscle cells and fibroblasts, inhibition of the synthesis of extracellular matrix proteins, immunomodulation, inhibition of mast cell degranulation and platelet aggregation and suppression of neutrophil adhesion and superoxide anion (O-2) production. Some calcium channel blockers also have analgesic effects.

Current therapeutic uses of calcium channel blockers include (but are not limited to) hypertension, angina, arrhythmia and subarachnoid hemorrhage. Calcium channel blockers may further relieve or prevent reactive vasodilation of migraine sufferers by inhibiting the vasoconstriction during the prodromal phase.

There are two main classes of calcium channel blockers: dihydropyridines (e.g., nifedipine, nicardipine, amlodipine, felodipine and nimodipine) and nondihydropyridines which include diltiazem (a benzothiazepine) and verapamil (a phenylalkylamine). Flunarizine is an antihistamine with calcium channel blocking activity.

In an embodiment, the calcium channel blocker can be selected from the group consisting of an amlodipine, anipamil, barnidipine, benidipine, bepridil, darodipine, diltiazem, efonidipine, felodipine, isradipine, lacidipine, lercanidipine, lidoflazine, manidipine, mepirodipine, nicardipine, nifedipine, niludipine, nilvadipine, nimodipine, nisoldipine, nitrendipine, perhexyline, tiapamil, verapamil, pharmaceutically acceptable salts, isomers, analogs and derivatives thereof.

### Cholinergic Drugs

Cholinergic drugs produce the same effects as acetylcholine. Acetylcholine is the most common neurohormone of the parasympathetic nervous system, the part of the peripheral nervous system responsible for the every day work of the body. A cholinergic agent, also known as a parasympathomimetic agent, is a chemical which functions to enhance the effects mediated by acetylcholine in the central nervous system, the peripheral nervous system, or both. These include acetylcholine receptor agonists muscarine and nicotine, as well as anticholinesterases.

Suitable cholinergic drugs in accordance with the present invention are selected from a cholinergic agonist of acetylcholine, bethanechol, carbachol, methacholine, and pilocarpine, or an anticholinesterase of ambenonium, neostigmine, physostigmine, pyridostigmine, dyflos, and ecothinopate, and pharmaceutically acceptable salts, isomers, analogs and derivatives thereof.

### Nitric Oxide Donors

Nitric oxide is an inorganic free radical, which has the chemical formula of N═O and abbreviated to NO, and is a remarkably versatile biological messenger. The chemical properties of NO are cricial in defining its biological roles, both as a transcellular signal in the cardiovascular and nervous systems and as a cytotoxic antipathogenic agent released during an inflammatory response. Endogenous NO is synthesized from the amino acid L-arginine by three isoforms of the enzyme NO synthase (NOS). The endothelial (ENOS) and neuronal (NNOS) isoforms that synthesize NO for transcellular signaling are constitutively expressed tightly regulated by a number of cofactors. These NOS isoforms typically synthesize small amounts of NO and require activation by Ca²⁺-calmodulin, making them sensitive to agents and processes that increase intracellular calcium levels. The NO generated diffuses to neighboring target cells where it acts primarily through activation of soluble guanylate cyclase (sGC) to generate cGMP from GTP, and bring about the cellular response through a reduction in intracellular calcium levels.

In an embodiment, the nitric oxide donors can be selected from several classes, including, but not limited to inorganic nitrites and nitrates (e.g., sodium nitrite), organic nitrites and nitrates, sodium nitroprusside, molsidomine and its metabolites, diazeniumdiolates, S-nitrosothiols, mesoionic oxatriazole and derivatives thereof, iron-sulphur nitrosyls, Sinitrodil, FK-409 (4-Ethyl-2-[(Z)-hydroxyiminol]-5-nitro-3(E)-hexeneamide) and derivatives thereof and hybrid NO donor drugs.

In an embodiment, the organic nitric oxide donor includes at least one organic nitrate, which includes esters of nitric acid and may be an acyclic or cyclic compound. For instance, the organic nitrate may be ethylene glycol dinitrate; isopropyl nitrate; amyl nitrite, amyl nitrate, ethyl nitrite, butyl nitrite, isobutyl nitrite, octyl nitrite, glyceryl-1-mononitrate, glyceryl-1,2-dinitrate, glyceryl-1,3-dinitrate, nitroglycerin, butane-1,2,4-triol-trinitrate; erythrityl tetranitrate; pentaerythrityl tetranitrate; sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate (triethanolamine trinitrate diphosphate) propatylnitrate, nitrite esters of sugars, nitrate esters of sugars, nitrite esters of polyols, nitrate esters of polyols, nicorandil, apresoline, diazoxide, hydralazine, hydrochlorothiazide, minoxidil, pentaerythritol, tolazoline, scoparone (6,7-dimethoxycoumarin) and pharmaceutically acceptable salts, isomers, analogs and derivatives thereof

In one embodiment, vasoactive drugs that act via eNOS activity enhancement, such as sildenafil, vardenafil and tadalafil are also regarded “nitric oxide donors.”

In an embodiment, the active agent is an antipruritic. Suitable antipruritics include but are not limited to menthol, methdilazine, trimeprazine, urea and derivatives, esters, salts and mixtures thereof.

In an embodiment, the therapeutic agent is an additional antipsoriatic agent. Suitable additional antipsoriatic agents include but are not limited to 6-aminonicotinamide, 6-aminonicotinic acid, 2-aminopyrazinamide, anthralin, 6-carbamoylnicotinamide, 6-chloronicotinamide, 2-carbamoylpyrazinamide, coiticosteroids, 6-dimethylaminonicotinamide, dithranol, 6-formylaminonicotinamide, 6-hydroxy nicotinic acid, 6-substituted nicotinamides, 6-substituted nicotinic acid, 2-substituted pyrazinamide, tazarotene, thionicotinamide, trichothecene mycotoxins and derivatives, esters, salts and mixtures thereof.

In an embodiment, the active agent is an antirosacea agent. Suitable antirosacea agents include but are not limited to azelaic acid, metronidazole, sulfacetamide and derivatives, esters, salts and mixtures thereof. Certain nonsteroidal anti-inflammatory agents, such as salicylic acid, salycilates, piroxicam and diclofenac are also useful for the treatment of Rosacea.

In an embodiment, the therapeutic agent is an antiseborrheic agent. Suitable antiseborrheic agents include but are not limited to glycolic acid, salicylic acid, selenium sulfide, zinc pyrithione, a dicarboxylic acid, such as azelaic acid and derivatives, esters, salts and mixtures thereof.

In an embodiment, the therapeutic agent is an antiviral agent. Suitable antiviral agents include but are not limited to acyclovir, gancyclovir, ribavirin, amantadine, rimantadine nucleoside-analog reverse transcriptase inhibitors, such as zidovudine, didanosine, zalcitabine, tavudine, lamivudine and vidarabine, non-nucleoside reverse transcriptase inhibitors, such as nevirapine and delavirdine, protease inhibitors, such as saquinavir, ritonavir, indinavir and nelfinavir, and interferons and derivatives, esters, salts and mixtures thereof.

In an embodiment, the therapeutic agent is a chemotherapeutic agent. Suitable chemotherapeutic agents include but are not limited to daunoribicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A, XR9576 and derivatives, esters, salts and mixtures thereof.

### Steroids

In an embodiment, the therapeutic agent is a corticosteroid. Suitable corticosteroids include but are not limited to alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, difluorosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, .alpha.-methyl dexamethasone, methylprednisolone, methylprednisolone acetate, mometasone furoate, paramethasone, prednisolone, prednisone, pregnenolone, progesterone, spironolactone, triamcinolone, triamcinolone acetonide and derivatives, esters, salts and mixtures thereof.

The steroid is selected from the group consisting of:
(i) a steroid compound containing a cyclopenta[a]phenanthrene skeleton;
(ii) a steroid compound containing a cyclopenta[a]phenanthrene skeleton carrying one or more functional groups selected from halogens, alkyl groups, aryl groups, benzyl groups, carboxy groups and alkoxy groups;
(iii) a steroid compound selected from the families of (a) cardanolides, (b) bufanolides, (c) spirostans, (d) furostans, (e) steroid alkaloids, (f) steroid lactones, (g) oxo-steroids, (h) steroid-alcohols and (i) steroid-amines;
(iv) a steroid compound, where one or more of the cyclopenta[a]phenanthrene rings is contracted by loss of an unsubstituted methylene group;
(v) a steroid compound, where one or more of the cyclopenta[a]phenanthrene rings is expanded by inclusion of a methylene group;
(vi) a steroid compound containing a cyclopenta[a]phenanthrene skeleton and a carbocyclic or heterocyclic ring component fused to it;
(vii) a compound, wherein two or more steroid molecules are linked together covalently;
(viii) a compound selected from the group consisting of 5^{α}-pregnane, 5^{β}-pregnane, 5^{α}-cholane (allocholane), 5^{β}-cholane, 5^{α}-cholestane, 5^{β}-cholestane, 5^{α}-ergostane, 5^{β}-ergostane, 5^{α}-campestane, 5^{β}-campestane, 5^{α}-poriferastane, 5^{β}-poriferastane, 5^{α}-stigmastane, 5^{β}-stigmastane, 5^{α}-gorgostaneacrihellin, actodigin, alfacalcidol, aldosterone, androsterone, betamethasone, brassinolide, calcidiol, calciol, calcitriol, canrenone, clomegestone, cholesterol, cholic acid, corticosterone, cortisol, cortisol acetate, cortisone, cortisone acetate, cyproterone, deoxycorticosterone, dexamethasone, disogluside, ecdysone, ercalciol, ergosterol, estradiol, estriol, estrone, ethinylestradiol, fluazacort, fluocortin, fusidic acid, gestrinone, gonane, halometasone, hydrocortisone, lanosterol, lithocholic acid, mebolazine, medroxyprogesterone, meproscillarin, mespirenone, mestranol, naflocort, norenthisterone, norgesterone, norgestrel, oxandrolone, oxymetholone, pancuronium bromide, prednisolone, prednisone, progesterone, proscillardin, pseudotigogenin, roxibolone, sarsasapogenin, smilagenin, spironolactone, timobesone, testosterone, tigogenin triamcinolone, ursodeoxycholic acid;
(ix) an anti-inflammatory steroid;
(x) a steroid possessing immunomodulating and/or anti-inflammatory properties;
(xi) a steroid, selected from the group of low-potency anti-inflammatory steroids, medium potency anti-inflammatory steroids and high potency anti-inflammatory steroids;
(xii) an anti-inflammatory steroid, selected from the group consisting of hydrocortisone, hydrocortisone acetate, desonide, betamethasone valerate, clobetasone-17-butyrate, flucinonide, fluocinolone acetonide, alcometasone dipropionate, mometasone furoate, prednicarbate, triamcinolone acetonide, betamethasone-17-benzoate, methylprednisolone aceponate, betamethasone dipropionate, halcinonide, triamcinolone acetonide, halobetasol, clobetasol-17-propionate;
(xiii) a steroid that positively affects the McKenzie vasoconstrictor assay;
(xiv) a steroid hormone;
(xv) a steroid hormone, selected from the group consisting of an androgen, an estrogen and a progestogen;
(xvi) an androgen, selected from the group consisting of testosterone, testosterone cipionate, testosterone decanoate, testosterone enantate, testosterone isocaproate, testosterone phenylpropionate, testosterone propionate, testosterone undecylate, 5α-dihydrotestosterone, dehydroepiandrosterone (also termed prasterone and DHEA), androstenedione, androstanediol, androsterone, androstenolone, prasterone enantate, prasterone sodium sulfate, ommeloxifene, mesterolone, fluoxymesterone, methyltestosterone, gestrinone, delmadinone, delmadinone acetate, chlormadinone, chlonnadinone acetate, danazol and testolactone;
(xvii) an estrogen selected from the group consisting of estradiol, estradiol benzoate, estradiol cipionate, estradiol dipropionate, estradiol enantate, estradiol hexahydrobenzoate, estradiol phenylpropionate, estradiol valerate, polyestradiol phosphate, estriol, estriol sodium succinate, estriol succinate, polyestriol phosphate, quinestradol, ethinylestradiol, estrapronicate, mestranol, estrapronicate and equilin;
(xviii) a progestogen, selected from the group consisting of progesterone, norethisterone, norethisterone acetate, norethisterone enantate, medroxyprogesterone acetate, delmadinone acetate, flugestone acetate, dydrogesterone, desogestrel, norgestrel, levonorgestrel, dydrogesterone, gestodene, chlonnadinone acetate, dienogest, drospirenone, lynestrenol, tybolone, cyproterone acetate, megestrol acetate, nomegestrol acetate;
(xix) an inhibitor of a steroid hormone;
(xx) an inhibitor of a steroid hormone selected from the group consisting of finasteride, dutasteride and spironolactone;
(xxi) a vitamin D;
(xxii) a steroid that exhibits qualitatively the biological activity of calciol;
(xxiii) a vitamin D selected from the group consisting of cholecalciferol, 25-hydroxycholecalciferol, 1α,25-dihydroxycholecalciferol, ergocalciferol, 1α,25-dihydroxyergocalciferol, 22,23-dihydroergocalciferol, 1,24,25-trihydroxycholecalciferol, previtamin D₃, tachysterol₃ (also termed tacalciol);
(xxiv) a vitamin D₃ analogue;
(xxv) isovitamin D₃, dihydrotachysterol₃, (1S)-hydroxycalciol, (24R)-hydroxycalcidiol, 25-fluorocalciol, ercalcidiol, ertacalciol, (5E)-isocalciol, 22,23-dihydroercalciol, (24S)-methylcalciol, (5E)-(10S)-10,19-dihydroercalciol, (24S)-ethylcalciol and (22E)-(24R)-ethyl-22,23-didehydrocalciol;
(xxvi) a vitamin D₃ analogue selected from calcipotriol, tacalcitol, maxacalcitol, and calcitriol;
(xxvii) a phytosteroid or a phytosterol;
(xxviii) a steroid derived or extracted from one of the families of phytosteroids, phytosterols, phytostanols, ecdysones, withanolids, sterines, steroid saponins and soflavonoids;
(xxix) a steroid selected from the group consisting of alpha-sitosterol, beta-sitosterol, stigmastanol, campesterol, alpha-sitostanol, beta-sitostanol, stigmastanol, campestanol, avenosterol, brassicasterol, desmosterol, chalinosterol, beta-ecdysone, whithaferin A, beta-sitosterine, stigmasterine, campesterine, ergosterine, diosgenin, daidzein, glycitein, genistein, muristerone, poriferasterol, clionasterol, campestanol, and cycloaitenol;
(xxx) a plant oil or a plant extract, which contains a steroid;
(xxxi) a plant oil or a plant extract, selected from the group consisting of nuts seeds, sprouted seeds and grains (such as alfalfa), St. Mary's thistle, ginkgo biloba, saw palmetto, panax, siberian ginseng, foeniculum vulgare, cimicifuga racemosa, licorice root, red clover, sage, sarsaparilla, sassafras, angelica sinensis achillea millefolium, anemone pratensis, angelica sinensis, glycyrrhiza glabra, hypericum perforatum, larrea, panax, piscidia erythrina, plantago psyllium, serenoa repens, symphytum, taraxacum officinale, trifolium pratense, turnera spp., tussilago farfara, valeriana officinalis, viburnum prunifolium, calendula officinalis;
(xxxii) any one of the compounds exemplified in the present specification; and salts thereof.

In the context, steroids are compounds possessing the skeleton of cyclopenta[a]phenanthrene or a skeleton derived therefrom by one or more bond scissions or ring expansions or contractions. Methyl groups are normally present at C-10 and C-13. An alkyl side chain may also be present at C-17. Sterols are steroids carrying a hydroxyl group at C-3 and most of the skeleton of cholestane. Additional carbon atoms may be present in the side chain.

Steroids are numbered and rings are lettered as in formula 1. If one of the two methyl groups attached to C-25 is substituted it is assigned the lower number (26); if both are substituted, that carrying the substituent cited first in the alphabetical order is assigned the lower number.

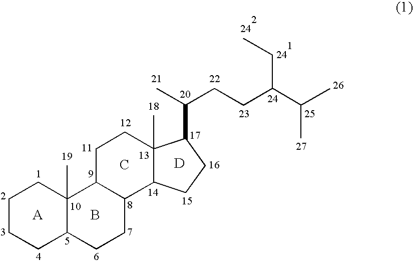

The steroids can have substituents on the steroid side chain as exemplified in formula 4-7:

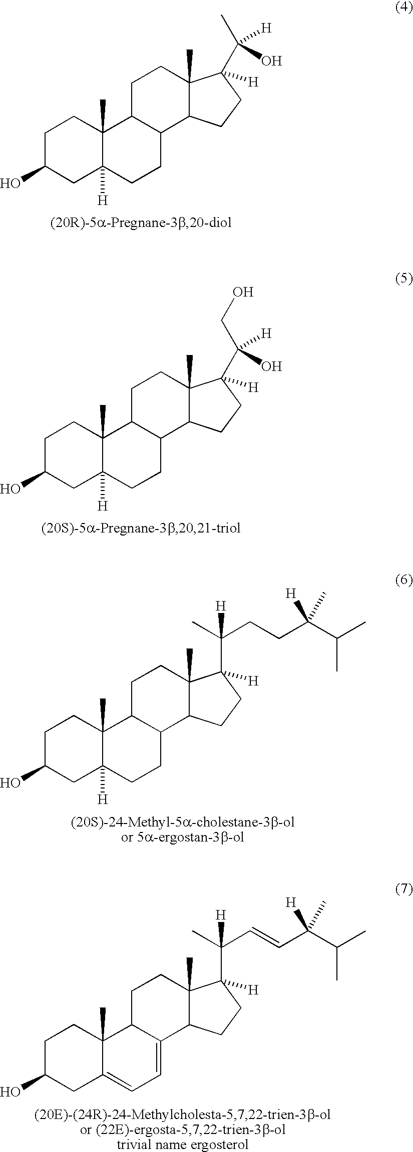

The steroids can have the formalae as exemplified in formula 9-18. In one or more embodiments, the steroid or sterol has no substitution at C-17, as exemplified by gonane, e.g., formulae 9 and 10, estrange (also termed oestrane), e.g. formulae 11 and 12, and androstane, e.g., formulae 13 and 14. In one or more embodiments, the steroid or sterol has methyl groups at both C-10 and C-13 and a side chain Rat C-17 (formulae 15 and 16), as exemplified in Table 1.

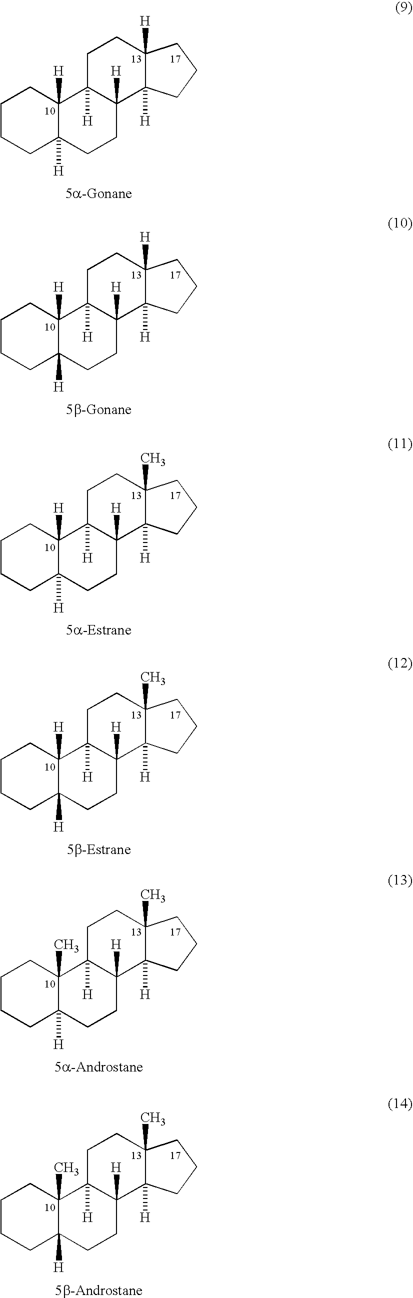

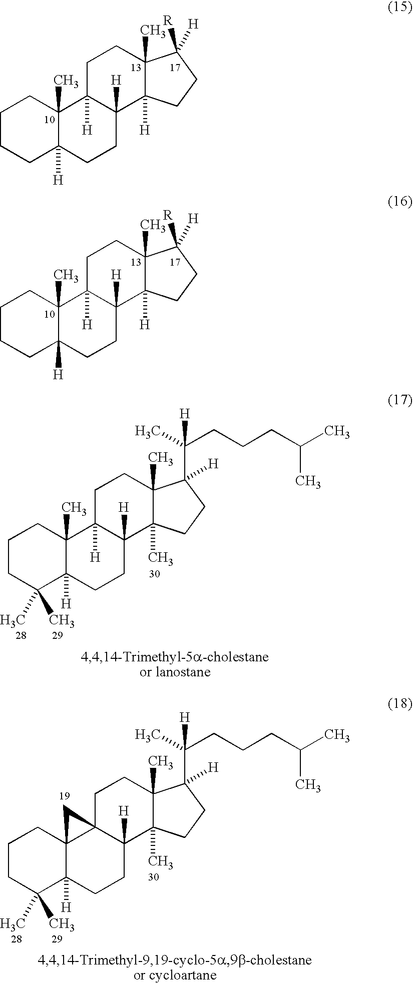

TABLE 14
Hydrocarbons with side chain at C-17

| Side chain | 5α-Series (15) | 5^{β}-Series (16) |
|---|---|---|
| 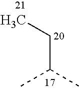 | 5α-pregnane(allopregnane) | 5^{β}-pregnane |
| 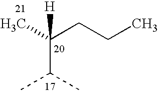 | 5α-cholane(allocholane) | 5^{β}-cholane |
| 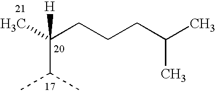 | 5α-cholestane | 5^{β}-cholestane(coprostane) |
| 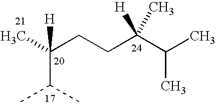 | 5α-ergostane | 5^{β}-ergostane |
| 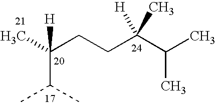 | 5α-campestane | 5^{β}-campestane |
| 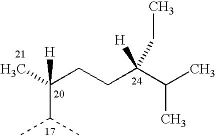 | 5α-poriferastane | 5^{β}-poriferastane |
| 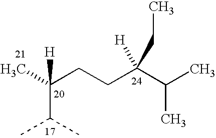 | 5α-stigmastane | 5^{β}-stigmastane |
| 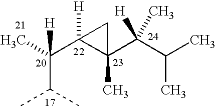 | 5α-gorgostane | 5^{β}-gorgostane |

Examples of unsaturated steroids and sterols are provided in formulae 19-22:

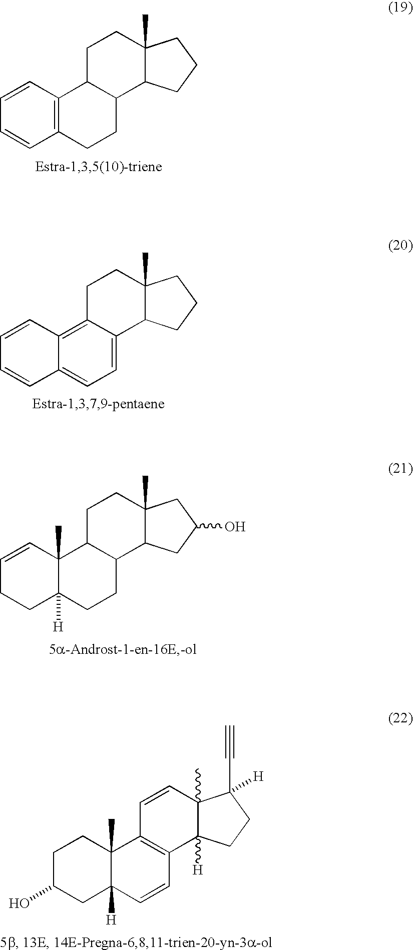

The stereochemistry of double bonds in the side chain is indicated using the E,Z convention. The same applies to the seco compounds of the vitamin D series (example in formula 23). In certain cases, the steroid has two carbon chains attached at position 17, e.g. 17-methyl-5α-pregnane 24, 17-methyl-5α,17β-pregnane 25, and 17-ethyl-5-cholestane and 17-(2-bromoethyl)-5α, 17α-cholestane 26. Other examples of a steroid that has two carbon chains attached at position 17, are 17,17-dimethyl-5α-androstane 27 and 17β-methyl-17α-propyl-5α-androstane 28. In certain embodiments, the carbon skeleton of a steroid a carbon atom is replaced by a hetero atom, as exemplified by 17β-hydroxy-4-oxaandrost-5-en-3-one 29. Yet, in additional embodiments, an additional ring is formed by means of a direct link between any two carbon atoms of the steroid ring system or the attached side chain, as exemplified by formulae 30, 31 and 32.

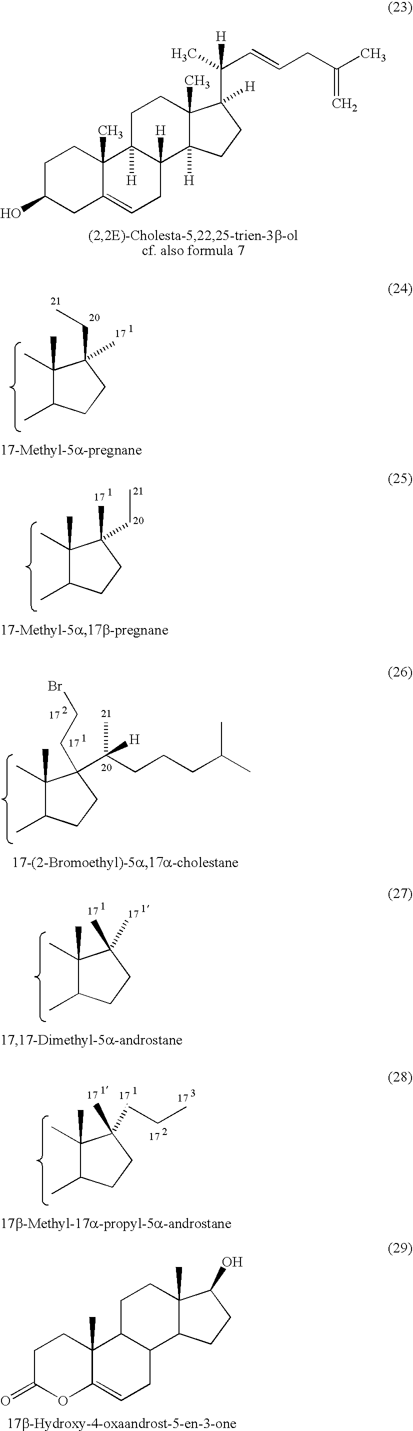

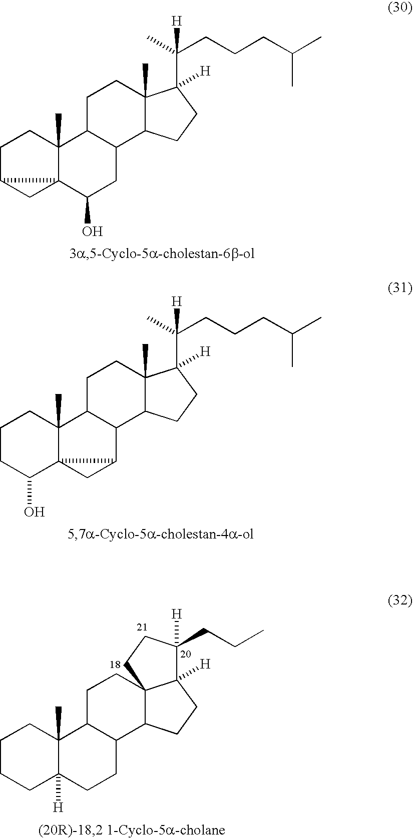

Many important naturally occurring steroids contain one or more additional heterocyclic ring(s), fused or attached to ring D, formed by modifications of the side chain. These steroids can be grouped into the following families: (a) cardanolides, e.g., 5β-cardanolide 33, 3β,14-dihydroxy-50-card-20(22)-enolide (digitoxigenin) 34 and 3β,5,14-trihydroxy-19-oxo-5β-card-20(22)-enolide (strophanthidin) 35, as well as epoxycardanolides, containing a 14,21- or a 16,21-oxygen bridge, as shown in 36, (b) bufanolides, e.g., structures 37-39, (c) spirostans, e.g., structures 40-43, (d) furostans, e.g., structures 44-45, and (e) steroid alkaloids.

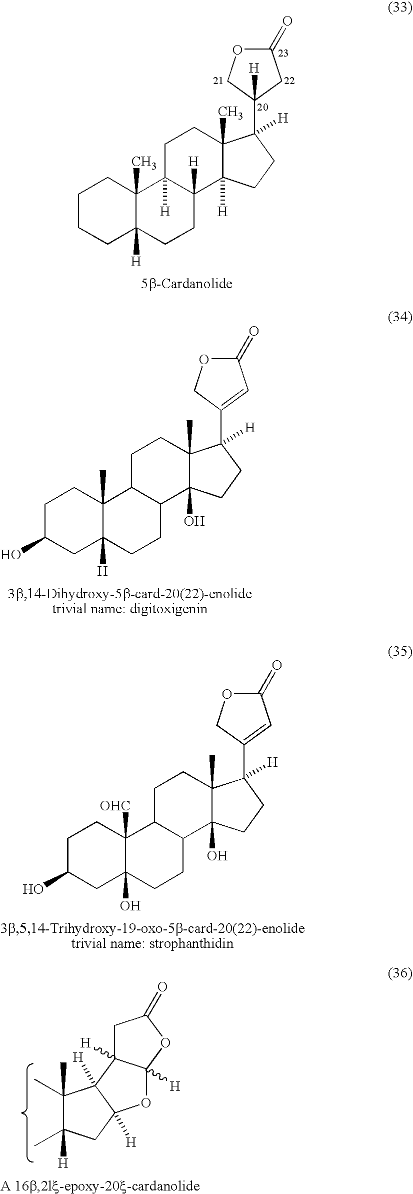

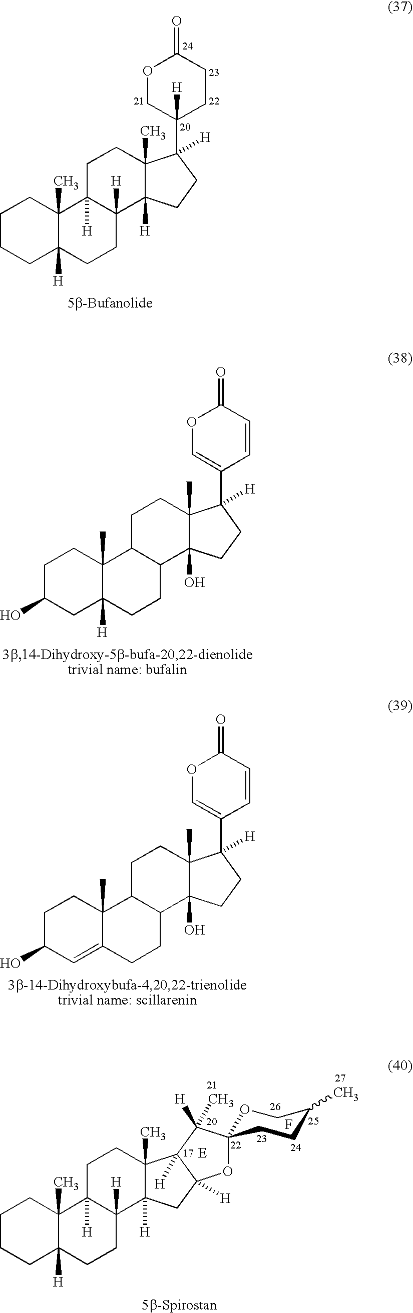

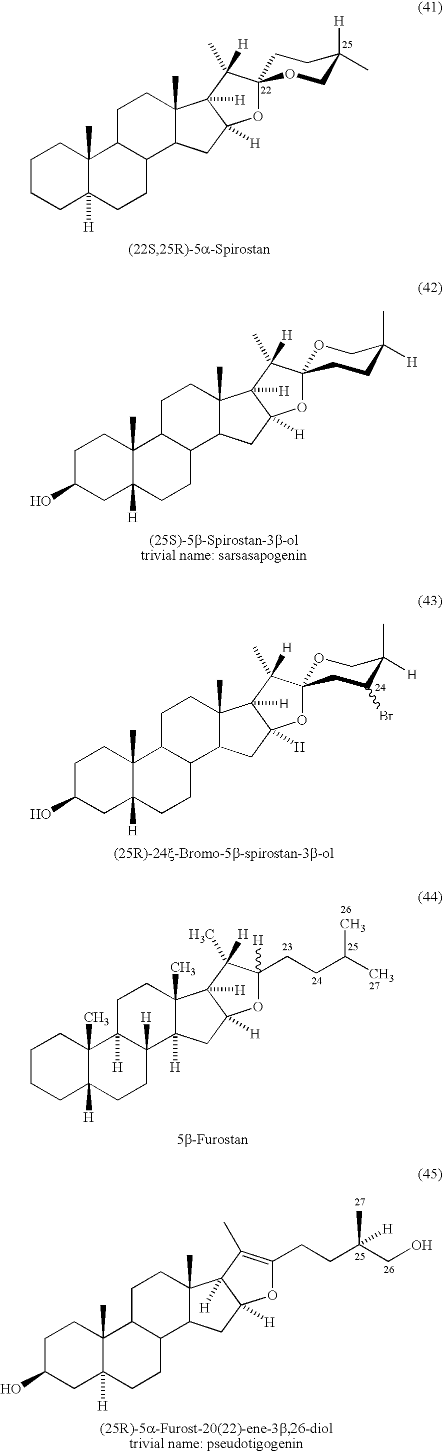

Several biologically important steroids are derivatives of the parent hydrocarbons carrying various functional groups. Some of the common functional groups include but are not limited to halogens, alkyl groups, aryl groups, benzyl groups, carboxy groups and alkoxy groups.

In one or more embodiments, the steroid is selected from the group consisting of an acid, a salt of an acid, as exemplified in formulae 46-49, and esters, as exemplified in formulae 50 and 51. In one or more embodiments, the steroid is a lactone, as exemplified in formulae 52-54.

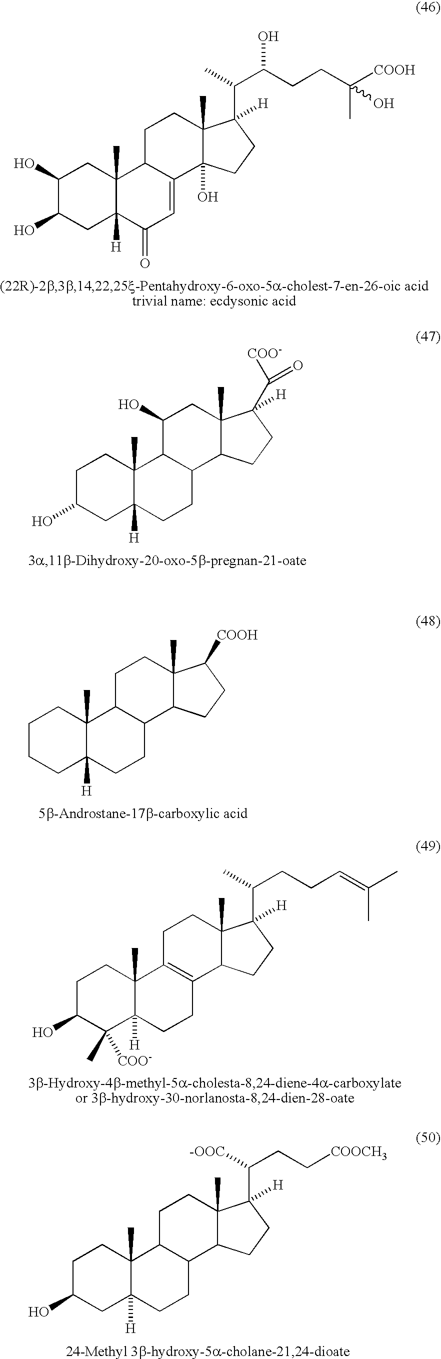

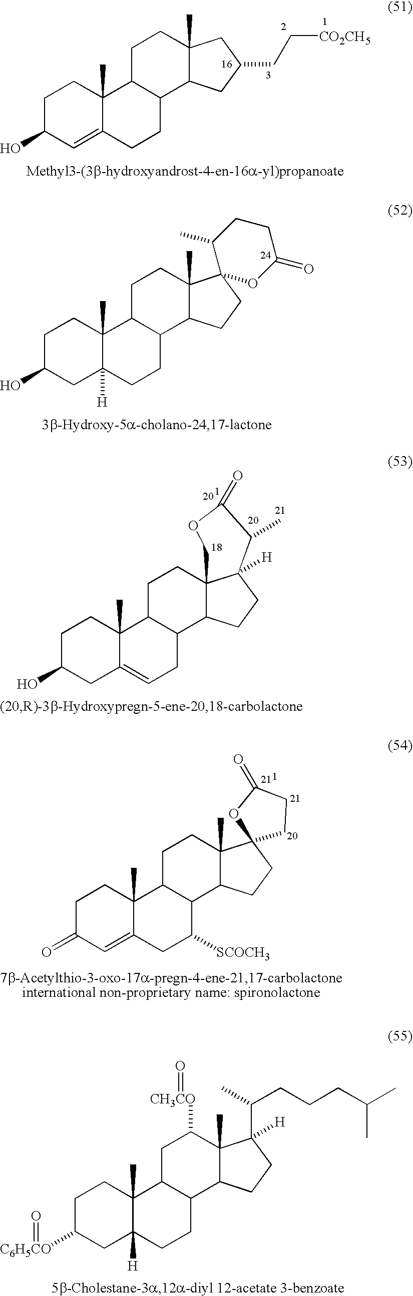

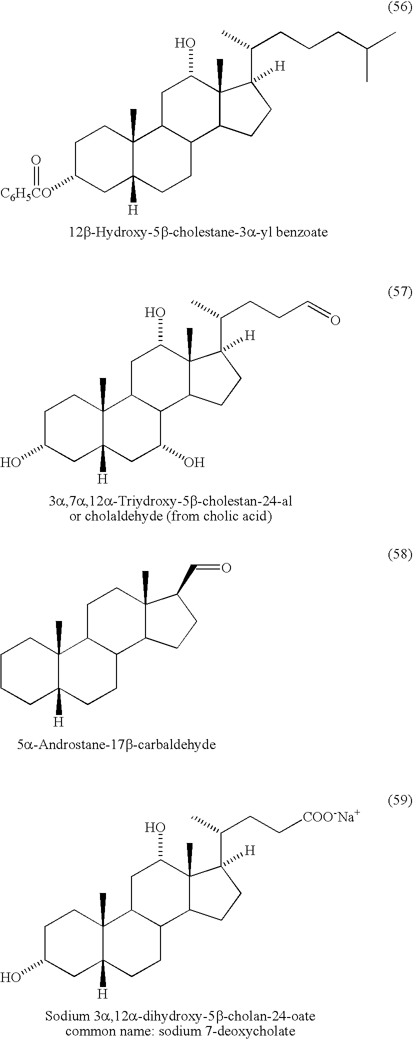

In one or more embodiments, the steroid is an ester of a steroid alcohol, as exemplified by 5-cholestan-3-yl acetate, 5-cholestane-3,12-diyl diacetate, 3-oxoandrost-4-en-17-yl acetate (trivial name testosterone acetate), 17-hydroxy-20-oxopregn-5-en-3-yl sulfate, 3-acetoxy-5-cardanolide, 3-benzoyloxy-11-hydroxy-20-oxo-5-pregnan-21-oate (monobenzoate of 47), 3-acetoxy-5-cholano-24,17-lactone (acetate of 52), 3-O-acetylcholic acid, 17-O-benzoylestradiol-17,3-O-linolenoylcholesterol, as well as in formulae 55 and 56.

In one or more embodiments, the steroid is an oxo compound. The oxo compound can be an aldehde, as exemplified by 5-androstan-19-al, 5-cholan-24-al, 3-formyl-5-cholan-24-oic acid and by formulae 57 and 58, or a ketone, as exemplified by 5-androstan-3-one, pregn-5-ene-3,20-dione and 11-oxo-5-cholan-24-oic acid.

In one or more embodiment, the steroid is an alcohol as exemplified by 5-cholestane-3,11-diol, 3-hydroxy-5-androstan-17-one (trivial name: androsterone) and by formulae 59.

In additional embodiments, the steroid is an amine as exemplified by androst-5-en-3-amine and formula 60, an ether as exemplified by 17-methoxyandrost-4-en-3-one, (20S)-3,17,20-trimethoxy-5-pregnane, (20S)-3,17-dimethoxy-5-pregnan-20-ol, 21-O-methylcortisol and formula 61, an acetal or a ketal of an oxo steroid (also named as dialkoxy steroids) as exemplified by 3,3-dimethoxycholest-4-ene, 2,3-(methylenedioxy)pregn-5-ene and formula 62.

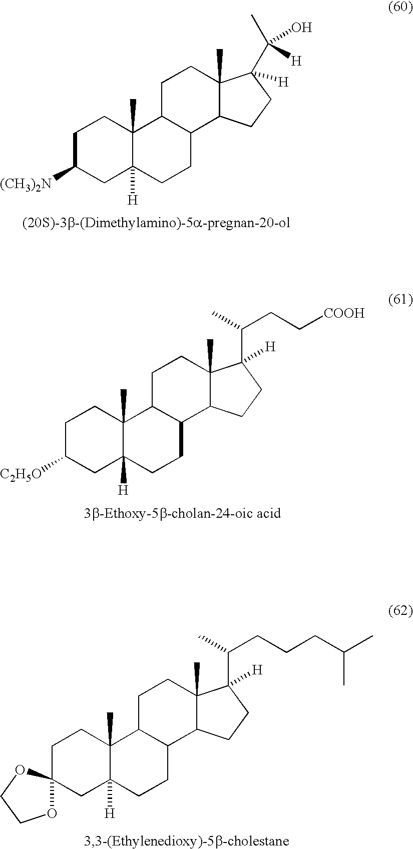

Examples of trivial names retained for important steroid derivatives, these being mostly natural compounds of significant biological activity, are given in Table 15.

TABLE 15
Trivial names of some important steroid derivatives

| Trivial name | Systematic steroid name |
|---|---|
| Aldosterone | 18,11-hemiacetal of 11β,21-dihydroxy-3,20-dioxopregn-4-en- |
| | 18-al or 11β,18-epoxy-18ξ,21-dihydroxypregn-4-ene-3,20- |
| | dione |
| Androsterone | 3α-hydroxy-5α-androstan-17-one |
| Brassinolide | (22R,23R)-2α,3α,22,23-tetrahydroxy-6,7-seco-5α- |
| | cmpestano-6,7-lactone |
| Calcidiol (93) | (5Z,7E)-(3S)-9,10-secocholesta-5,7,10(19)-triene-3,25-diol |
| Calciol = cholecalciferol (92) | (5Z,7E)-(3S)-9,10-secocholesta-5,7,10(19)-trien-3-ol |
| Calcitriol (94) | (5Z,7E)-(1S,3R)-9,10-secocholesta-5,7,10(19)-triene-1,3,25- |
| | triol |
| Cholesterol | cholest-5-en-3β-ol |
| Cholic acid | 3α,7α,12α-trihydroxy-5β-cholan-24-oic acid |
| Corticosterone | 11β,21-dihydroxypregn-4-ene-3,20-dione |
| Cortisol | 11β,17,21-trihydroxypregn-4-ene-3,20-dione |
| Cortisol acetate | 21-O-acetylcortisol |
| Cortisone | 17,21-dihydroxypregn-4-ene-3,11,20-trione |
| Cortisone acetate | 21-O-acetylcortisone |
| Deoxycorticosterone | 21-hydroxypregn-4-ene-3,20-dione (i.e. the 11-deoxy |
| | derivative of corticosterone) |
| Ecdysone | (22R)-2β,3β,14α,22,25-pentahydroxy-5β-cholest-7-en-6-one |
| Ercalciol = ergocalciferol | (5Z,7E,22E)-(3S)-9,10-secoergosta-5,7,10(19),22-tetren-3-ol |
| Ergosterol (7) | (22E)-ergosta-5,7,22-trien-3β-ol |
| Estradiol-17α | estra-1,3,5(10)-triene-3,17α-diol |
| Estradiol-17β | estra-1,3,5(10)-triene-3,17β-diol |
| Estriol | estra-1,3,5(10)-triene-3,16α,17β-triol |
| Estrone | 3-hydroxyestra-1,3,5(10)-trien-17-one |
| Lanosterol | lanosta-8,24-dien-3β-ol |
| Lithocholic acid | 3α-hydroxy-5β-cholan-24-oic acid |
| Progesterone | pregn-4-ene-3,20-dione |
| Pseudotigogenin | (25R)-5α-furost-20(22)-ene-3β,26-diol |
| Sarsasapogenin | (25S)-5β-spirostan-3β-ol |
| Smilagenin | (25R)-5β-spirostan-3β-ol |
| Testosterone (63) | 17β-hydroxyandrost-4-en-3-one |
| Tigogenin | (25R)-5α-spirostan-3β-ol |

Additional non-limiting examples of steroids that are applicable are provided in formulae 63-79.

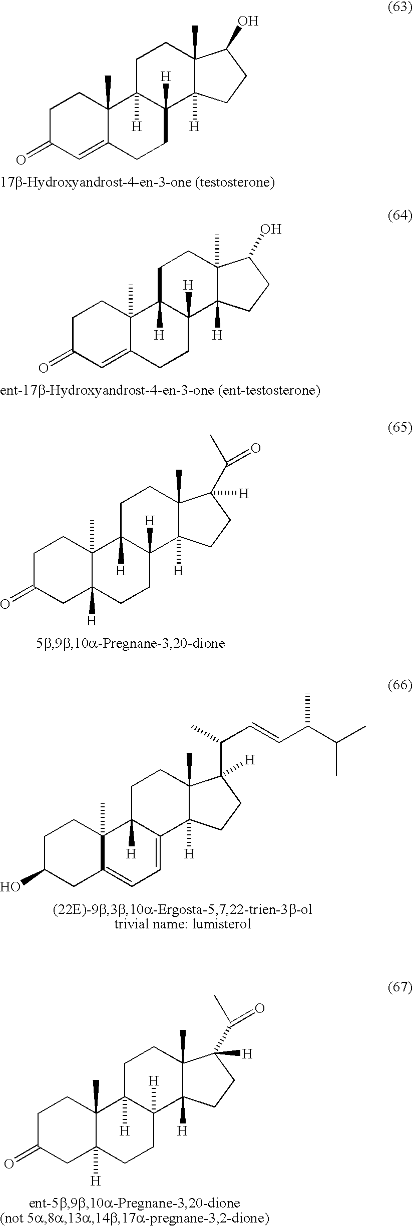

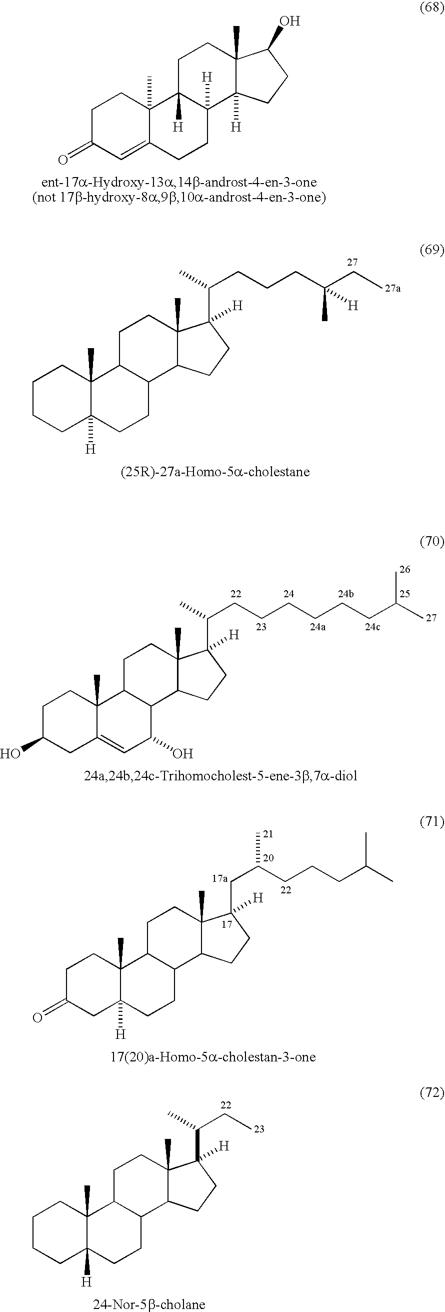

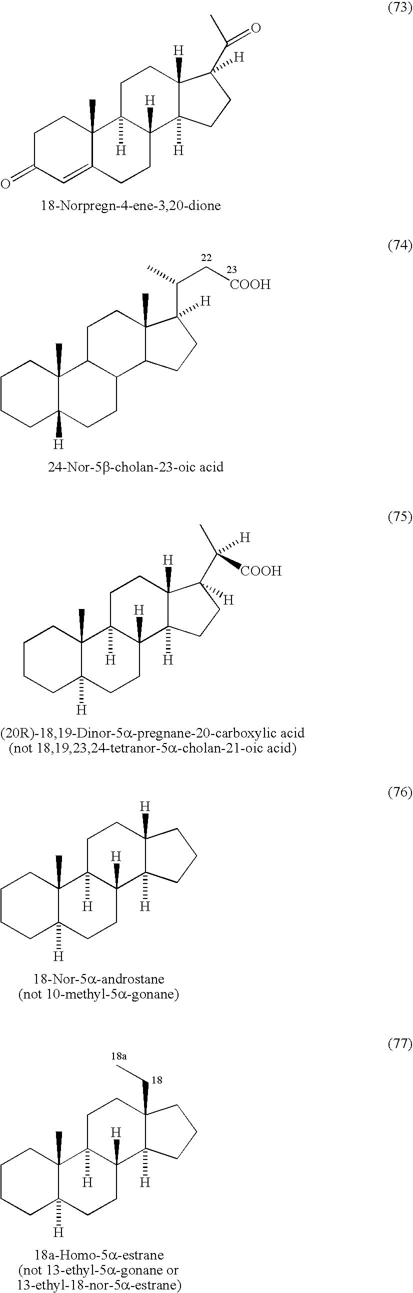

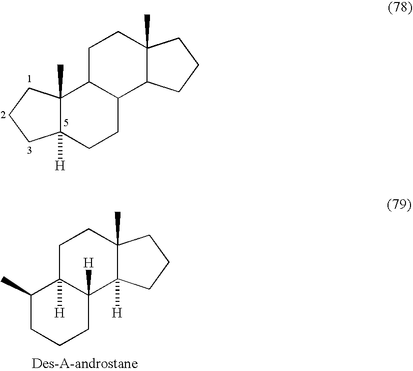

In one or more embodiments, the steroid is a compound, in which one or more of the cyclopenta[a]phenanthrene rings is contracted by loss of an unsubstituted methylene group, as exemplified by 4-nor-5-androstane (78), where C-4 is missing. In other embodiments one or more of the cyclopenta[a]phenanthrene rings is expanded by inclusion of a methylene group, as exemplified by formulae 80-86.

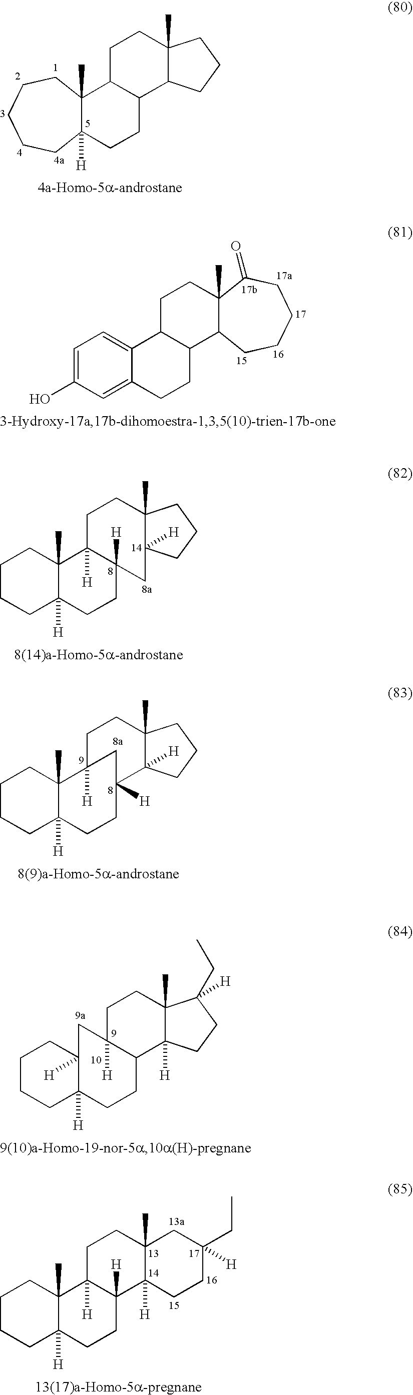

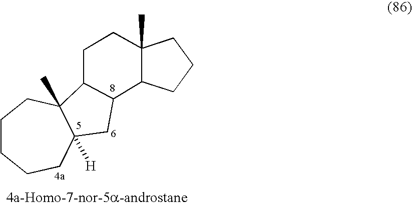

In one or more embodiments, the steroid contains additional rings that are formed within, or on, a steroid nucleus. In additional embodiments, the steroids contains a bivalent bridge such as —O—O—, —[CH₂]ₙ—, linking non-adjacent ring positions as exemplified by formulae 99-102.

In one or more embodiments, the steroid contains a cyclopenta[a]phenanthrene skeleton and a carbocyclic or heterocyclic ring component fused to it, as exemplified by formulae 103-111, and in other embodiments, an additional ring is linked to the cyclopenta[a]phenanthrene skeleton through a spiro system, as exemplified by formula 112.

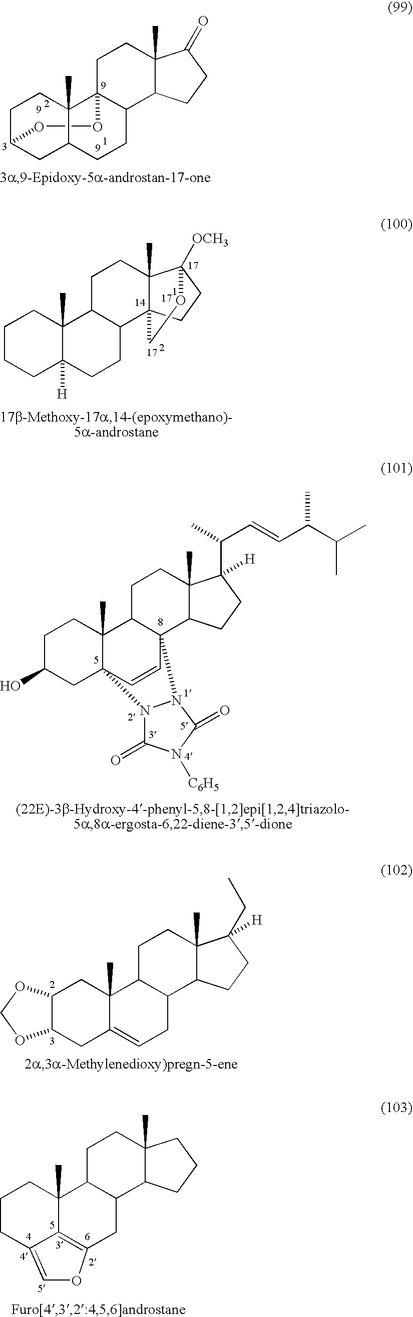

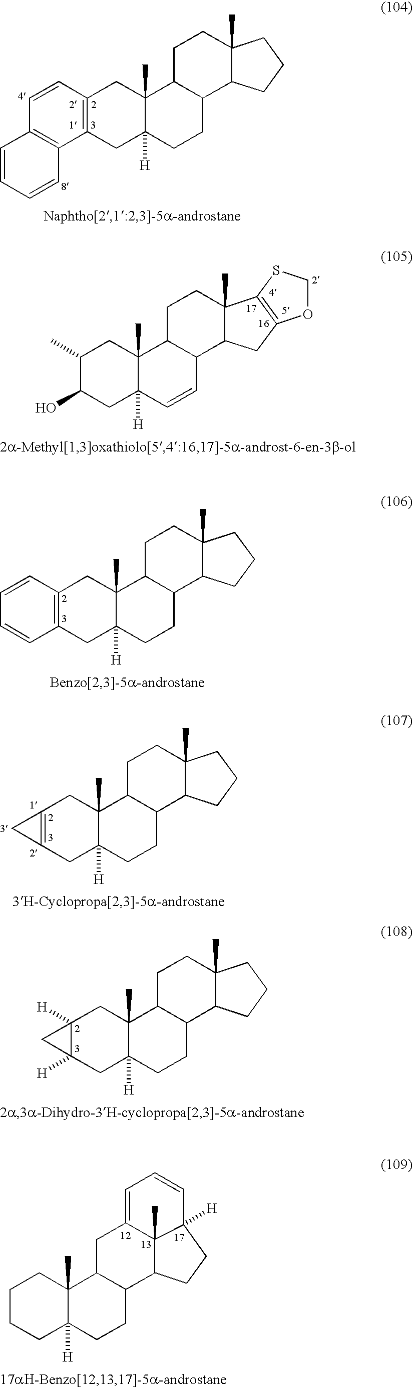

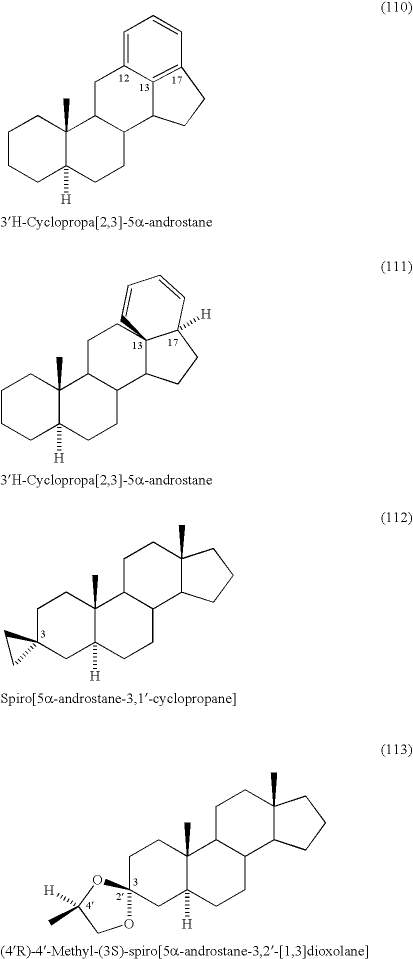

Yet, in certain embodiments, two or more steroid molecules are linked together covalently, as exemplified by formulae 3a and 3b.

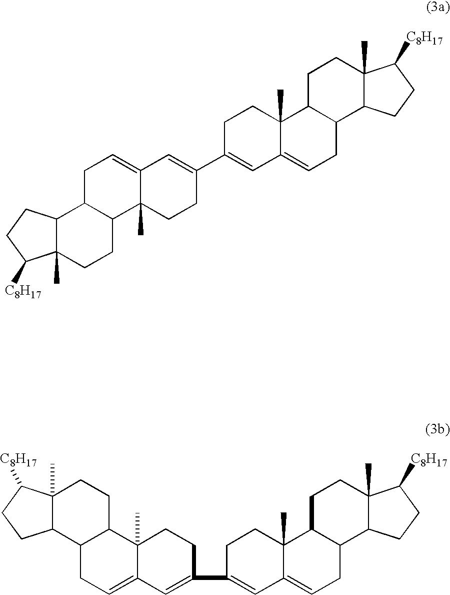

Table 16 provides examples of steroids that are useful.

TABLE 16
Exemplary steroids that are useful.

| | | Molecular |
|---|---|---|
| Trivial name | Chemical name | formula |
| Acrihellin | 5,14-dihydroxy-3β-[(3-methylcrotonoyl)oxy]-19-oxo- | C₂₉H₃₈O₇ |
| | 5β-bufa-20,22-dienolide | |
| Actodigin | 3β-(β-D-glucopyranosyloxy)-14-hydroxy-24-nor- | C₂₉H₄₄O₉ |
| | 5β,14β-chol-20(2)-eno-21,23-lactone | |
| Alfacalcidol | (5Z,7E)-(1S,3R)-9,10-secocholesta-5,7,10(19)-triene-,3- | C₂₇H₄₄O₂ |
| | diol | |
| Betamethasone | 9-fluoro-11β,17,21-trihydroxy-16β-methylpregna-1,4- | C₂₂H₂₉FO₅ |
| | diene-3,20-dione | |
| Canrenone | 3-oxo-17α-pregna-4,6-diene-21,17-carbolactone | C₂₂H₂₈O₃ |
| Clomegestone | 6-chloro-17-hydroxy-16α-methylpregna-4,6-diene-3,20- | C₂₂H₂₉ClO₃ |
| | dione | |
| Cyproterone | 6-chloro-1β,2β-dihydro-17-hydroxy-3′H- | C₂₂H₂₇ClO₃ |
| | cyclopropa[1,2]pregna-4,6diene-3,20-dione | |
| Dexamethasone | 9-fluoro-11β,17,21-trihydroxy-16α-methylpregna-1,4- | C₂₂H₂₉FO₅ |
| | diene-3,20-dione | |
| Disogluside | (25R)-3β-(β-D-glucopyranosyloxy)spirost-5-ene | C₃₃H₅₂O₈ |
| Ethinylestradiol | 19-nor-17α-pregna-1,3,5(10)-trien-20-yne-3,17-diol | C₂₀H₂₄O₂ |
| Fluazacort | 21-acetoxy-9-fluoro-11β-hydroxy-2′-methyl-16bH- | C₂₅H₃₀FNO₆ |
| | oxazolo[5′,4′:16,17]pegna- -1,4-diene-3,20-dione | |
| Fluocortin | 6α-fluoro-11β-hydroxy-16α-methyl-3,20-dioxopregna- | C₂₂H₂₇FO₅ |
| | 1,4-dien-21-oic acid | |
| Fusidic Acid | (17Z)-ent-16α-acetoxy-3β,11β-dihydroxy-4β,8,14- | C₃₁H₄₈O₆ |
| | trimethyl-18-nor-5β,10α-cholesta- -17(20),24-dien-21- | |
| | oic acid | |
| Gestrinone | 17-hydroxy-18α-homo-19-nor-17α-pregna-4,9,11-trien- | C₂₁H₂₄O₂ |
| | 20-yn-3-one | |
| Halometasone | | |
| | 2-chloro-6α,9-difluoro-11β,17,21-trihydroxy-16α- | C₂₂H₂₇ClF₂O₅ |
| | methylpregna-1,4-diene-3,20-dione | |
| Hydrocortisone | 11β,17,21-trihydroxypregn-4-ene-3,20-dione | C₂₁H₃₀O₅ |
| Mebolazine | 17β-hydroxy-2α,17-dimethyl-5α-androstan-3-one azine | C₄₂H₆₈N₂O₂ |
| Medroxyprogesterone | 17-hydroxy-6α-methylpregn-4-ene-3,20-dione | C₂₂H₃₂O₃ |
| Meproscillarin | 3β-(6-deoxy-4-O-methyl-α-L-mannopyranosyloxy)-14- | C₃₁H₄₄O₈ |
| | hydroxybufa-4,20,22-rienolide | |
| Mespirenone | 7α-acetylthio-15α,16α-dihydro-3-oxo-3′H- | C₂₅H₃₀O₄S |
| | cyclopropa[15,1]-17α-pregna- -1,4-diene-21,17- | |
| | carbolactone | |
| Mestranol | 3-methoxy-19-nor-17α-pregna-1,3,5(10)-trien-20-yn-17- | C₂₁H₂₆O₂ |
| | ol | |
| Naflocort | 9-fluoro-1′,4′-dihydro-11β,21-dihydroxy-16bH- | C₂₉H₃₃FO₄ |
| | naphtho[2′,3′:16,17]prena- -1,4-diene-3,20-dione | |
| Norenthisterone | 17-hydroxy-19-nor-17α-pregn-4-en-20-yn-3-one | C₂₀H₂₆O₂ |
| Norgesterone | 17-hydroxy-19-nor-17α-pregna-5(10),20-dien-3-one | C₂₀H₂₈O₂ |
| Norgestrel | rac-17-hydroxy-18α-homo-19-nor-17α-pregn-4-en-20- | C₂₁H₂₈O₂ |
| | yn-3-one | |
| Oxandrolone | 17β-hydroxy-17α-methyl-2-oxa-5α-androstan-3-one | C₁₉H₃₀O₃ |
| Oxymetholone | 17β-hydroxy-2-(hydroxymethylene)-17α-methyl-5α- | C₁₉H₂₈O₃ |
| | androstan-3-one | |
| Pancuronium | 1,1′-(3α,17β-diacetoxy-5α-androstane-2β,16β- | C₃₅H₆₀Br₂N₂O₄ |
| bromide | diyl)bis(-methylpiperidinium) dibromide | |
| Prednisolone | 11β,17,21-trihydroxypregna-1,4-diene-3,20-dione | C₂₁H₂₈O₅ |
| Prednisone | 17,21-dihydroxypregna-1,4-diene-3,11,20-trione | C₂₁H₂₆O₅ |
| Proscillardin | 3β-(6-deoxy-α-L-mannopyranosyloxy)-14-hydroxybufa- | C₃₀H₄₂O₈ |
| | 4,20,22-trienolide | |
| Roxibolone | 11β,17β-dihydroxy-17α-methyl-3-oxoandrosta-1,4- | C₂₁H₂₈O₅ |
| | diene-2-carboxylic acid | |
| Spironolactone | 7α-acetylthio-3-oxo-17α-pregn-4-ene-21,17- | C₂₄H₃₂O₄S |
| | carbolactone | |
| Timobesone | S-methyl 9-fluoro-11β,17α-dihydroxy-16β-methyl-3- | C₂₂H₂₉FO₄S |
| | oxoandrosta- -1,4-diene-17β-carbothioate | |
| Triamcinolone | 9-fluoro-11β,16α,17,21-tetrahydroxypregna-1,4-diene- | C₂₁H₂₇FO₆ |
| | 3,20-dione | |
| Ursodeoxycholic | 3α,7β-dihydroxy-5β-cholan-24-oic acid | C₂₄H₄₀O₄ |
| acid | | |

Mixtures of these steroids may also be employed.

The steroid is included in the composition in a concentration that provides a desirable ratio between the efficacy and safety. Typically, steroids are included in the composition in a concentration between about 0.005% and about 12%. However, in some embodiments, the concentration is between about 0.005% and about 0.5%, in other embodiment between about 0.5% and about 2%, and in additional embodiments between about 2% and about 5% or between about 5% and about 12%.

In one or more embodiments, the steroid possesses immunomodulating and/or anti-inflammatory properties. Without being bound to a specific theory, immunomodulating and/or anti-inflammatory steroids act, among other mechanisms, through inhibition of the activity of phospholipase A₂. They also may have anti-proliferative effects on keratinocytes and other cell types. They can suppress collagen synthesis by fibroblasts, but this may lead to adverse effects. Anti-inflammatory steroids are roughly grouped according to relative anti-inflammatory activity, but activity may vary considerably depending upon the vehicle, the site of application, disease, the individual patient and whether or not an occlusive dressing is used, as exemplified in the Table 17 below.

TABLE 17
Exemplary anti-inflammatory steroids that are useful.

| | | Typical concentration |
|---|---|---|
| Relative Potency | Generic Name | in topical products |
| Low Potency | Hydrocortisone | 0.5%-1% |
| | hydrocortisone acetate | 0.5-1.0% |
| | Desonide | 0.02-0.2% |
| Medium Potency | Betamethasone valerate | 0.05%-0.1% |
| | Prednicarbate | 0.02-0.2% |
| | Clobetasone-17-butyrate | 0.05% |
| | Flucinonide | 0.01%-0.05% |
| | Fluocinolone acetonide | 0.01-0.01% |
| | Alcometasone dipropionate | 0.01% |
| | Mometasone furoate | 0.1% |
| | Triamcinolone acetonide | 0.025%-0.1% |
| High Potency | Betamethasone-17-benzoate | 0.025% |
| | Methylprednisolone aceponate | 0.1% |
| | Betamethasone dipropionate | 0.025%, 0.05% |
| | Halcinonide | 0.1% |
| | Triamcinolone acetonide | 0.5% |
| Highest Potency | Halobetasol | 0.05% |
| | Clobetasol-17-propionate | 0.05% |

In one or more embodiments, the steroid is selected from the group of low-potency anti-inflammatory steroids, medium potency anti-inflammatory steroids and high potency anti-inflammatory steroids.

In one or more embodiments, the anti-inflammatory steroid is included in the composition at a concentration between about 0.005% and about 1%.

The McKenzie vasoconstrictor assay, as described, for example, in the British Journal of Dermatology 1975; 93:563-71 and versions thereof, has been the primary method used for classifying the potency of a product, containing an anti-inflammatory steroids. Thus, in one or more embodiments, the anti-inflammatory steroid is a steroid that positively affects the vasoconstrictor assay.

In one or more embodiments, the steroid is a hormone. Hormones are known to affect a variety of biological processes in any organism, and thus, their inclusion in the composition, which is intended for local treatment of the skin, the vagina, the rectum as well as other body surfaces and cavities provided an advantageous treatment modality. Such compositions containing hormones can be further administered systemically, via the transdermal or transmucosal route, in order to alleviate a disorder that is affected by the specific hormone, or in order to tune the hormonal balance of the body in order to attain certain effects controlled by hormones, such as contraception and birth induction.

In one or more embodiments, the steroid hormone is a male hormone or an androgen. Non-limiting examples of male hormones/androgens include testosterone, testosterone cipionate, testosterone decanoate, testosterone enantate, testosterone isocaproate, testosterone phenylpropionate, testosterone propionate, testosterone undecylate, 5α-dihydrotestosterone, dehydroepiandrosterone (also termed prasterone and DHEA), androstenedione, androstanediol, androsterone, androstenolone, prasterone enantate, prasterone sodium sulfate, ormeloxifene, mesterolone, fluoxymesterone, methyltestosterone, gestrinone, delmadinone, delmadinone acetate, chlormadinone, chlormadinone acetate, danazol and testolactone.

In one or more embodiments, the steroid hormone is a female hormone or an estrogen. Non-limiting examples of female hormones/estrogens include estradiol, estradiol benzoate, estradiol cipionate, estradiol dipropionate, estradiol enantate, estradiol hexahydrobenzoate, estradiol phenylpropionate, estradiol valerate, polyestradiol phosphate, estriol, estriol sodium succinate, estriol succinate, polyestriol phosphate, quinestradol, ethinylestradiol, estrapronicate, mestranol, estrapronicate and equilin.

In one or more embodiments, the steroid hormone is a progestogen. Non-limiting examples of progestogens include progesterone, norethisterone, norethisterone acetate, norethisterone enantate, medroxyprogesterone acetate, delmadinone acetate, flugestone acetate, dydrogesterone, desogestrel, norgestrel, levonorgestrel, dydrogesterone, gestodene, chlormadinone acetate, dienogest, drospirenone, lynestrenol, tybolone, cyproterone acetate, megestrol acetate, nomegestrol acetate.

Yet, in additional embodiments, the steroid an inhibitor of a steroid hormone. Non-limiting examples of such inhibitors are finasteride, dutasteride and spironolactone.

In one or more embodiments, the steroid is a vitamin D. The term vitamin D is used to describe all steroids that exhibit qualitatively the biological activity of calciol (vitamin D₃). Non limiting examples of vitamin D compounds are provided in Table 5.

Yet, in additional embodiments, the steroid is a vitamin D₃ analogue. Exemplary vitamin D₃ analogs include calcipotriol, tacalcitol, maxacalcitol, and calcitriol, with calcipotriol being especially preferred. Vitamin D₃ analogues and derivatives are known to degrade at low pH levels. Therefore, in certain preferred embodiments, the steroid is a vitamin D₃ or an analogue or a derivative thereof, the pH is adjusted to the range between about 7 and about 10, or between about 7.5 and about 9. In one or more embodiments, the pH is adjusted using a buffering agent, suitable of maintaining a pH level between about 7 and about 10, or between about 7.5 and about 9.

TABLE 5
Examples of vitamin D compounds

| Vitamin D name | Systematic steroid name |
|---|---|
| Cholecalciferol (also termed calciol, | (5Z,7E)-(3S)-9,10-seco-5,7,10(19)- |
| cholecalciferol, vitamin D₃ and colecalciferol) | cholestatrien-3-ol |
| 25-Hydroxycholecalciferol (also termed | (5Z,7E)-(3S)-9,10-seco-5,7,10(19)- |
| calcidiol | cholestatriene-3,25-diol |
| 1α,25-Dihydroxycholecalciferol (also termed | (5Z,7E)-(1S,3R)-9,10-seco-5,7,10(19)- |
| calcitriol) | cholestatriene-1,3,25-triol |
| Ergocalciferol (also termed ercalciol and | (5Z,7E,22E)-(3S)-9,10-seco-5,7,10(19),22- |
| ergocalciferol) | ergostatetraen-3-ol |
| 1α,25-Dihydroxyergocalciferol (also termed | (5Z,7E,22E)-(1S,3R)-9,10-seco-5,7,10(19),22- |
| ercalcitriol) | ergostatetraen-1,3,25-triol |
| 22,23-Dihydroergocalciferol (also termed | (5Z,7E)-(3S)-9,10-seco-5,7,10(19)- |
| (24S)-methylcalciol and 22,23- | ergostatrien-3-ol |
| dihydroercalciol) | |
| 1α,24R,25-Trihydroxycholecalciferol (also | (5Z,7E)-(1S,3R,24R)-9,10-seco-5,7,10(19)- |
| termed calcitetrol) | cholestatriene-1,3,24,25-tetrol |
| Previtamin D₃ (also termed precalciferol and | (6Z)-(3S)-9,10-seco-5(10),6,8-cholestatrien-3- |
| (6Z)-tacalciol) | ol |
| Tachysterol₃ (also termed tacalciol) | (6E)-(3S)-9,10-seco-5(10),6,8-cholestatrien-3- |
| | ol |
| Isovitamin D₃ (also termed (5E)-isocalciol) | (5E,7E)-(3S)-9,10-seco-1(10),5,7- |
| | cholestatrien-3-ol |
| Dihydrotachysterol₃ (also termed | (5E,7E)-(3S,10S)-9,10-seco-5,7-cholestadien- |
| dihydroercalciol) | 3-ol |

Further examples of vitamin D compounds include, but are not limited to (1S)-Hydroxycalciol (also termed 1α-hydroxycholecalciferol and alfacaleidol), (24R)-Hydroxycalcidiol (also termed 24(R),25-dihydroxycholecalciferol), 25-Fluorocalciol (also termed 25-fluorocholecalciferol), Ercalcidiol (also termed 25-hydroxyergocalciferol), Ertacalciol (also termed tachysterol₂, (5E)-Isocalciol (also termed isovitamin D₃, 22,23-Dihydroercalciol), (24S)-methylcalciol (also termed vitamin D₄), (5E)-(10S)-10,19-Dihydroercalciol, (also termed dihydrotachysterol₂, hytakerol, and dihydrotachysterol), (24S)-Ethylcalciol (also termed vitamin D₅) and (22E)-(24R)-Ethyl-22,23-didehydrocalciol, (also termed vitamin D₆).

In one or more embodiments, the steroid is a phytosteroid or a phytosterol. As used herein, the term “phytosteroid” or “phytosterol” includes all steroids that are obtained, derived or extracted from plant sources. Non-limiting examples of families of phytosteroids and phytosterols include ecdysones, withanolids, sterines, steroid saponins and soflavonoids. Non-limiting examples of phytosteroid and phytosterol compounds include alpha-sitosterol, beta-sitosterol, stigmastanol, campesterol, alpha-sitostanol, beta-sitostanol, stigmastanol, campestanol, avenosterol, brassicasterol, desmosterol, chalinosterol, beta-ecdysone, whithaferin A, beta-sitosterine, stigmasterine, campesterine, ergosterine, diosgenin, daidzein, glycitein, genistein, muristerone, poriferasterol, clionasterol, campestanol, and cycloartenol, as well as all natural or synthesized forms and derivatives thereof, such as fatty acid esters, such as ferulic acid esters, oleoyl esters, and cinnamic acid esters, including isomers.

Plant oils and extracts which contain steroids are also useful. Non limiting examples of plants that contain steroids include nuts seeds, sprouted seeds and grains (such as alfalfa), St. Mary's thistle, ginkgo biloba, saw palmetto, panax, siberian ginseng, foeniculum vulgare, cimicifuga racemosa, licorice root, red clover, sage, sarsaparilla, sassafras, angelica sinensis achillea millefolium, aneimone pratensis, angelica sinensis, glycyrrhiza glabra, hypericum perforatum, larrea, panax, piscidia erythrina, plantago psyllium, serenoa repens, symphytum, taraxacum officinale, trifolium pratense, turnera spp., tussilago farfara, valeriana officinalis, viburnum prunifolium, calendula officinalis

In one or more embodiments, the steroid is a compound that is positively identified using a laboratory method, suitable of detecting a steroid.

Steroids in Combination with Other Agents

Several disorders of the skin, a body cavity or mucosal surface (e.g., the mucosa of the nose, mouth, eye, ear, vagina or rectum) involve a combination of inflammation, cell proliferation and differentiation abnormalities, and other biological abnormalities that can be effected by a steroid; and other etiological factors that require an additional therapeutic modality. For example, psoriasis involves inflammation as well as excessive cell proliferation and inadequate cell differentiation. Atopic dermatitis involves inflammation, skin dryness and keratinocyte growth abnormality. Bacterial, fungal and viral infections involve pathogen colonization at the affected site and inflammation. Likewise, hair growth disorders and other pilosebaceous disorders involve an impaired hormonal balance (which can be affected by a steroid hormone or a steroid hormone antagonist), together with other etiological factors, that can be affected a non-steroidal active agent. Hence, in many cases, the inclusion of an additional therapeutic agent in the foamable pharmaceutical composition, contributes to the clinical activity of the steroid. Thus, in one or more embodiments, the foamable composition further includes at least one additional therapeutic agent, in a therapeutically effective concentration.

In one or more embodiments, the at least one additional non-steroidal therapeutic agent is selected from the group consisting of an anti-infective, an antibiotic, an antibacterial agent, an antifungal agent, an antiviral agent, an antiparasitic agent, a nonsteroidal anti-inflammatory drug, an immunosuppressive agent, an immunomodulator, an immunoregrlilating agent, a hormonal agent, vitamin A, a vitamin A derivative, vitamin B, a vitamin B derivative vitamin C, a vitamin C derivative, vitamin F, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, an anti-wrinkle agent, a radical scavenger, a metal oxide (e.g., titanium dioxide, zinc oxide, zirconium oxide, iron oxide), silicone oxide, an anti wrinkle agent, a skin whitening agent, a skin protective agent, a masking agent, an anti-wart agent, a refatting agent, a lubricating agent and mixtures thereof.

In certain cases, the disorder to be treated involves unaesthetic lesions that need to be masked. For example, rosacea involves papules and pustules, which can be treated with a steroid, as well as erythema, telangiectasia and redness, which do not respond to treatment with a steroid. Thus, in one or more embodiments, the additional active agent is a masking agent, i.e., a pigment. Non limiting examples of suitable pigments include brown, yellow or red iron oxide or hydroxides, chromium oxides or hydroxides, titanium oxides or hydroxides, zinc oxide, FD&C Blue No. 1 aluminum lake, FD&C Blue No. 2 aluminum lake and FD&C Yellow No. 6 aluminum lake.

In an embodiment, the active agent is a hair growth regulator. Suitable hair growth regulators include but are not limited to N-acetylgalactosamine, N-acetylglucosamine, N-acetylmannosamine, acitretin, aminexil, ascomycin, asiatic acid, azelaic acid, benzalkonium chloride, benzethonium chloride, benzydamine, benzyl nicotinate, benzoyl peroxide, benzyl peroxide, betulinic acid, betulonic acid, calcium pantothenate, celastrol, cepharanthine, chlorpheniramine maleate, clinacycin hydrochloride, crataegolic acid, cromakalin, cyproterone acetate, diazoxide, diphenhydramine hydrochloride, dutasteride, estradiol, ethyl-2-hydroxypropanoate, finasteride, D-fucono-1,5-lactone, furoate, L-galactono-1,4-lactone, D-galactosamine, D-glucaro-1,4-lactone, D-glucosamine-3-sulphate, hinokitiol, hydrocortisone, 2-hydroxypropionic acid, isotretinoin, itraconazole, ketoconazole, latanoprost, 2-methyl propan-2-ol, minocyclin, minoxidil, mipirocin, mometasone, oleanolic acid, panthenol, 1,10-phenanthroline, phenyloin, prednisolone, progesterone, propan-2-ol, pseudoterins, resorcinol, selenium sulfide, tazarotene, triclocarbon, triclosan, triiodothyronine, ursolic acid, zinc pyrithione and derivatives, esters, salts and mixtures thereof.

In an embodiment, the therapeutic agent is a hormone. Suitable hormones include but are not limited to methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5a-dihydrotestosterone, testolactone, 17a-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5a-pregnan-3b,20a-diol sulfate, 5a-pregnan-3b,20b-diol sulfate, 5a-pregnan-3b-ol-20-one, 16,5a-pregnen-3b-ol-20-one, 4-pregnen-20b-ol-3-one-20-sulfate, acetoxypregnenolone, anagestone acetate, cyproterone, dihydrogesterone, fluorogestone acetate, gestadene, hydroxyprogesterone acetate, hydroxymethylprogesterone, hydroxymethyl progesterone acetate, 3-ketodesogestrel, megestrol, melengestrol acetate, norethisterone, progestins and derivatives, esters, salts and mixtures thereof.

In an embodiment, the therapeutic agent is a hydroxyacid. Suitable hydroxy acids include but are not limited to agaricic acid, aleuritic acid, allaric acid, altraric acid, arabiraric acid, ascorbic acid, atrolactic acid, benzilic acid, citramalic acid, citric acid, dihydroxytartaric acid, erytliraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllacetic acid, mucic acid, phenyllacetic acid, pyruvic acid, quinic acid, ribaric acid, ribonie acid, saccharic acid, talaric acid, tartaric acid, tartronic acid, threaric acid, tropic acid, uronic acids, xylaric acid and derivatives, esters, salts and mixtures thereof.

### Keratolytic

In an embodiment, the active agent is a keratolytic agent. The term “keratolytic agent” is used herein to mean a compound which loosens and removes the stratum corneum of the skin, or alters the structure of the keratin layers of skin. Keratolytic agents are used in the treatment of many dermatological disorders, which involve dry skin, hyperkeratiinization (such as prsoriasis), skin itching (such as xerosis), acne and rosacea. Suitable keratolytic agents include but are not limited to N-acetylcysteine, azelaic acid, cresols, dihydroxy benzene compounds, such as resorcinol and hydroquinone, alpha-hydroxy acids, such as lactic acid and glycolic acid, phenol, pyruvic acid, resorcinol, sulfur, salicylic acid, retinoic acid, isoretinoic acid, retinol, retinal, urea and derivatives, esters, salts and mixtures thereof.

The term “keratolytic agent” refers herein to a compound which loosens and removes the stratum corneum of the skin, or alters the structure of the keratin layers of skin.

Suitable keratolytic agents also include alpha-hydroxy acids. Alfa hydroxy acids are keratolytic, and they are also capable of trapping moisture in the skin and initiating the formation of collagen. Suitable hydroxy acids include but are not limited to agaricic acid, aleuritic acid, allaric acid, altraric acid, arabiraric acid, ascorbic acid, atrolactic acid, benzilic acid, citramalic acid, citric acid, dihydroxytartaric acid, erythraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllacetic acid, mucic acid, phenyllacetic acid, pyruvic acid, quinic acid, ribaric acid, ribonic acid, saccharinc acid, talaric acid, tartaric acid, tartronic acid, threaric acid, tropic acid, uronic acids, xylaric acid and derivatives, esters, salts and mixtures thereof.

Yet, another preferred keratolytic agent is urea, as well as derivatives thereof. Urea possesses both keratolytic and skin-hydration properties which are beneficial to the damaged tissue of the skin.

Another preferred group of keratolytic agents, suitable for inclusion in the therapeutic composition is beta-hydroxy acids, such as salicylic acid (o-hydroxybenzoic acid). Beta hydroxyl acids are keratolytic, and they are also have anti-inflammatory and antibacterial properties.

Short chain carboxylic acids (carboxylic acids having up to 6 carbon atoms in their skeleton) are also suitable for inclusion in the therapeutic composition as keratolytic agents. Examples of short chain carboxylic acid include, but are not limited to formic acid, acetic acid, propionic acid, butyric acid (Butanoic acid), valeric acid (pentanoic acid) and caproic acid (hexanoic acid). In the context, di-carboxylic acids having up to 6 carbon atoms in their skeleton are also suitable under the definition of short chain carboxylic acids having up to 6 carbon atoms in their skeleton. Non-limiting examples of suitable dicarboxylic acids are malonic acid (propanedioic acid), succinic acid (butanedioic acid), glutaric acid (Pentanedioic acid) and adipic acid (Hexanedioic acid). Also suitable under the definition of short chain carboxylic acid are unsaturated short chain carboxylic acids, i.e., short chain carboxylic acids, having one or more double bonds in their carbon skeleton; and halogenated short chain carboxylic acids, such as fluoroethanoic acid (CH2FCO2H), chloroethanoic acid (CH2ClCO2H) and dichloroethanoic acid (CHCl2CO2H). Dicarboxylic acids, having between about 6 and about 14 carbon atoms in their carbon atom skeleton also possess leratolytic properties. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid.

Another group of keratolytic agents include phenol and substituted phenolic compounds. Such compounds are known to dissolve and loosen the intracellular matrix of the hyperkeratinized tissue. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic.

Vitamin A and its derivatives, such as retinol, retinal, retinoic acid, retinyl acetate, retinyl palmitate, retinyl ascorbate, isotretinoin, tazarotene, adapalene, 13-cis-retinoic acid, acitretin all-trans beta carotene, alpha carotene, lycopene, 9-cis-beta-carotene, lutein and zeaxanthin are another class of keratolytic agents, which alter the structure of the skin and promote peeling.

In certain embodiments, the keratolytic agent includes at least two keratolytic agents. At least two or more keratolytic agents in the therapeutic composition, a safe and effective peeling agent is attained, which breaks down the keratin layer of the skin, where the microorganisms reside. As a result of such breaking down of the keratin layer, the microorganisms cannot further survive in the infected area. The combination of at least two keratolytic agents enables a selective breaking down of keratin in infected skin areas, while non-infected skin areas are not affected. This phenomenon is explained by the fact that the keratin layer in infected skin areas is deformed and thus it is more vulnerable to keratolytic disintegration. Furthermore, combining at least two keratolytic agents facilitates use of each agent in a substantially minimally-irritating concentration, thus decreasing the overall irritation of the therapeutic composition.

In one or more embodiments, the keratolytic agent includes at least two keratolytic agents, from different families of chemicals. Thus, in preferred embodiments, the keratolytic agent includes at east two agents, from different chemical families, selected from the group consisting of: (1) an alpha-hydroxy acid; (2) a beta-hydroxy acid; (3) a short-chain carboxylic acid; (4) a hydroxyl benzene; (5) a vitamin A derivative; and (6) urea. As detailed above, each of these keratolytic agent families possess, in addition to their keratolytic property, additional therapeutically-beneficial feature, such as anti-inflammatory, skin hydration and antibacterial properties for readily contributing to the overall therapeutic benefit of the therapeutic composition.

In an embodiment, the active agent is a lactam. Suitable lactams include but are not limited to L-galactono-1,4-lactam, L-arabino-1,5-lactam, D-fucono-1,5-lactam, D-glucaro-1,4-lactam, D-glucurono-6,3-lactam, 2,5-tri-O-acetyl-D-glucurono-6,3-lactam, 2-acetamido-2-deoxyglucono-1,5-1-actam, 2-acetamido-2-deoxygalactono-1,5-lactam, D-glucaro-1,4:6,3-dilactam-, L-idaro-1,5-lactam, 2,3,5,tri-O-acetyl-D-glucaro-1,4-lactam, 2,5-di-O-acetyl-D-glucaro-1,4:6,3-dilactam, D-glucaro-1,5-lactam methyl ester, 2-propionoamide-2-deoxyglucaro-1,5-lactam and derivatives, esters, salts and mixtures thereof.

### Nonsteroidal Anti-Inflammatory Agent

In an embodiment, the therapeutic agent is a non-steroidal anti-inflammatory agent.

Inflammation is defined as “redness, swelling, and fever in a local area of the body, often with pain and disturbed function, in reaction to an infection or to a physical or chemical injury” (Random House Webster's Dictionary). Typical symptoms of disorders of the skin, body surfaces, body cavities and mucosal surfaces (e.g., the mucosa of the nose, mouth, eye, ear, respiratory system, vagina or rectum) that involve inflammation, as at least one of their etiological factors, include redness (rash, erythema), tissue thickening and/or swelling (oedema), itch (pruritus), blistering and exudate. Inflammatory disorders can by short term or long tem (chronic). Inflammation typically involves overproduction of pro-inflammatory cytokines, such as TNF-alpha, TNF-beta, interleukin-1, interleukin-4, interleukin-6, interleukin-10, interleukin-12, IFN-gamma from T cells, or increased release of cytokines and pro-inflammatory mediators from mast cells.

In the context, a nonsteroidal immunomodulating agent (also termed herein “nonsteroidal anti-inflammatory agent” and “NSAID”) is a pharmaceutically active compound, other than a corticosteroid, which affects the immune system in a fashion that results in a reduction, inhibition, prevention, amelioration or prevention of an inflammatory process and/or the symptoms of inflammation and or the production pro-inflammatory cytokines and other pro-inflammatory mediators, thereby treating or preventing a disease that involves inflammation.

In one or more embodiments, the NSAID is an inhibitor of the cyclooxygenase (COX) enzyme. Two forms of cyclooxygenase are known today: the constitutive cyclooxygenase (COX-1); and the inducible cyclooxygenase (COX-2), which is proinflammatory. Thus, in one or more embodiments, the NSAID is selected from the group consisting of a COX-1 inhibitor, a COX-2 inhibitor or a non-selective NSAID, which simultaneously inhibits both COX-1 and COX-2.

The term “selective COX-2 inhibitor” relates o a compound able to inhibit cyclooxygenase-2 without significant inhibition of COXe-1. Typically, it includes compounds that have a COX-2 IC₅₀ of less than about 0.2 micro molar, and also have a selectivity ratio of COX-2 inhibition over COX-1 inhibition of at least 50, and more typically, of at least 100, Inhibitors of the cyclooxygenase pathway in the metabolism of arachidonic acid used in the present invention may inhibit enzyme activity through a variety of mechanisms. By the way of example, and without limitation, the inhibitors used in the methods described herein may block the enzyme activity directly by acting as a substrate for the enzyme.

Selective COX-2 Inhibitors include, in an exemplary manner diaryl-substituted furanones (e.g., Rofecoxib); diaryl-substituted pyrazoles (e.g., Celecoxib); indole acetic acids (e.g., Etodolac); and sulfonanilides (e.g., Nimesulide) and salts and derivatives thereof.

In one or more embodiments, the selective COX-2 inhibitor is selected from the group consisting of celecoxib, deracoxib, valdecoxib, rofecoxib, lumiracoxib, etoricoxib, meloxicam, parecoxib, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)-phenyl]-3(2H)-pyridazinone, 2-[(2,4-dichloro-6-methylphenyl)amino]-5-ethyl-1-benzeneacetic acid, (3 Z)-3-[(4-chlorophenyl)[4-(methylsulfonyl)phenyl]met-hylene]dihydro-2(3H)-furanone, and (S)-6,8-dichloro-2-(trifluoromethyl)-2H-1-benzopyran-3-carboxylic acid.

In additional embodiments, the selective COX-2 inhibitor is selected from the group consisting of ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam, and 2-fluoro-a-methyl[1,1′-biphenyl]-4-ace-tic acid, 4-(nitrooxy)butyl ester.

In one or more embodiments, the NSAID is salicylic acid a salicylic acid derivatives. Exemplary salicylic acid derivative include, in a non limiting fashion, aspirin, sodium salicylate, choline magnesium trislicylate, salsalate, diflunisal, salicylsalicylic acid, sulfasalazine, olsalazine, esters of salicylic acid with a carboxylic acid, esters of salicylic acid with a dicarboxylic acid, esters of salicylic acid with a fatty acid, esters of salicylic acid with a hydroxyl fatty acid, esters of salicylic acid with an essential fatty acid, esters of salicylic acid with a polycarboxylic acid, and any compound wherein salicylic acid is linked to an organic moiety through a covalent bond.

In one or more embodiments, the NSAID is para-aminophenol (e.g., acetaminophen) and salts and derivatives thereof.

In one or more embodiments, the NSAID is an indole or an indole—acetic acid derivative (e.g., indomethacin, sulindac, etodolac) and salts and derivatives thereof.

In one or more embodiments, the NSAID is an aryl acetic acids (e.g., tolmetin, diclofenac, ketorolac) and salts and derivatives thereof.

In one or more embodiments, the NSAID is an arylpropionic acid and salts and derivatives thereof. Exemplary arylpropionic acid derivative include, in a non limiting fashion, are ibuprofen, naproxen, flubiprofen, ketoprofen, fenoprofen, oxaprozin.

In one or more embodiments, the NSAID is anthranilic acids or an anthranilic acid derivative, also termed “fenamates” (e.g., mefenamic acid, meclofenamic acid) and salts and derivatives thereof.

In one or more embodiments, the NSAID is selected from the group of enolic acids, enolic acid salts, enolic acid esters, amides, anhydrides and salts and derivatives thereof. Non-limiting examples of enolic acid derivatives include oxicams (piroxicam, tenoxicam) and pyrazolidinediones (phenylbutazone, oxyphenthratrazone)

Yet, in additional embodiments, the NSAID is an alkanone (e.g., nabumetone).

Certain imidazole drugs (e.g., ketoconazole) also possess anti-inflammatory properties, (See: J Am Acad. Dermatol. 1991 August; 25(2 Pt 1):257-61).

Another group of nonsteroidal immunomodulating agents includes agents, which inhibit pro-inflammatory cytokines, such as TNF-alpha, TNF-beta, interleukin-1, interleukin-4, interleukin-6, interleukin-10, interleukin-12 and IFN-gamma from T cells, which are especially important in the induction of inflammation or inhibit the release of cytokines and pro-inflammatory mediators from mast cells.

Agents that are used to affect the untoward influence of pro-inflammatory cytokines are chemically or biologically-originated materials that suppress the pro-inflammatory effect of a pro-inflammatory cytokine via various mechanisms, including, but not limited to (a) inhibiting the formation of a pro-inflammatory cytokine; (b) suppressing the interaction of a pro-inflammatory cytokine with its receptors; or (c) neutralization the proinflammatory cytokine by direct or indirect interaction.

Examples of chemical anti TNF-α agents are known pharmaceutical materials, such as pentoxifylline, propentofylline, torbafylline (and other related xanthines), amiloride, chloroquine, thalidomide and structural analogs thereof. Examples for biological anti-TNF-α agents are anti-TNF-α antibodies and soluble TNF-α receptors. Additional compounds are those that impair the signal transduction cascade from the receptor to other functional organs of the living cell. Such active agents, as well additional compounds, which are capable of inhibiting the production or otherwise suppressing the pro-inflammatory effects of TNF-α can be used in the composition.

Immunosuppressant agents, immunoregulating agents and immunomodulators constitute an additional class of nonsteroidal anti-inflammatory agents, which are used. Such agents are chemically or biologically-derived agents that modify the immune response or the functioning of the immune system (as by the stimulation of antibody formation or the inhibition of white blood cell activity). Immunosuppressant agents and immunomodulators include, among other options, cyclic peptides, such as cyclosporine, tacrolimus, tresperimus, pimecrolimus, sirolimus (rapamycin), verolimus, laflunimus, laquinimod and imiquimod. In one or more embodiments, the non steroidal immunomodulating agent is a calcineurin Inhibitor.

In one or more embodiments, the NSAID is a nitric oxide inhibitor. Nitric oxide (NO) is a potent secondary messenger that is both highly reactive and highly diffusible. It is generated physiologically by a family of enzymes, referred to as NO synthases (NOS). Overproduction of NO plays a key role in the pathology of a wide range of disorders including disorders that involve inflammation, and NOS inhibitors have been suggested as anti-inflammatory agents. Agents that neutralize NO (also called “NO scavengers”) are considered as potential anti-inflammatory agents as well.

Also useful are compounds that inhibit or slow down the migration of leucocytes (white blood cell), e.g., macrophages, neutrophils, and monocytes towards an afflicted skin surface or mucosal membrane, which is known to accelerate the inflammatory process.

Among other inhibitors of leucocyte chemoaxis, dicarboxylic acids, having between about 6 and about 14 carbon atoms in their carbon atom skeleton are particularly useful in the treatment of disorders of the skin and mucosal membranes that involve inflammation. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 11,14-tetradecanedioic acid. Thus, in one or more embodiments, dicarboxylic acids, having between about 6 and about 14 carbon atoms in their carbon atom skeleton, as well as their salts and derivatives (e.g., esters, amides, mercapto-derivatives, anhydraides), are useful immunomodulators in the treatment of disorders of the skin and mucosal membranes that involve inflammation. Azelaic acid and its salts and derivatives are preferred.

Certain preferred dicarboxylic acid derivatives include a dicarboxylic acid wherein at least one ester moiety of the compound comprises a keratolytic agent, selected from the group consisting of alpha-hydroxy acids and derivatives thereof, beta-hydroxy acids and derivatives thereof, hydroxybenzoic acid and their ester, anhydride and amine derivatives, alkylhydroxybenzoate, dihydroxy benzene and their ester, anhydride and amide derivatives, cresols and their ester, anhydride and amide derivatives. Keratolytic agents also include alcohol derivatives of Vitamin A (retinoic acid), e.g., retinol and derivatives thereof, as provided in U.S. Pat. No. 6,180,669. Additional preferred dicarboxylic acid derivatives comprise at least one ester of a active alcohol moiety, selected from the groups of steroid hormones, corticosteroids, vitamin E and vitamin D, as provided in US Patent Application 20040191196.

In one or more embodiments, the NSAID is an ion channel modulator. Ion channels are protein macromolecules located in the cell membranes that enable the selective movement of sodium, potassium, and calcium from outside the cell to inside the cell and vice-versa.

In one or more embodiments, the NSAID is a potassium ion channel modulator. It has been shown that the potassium ion channel modulator play important roles in controlling T-cell activation and thus, they can be used to control inflammation.

In one or more embodiments, the potassium ion channel modulator is selected from the group consisting of dendrotoxin, dendrotoxin I, dendrotoxin K, alpha-dendrotoxin, beta-dendrotoxin, gamma-dendrotoxin, margatoxin, stichodactyla toxin, tityustoxin K, apamin, charylotoxin, clotrimazole, dequalinium chloride, iberiotoxin, kaliotoxin, neuropeptide Y, noxiustoxin, tolbutamide, chlorpropamide, glibenclamide, glipizide, nategliniide, repagliniide, glyburide, tolazamide, nicorandil, fampridine and penitrem A, or is a pharmaceutically acceptable salt or prodrug thereof.

In an embodiment, the potassium ion channel modulator is selected from the list of potassium ion channel modulators, provided in WO 2004/093895.

In one or more embodiments, the NSAID is a sodium ion channel modulator. In one or more embodiments, the sodium ion channel blocker is selected from the group consisting of disopyramide, procainimide, quinidine, tocamide, mexiletene, lidocane, phenyloin, fosphenyloin, flecamide, propafenone, morcizine, lubeluzole, carbamazepine, sipatrigine, riluzole, tetrodotoxin, spheroidine, maculotoxin, vinpocetine, anthopleurin-c, lamotrigine, crobenetine, lifarizine, lanodipine, lomerizine, encamide, and flunarizine or is an isomer, a pharmaceutically acceptable salt, ester, or prodrug thereof.

In an embodiment, the potassium ion channel modulator is selected from the list of potassium ion channel modulators, provided in U.S. Pat. Appl. 20040224940 and 20040220187.

In one or more embodiments, the NSAID is a modulator of serotonin (5-hydroxytryptamine, 5-HT) activity. 5-HT is known to affect inflammation through its modulation effect on cytokine production (Cloez-Tayarani et al. Int. Immunol. 2003, 15 233). In certain embodiments, the serotonin activity modulator is a serotonin reuptake inhibitor. It has been shown that serotonin reduces inflammation and assists healing of experimental skin wounds, and thus, serotonin reuptake inhibitor can be used to control inflammation and associated disorders.

In one or more embodiments, the serotonin reuptake inhibitor is selected from the group consisting of citalopram, fluoxetine, fluvoxamine, paroxetine, escitalopram oxalate, sertraline, norfluoxetine and N-demethylsertraline.

In an embodiment, the serotonin reuptake inhibitor is selected from the list of potassium ion channel modulators, provided in US Pat Appl. 20040171664.

In one or more embodiments, the NSAID is an antioxidant. Reactive oxygen species play an important role in mediating skin inflammation, and antioxidants may provide protection.

Non-limiting examples of antioxidant agents include 21-[4-[2-amino-6-(diethylamino)-4-pyrimidinyl]-11-piperazinyl]-17α-hydroxypregna-4,9(11)-diene-3,20-dione, 17α-hydroxy-21-[4-[2,6-bis(dimethylamino)-4-pyrimidinyl]-1-piperazinyl]pregna-4,9(11)-diene-3,20-dione, 21-[4-[2-(diethylamino)-6-(1-pyrrolidinyl)-4-pyrimidinyl]-1-piperazinyl]-17α-hydroxypregna-4,9(11)-diene-3,20-dione, 17α-hydroxy-21-[4-[2-(diethylamino)-6-(4-methyl-1-piperazinyl(4-pyrimidinyl)]-1-piperazinyl]pregna-4,9(11)-diene-3,20-dione, 17 α-hydroxy-21-[4-[2,6-bis(diethylamino)-4-pyrimidinyl] 1-piperazinyl]pregna-4,9(11)-diene-3,20-dione, 1α-hydroxy-21-[4-[2-(diethylamino-)-6-(1-piperidinyl)-4-pyrimidinyl]-1-piperazinyl]pregna-4,9(11)-diene-3,20-dione, 21-[4-[2,6-bis(diethylamino)-b-4-pyrimidinyl)-4-pyrimidinyl]-1-piperazinyl]-1-piperazinyl]-17α-hydroxy-16 α-methylpregna-1,4,9(11)-triene-3,20-dione, 17α-hydroxy-21-[4-[2,6-bis(4-methyl-1-piperazinyl]pregna-4,9(11)-diene-3,20-dione, 17α-hydroxy-6α-methyl-21[4-2,6-bis-(1-pyrrolidinyl-4-pyrimidinyl]-1-piperazinyl]pregna-1,4,9(11)-triene-3,20-dione, 21-[4-2,6-bis(diethylamino)-4-pyrimidinyl]-1-piperazinyl]-1-1α,17α-dihydroxypregn-4-ene-3,20-dione, 21-[4-[2,6-bis(diethylamino)-4-pyrimidinyl[-1piperazinyl]-17α-hydroxypregn-4-ene-3,20-dione, 21-[4-[2,6-bis(diethylamino)-4-pyrimidinyl]-1-piperazinyl]-17 α-hydroxy-6α-methylpregna-1,4,9(11)-triene-3,20-dione, 17α-hydroxy-21-[4-[2,6-bis(1-pyrrolidinyl)-4-pyrimidinyl]-1-piperazinyl]pregna-4,9(11)-diene-3,20-dione, 21-[4-[2,6-bis(diethylamino)-4-pyrimidinyl]-1-piperazinyl]-11α-hydroxypregn-4-ene-3,20-dione, 21-[4-[2,6-bis(diethylamino)-4-pyrimidinyl]-1-piperazinyl]-11α,17 α-dihydroxypregn-4-ene-3,20-dione, 17α-hydroxy-16 α-methyl-21-[4-[2,6-bis-(1-pyrrolidinyl)-4-pyrimidinyl]-1-piperazinyl]pregna-1,4,9(−11)-triene-3,20-dione, 17α-hydroxy-21-[4-[2,6-bis(1-pyrrolidinyl)-4-pyrimidinyl]-1-piperazinyl]pregna-1,4,9(11)-triene-3,20-dione, 21-[4-[2,6-bis(diethylamino)-4-pyrimidinyl]-1-piperazinyl]-17α-hydroxypregna-1,4,9(11)-triene-3,20-dione, 21-[4-[4,6-bis(diethylamino)-2-pyrimidinyl]-1-piperazinyl]-17α-hydroxypregna-1,4,9(11)-triene-3,20-dione, 21-[4-[2,6-bis(diethylamino)-4-pyrimidinyl]-1-piperazinyl]-16α-methylpregna-1,4,9(11)-triene-3,20-dione, 21-[4-[2,6-bis(diethylamino)-4-pyrimidinyl]-1-piperazinyl]-11.alpha.-hydroxy-16α-methylpregna-1,4-dien-e-3,20-dione, 21-[4-[2,6-bis(diethylamino)-4-pyrimidinyl]-1-piperazinyl]-1-6α-methylpregna-1,4-diene, 3,20-dione, 16α-methyl-21-[4-[2,6-bi-s(1-pyrrolidinyl)-4-pyrimidinyl]-1-piperazinyl]pregna-1,4,9(11)-triene-3,2-O-dione, 11α-hydroxy-16α21-[4-[2,6-bis(1-pyrroidinyl)-4-pyrimidinyl]piperazinyl]pregna-1,4-diene-3,20-dione, 16α-methyl-21-[4-[2,6-bis(1-pyrrolidinyl)-4-pyrimidinyl]-1-piperazinyl]pregna-1,4-diene-3,20-dione, 16.alpha.-methyl-21-[4-[2,6-bis(4-morpholino)-4-pyrimidinyl]-1-piperazinyl]pregna-1,4,9(11)-triene-3,20-dione, 11α-hydroxy-16α-methyl-21-[4-[2,6-bis(4-morpholino)-4-pyrimidinyl]-1-piperazinyl]pregna-1,4-diene-3,20-dione, 16.alpha.-methyl-21-[4-[2-,6-bis(4-morpholino(4-pyrimidinyl]-1-piperazinyl]pregna-1,4-diene-3,20-dione, 21-[4-[2,6-bis(allylamino)-4-pyrimidinyl]-1-piperazinyl[-16α-methylpregna-1,4,9(11)-triene-3,20-dione, 21-[4-[2,6-bis(allylamino)-4-pyrimidinyl]-1-piperazinyl]-11α-hydroxy-16α-methylpregna-1,4-ene-3,2-O-dione, 21-[4-[2,6-bis(allylamino)-4-pyrimidinyl]-1-piperazinyl]-16α-methylpregna-1,4-ene-3,20-dione, 21-[4-[2,6-bis(1-pyrrolidinyl)-4-pyrimidinyl]-1-piperazinyl]pregn-4-ene-3,11,20-trione, 21-[4-[2,6-bis(1-pyrrolidinyl)-4-pyrimidinyl]-1-piperazinyl]pregna-4,9(11)-diene-3,20-dione, 21-[4-[2,6-bis(1-pyrrolidinyl)-4-pyrimidinyl]-1-piperazinyl]pregna-1,4-diene-3,20-dione, 21-[4-(2,6-bis(1-pyrrolidinyl)-4-pyrimidinyl)-1-piperazinyl]pregna-4,9(11)-diene-3,20-dione, 21-[4-(2,6-bis(4-morpholino)-4-pyrimidinyl)-1-piperazinyl]-17α-hydroxypregna-4,9(11)-diene-3,20-dione, 21-[4-(2,6-bis(1-pyrrolidinyl)-4-pyrimidinyl)-1-piperazinyl]pregna-4-en-3-one, 21-[4-(2,6-bis(1-pyrrolidinyl)-4-pyrimidinyl)-1-piperazinyl]pregn-4-en-3-one, 16α-methyl-21-[4-[2,6-bis(1-pyrrolidinyl)-4-pyrimidinyl]-1-piperazinyl]pregna-1,4,9(11)-triene-3,20-dione, 21-[4-(2,6-bis(1-pyrrolidinyl)-4-pyrimidinyl)-1-piperazinyl]pregna-1,4,9(11)-triene-3,20-dione, 21-[4-(2,6-bis(1-pyrrolidinyl)-4-pyrimidinyl)-1-piperazinyl]-20-methylpregna-1,4-dien-3-one, 21-[4-(2,6-bis(1-pyrrolidinyl)-4-pyrimidinyl)-1-piperazinyl]pregna-1,4,9(11),16-tetraene-3,20-dione, 21-[4-[2,6-bis(4-morpholino)-4-pyrimidinyl]-1-piperazinyl]pregna-1,4-diene-3,20-dione, 21-[4-[2,6-bis(diethylamino)-4-pyrimidinyl]-1-piperazinyl]-6α-fluoro-17α-hydroxy-16β-methylpregna-4,9(11)-diene-3,20-dione, 6α-fluoro-17α-hydroxy-16-methyl-21-[4-[2,6-bis(1-pyrrolidinyl)-4-pyrimidinyl]-1-piperazinyl]pregna-4,9(11)-diene-3,20-dione, 16α-methyl-21-[4-[2,6-bis(1-pyrrolidinyl)-4-pyrimidinyl]-1-piperazinyl]pregna-1,4-diene-3,20-dione, 21-[4-(2,6-bis(1-pyrrolidinyl)-4-pyrimidinyl)-1-piperazinyl]-16α,17α-dimethylpregna-1,4,9(11)-riene-3,2-0-dione, 3β-hydroxy-16α-methyl-21-[4-[2,6-bis(1-pyrrolidinyl)-4-1-pyrimidinyl]-1-piperazinyl]-pregn-5-en-20-one, 16α-methyl-21-[4-[2,-6-bis-(1-pyrrolidinyl)-4-pyrimidinyl]-1-piperazinyl]pregna-1,4,6,9(11)-tetraene-3,20-dione, 3β-hydroxy-16α-methyl-21-[4-[2,6-bis(1-pyrrolidinyl)-4-pyrimidinyl]-1-piperazinyl]pregn-5-en-20-one, 16α-methyl-17β-(1-oxo-4-[4-[2,6-bis(1-pyrrolidinyl)-4-pyrimidinyl]-1-piperazinyl]butyl)-androsta-4,9(11)-dien-3-one, tocopherol, vitamin C, beta-carotene, lycopene, coenzyme Q, idebenone, lipoic acid, and ginkgo biloba; or is an isomer, a pharmaceutically acceptable salt, ester, or prodrug thereof.

In one or more embodiments, the NSAID is a cannabinoid. Cannabnoids are known to affect inflammation through suppression of runaway inflammation and other untoward effects of immune system activation, as well as pain.

In certain embodiments, the cannabinoid agent is selected from the group consisting of: 2-arachidonylglycerol; N-arachidonyl-1-(2,3-dichlorobenzoyl)-2-methyl-3-(2-[1-morpholino]ethyl)-5-methoxyindole; 2-methyl-1-propyl-3′-(1-naphthoyl)indole; 1-methoxy-N,N-dimethylmethanamide; 1-methoxy-endo-4-hydroxy-9-oxabicyclo(3.3.1)nonane; dronabinol; (2-methyl-1-propyl-1H-indol-3-yl)-1-naphthalenylmethanone; 3-(1,1-dimethylbutyl)-6a,7,10,10a-tetrahydro-6,6,9-6h-dibenzo[b,d]pyran; [2,3-dihydro-5-methyl-3(4-morpholinylmethyl)pyrrolo[1,2,3-de]methane; 5-(1,1-dimethylheptyl)-2-[(1R,2R,5R)-5-hydroxy-2-(3-hydroxypropyl)cyclohexyl]phenol; 5-(4-chlorophenyl)-1-(2,4-dichlorophenyl)-4-methyl-N-1-piperidinyl-1H-pyr-azole-3-caroxamide; [6-methoxy-2-(4-methoxyphenyl)benzo[b]furan-3-yl](4-cyanophenyl)methanone; [6-iodo-2-methyl-1-[2-(4-morpholinyl)ethyl]-1H-indol-3-yl](4-methoxy phenyl)methanone; 5-(4-chloro-3-methylphenyl)-1-[(4-methylphenyl)methyl]-N-(1,3,3-trimethyl-bicyclo[2.2.1]hept-2-yl)-(1S-endo)-1H-pyrazole-3-carboxamide; 1-(2,4-dichlorophenyl)-5-(4-iodophenyl)-4-methyl-n-1-piperidinyl-1H-pyraz-ole-3-carboxamide; 1-(2,4-dichlorophenyl)-5-(4-iodophenyl)-4-methyl-N-4-morpholinyl-1H-pyraz-ole-3-carboxamide; 3-(6-azido-2-hexynyl)-6a,7,10,10a-tetrahydro-6,6,9-trimethyl-(6aR,10aR)-6-H-dibenzo[b,d]pyran-1-ol; 3-[(2Z)-6-azido-2-hexynyl]-6a,7,10,10a-tetrahydro-6,6,9-trimethyl-(6aR,10-aR)-6H-dibenzo[b,d]pyran-1-ol; (−)-6,7-dichloro-1,4-dihydro-5-[3-(methoxymethyl)-5-(3-pyridinyl)-4H-1,2,-4-triazol-4-yl]-2,3-quinoxalinedione; (2R,4S)-rel-5,7-dichloro-1,2,3,4-tetrahydro-4-[[(phenylamino)carbonyl]ami-no]-2-quinolinecarboxylic acid; (2R,6S)-1,2,3,4,5,6-hexahydro-3-[(2S)-2-methoxypropyl]-6,11,11-trimethyl-2,6-methano-3-benzazocin-9-ol; (3E)-2-amino-4-(phosphonomethyl)-3-heptenoic acid; (3R,4S)-rel-3,4-dihydro-3-[4-hydroxy-4-(phenylmethyl)-1-piperidinyl]-2H-1-1-benzopyran-4,7-diol; (3S,4aR,6S,8aR)-decahydro-6-(phosphonomethyl)-3-isoquinoline carboxylic acid; (R)-9-bromo-2,3,6,7-tetrahydro-2,3-dioxo-N-phenyl-1H,5H-pyrido[1,2,-3-de]quinoxaline-5-acetamide; (.alpha.R)-.alpha.-amino-5-chloro-1-(phosphonomethyl)-1H-benzimidazole-2-propanoic acid; [2-(8,9-dioxo-2,6-diazabicyclo[5.2.0]non-1(7)-en-2-yl)ethyl]-phosphonic acid; [5-(aminomethyl)-2-[[[(5S)-9-chloro-2,3,6,7-tetrahydro-2,3-dioxo-1H,5H-py-rido[1,2,3-de]quinoxalin-5-ylacetyl]amino]phenoxy]-acetic acid; 1,4-dihydro-6-methyl-5-[(methylamino)methyl]-7-nitro-2,3-quinoxaline-dion-e monohydrochloride; 1-[2-(4-hydroxyphenoxy)ethyl]-4-[(4-methylphenyl)methyl]-4-piperidinol hydrochloride; 1-[4-(1H-imidazol-4-yl)-3-butynyl]-4-(phenylmethyl)-piperidine; 1-aminocyclopentane-carboxylic acid (ACPC); 2-[(2,3-dihydro-1H-inden-2-yl)amino]-acetamide monohydrochloride; 2-hydroxy-5-[[(pentafluorophenyl)methyl]amino]-benzoic acid (PBAS); 2-methyl-6-(phenylethynyl)-pyridine (MPEP); 3-(phosphonomethyl)-L-phenylalanine; 3-[(1E)-2-carboxy-2-phenylethenyl]-4,6-dichloro-1H-indole-2-carboxylic acid; 4,6-dichloro-3-[(E)-(2-oxo-1-phenyl-3-pyrrolidinylidene)methyl]-1H-indole-2-carboxylic acid; 6-chloro-2,3,4,9-tetrahydro-9-methyl-2,3-dioxo-1H-indeno[1,2-b]pyrazine-9-1-acetic acid; 7-chlorothiokynurenic acid; 8-chloro-2,3-dihydropyridazino[4,5-b]quinoline-1,4-dione 5-oxide salt with 2-hydroxy-N,N,N-trimethyl-ethanaminium; aptiganel; besonprodil; budipine; conantokin G; delucemine; dexanabinol; felbamate; fluorofelbamate; gacyclidine; glycine; ipenoxazone; kaitocephalin; lanicemine; licostinel; midafotel; milnacipran; N′-[2-chloro-5-(methylthio)phenyl]-N-methyl-N-[3-(methylthio)phenyl]-guan-idine; N′-[2-chloro-5-(methylthio)phenyl]-N-methyl-N-[3-[(R)-methylsulfiny-1]phenyl]-guanidine; neramexane; orphenadrine; remacemide; topiramate; alpha.-amino-2-(2-phosphonoethyl)-cyclohexanepropanoic acid; alpha.-amino-4-(phosphonomethyl)-benzeneacetic acid; 8-[4-(1,1-dimethylheptyl)-2-hydroxyphenyl]decahydro-2-naphthalene methanol; 5,6,6a,7,8,9,10,10a-octahydro-6-methyl-3-[(1R)-1-methyl-4-pheny-1 butoxy]-1,9-phenanthridinediol; Desacetyl-L-nantradol; R-(+)-methanandamide; 11-hydroxy-9,15-dioxoprosta-8,12,13-dienoic acid; 2-[3-methyl-6-(1-methylethenyl)-2-cyclohexen-1-yl]-5-pentyl-(1R-trans)-1,-3-benzenediol (cannabidiol); 3-amyl-1-hydroxy-6,6,9-trimethyl-6H-dibenzo[b,d]pyran (cannabinol); 3-(1,1-dimethylheptyl)-6a,7,8,9,10,10a-hexahydro-1-hydroxy-6,6-dimethyl-(−6aR,9R,10aR)-6H-dibenzo[b,d]pyran-9-methanol; 7-(1,1-dimethylheptyl)-1,2,3,4,4a,9b-hexahydro-2,2-dimethyl-4-methylene-1-,3-methanodibenzofuran-9-ol; 7-(1,1-dimethylheptyl)-1,2,3,4,4a,9b-hexahydro-2,2-dimethyl-4-methylene-1-(s),3-methanodibenzofuran-9-ol; 2-[4-[(acetyloxy)methyl]-6,6-dimethylbicyclo[3.1.1]hept-3-en-2-yl]-5-(11-1-dimethylheptyl)-diacetate[1R-(1a,2a,5a)]-1,3-benzenediol; 2-[4-[(acetyloxy)methyl]-6,6-dimethylbicyclo[3.1.1]hept-3-en-2-yl]-5-(1,1-1-dimethylheptyl)-diacetate[1S-(1a,2a,5a)]-1,3-benzenediol; 5-(1,1-dimethylheptyl)-2-[4-(hydroxy ethyl)-6,6-dimethylbicyclo[3.1.1]hep-t-3-en-2-yl]-[1S-(1a,2a,5a)]-1,3-benzenediol; and 5-(1,1-dimethylheptyl)-2-[4-(hydroxymethyl)-6,6-dimethylbicyclo[3.1.1]hep-t-3-en-2-yl]-[1R-(1a,2a,5a)]-1,3-benzenediol; or is an isomer, a pharmaceutically acceptable salt, ester, or prodrug thereof.

In one or more embodiments, the NSAID is an angiotensin II receptor antagonist. Angiotensin II receptor antagonists are known to affect inflammation and pain, as shown, for example in J Pharmacol Exp Ther. 2003 October; 307(1):17-23. Epub 2003 Aug. 27.

In certain embodiments, the angiotensin II receptor antagonist is selected from the group consisting of candesartan, eprosartan, irbesartan, losartan, olmesartan, tasosartan, telmisartan, valsartan, saralasin, and 1-[[4-(dimethylamino)-3-methylphenyl]methyl]-5-(diphenylac-etyl)-4,5,6,7-tetrahydro-1H-imidazo[4,5-c]pyridine-6-carboxylic acid ditrifluoroacetate, or an isomer, a pharmaceutically acceptable salt, ester, or prodrug thereof.

In one or more embodiments, the NSAID is an UDP-glucuronosyltransferase inhibitor (UGT inhibitor).

In certain embodiments, the UGT inhibitor is selected from the group consisting of epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, allspice berry oil, N-vanillylnonanamide, clovebud oil, peppermint oil, silibinin and silymarin.

Mixtures of these non-steroidal immunomodulators may also be employed.

Suitable non-steroidal anti-inflammatory agent include but are not limited to azelaic acid, oxicams, piroxicam, isoxicam, tenoxicam, sudoxicam, CP-14,304, salicylates, aspirin, disalcid, benorvlate, trilisate, safapryn, solprin, diflunisal, fendosal, acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trinmethazone and derivatives, esters, salts and mixtures thereof.

### Insecticide

In an embodiment, the therapeutic agent is insecticide. In the context of one or more embodiments “insecticide, is used herein to mean a compound which kills, inhibits the growth of, impeded the proliferation of or repels insects or to kill or prevent the growth of parasite arthropods, such as insects, arachnids and crustaceans, or a compound used to repel or prevent infestation by these parasite arthropods.

The term insecticides include, for example, agents that can kill lice, flees, ticks, mites, scabies and mousquitos, as well as agents that repel such insects. Suitable insecticides include but are not limited to DDT, lindane, malathion, pennethrin, allethrin, biopermethrin, transpennethrin, phenothrin, diethyl-m-toluamide, dimethyl phthalate, piperonyl butoxide, pyrethroids and derivatives, esters, salts and mixtures thereof.

In one or more embodiments, the insecticide is an antibiotic insecticide. Examples of antibiotic insecticides include allosamidin, thuringiensin, spinosad, avermectin insecticides, such as abamectin, doramectin, emamectin, eprinomectin, ivermectin and selamectin, milbemycin insecticides, such as lepimectin, milbemectin, milbemycin oxime and moxidectin, and arsenical insecticides.

In one or more embodiments, the insecticide is a botanical insecticide, such as anabasine, azadirachtin, d-limonene, nicotine, pyrethrins, cinerins, jasmolin, quassia, rotenone, ryania and sabadilla.

In one or more embodiments, the insecticide is a carbamate insecticide. Examples of carbamate insecticides include bendiocarb, carbaryl, benzofuranyl methylcarbamate insecticides, such as benfuracarb, carbofuran, carbosulfan, decarbofuran and furathliocarb, dimethylcarbamate insecticides, such as dimetan, dimetilan, hyquincarb and pirimicarb, oxime carbamate insecticides, such as alanycarb, aldicarb, aldoxycarb, butocarboxim, butoxycarboxim, methomyl, nitrilacarb, oxamyl, tazimcarb, thiocarboxime, thiodicarb and thiofanox, and phenyl methylcarbamate insecticides, such as allyxycarb, aminocarb, bufencarb, butacarb, carbanolate, cloethocarb, dicresyl, dioxacarb, ethiofencarb, fenethacarb, fenobucarb, isoprocarb, methiocarb, metolcarb, mexacarbate, promacyl, promecarb, propoxur, trimethacarb and xylylcarb.

In one or more embodiments, the insecticide is a dinitrophenol insecticides. Examples of dinitrophenol insecticides include dinex, dinopropand dinosam.

In one or more embodiments, the insecticide is a fluorine insecticide, such as barium hexafluorosilicate, cryolite, sodium fluoride, sodium hexafluorosilicate and sulfluramid.

In one or more embodiments, the insecticide is a formamidine insecticide, such as amitraz, chlordimeform, formetanate and formparanate.

In one or more embodiments, the insecticide is an insect growth regulator, Examples of insect growth regulators include chitin synthesis inhibitors, such as bistrifluoron, buprofezin, chlorfluazuron, cyromazine, diflubenzuron, flucycloxuron, flufenoxuron, hexaflumuron, lufenuron, novaluron, noviflumuron, penfluoron, teflubenzuron and triflumuron, juvenile hormone mimics, such as epofenonane, fenoxycarb, hydroprene, kinoprene, methoprene, pyriproxyfen and triprene, juvenile hormones, moulting hormone agonists, such as chromafenozide, halofenozide, methoxyfenozide and tebufenozide, moulting hormones, such as α-ecdysone and ecdysterone, moulting inhibitors, such as diofenolan, precocenes, and dicyclanil.

In one or more embodiments, the insecticide is a nereistoxin analogue insecticide, such as bensultap, cartap, thiocyclam and thiosultap.

In one or more embodiments, the insecticide is a nicotinoid insecticide. Examples of nicotinide insecticides include flonicamid, nitroguanidine insecticides, such as clothianidin, dinotefuran, imidacloprid and thiamethoxam, nitromethylene insecticides, such as nitenpyram and nithiazine, and pyridylmethylamine insecticides, such as acetamiprid, imidacloprid, nitenpyram and thiacloprid.

In one or more embodiments, the insecticide is an organochlorine insecticide. Examples of organochlorine insecticides include bromo-DDT, camphechlor, DDT, lindane, methoxychlor, pentachlorophenol, cyclodiene insecticides, such as aldrin, bromocyclen, chlorbicyclen, chlordane, chlordecone, dieldrin, dilor, endosulfan, endrin, heptachlor, isobenzan, isodrin, kelevan and mirex.

In one or more embodiments, the insecticide is an organophosphorus insecticide. Examples of organophosphorus insecticides include organophosphate insecticides such as bromfenvinfos, chlorfenvinphos, crotoxyphos, dichlorvos, dicrotophos, dimethylvinphos, fospirate, heptenophos, methocrotophos, mevinphos, monocrotophos, naftalofos, phosphamidon, propaphos and tetrachlorvinphos, organothiophosphate insecticides, such as dioxabenzofos, fosmethilan, phenthoate, acethion, amiton, cadusafos, chlorethoxyfos, chlormephos, demephion, demephion, demeton, disulfoton, ethion, ethoprophos, isothioate, malathion, methacrifos, oxydemeton-methyl, oxydeprofos, oxydisulfoton, phorate, sulfotep, terbufos and thiometon, aliphatic amide organothiophosphate insecticides, such as amidithion, cyanthoate, dimethoate, ethoate-methyl, formothion, mecarbam, omethoate, prothoate, sophamide and vamidothion, oxime organothiophosphate insecticides, such as chlorphoxim, phoxim and phoxim-methyl, heterocyclic organothiophosphate insecticides, such as azamethiphos, coumaphos, coumithoate, dioxathion, endothion, menazon, morphothion, phosalone, pyraclofos, pyridaphenthion and quinothion, benzothiopyran organothiophosphate insecticides, such as dithicrofos and thicrofos, benzotriazine organothiophosphate insecticides, such as azinphos-ethyl and azinphos-methyl, isoindole organothiophosphate insecticides, such as dialifos and phosmet, isoxazole organothiophosphate insecticides, such as isoxathion and zolaprofos, pyrazolopyrimidine organothiophosphate insecticides, such as chlorprazophos and pyrazophos; pyridine organothiophosphate insecticides, such as chlorpyrifos and chlorpyrifos-methyl, pyrimidine organothiophosphate insecticides, such as butathiofos, diazinon, etrimfos, lirimfos, pirimiphos-ethyl, pirimiphos-methyl, primidophos, pyrimitate and tebupirimfos, quinoxaline organothiophosphate insecticides, such as quinalphos and quinalphos-methyl, thiadiazole organothiophosphate insecticides, such as athidathion, lythidathion, methidathion and prothidathion, triazole organothiophosphate insecticides, such as isazofos and triazophos, phenyl organothiophosphate insecticides, such as azothoate, bromophos, bromophos-ethyl, carbophenothion, chlorthiophos, cyanophos, cythioate, dicapthon, dichlofenthion, etaphos, famphur, fenchlorphos, fenitrothion, fensulfothion, fenthion, fenthion-ethyl, heterophos, jodfenphos, mesulfenfos, parathion, parathion-methyl, phenkapton, phosnichlor, profenofos, prothiofos, sulprofos, temephos, trichlormetaphos-3 and trifenofos, phosphonate insecticides, such as butonate and trichlorfon, phosphonothioate insecticides such as mecarphon, phenyl ethylphosphonothioate insecticides, such as fonofos and trichloronat, phenyl phenylphosphonothioate insecticides, such as cyanofenphos, EPN and leptophos, phosphoramidate insecticides, such as crufomate, fenamiphos, fosthietan, mephosfolan, phosfolan and pirimetaphos, phosphoramidothioate insecticides, such as acephate, isocarbophos, isofenphos, methamidophos and propetamphos, and phosphorodiamide insecticides, such as dimefox, mazidox, mipafox and schradan.

In one or more embodiments, the insecticide is an oxadiazine insecticide, such as indoxacarb.

In one or more embodiments, the insecticide is a phthalimide insecticide, such as dialifos, phosmet and tetramethrin.

In one or more embodiments, the insecticide is a pyrazole insecticide, such as acetoprole, ethiprole, fipronil, pyrafluprole, pyriprole, tebufenpyrad, tolfenpyrad and vaniliprole.

In one or more embodiments, the insecticide is a pyrethroid insecticide. Examples of pyrethroid insecticides include pyrethroid ester insecticides, such as acrinathrin, allethrin, bioallethrin, barthrin, bifenthrin, bioethanomethrin, cyclethrin, cycloprothrin, cyfluthrin, beta-cyfluthrin, cyhalothrin, cypennethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, furethrin, imiprothrin, metofluthrin, permethrin, biopennethrin, transpennethrin, phenothrin, prallethrin, profluthrin, pyresmethrin, resmethrin, bioresmethrin, cismethrin, tefluthrin, terallethrin, tetramethrin, tralomethrin and transfluthrin, and pyrethroid ether insecticides, such as etofenprox, flufenprox, halfenprox, protrifenbute and silafluofen.

In one or more embodiments, the insecticide is a pyrimidinamine insecticide, such as flufenerim and pyrimidifen.

In one or more embodiments, the insecticide is a pyrrole insecticide, such as chlorfenapyr.

In one or more embodiments, the insecticide is a tetronic acid insecticide, such as spiromesifen and spirotetramat.

In one or more embodiments, the insecticide is a thiourea insecticide, such as diafenthiuron.

In one or more embodiments, the insecticide is a urea insecticide, such as flucofuron and sulcofuron.

Yet, in additional embodiments, the insecticide is an unclassified insecticide, such as closantel, crotamiton, fenazaflor, fenoxacrim, flubendiamide, hydramethylnon, isoprothiolane, malonoben, metaflumizone, metoxadiazone, nifluridide, pyridaben, pyridalyl, rafoxanide, triarathene and triazamate.

The above listed insecticides, as well as others not listed, are suitable for use in the composition. It is preferred to use insecticides that are approved by the FDA or other health authorities for the treatment of animals and humans.

Non-limiting examples of approved insecticides include hexachlorobenzene, carbamate, naturally occurring pyrethroids, permethrin, allethrin, bioalethrin, phenothrin, malathion and piperonyl butoxide. In a preferred embodiment the insecticide is selected from the group consisting of hexachlorobenzene, carbamate, naturally occurring pyrethroids, permethrin, allethrin, bioalethrin, phenothrin, malathion and piperonyl butoxide.

In one or more embodiments, the insecticide is a naturally occurring insecticide compound. As used herein, the term “naturally-occurring insecticide” includes all insecticides that are obtained, derived or extracted from plant or vertebrate sources.

In the context, an agent that kills or otherwise affects parasites, such as protozoa is also termed an insecticide (for the purpose of this application terminology only). Exemplary antiparasites are mebendazole, thiabendazole, metronidazole, and praziquantel.

Mixtures of these insecticides may also be employed.

The insecticide is included in the composition in a concentration that provides a desirable ratio between the efficacy and safety. Typically, insecticides are included in the composition in a concentration between about 0.05% and about 12% by weight, depending on their potency against the parasitic arthropod to be eradicated. In some embodiments, the concentration is between about 0.5% and about 2% by weight; in other embodiment the concentration is between about 2% and about 5% by weight; and in other embodiments the concentration is between about 5% and about 12% by weight.

In one or more embodiments, the insecticide and the silicone work together for example the former by killing eggs and the latter by discouraging them from sticking to a surface.

In one or more embodiments, the insecticide is encapsulated in particles, microparticles, nanoparticles, microcapsules, spheres, microspheres, nanocapsules, nanospheres, liposomes, niosomes, polymer matrix, nanocrystals or microsponges, and may be manufactured according to known methods.

### Vasodilators

In an embodiment, the therapeutic agent is a vasodilator. Suitable vasodilators include but are not limited to agents that modulate the activity of the enzyme nitric oxide synthase, nicotinic acid, ethyl nicotinate, amyl nitrite, amyl nitrate, ethyl nitrite, butyl nitrite, isobutyl nitrite, glyceryl trinitrate, octyl nitrite, sodium nitrite, sodium nitroprusside, clonitrate, erythrityl tetranitrate, isosorbide mononitrate, isosorbide dinitrate, mannitol hexanitrate, pentaerytllritol tetranitrate, penetrinitol, triethanolamine trinitrate, troInitrate phosphate (triethanolamine trinitrate diphosphate), propatylnitrate, nitrite esters of sugars, nitrite esters of polyols, nitrate esters of sugars, nitrate esters of polyols, nicorandil, apresoline, diazoxide, hydralazine, hydrochlorothiazide, minoxidil, pentaerythritol, tolazoline, scoparone, a beta-adrenergic blocker, an alpha-adrenoceptor blocker, a prostaglandin, sildenafil, dipyridamole, catecholamine, isoproternol, furosemide, prostaglandin, prostacyclin, enalaprilat, morphine, acepromazine, prazosin (α-blocker), enalapril, Captopril, amlodipine, minoxidil, tadalafil, vardenafil, phenylephrin, etilefein, caffeine, capsaicin, an extract capsicum, achillea millefolium (Yarrow), allium sativum (garlic), amoracia rusticana (horseradish), berberis vulgaris (barberry), cimicifuga racemosa (black cohosh), coleus forskholii (coleus), coptis (goldenthread), crataegus (hawthorn), eleutherococcus senticosus (siberian ginseng), ginkgo biloba(ginkgo), melissa offiicnalis (lemon balm), olea europaea (olive leaf), panax ginseng (Chinese ginseng), petroselinum crispum (parsley), scutellaria baicalensis (baical skullcap), tilia europaea (linden flower), trigonella foenum-graecum (fenugreek), urtica dioica (nettles), valeriana officinalis (valerian), viburnum (cramp, bark, black haw), veratrum viride (American hellebore), verbena officinalis (vervain), xanthoxylum americanum (prickly ash), zingiber officinale (ginger), rauwolfia serpentina (Indian snakeroot), viscum album, wild yam, sasparilla, licorice, damiana, yucca, saw palmetto, gotu kola (centella asiatica), yohimbine and salts, hazel nut, brazil nut and walnut, and derivatives, esters, salts and mixtures thereof.

In an embodiment, the therapeutic agent is a vasoconstrictor. Suitable vasodilators include but are not limited to ephedrine, epinephrine, phenylephrine, angiotensin, vasopressin; an extract ephedra sinica (ma huang), polygonum bistorta (bistort root), hamamelis virginiana (witch hazel), hydrastis canadensis (goldenseal), lycopus virginicus (bugleweed), aspidosperma quebracho (quebracho blanco), cytisus scoparius (scotch broom) and cypressand and derivatives, esters, salts and mixtures thereof.

### Retinoid

In an embodiment, the active agent is a retinoid.

In the context, a retinoids is a compound a class of compounds consisting of found isoprenoid units joined in a head-to-tail manner, and derivatives, salts, structural analogs and functional analogs thereof, as reviewed herein in a non-limiting fashion. Typically, retinoids may be formally derived from a monocyclic parent compound containing five carbon-carbon double bonds and a functional group at the terminus of the acyclic portion.

Suitable, but non-limiting, retinoids for use in the present invention are listed below.

It is convenient to omit the explicit representation of C and H atoms in the parent skeletal structure of retinoids as follows:

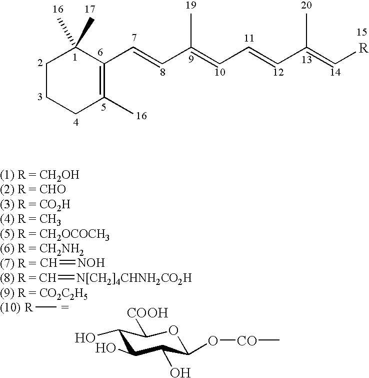

Compound (1) (2E,4E,6E,8E)-3,7-dimethyl-9-(2,6,6-trmethylcyclohex-1-en-1-yl)nona-2,4,6,8-tetraen-1-ol is also known as vitamin A, vitamin A alcohol, retinal, vitamin A₁, vitamin A₁ alcohol, axerophthol or axerol. Compound (2) also known as vitamin A aldehyde, vitamin A₁ aldehyde, retinene or retinene, and retinal or, if liable to be confused with the adjective retinal (pertaining to the retina), retinaldehyde. Compound (3) also known as tretinoin (see note), vitamin A acid or vitamin A₁ acid should be designated retinoic acid. Compound (4), is known as axerophthene. Functional substitution at the 15 position of the basic hydrocarbon is denoted by the use of the group names retinyl (R is CH₂—) or retinylidene (R is CH═), with retention of the original numbering of the basic hydrocarbon. For example (5) is retinyl acetate and (6) is retinylamine. Derivatives of retinal include for example Compound (7)—retinal oxime and Compound (8)—N⁶-retinylidene-L-lysine. Other derivatives of retinoic acid, named as carboxylic acid derivatives Compound (9)—ethyl retinoate and Compound (10)—1-O-retinoyl-b-D-glueopyranuronic acid.

Retinoids that differ in hydrogenation level from the parent structure (displayed above) are named by use of the prefixes ‘hydro’ and ‘dehydro’ together with locants specifying the carbon atoms at which hydrogen atoms have been added or removed. Examples of such retinoid compounds are Compound (11)-3,4-Didehydroretinol (also known as dehydroretinol or vitamin A₂) and Compound (12)-4,5-Didehydro-5,6-dihydroretinol (also known as alpha-vitamin A).

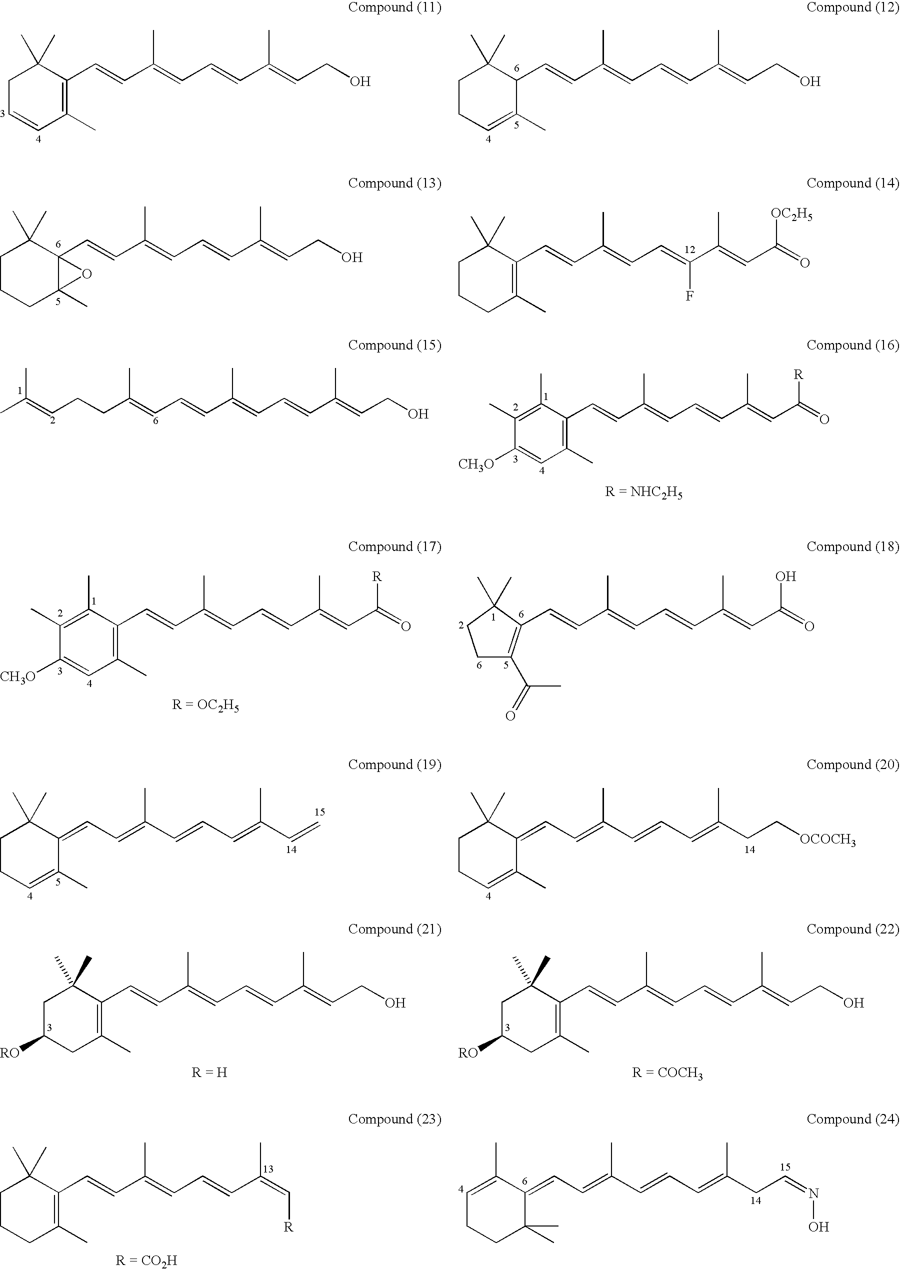

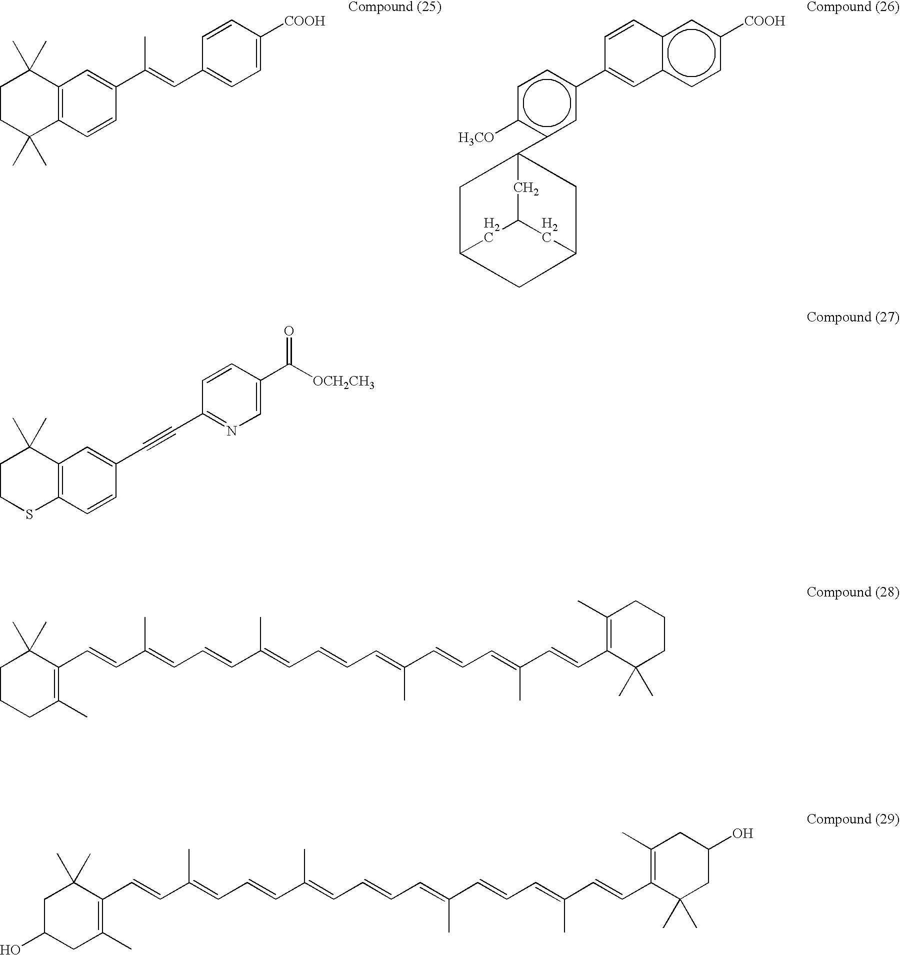

Substituted derivatives of retinoids are exemplified by Compound (13)—5,6-Epoxy-5,6-dihydroretinol (also known as hepaxanthin) and Compound (14)—Ethyl 12-fluororetinoate. Seco Retinoids are exemplified by Compound (15)—1,6-Seco-1,2-didehydroretinol, also known as g-vitamin A, and Nor Retinoids, which result from the elimination of a CH₃, CH₂, CH or C group from a retinoid are exemplified by Compound (16)—N-Ethyl-3-methoxy-2-methyl-17-nor-1,2,3,4-tetradehydroretinamide (also known as motretinide), Compound (17)—Ethyl 3-methoxy-2-methyl-17-nor-1,2,3,4-tetradehydroretinoate (also known as etretinate), acitretin (Compound (17), wherein R═H) and Compound (18)—5-Acetyl-4,18-dinor-retinoic acid. Retro Retinoids are exemplified by Compound (19)—4,5-Didehydro-15,5-retro-deoxyretinol (also known as anhydro vitamin A and Compound (20)—4,14-retro-Retinyl acetate. Stereoisomers of retinoids are exemplified by Compound (21)—(3R)-3-Hydroxyretinol and Compound (22)—(3R)-3-Acetoxyretinol. Other stereochemical isomers can are exemplified by Compound (23)—13-cis-Retinoic acid or (7E,9E,11E,13Z)-retinoic acid (also known as isotretinoin) and Compound (24)—(6E,8E,10E,12E,15Z)-4,14-retro-Retinaloxime.

‘Arotinoids or ‘retinoidal benzoic acid derivatives’ contain, aromatic rings replacing either the basic ^{β}-ionone type ring structure or unsaturated bonds of the tetraene side chain of the parent retinoid skeleton, as exemplified by Compound (25) and Compound (26)—6-[3-(1-adamantyl)-4-methoxyphenyl]-2-naphthoic acid, also known as adapalene. Several artinoids, possessing potent retinoid properties, including but not limited to short retinoids, short heterocyclic retinoids, isoxazole-containing retinoids, heterocyclic isoxazole-containing retinoids, isoxazoline-containing retinoids, stilbene retinoid analogs, are disclosed in Pure Appl. Chem., Vol. 73, No. 9, pp. 1437-1444, 2001.

Tazarotene (Ethyl 6-[2-(4,4-dimethylthiochroman-6-yl)ethynyl]nicotinate) is exemplary to a retinoid precursor—Compound (27), suitable as retinoid for use in the present invention.

Yet, other non-limiting exemplary retinoid precursors are carotenes, such as all-trans beta carotene—Compound (28), alpha carotene, lycopene and 9-cis-beta-carotene, as well as xanthophils (also termed “oxicarotenoids”), such as lutein and zeaxanthin—Compound (29).

Salts and derivatives of retinoid compounds are also suitable as “retinoid” for use in the present invention.

Retinoid compounds can be ascertained recognized and identified by methods known in the art. One method involves the use of competitive nuclear retinoic acid (RA and RX) receptor binding assays for identifying compounds which bind directly to the receptors. For instance, J. J. Repa et al., “All-trans-retinol is a ligand for the retinoic acid receptors”, Proc. Natl. Acad. Sci. USA, Vol. 90, pp. 7293-7297, 1993, discloses a competitive RA receptor binding assay based on human neuroblastoma cell nuclear extracts. H. Tonna et al. ((1994) “Biologic activities of retinoic acid and 3,4-dehydroretinoic acid in human keratinoacytes are similar and correlate with receptor affinities and transactivation properties,” J. Invest. Dermatology, Vol. 102, pp. 49-54) discloses assays for measuring binding affinities for the nuclear retinoic acid receptors and for measuring transcriptional activation induction. M. F. Boehm et al. ((1994) “Synthesis of high specific activity [.sup.3H]-9-cis-retinoic acid and its application for identifying retinoids with unusual binding properties,” J. Med. Chem., Vol. 37, pp. 408-414) discloses a ligand-binding assay and a receptor/reporter cotransfection assay for monitor regulation of gene expression. EP 0 552 612 A2, published Jul. 28, 1993, describes ligand-binding trapping assays based on incubation of radiolabeled compounds with transfected COS-1 cells which express RA and RX receptors.

Mixtures of these retinoids may also be employed.

Suitable retinoids include but are not limited to retinol, retinal, retinoic acid, all-trans retinoic acid, isotretinoin, tazarotene, adapalene, 13-cis-retinoic acid, acitretin all-trans beta carotene, alpha carotene, lycopene, 9-cis-beta-carotene, lutein and zeaxanthin.

In an embodiment, the therapeutic agent is selected from the group consisting of an immunosuppressants and immunoregulating agents. Suitable immunosuppressants and immunoregulating agents include but are not limited to cyclic peptides, such as cyclosporine, tacrolimus, tresperimus, pimecrolimus, sirolimus (rapamycin), verolimus, laflunimus, laquinimod, imiquimod derivatives, esters, salts and mixtures thereof. In one or more embodiments, the immunomodulator is a calcineurin Inhibitor.

In an embodiment, the therapeutic agent is a wart remover. Suitable wart removers include but are not limited to imiquimod, podophyllotoxin and derivatives, esters, salts and mixtures thereof.

### Vitamin

The term vitamin includes those vitamins and derivatives thereof (including salts) which are officially recognized as vitamins, and those vitamins which were once recognized or designated as vitamins but are now classified in another way (e.g. vitamin F) and pseudo vitamins including those substances which are a member of a group or complex but are not formally recognized (e.g. para-amino benzoic acid (PABA), which is claimed to prevent greying hair and to be useful as an anti aging supplement) and also vitamin mimetics, which have vitamin like properties or effects.

Suitable vitamins include vitamin A, vitamins of the B complex B1, B2, B3, B5, B6, B7, 19, B12, vitamin C, vitamins D1-D4, vitamin E, vitamin K and so called vitamin F and a derivative thereof and combinations thereof.

Vitamin A is a fat-soluble vitamin and describes compounds that exhibit the biological activity of retinol. The two main components in foods are retinol and the carotenoids. ‘Retinoid’ refers to the chemical entity retinol or other closely related naturally occurring derivatives. These include: retinal (retinaldehyde); retinoic acid; and retinyl esters (e.g. retinyl acetate, retinyl palmitate, retinyl propionate). Retinoids also include structurally related synthetic analogues which may or may not have retinol-like activity. Vitamin A (in the form of retinal) is essential for normal function of the retina and particularly for visual adaptation to darkness. Other forms (retinol, retinoic acid) are necessary for maintenance of the structural and functional integrity of epithelial tissue and the immune system, cellular differentiation and proliferation, bone growth, testicular and ovarian function and embryonic development. It may act also as a co-factor in biochemical reactions. Deficiency can amongst other things result in skin dryness and papular eruptions. Vitamin A and its derivatives have the ability to normalize keratinization. Note that vitamin C may ameliorate the toxic effects of vitamin A; that large doses increase the need for vitamin E; and that vitamin E protects against the oxidative destruction of vitamin A. Retinol is susceptible to breakdown from oxygen and light. Synthetic retinoids may be used for skin problems (e.g. acne).

According to certain embodiments the retinoid is selected from the group consisting of: (1) a compound consisting of four isoprenoid units joined in a head-to-tail manner, a compound having the formula:

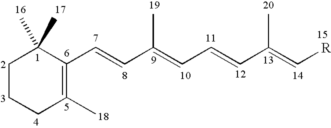

where R is selected from the group consisting of H, alkyl, aryl, alkenyl, benzyl, CH2OH, CH2NH2, CHO, CH═NOH, CO2H, CH═N[CH2]4CHNH2CO2H, CH3, CO2C2H5, CH₂OCOCH3, a heteroatom, a saccharide and a polysaccharide; (2) a compound selected from the group consisting of a hydro retinoid, a dehydro retinoid, 3,4-Didehydroretinol, 4,5-Didehydro-5,6-dihydroretinol, a substituted derivative of a retinoid, 5,6-epoxy-5,6-dihydroretinol, ethyl 12-fluororetinoate, a seco retinoid, 1,6-Seco-1,2-didehydroretinol, a nor retinoid, (3) a compound which results from the elimination of a CH3, CH2, CH or C group from a retinoid, N-ethyl-3-methoxy-2-methyl-17-nor-1,2,3,4-tetradehydroretinamide, ethyl 3-methoxy-2-methyl-17-nor-1,2,3,4-tetradehydroretinoate, 5-acetyl-4,18-dinor-retinoic acid, a retro retinoid, 4,5-didehydro-15,5-retro-deoxyretinol, 4,14-retro-retinyl acetate, a stereoisomer of a retinoid, (3R)-3-hydroxyretinol, (3R)-3-Acetoxyretinol, (7E,9E,11E,13Z)-retinoic acid, (6E,8E,10E,12E,15Z)-4,14-retro-retinaloxime, an arotinoids, a retinoidal benzoic acid derivative, 6-[3-(1-adamantyl)-4-methoxyphenyl]-2-naphthoic acid, a short retinoid, a short heterocyclic retinoid, an isoxazole-containing retinoids, a heterocyclic isoxazole-containing retinoid, an isoxazoline-containing retinoid, a stilbene retinoid analog, a retinoid precursor, (ethyl 6-[2-(4,4-dimethylthiochroman-6-yl)ethynyl]nicotinate, a carotene, a xanthophil and an oxicarotenoid; (4) a compound selected from the group consisting of retinol, retinal, retinoic acid, all-trans retinoic acid, isotretinoin, tazarotene, adapalene, 13-cis-retinoic acid, acitretin, all-trans beta carotene, alpha carotene, lycopene, 9-cis-beta-carotene, lutein and zeaxanthin; (5) a compound that is positively identified using a laboratory method, suitable of detecting a retinoid, and salts and derivatives thereof.

Vitamin B is known as the vitamin B complex and comprises B1 (thiamine), B2 (riboflavin), B3 (niacin), B5 (pantothenic acid), B6 (pyridoxine), B7 (biotin), B9 (folic acid) and B12 (cyanocobalamin). Adequate amounts of all B vitamins are required for optimal functioning; deficiency or excess of one B may lead to abnormalities in the metabolism of another.

Thiamine is a water soluble vitamin and is also known as aneurine and functions as a co-enzyme in the oxidative decarboxylation of alpha ketoacids (involved in energy production) and in the transketolase reaction of the pentose phosphate pathway (involved in carbohydrate metabolism). Thiamine is also important in nerve transmission (independently of co-enzyme function). It may also act as an insect repellant.

Riboflavin is a water soluble vitamin and functions as a component of two flavin co-enzymes—flavin mononucleotide (FMN) and flavin adenine dinucleotide (FAD). It participates in oxidation-reduction reactions in numerous metabolic pathways and in energy production. Examples include: the oxidation of glucose, certain amino acids and fatty acids; reactions with several intermediaries of the Krebs cycle; conversion of pyridoxine to its active co-enzyme; and conversion of tryptophan to niacin. Riboflavin has a role as an antioxidant. It may be involved in maintaining the integrity of erythrocytes. Common forms are riboflavin, riboflavin butyrate and flavin adenine dinucleotide.

Niacin is a water-soluble vitamin and describes the compounds that exhibit the biological properties of nicotinamide. It occurs as nicotinamide and nicotinic acid. It is sometimes known as niacinamide. An example of a derivative is benzyl nicotinate. Niacin functions as a component of two co-enzymes, nicotinamide adenine dinucleotide (NAD) and nicotinamide adenine dinucleotide diphosphate (NADP). These co-enzymes participate in many metabolic processes including glycolysis, tissue respiration, lipid, amino acid and purine metabolism. It has been shown to have ant-inflammatory properties that result in the improvement of acne. Topically it has showed benefit for various skin conditions including psoriasis and rosacea. It has also been said to have a photo protection role, perhaps through anti-oxidant activity and reduces or prevents UV damage to cells and UV induced disorders.

Pantothenic acid is also a water soluble vitamin and functions mainly as a component of co-enzyme A and acyl carrier protein. Co-enzyme A has a central role as a co-factor for enzymes involved in the metabolism of lipids, carbohydrates and proteins; it is also required for the synthesis of cholesterol, steroid hormones, acetyl choline and porphyrins. As a component of acyl carrier protein, pantothenic acid is involved in various transfer reactions and in the assembly of acetate units into longer-chain fatty acids. Pantothenic acid has been used for a wide range for disorders such as acne, alopecia, allergies, burning feet, asthma, grey hair, dandruff, and cholesterol lowering. Panthenol the alcoholic form functions as a humetic. Examples of pantothenic acid derivatives are calcium pantothenate, D-pantothenyl alcohol, pantothenyl ethyl ether, and acetylpentothenyl ethyl ether.

Vitamin B6 is water soluble vitamin. Vitamin B6a generic term used to describe the compounds that exhibit the biological activity of pyridoxine. It occurs in food as pyridoxine, pyridoxal and pyridoxamine. Vitamin B6 is converted in erythrocytes to pyridoxal phosphate and, to a lesser extent, pyridoxamine phosphate. It acts as a co-factor for enzymes which are involved in more than 100 reactions that affect protein, lipid and carbohydrate metabolism. Pyridoxal phosphate is also involved in: the synthesis of several neurotransmitters; the metabolism of several vitamins (e.g. the conversion of tryptophan to niacin); haemoglobin and sphingosine formation. Lack of Vitamin B6 may affect vitamin C. Examples are pyridoxine hydrochloride and pyridoxine dioctanate.

Biotin is a water soluble vitamin which was formerly known as vitamin H or co-enzyme R. Biotin functions as an integral part of the enzymes that transport carboxyl units and fix carbon dioxide. Biotin enzymes are important in carbohydrate and lipid metabolism, and are involved in gluconeogenesis, fatty acid synthesis, propionate metabolism and the catabolism of amino acids. Biotin has been claimed to be of value in the treatment of brittle finger nails, acne, seborrhoeic dermatitis, hair fragility and alopecia.

Folic acid (pteroylglutamic acid) is a water soluble vitamin and is the parent compound for a large number of derivatives collectively known as folates. Folate is the generic term used to describe the compounds that exhibit the biological activity of folic acid; it is the preferred term for the vitamin present in foods which represents a mixture of related compounds (folates). Folates are involved in a number of single carbon transfer reactions, especially in the synthesis of purines and pyrimidines (and hence the synthesis of DNA), glycine and methionine. They are also involved in some amino acid conversions and the formation and utilization of formate. Deficiency leads to impaired cell division (effects most noticeable in rapidly regenerating tissues).

Vitamin B12 is a water-soluble vitamin and it is the generic term used to describe the compounds that exhibit the biological activity of cyanocobalamin. It includes a range of cobalt-containing compounds, known as cobalamins. Cyanocobalamin and hydroxocobalamin are the two principal forms in clinical use. Vitamin B12 is involved in the recycling of folate co-enzymes and the degradation of valine. It is also required for nerve myelination, cell replication, haematopoiesis and nucleoprotein synthesis.

Vitamin C is a water-soluble vitamin and describes the compounds that exhibit the biological activity of ascorbic acid. These include L-ascorbic acid (ascorbic acid) and L-dehydroascorbic acid (dehydroascorbic acid). The functions of vitamin C are based mainly on its properties as a reducing agent. It is required for: the formation of collagen and other organic constituents of the intercellular matrix in bone, teeth and capillaries; and the optimal activity of several enzymes—it activates certain liver-detoxifying enzyme systems (including drug-metabolizing enzymes) and is involved in the synthesis of carnitine and norepinephrine (noradrenaline) and in the metabolism of folic acid, histamine, phenylalanine, tryptophan and tyrosine. Vitamin C also acts: as an antioxidant (reacting directly with aqueous free radicals)-which is important in the protection of cellular function; and to enhance the absorption of non-haem iron. It can function as a whitening agent. Vitamin C may assist with wound healing. Vitamin C can spare vitamin E and vice versa and it may reduce toxic effects of vitamin A. Vitamin C is unstable in solution especially alkaline solution and readily undergoes oxidation on exposure to air. Oxidation is accelerated by light and heat. Cosmetic forms include calcium ascorbate, magnesium ascorbate, sodium ascorbate, sodium ascorbyl phosphate, ascorbyl palmitate, magnesium ascorbyl phosphate, L-ascorbic acid and magnesium-L-ascorbyl-2-phosphate. L-ascorbic acid palmitate, L-ascorbic acid 2-sulfate, L-ascorbic acid phosphate, and DL-.alpha.-tocopherol-L-ascorbic acid phosphate diester dipotassium. L-ascorbic acid is the most bioactive form and has been found to have many skin benefits but it is unstable in the presence of water and oxygen. Inclusion of ascorbic acid in the vitamin carrier, wherein the composition does not contain or is essentially free of water or wherein water is not freely available due to the hygroscopic properties of the composition r and or is not exposed to air during storage makes it possible to derive stable products with the most bioactive form of vitamin C.

Vitamin D is a fat-soluble vitamin and describes all sterols that exhibit the biological activity of cholecalciferol. These include: vitamin D₁ (calciferol), vitamin D₂ (ergocalciferol) vitamin D₃ (cholecalciferol), 1 (OH)D₃ (1 Hydroxycholecalciferol; alfacalcidol), 25(OH)D₃ (25 Hydroxycholecalciferol; calcifediol), 1,25(OH)₂D₃ (1,25, Dihydroxycholecalciferol; calcitriol), 24,25(OH)₂D₃ (24,25, Dihydroxycholecalciferol) and dihydrotachysterol, calcipotriene, 25-hydroxycholecalciferol, 11,25-dihydroxycholecalciferol, 1α,25-dihydroxyergocalciferol, 22,23-dihydroergocalciferol, 1,24,25-trihydroxycholecalciferol, previtamin D₃, tachysterol₃ (also termed tacalciol), isovitamin D₃, dihydrotachysterol₃, (1S)-hydroxycalciol, (24R)-hydroxycalcidiol, 25-fluorocalciol, ercalcidiol, ertacalciol, (5E)-isocalciol, 22,23-dihydroercalciol, (24S)-methylcalciol, (5E)-(10S)-10,19-dihydroercalciol, (24S)-ethylcalciol and (22E)-(24R)-ethyl-22,23-didehydrocalciol. Vitamin D is essential for promoting the absorption and utilisation of calcium and phosphorus, and normal calcification of the skeleton. Along with parathyroid hormone and calcitonin, it regulates serum calcium concentration by altering serum calcium and phosphate blood levels, as needed, and mobilizing calcium from bone. It maintains neuromuscular function and various other cellular processes, including the immune system. Calcipotriene, as well as other vitamin C forms is useful in the treatment of psoriasis.

Vitamin E is a fat-soluble vitamin and describes all tocopherol and tocotrienol derivatives that exhibit the biological activity of alpha tocopherol. Those used commercially are d-alpha tocopherol (natural vitamin E), d-alpha tocopherol acetate, d-alpha tocopherol succinate, d,l-alpha tocopherol (synthetic vitamin E), d,l-alpha tocopherol acetate and d,l-alpha tocopherol succinate. Vitamin E is an antioxidant, protecting polyunsaturated fatty acids in membranes and other critical cellular structures from free radicals and products of oxidation. It works in conjunction with dietary selenium (a co-factor for glutathione peroxidase), and also with vitamin C and other enzymes, including superoxide dismutase and catalase. Vitamin E is not very stable. It may have an anti-inflammatory effect and some studies state that it improves immune function in the elderly. It is also said to reduce oxidative damage and to improve lung function. Vitamin E can spare vitamin C and vice versa. It is said to be photo protective and to have an anti aging effect on skin showing reduced wrinkles and tumors

Vitamin K is a fat soluble vitamin and describes 2-methyl-1,4-naphthaquinone and all derivatives that exhibit qualitatively the biological activity of phytomenadione. The form of vitamin K present in foods is phytomenadione (vitamin K₁). The substances synthesized by bacteria are known as menaquinones (vitamin K₂). The parent compound of the vitamin K series is known as menadione (vitamin K₃); it is not natural substance and is not used in humans. Menadiol sodium phosphate is water-soluble derivative of menadione. Vitamin K is an essential co-factor for the hepatic synthesis of proteins involved in the regulation of blood clotting. These are: prothrombin (factor II), factors VII, IX, X and proteins C, S and Z. Vitamin K is responsible for the carboxylation of the bone protein, osteocalcin, to its active form. Osteocalcin regulates the function of calcium in bone turnover and mineralisation. Vitamin K is also required for the biosynthesis of some other proteins found in plasma and the kidney. It is reported to speed up resolution of bruising to decrease future bruising and correct aspects of photoaging.

Pseudo vitamins: Vitamin F was the designation originally given to essential fatty acids that the body cannot manufacture. They were “de-vitaminized” because they are fatty acids. Fatty acids are a major component of fats which, like water, are needed by the body in large quantities and thus do not fit the definition of vitamins which are needed only in trace amounts. Herbalists and naturopaths have named various therapedic chemicals “vitamins”, even though they are not, including vitamin T, S-Methylmethionine (vitamin U) and vitamin X. Some authorities say that ubiquinone, also called coenzyme Q10, is a vitamin. Ubiquinone is manufactured in small amounts by the body, like vitamin D. Pangamic acid, vitamin B15; the related substance dimethylglycine is quite wrongly referred to as vitamin B15 but also labeled B16. The toxins laetrile and amygdaline are sometimes referred to as vitamin B17. Both pangamic acid and laetrile were first proposed as vitamins by Ernst T. Krebs; neither are recognized by the medical community. Flavonoids are sometimes called vitamin P. Animal, bird, and bacterial growth factors have been designated vitamins such as para-aminobenzoic acid (PABA) vitamin B₁₀, the folacin (see folic acid) pteryl-heptaglutamic acid vitamin B₁₁ or vitamin Bc-conjugate and orotic acid as vitamin B₁₃. A few substances were once thought to be B-complex vitamins and are referred to as B-vitamins in older literature, including B₄ (adenine) and B₈ (adenylic acid), but are no longer recognized as such. An antitumor pterin phosphate named Vitamin B-14 and later abandoned.

Vitamins as anti oxidants. The antioxidant vitamins can be divided into those that are water-soluble and exist in aqueous solution—primarily vitamin C—and those that are fat-soluble and exist in membranes or lipoproteins—vitamin E and betacarotene. Lipid membranes are particularly vulnerable to oxidative breakdown by free radicals. Vitamin E protects cell membranes from destruction by undergoing preferential oxidation and destruction. Some quinones, such as ubiquinone (co-enzyme Q) also appear to have antioxidant properties. All these substances can act as free radical scavengers and can react directly with free radicals. Riboflavin also has a role as an antioxident.

They are believed to protect against certain diseases by preventing the deleterious effects of free-radical-mediated processes in cell membranes and by reducing the susceptibility of tissues to oxidative stress. An article by MP Ludo entitled “Antioxidants and Vitamins in Cosmetics” Clinics in Dermatology (2001): 19:467-473 discusses the benefits of vitamins and derivatives in cosmetics. Note that carotenoids and flavonoids also act as antioxidants

Synergism between vitamins is known, for example, synergism between vitamin A and vitamin E is described by Gallarate, Carlotti, Trotta, and Bovo in the International Journal of Pharmacuetics 188 (1999) 233-241 discussing a study on the stability of ascorbic acid. Any synergism known in the literature between vitamins to potentiate or facilitate their action can be used in the present invention. Details of the solubility of vitamins can be found for example in the Merck Index and other similar reference works and databases. According to one or more embodiments a hygroscopic vitamin containing composition comprises:
(a) a silicone
(b) at least one hygroscopic substance at a sufficient concentration to provide an Aw value of the hygroscopic vitamin containing composition of less than 0.9; and
(c) a vitamin or a derivative thereof or a combinations thereof

According to one or more embodiments a foamable vitamin composition comprises:
(b) a silicone
(c) a therapeutically effective concentration of a vitamin;
(d) about 50% to about 98% of a solvent selected from the group consisting of (1) a propylene glycol or derivative; and (2) a polyethylene glycol or derivatives and mixtures thereof;
(e) 0% to about 48% of a secondary solvent;
(f) an Accommodating Agent or Complex;
(g) about 0.01% to about 5% by weight of at least one polymeric agent; and
(h) a liquefied or compressed gas propellant at a concentration of about 3% to about 25% by weight of the total composition.

In one or more embodiments there is provided a method of treating a disorder or condition of mammalian subject, comprising: administering a foamable vitamin composition to a target site, the composition comprising:
(b) a silicone;
(c) a therapeutically effective concentration of a vitamin;
(d) about 50% to about 98% of a polar solvent selected from the group consisting of (1) a propylene glycol or derivatives; and (2) a polyethylene glycol or derivatives and mixtures thereof;
(e) 0% to about 48% of a secondary polar solvent;
(f) an Accommodating Agent or Complex;
(g) about 0.01% to about 5% by weight of at least one polymeric agent; and
(h) a liquefied or compressed gas propellant at a concentration of about 3% to about 25% by weight of the total composition.

In one or more embodiments the vitamin is selected from the group consisting of vitamin A, B1, B2, B3, B5, B6, B7, B9, B12, PABA, C, D1-D4, E, K and F and a derivative thereof.

In another embodiment the vitamin or a derivative thereof is susceptible to oxidation.

In a further embodiment the vitamin or a derivative thereof is soluble in water.

In another embodiment the vitamin is selected from the group consisting of vitamin B1, B2, B3, B5, B6, B7, B9, B12, PABA and C and a derivative thereof.

In an embodiment the vitamin is vitamin B3 or a derivative thereof or combinations thereof.

In an embodiment the vitamin is vitamin C or a derivative thereof or combinations thereof.

In an embodiment the vitamin is the vitamin is vitamin K or a derivative thereof or combinations thereof.

In an embodiment the vitamin is vitamin A or a derivative thereof or combinations thereof.

In an embodiment the vitamin is vitamin E or a derivative thereof or combinations thereof.

In an embodiment the vitamin is the vitamin is vitamin F or a derivative thereof or combinations thereof.

In one or more embodiments the vitamin is a combination of two or more vitamins selected from the group comprising vitamin A, B3, C, K, E, and F and a derivative thereof.

In an embodiment the vitamin or a derivative thereof or combinations thereof comprises an antioxident.

In another embodiment the vitamin or a derivative thereof or combinations thereof improves stimulates or promotes target site metabolism.

In a still further embodiment the vitamin or a derivative thereof or combinations thereof alleviates, ameliorates, treats, prevents, retards or otherwise has a beneficial effect on a skin or boy cavity condition.

In an embodiment the skin condition is selected from the group consisting of skin pigmentation, dry skin, a wound, acne, psoriasis and skin aging.

In one or more embodiments the vitamin is a combination of two or more vitamins selected from the group comprising vitamin B3, E and C and a derivative thereof.

In an embodiment, the therapeutic agent is a photodynamic therapy (PDT) agent. Suitable PDT agents include but are not limited to modified porphyrins, chlorins, bacteriochlorins, phthalocyanines, naphthalocyanines, pheophorbides, purpurins, m-THPC, mono-L-aspartyl chlorin e6, bacteriochlorins, phthalocyanines, benzoporphyrin derivatives, as well as photosensitiser precursors, such as aminolevulinic acid and derivatives, esters, salts and mixtures thereof.

In an embodiment, the therapeutic agent is an antioxidant or a radical scavenger. Suitable antioxidants and radical scavengers agents include but are not limited to ascorbic acid, ascorbyl esters of fatty acids, magnesium ascorbyl phosphate, sodium ascorbyl phosphate, ascorbyl sorbate, tocopherol, tocopheryl sorbate, tocopheryl acetate, butylated hydroxy benzoic acid, 6-hydroxy-2,5,7,8-tetramethylchroman-2-carboxylic acid, gallic acid, propyl gallate, uric acid, sorbic acid, lipoic acid, diethylhydroxylamine, amino-guanidine, glutathione, dihydroxy fumaric acid, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and polyunsaturated oils, containing omega-3 and omega-6 fatty acids (e.g., linoleic and linolenic acid, gamma-linoleic acid, eicosapentaenoic acid and docosahexaenoic acid and derivatives, esters, salts and mixtures thereof.

In an embodiment, the therapeutic agent is a self-tanning agent, such as dihydroxyacetone.

In an embodiment the therapeutic agent is an agent, capable of treating hyperhidrosis. Suitable hyperhidrosis agents include but are not limited to anticholinergic drugs, boric acid, tannic acid, resorcinol, potassium permanganate, formaldehyde, glutaraldehyde, methenamine, a Lewis acid, aluminum chloride, aluminum chlorohydrates, zirconium chlorohydrates, aluminum-zirconium-Glycine (AZG) complex, aluminum hydroxybromide, a glycopyrrolate compound, a 5-alpha-reductase inhibitor, finasteride, epristeride, flutamide, spironolactone, saw palmetto extract, cholestan-3-one, a mono- and dicarboxylic acid having 4 to 18 carbon atoms, botulinum toxin, a 5-HT2C receptor antagonist, a 5-HT2C receptor antagonist, ketanserin, ritanserin, mianserin, mesulergine, cyproheptadine, fluoxetine, mirtazapine, olanzapine and ziprasidone.

In an embodiment, the additional therapeutic agent is a sunscreen agent. Suitable sunscreen agents include but are not limited to titanium dioxide, zinc oxide, zirconium oxide, iron oxide, p-aminobenzoic acid and its derivatives (ethyl, isobutyl, glyceryl esters; p-dimethylaminobenzoic acid); anthranilic acid derivatives (i.e., o-amino-benzoates, methyl, menthyl, phenyl, benzyl, phenylethyl, linalyl, terpinyl, and cyclohexenyl esters); salicylates (amyl, phenyl, octyl, benzyl, methyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid, o- and p-hydroxybiphenyldisulfonates, coumarin derivatives (7-hydroxy, 7-methyl, 3-phenyl), diazoles (2-acetyl-3-bromoindazole, phenyl benzoxazole, methyl naphthoxazole, quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane.

In an embodiment, the additional therapeutic agent is a figure-forming agent and an agent, capable of treating cellulite. Suitable such agents include but are not limited to baldderwack extract, butcher's broom, cayenne, dandelion, red clover, ginkgo biloba, horse chestnut, witch hazel and borage oil, caffeic acid, nicotinic acid, theophiline and pentoxyphilline and salts and derivatives thereof.

Several disorders of the skin, body cavity or mucosal surface (e.g., the mucosa or the cavity of the nose, mouth, eye, ear, vagina or rectum) involve a combination of etiological factors. For example, fungal and bacterial infections and that are inflamed and have symptoms of redness and/or itching warrant therapy that combines an anti-infective agent and an anti-inflammatory agent. Thus, in several cases, combining at least two active agents that treat different etiological factors results in a synergistic effect and consequently higher success rate of the treatment.

In certain cases, the composition contains two active agents, where each of the active agents require a different pH environment in order to remain stable. For example, corticosteroids are typically stable at acidic pH values (they have a maximum stability at a pH of about 4-6) and of vitamin D analogues are typically stable at basic pH values (they have a maximum stability at pH values above about 8). In order to circumvent the problem of instability in such cases it is preferred that the composition is substantially non-aqueous. The term “substantially non-aqueous” is intended to indicate that the composition has a water content below about 5%, preferably below about 2%, such as below about 1.5%.

### Fields of Applications

The foamable carrier is suitable for treating any infected surface. In one or more embodiments, foamable carrier is suitable for administration to the skin, a body surface, a body cavity or mucosal surface, e.g., the cavity and/or the mucosa of the nose, mouth, eye, ear, respiratory system, vagina or rectum (severally and interchangeably termed herein “target site”).

By selecting a suitable active agent, or a combination of at least two active agents, the foam able composition is useful in treating an animal or a human patient having any one of a variety of dermatological disorders, including dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, eethyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo.

Likewise, the foamable composition is suitable for treating a disorder of a body cavity or mucosal surface, e.g., the mucosa of the nose, mouth, eye, ear, respiratory system, vagina or rectum. Non limiting examples of such conditions include chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

In an embodiment, the composition is useful for the treatment of an infection. In one or more embodiments, the composition is suitable for the treatment of an infection, selected from the group of a bacterial infection, a fungal infection, a yeast infection, a viral infection and a parasitic infection.

In an embodiment, the composition is useful for the treatment of wound, ulcer and burn. This use is particularly important since the composition creates a thin, semi-occlusive layer, which coats the damaged tissue, while allowing exudates to be released from the tissue.

The composition is also suitable for administering a hormone to the skin or to a mucosal membrane or to a body cavity, in order to deliver the hormone into the tissue of the target organ, in any disorder that responds to treatment with a hormone.

In light of the hygroscopic nature of the composition, it is further suitable for the treatment and prevention of post-surgical adhesions. Adhesions are scars that form abnormal connections between tissue surfaces. Post-surgical adhesion formation is a natural consequence of surgery, resulting when tissue repairs itself following incision, cauterization, suturing, or other means of trauma. When comprising appropriate protective agents, the foam is suitable for the treatment or prevention of post surgical adhesions. The use of foam is particularly advantageous because foam can expand in the body cavity and penetrate into hidden areas that cannot be reached by any other alternative means of administration.

In one or more embodiments there is provided a composition, wherein the foam demonstrates at least eighteen of the following properties:
(a) a foam quality of 4-6;
(b) a color of white to off-white; or a yellowish color:
(c) no odor or faint odor; or substantially masked odor;
(d) a foam quality of 4-6 after one freeze-thaw cycle;
(e) a foam quality of 4-6 after two freeze-thaw cycles;
(f) a foam quality of 5-6 after three freeze-thaw cycles;
(g) a foam quality of 4-6 after four freeze-thaw cycles;
(h) a foam quality of 4-6 after about 3 weeks' storage at 30° C.;
(i) a foam quality of 4-6 after about 3 weeks' storage at 40° C.;
(j) a foam quality of 4-6 after about 3 months' storage at 30° C.;
(k) a foam quality of 4-6 after about 3 months' storage at 40° C.;
(l) a collapse time of more than 50 seconds;
(m) a collapse time of more than 120 seconds; and
(n) a collapse time of more than 180 seconds;
(o) a collapse time of more than 300 seconds;
(p) a foam hardness in the range of about 5 g to about 100 g;
(q) a foam hardness in the range of about 15 g to about 55 g;
(r) a foam hardness in the range of about 30 g to about 85 g;
(s) a density of less than 0.5 g;
(t) a density of less than 0.3 g;
(u) a density of less than 0.2 g;
(v) a total focus group score of 170 or more.

In a preferred embodiment it demonstrates one or more of the following: nineteen; twenty; twenty one; twenty two properties and in a more preferred embodiment it demonstrates all of the properties.

In one or more embodiments there is provided a composition, wherein the foam provides at least two of the following traits:
(a) increased solubility of the active agent;
(b) increased delivery of the active agent;
(c) the composition provides enhanced skin barrier build up;
(d) the composition provides increased penetration of the active agent whilst replenishing the skin;
(e) the composition prolongs the delivery of the active agent whilst replenishing the skin.

In one or more embodiments there is provided a composition, wherein the foam provides at least two of the following traits:
(a) the composition is able to at least partially solubilize the active agent;
(b) the composition is able to substantially solubilize the active agent.
(c) the active agent is at least partially soluble in PEG or PG or mixtures thereof;
(d) the active agent is at least partially soluble in a solvent substantially miscible in PEG or PG or mixtures thereof e) the active agent is at least partially soluble in a hydrophilic solvent;
(e) the active agent is at least partially soluble in an oil and is distributed uniformly in the composition.

### Dual Chamber

Dual and Multi Chamber devices and heads suitable for use with the formulations described herein where a first formulation is stored in a first canister and a second formulation is stored in a second canister are described in U.S. Pat. No. 6,305,578 entitled DEVICE FOR MIXING, FOAMING AND DISPENSING LIQUIDS FROM SEPARATE COMPRESSED-GAS CONTAINERS and in US Publication 2007-0069046 and entitled APPARATUS AND METHOD FOR RELEASING A MEASURE OF CONTENT FROM A PLUARITY OF CONTAINERS all of which are incorporated herein by reference in their entirety. More particularly any of the devices and uses described are applicable herein and are incorporated by reference.

In an embodiment the dual chamber device is as described in U.S. Pat. No. 6,305,578 for example,

a compressed gas container apparatus, having
at least two compressed gas containers, disposed side by side, each for one foamable liquid product which contains a liquefied propellant gas, wherein
both compressed gas containers are each provided with a valve,
both valves are actuatable in common by a top fitting, and
each valve is provided through the top fitting with a connecting conduit,
the connecting conduits discharge into a mixing chamber, and
an expansion conduit adjoins the mixing chamber and on its end has a foam dispensing opening, characterized in that
the connecting conduits and the mixing chamber have such small cross-sectional areas that when a product is dispensed, the products flowing through the connecting conduits) and the mixing chamber remain in a liquid phase.

In an embodiment the dual dispenser head is as described in US Publication 2007-0069046 for example:

a dispenser head for use with a plurality of containers, comprising:
(a) an actuator, wherein the dispensing head is structured and positioned to be an actuator or comprises an actuator button disposed within the dispensing head to simultaneously actuate the plurality of containers
(b) a flow guide comprising
   (A) a plurality of flow conduits disposed within the flow guide; and
   (B) for each of the plurality of flow conduits,
      (ii) an inlet through a wall of the flow guide connecting with a flow conduit; and
      (iii) an outlet from a flow conduit through a wall of the flow guide;
   (C) and for each of the plurality of inlets and containers, a linker, each to link an inlet and a container to allow the contents of the container upon actuation to pass through the inlet and through the flow conduit to reach and pass through the outlet;
   (D) and wherein the flow guide is structured and positioned to allow simultaneous flow communication between each of the plurality of flow conduits and wherein the plurality of outlets are structured and positioned to allow substantially contemporaneously dispensing and/or combining of the content from a plurality of containers external to the dispensing head.

In one or more embodiments there is provided a kit comprising a dual chamber device or dual dispenser head, a first canister comprising a first foamable formulation comprising a first API and a second canister comprising a second foamable formulation comprising a second API wherein each canister is connectable to the said device or head. The first foamable formulation may be any of the silicone emulsion formulations described herein and the second foamable formulation may also be any of the silicone emulsion formulations described herein. In another embodiment the second foamable formulation is one of the foamable compositions described in the paragraph below. In an embodiment the first API is a steroid and the second API is a vitamin D derivative and the each formulation is adapted to carry an effective amount of steroid and vitamin D derivative, respectively, such that each formulation and API is sufficiently chemically and physically stable for pharmaceutical use.

Other foamable compositions are described in: U.S. Publication No. 05-0232869, published on Oct. 20, 2005, entitled NONSTEROIDAL IMMUNOMODULATING KIT AND COMPOSITION AND USES THEREOF; U.S. Publication No. 05-0205086, published on Sep. 22, 2005, entitled RETINOID IMMUNOMODULATING KIT AND COMPOSITION AND USES THEREOF; U.S. Publication No. 06-0018937, published on Jan. 26, 2006, entitled STEROID KIT AND FOAMABLE COMPOSITION AND USES THEREOF; U.S. Publication No. 05-0271596, published on Dec. 8, 2005, entitled VASOACTIVE KIT AND COMPOSITION AND USES THEREOF; U.S. Publication No. 06-0269485, published on Nov. 30, 2006, entitled ANTIBIOTIC KIT AND COMPOSITION AND USES THEREOF; U.S. Publication No. 07-0020304, published on Jan. 25, 2007, entitled NON-FLAMMABLE INSECTICIDE COMPOSITION AND USES THEREOF; U.S. Publication No. 06-0193789, published on Aug. 31, 2006, entitled FILM FORMING FOAMABLE COMPOSITION; U.S. patent application Ser. No. 11/732,547, filed on Apr. 4, 2007, entitled ANTI-INFECTION AUGMENTATION OF FOAMABLE COMPOSITIONS AND KIT AND USES THEREOF; U.S. patent application Ser. No. 11/732,547, filed on Apr. 4, 2007, KERATOLYTIC ANTIFUNGAL FOAM; U.S. patent application Ser. No. 11/767,442, filed on Jun. 22, 2007, entitled FOAMABLE COMPOSITIONS AND KITS COMPRISING ONE OR MORE OF A CHANNEL AGENT, A CHOLINERGIC AGENT, A NITRIC OXIDE DONOR, AND RELATED AGENTS AND THEIR USES; U.S. patent application Ser. No. 11/825,406, filed on Jul. 5, 2007, entitled DICARBOXYLIC ACID FOAMABLE VEHICLE AND PHARMACEUTICAL COMPOSITIONS THEREOF; U.S. patent application Ser. No. 11/900,072, filed on Sep. 10, 2006, entitled FOAMABLE VEHICLE AND VITAMIN AND FLAVONOID PHARMACEUTICAL COMPOSITIONS THEREOF; and U.S. patent application Ser. No. 11/947,751, filed Nov. 29, 2007, entitled COMPOSITIONS WITH MODULATING AGENTS, all of which are incorporated herein by reference in their entirety. More particularly any of the active ingredients; the solvents; the surfactants; foam adjuvants; polymeric agents, penetration enhancers; preservatives, humectants; moisturizers; and other excipients as well as the propellants and methods listed therein can be applied herein and are incorporated by reference.

### Chemical Instability and Stability

By chemical instability of one or more active agents is meant that at least one of the one or more active agents is susceptible to one or more of inter alia reaction, breakdown, ionization or oxidation or the rate thereof is increased when incorporated into a pharmaceutical or cosmetic carrier that is non aqueous or substantially non aqueous.

Conversely by chemical stability of one or more active agents is meant that at least one of the one or more active agents is less susceptible to one or more of inter alia reaction, breakdown, ionization or oxidation or the rate thereof is impeded when incorporated into a pharmaceutical or cosmetic carrier that is non aqueous or substantially non aqueous.

### Creaming

Formulation of emulsion foam is a very delicate balance between the functional inactive ingredients, excipients, which contribute to droplet size, separating film, viscosity and stability. In order to assure accurate and continuous foam actuation, the Foam Formulation should be liquid and shakable in the canister, otherwise it will not flow easily and completely towards and through the valve. In the context of significant levels of silicone in foamable formulations it is possible as an exception for the composition to be marginally or apparently non shakable whilst the composition has a sufficient degree of flowability under pressure of the propellant that it is possible to obtain a good quality of foam.

Stability of emulsions and resilience to creaming is desired. In the context of foamable emulsion compositions in which silicone is a significant component it has been discovered that improved physical stability is obtained by an appropriate choice of product viscosity tlirough use of different blends of polyethylene glycols or propylene glycol plus a surfactant or surfactant system optionally in combination with stabilizing agents and or viscoelastic agents, which can provide suitable rheology whilst retaining the requirements of shakability or at least flowability and by controlling droplet size.

By creaming it is meant that particles of the disperse phase concentrate in the upper layer, form a cream-like concentrated emulsion. The creaming value is defined as the relative volume of the creamed phase and the total volume the sample. The expression used for calculation of the creaming volume is as follows:

$$\% Creaming = \frac{{V}_{Creamed Phase}}{{V}_{total}} \times 100$$

Creaming values are between 1% and 99%, accordingly. 100% means “no creaming” which is the desirable best score. 0% (Zero value) indicates phase separation and is the worst score.

By physically durable in the context of waterless or substantially waterless compositions it is intended that the formulation is capable of physically withstanding partially, or to some or to a substantial degree at least one of centrifugation at 3000 rpm for at least 10 minutes; or one, two or possibly more freeze thaw cycles; or a period of time at an elevated temperature of say 30° C. or say 40° C. for say one or two or possibly three months; or a prolonged period of time at room temperature for say three to six months or possibly longer.

In a preferred embodiment the emulsion composition should exhibit pseudoplastic rheological behavior.

By selective use of appropriate stabilizing surfactant, co-surfactants and optionally stabilizing polymers the silicone emulsion compositions can be stabilized.

By appropriate selection of agents, surfactants and solvent in a silicone waterless or substantially waterless emulsion composition to facilitate biocompatibility and to achieve the appropriate balance of physical properties, it is possible to prepare formulations that are resilient to creaming or to phase separation either partially or to some or to a substantial degree when subjected to centrifugation. That said there is a reservation that the use of non volatile liquid pentane to simulate the actual propellant used may result in some cases with creaming or separation which may not truly reflect the actual formulation with volatile propellant. Also in examining any formulation for physical aging there are two levels of consideration. The first is the ability of the emulsion to remain stable at room temperature and pressure for a reasonable time period after moderate mixing or shaking and to be reconsitutable and homogenous after separation has occurred by merely applying moderate mixing or shaking. In other words the emulsion formulation is fully “reversible”. Thus the product is viable provided the consumer shakes it moderately before use. On a different level it is possible—albeit an inventive and onerous task—to identify and fine tune emulsion formulations that can remain stable without separation for prolonged periods of time and such formulations are simulated by their resitance to creaming or separation.

### Methodology

A general procedure for preparing foamable compositions is set out in Internation Patent Publication No. WO 2004/037225, which is incorporated herein by reference.

The waterless formulas may be made in the following general methodology set out below with appropriate adjustments for each formulation as will be appreciated by someone skilled in the art.

### Silicone in Glycol Emulsion

1. Mix main solvent emulsifiers and foam adjuvants and heat to 75° C. to melt and dissolve the various ingredients with vigorous mixing.
2. Homogenize the formulation with vigorous mixing.
3. Add the silicones at 60° C. with vigorous mixing.
4. Cool to below 40° C. and add sensitive ingredients with mild mixing.
   Formulations with HPMC

This methodology is suitable, for all formulations described comprising hydroxypropylmethyl cellulose (HPMC) (Where the formulation is without polymer, the production starts at section 2).
1. Dissolve the polymers in the main solvent with heating or cooling as appropriate for specific polymer and with vigorous mixing.
2. Add to main solvent emulsifiers and foam adjuvants and heat to 75° C. to melt and dissolve the various ingredients with vigorous mixing.
3. Homogenize the formulation with vigorous mixing.
4. Add the silicones at 60° C. with vigorous mixing.
5. Cool to below 40° C. and add sensitive ingredients with mild mixing.
6. Cool to room temperature.
   Formulations with ASOS

This methodology is suitable for all formulation described herein that include aluminum starch octenylsuccinate (ASOS)
1. Add to main solvent emulsifiers and foam adjuvants and heat to 75° C. to melt and dissolve the various ingredients with vigorous mixing.
2. Homogenize the formulation with vigorous mixing
3. Add the silicones at 60° C. with vigorous mixing
4. Cool to below 40° C. and add sensitive ingredients (e.g., ASOS) with mild mixing.
5. Cool to room temperature.
   Formulations with Carbopol.

This methodology is suitable for all formulation described herein that include Carbopol.
1. Separate part fro the solvent and add the carbopol
2. Homogenize the carbopol at room temperature until complete (i.e., for a few minutes).
3. Add to the rest of main solvent emulsifiers and foam adjuvants and heat to 75° C. to melt and dissolve the various ingredients with vigorous mixing.
4. Homogenize the formulation with vigorous mixing
5. Add the silicones at 60° C. with vigorous mixing
6. Cool to below 40° C. and mix with carbopol mixture with vigorous mixing.
7. Cool to room temperature.
   Formulations with Microsponge Polymer.
1.) Prepare a silicone formulation as described above.
2.) When the formulation is at room temperature, the microsponge polymer is added and the formulation is mixed for about 5 to about 10 minutes until a uniform dispersion is obtained.

The canisters are then filled with the above waterless formula, sealed and crimped with a valve and pressurized with the propellant. A nonlimiting exemplary procedure includes the following steps:
1. Each aerosol canister 35×70 mm is filled with 30±5% g of the composition;
2. Each canister is closed with an aerosol valve, using a vacuum crimping machine;
3. Propellant (mix of propane, butane and isobutane) is added to each of the canisters.

### Production Under Vacuum

Optionally, the foamable formulation may be produced under nitrogen and under vacuum. Whilst the whole process can be carried out under an oxygen free environment, it can be sufficient to apply a vacuum after heating and mixing all the ingredients to obtain an emulsion or homogenous liquid. Preferably the production chamber is equipped to apply a vacuum but if not the formulation can be for example placed in a dessicator to remove oxygen prior to filing and crimping.

Loading and Testing of Canisters

An aerosol canister is filled with PFF and crimped with valve using vacuum crimping machine.

Pressurizing is then carried out using a gas mixture comprising n-Butane. Canisters are thereafter filled and preferably warmed for 30 seconds in a warm bath at 50° C. and well shaken immediately thereafter.

Each pressurized canister is subjected to bubble and crimping integrity testing by immersing the canister in a 60° C. water bath for 2 minutes. Canisters are observed for leakage as determined by the generation of bubbles. Canisters releasing bubbles are rejected.

### Tests

By way of non limiting example the objectives of hardness, collapse time, FTC stability tests and aging are briefly set out below as would be appreciated by a person skilled in the art.

### Hardness

LFRA100 instrument is used to characterize hardness. A probe is inserted into the test material. The resistance of the material to compression is measured by a calibrated load cell and reported in units of grams on the texture analyzer instrument display. Preferably at least three repeat tests are conducted. The textural characteristics of a dispensed foam can effect the degree of dermal penetration, efficacy, spreadability and acceptability to the user. The results can also be looked at as an indicator of softness. Note: the foam sample is dispensed into an aluminum sample holder and filled to the top of the holder.

### Collapse Time

Collapse time (CT) is examined by dispensing a given quantity of foam and photographing sequentially its appearance with time during incubation at 36° C. It is useful for evaluating foam products, which maintain structural stability at skin temperature for at least 1 minute. Thus foams which are structurally stable on the skin for at least one minute are termed “short term stable” compositions or foams.

### Viscosity

Viscosity is measured with Brookfield LVDV-II+PRO with spindle SC4-25 at ambient temperature and 10, 5 and 1 RPM. Viscosity is usually measured at 10 RPM. However, at about the apparent upper limit for the spindle of ˜>50,000 CP, the viscosity at 1 RPM may be measured, although the figures are of a higher magnitude.

### Freeze Thaw Cycles (FTC)

FTC (Freeze Thaw Cycles) To check the foam appearance under extreme conditions of repeated cycles of cooling, heating, (first cycle) cooling, heating (second cycle) etc., commencing with −10° C. (24 hours) followed by +40° C. (24 hours) measuring the appearance and again repeating the cycle for up to three times.

### Chemical Stability

The amount of active agent present is analyzed in foam expelled from various pressurized canisters containing foam formulations using HPLC. Analysis is carried out at zero time and at appropriate time intervals thereafter. The canisters are stored in controlled temperature incubators at 5° C., at 25° C., at, 40° C. and sometimes at 50° C. At appropriate time intervals canisters are removed and the amount of active agent in the foam sample is measured.

### Focus Group

Healthy volunteers selected at random were give a sample of foam formulation and applied it to the skin on their forearm and were asked to complete a questionnaire.

### Corneometer

Skin hydration is measured using a Comeometer® CM 825 instrument. (Courage+Khazaka, Koln, Germany). The measuring principle of the Comeometer® CM 825 is based on capacitance measurement of dielectric medium. Any change in the dielectric constant due to skin surface hydration alters the capacitance of a measuring capacitor. It can detect even slight changes in the skin hydration level.

### Bubble Size

Foams are made of gas bubbles entrapped in liquid. The bubble size and distribution reflects in the visual texture and smoothness of the foam. Foam bubbles size is determined by dispensing a foam sample on a glass slide, taking a picture of the foam surface with a digital camera equipped with a macro lens. The diameter of about 30 bubbles is measured manually relatively to calibration standard template. Statistical parameters such as mean bubble diameter, standard deviation and quartiles are then determined. Measuring diameter may also be undertaken with image analysis software. The camera used was a Nikon D40X Camera (resolution 10 MP) equipped with Sigma Macro Lens (ref: APO MACRO 150 mm F2.8 EX DG HSM). Pictures obtained are cropped to keep a squared region of 400 pixels×400 pixels.

The light microscope enables observing and measuring particles from few millimeters down to one micron. Light microscope is limited by the visible light wavelength and therefore is useful to measuring size of particles above 800 nanometers and practically from 1 micron (1,000 nanometers).

“Shakability” represents the degree to which the user is able to feel/hear the presence of the liquid contents when the filled pressurized canister is shaken. Shaking is with normal mild force without vigorous shaking or excessive force. When the user cannot sense the motion of the contents during shaking the product may be considered to be non shakable. This property may be of particular importance in cases where shaking is required for affecting proper dispersion of the contents.

### Shakability Scoring:

| | |
|---|---|
| Good shakability (conforms to required quality specification) | 2 |
| Moderate shakability (conforms to required quality specification) | 1 |
| Not or hardly shakable but may still be flowable and allow foam | 0 |
| formation of quality | |
| Is substantially not able to pass through valve | Block |

### Aging or Creaming by Centrifugation:

### 1. Principle of Test

The centrifugation used in this procedure serves as a stress condition simulating the aging of the liquid dispersion under investigation. Under these conditions, the centrifugal force applied facilitates the coalescence of dispersed globules or sedimentation of dispersed solids, resulting in loss of the desired properties of the formulated dispersion.

In the case of waterless silicone emulsion compositions which are inherently more susceptible to creaming by virtue of the silicone and by virtue of the waterless solvent, the presence of some creaming at the enormous centrifugal forces imposed on the formulations does not derogate from the fact that the compositions have not phase separated and can still be understood as being resistant to creaming and provides a good indication of the long term stability of the formulations. To the extent that good quality stable formulations are achieved, which are resistant to creaming or such that no creaming is observed, the formulations are considered as exceptionally stable. The procedure is as follows:
1. Following preparation of the experimental formulation/s, allow to stand at room temperature for ≧24 h.
2. Handle pentane in the chemical hood. Add to each experimental formulation in a 20-mL glass vial a quantity of pentane equivalent to the specified quantity of propellant for that formulation, mix and allow formulation to stand for at least 1 h and not more than 24 h.
3. Transfer each mixture to 1.5 mL microtubes. Tap each microtube on the table surface to remove entrapped air bubbles.
4. Place visually balanced microtubes in the centrifuge rotor and operate the centrifuge at about 300 rpm for 10 min. about 1,000 rpm for 10 min. or at about 3,000 rpm for 10 min or at about 10,000 rpm for 10 min. The centrifuge can be a BHG HEMLE Z 231 M.
5. Centrifugation can also be executed at a higher rpm for a shorter period or a lower rpm for a longer period bearing in mind the G force experienced by the formulations is many fold greater than the one G to which a formulation would be exposed to during its shelf life.

### BRIEF DESCRIPTION OF THE DRAWINGS

In the drawings:

FIG. 1 is a photomicrograph of a silicone in polyethylene glycol emulsion.

FIG. 2 is a photomicrograph of a silicone in polyethylene glycol emulsion.

FIG. 3 shows formulation f-041 inverted horizontally before and after addition of the propellant.

FIG. 4 shows formulation f-041 inverted at an angle after addition of the propellant.

FIG. 5 is a photomicrograph of silicone and hydroxypropyl cellulose in polyethylene glycol.

FIG. 6 is a photograph of formulation f-003 in a pressurized glass bottle with propellant AP 70 following shaking.

### EXAMPLES

The invention is described with reference to the following examples. This invention is not limited to these examples and experiments. Many variations will suggest themselves and may be carried out by one of ordinary skill in the art and are within the full intended scope of the description and claims herein. The following examples further exemplify the stable non-alcoholic foamable pharmaceutical carrier, pharmaceutical compositions thereof, methods for preparing the same, and therapeutic uses of the compositions. For the purpose of the Examples below it was sufficient to apply a vacuum only at the crimping stage although for long term stability preferably any vacuum should be applied during manufacture as well at a sufficient pressure so that any oxygen remaining in the formulation is virtually negligible. The examples are for the purposes of illustration only and are not intended to be limiting. Many variations are contemplated within the full scope the invention.

### Section 1

### Polyethylene Glycol (“PEG”) Platform

### Example 1

### PEG Vehicle with Different Silicones (No Polymer)

### Ex 1 Part A. Formulations

### PEG Vehicle with Different Silicones (No Polymer)

| | | f-072 | f-073 | f-074 |
|---|---|---|---|---|
| Ingredient | Function | W/W % | W/W % | W/W % |
| Methyl glucose sesqui | Emulsifier | 3.00 | 3.00 | 3.00 |
| stearate | | | | |
| PEG-200 | Solvent | 43.50 | 43.50 | 43.50 |
| PEG-400 | Solvent | 44.00 | 44.00 | 44.00 |
| Polydimethylsiloxane | Silicone | — | 5 | — |
| Polymer | | | | |
| Dow Corning(R) 345 | Silicone | 5 | — | — |
| Fluid | | | | |
| Cetyl dimethicone | Silicone | — | — | 5 |
| Steareth 2 | Emulsifier | 4.5 | 4.5 | 4.5 |
| Total | | 100.00 | 100.00 | 100.00 |
| Propellant* | | 8 | 8 | 8 |

*Propellant is mix of propane, butane and isobutane

### Ex 1 Part B. Results

### PEG Vehicle with Different Silicones (No Polymer)

| | f-072 | | f-073 | | f-074 | |
|---|---|---|---|---|---|---|
| | Time point | | | | | |
| Parameter | ZT | FTC | ZT | FTC | ZT | FTC |
| Foam quality | Good | Good | Good | Good | Good | Good |
| Odor** | N.O. | N.O. | N.O. | N.O. | N.O. | N.O. |
| Color | White | White | White | White | White | White |
| Hardness (g) | 26.90 | 16.54 | 14.25 | 14.36 | 59.49 | 80.72 |
| Density (g/mL) | 0.148 | 0.115 | 0.168 | 0.108 | 0.078 | 0.098 |
| Collapse (sec) | 300. | 300. | >300 | >300 | >300 | >300 |
| Viscosity 10 RPM (cP) | 5200 | N.M | 8243 | N.M | 8024 | N.M |
| Viscosity 1 RPM (cP) | N.M. | N.M | 31033 | N.M | N.M | N.M |
| Centrifuge 3k (% of creaming) | 15 | N.M | 95 | N.M | 20 | N.M |
| Centrifuge 10k (% of creaming) | 15 | | 95 | | 15 | |
| Shakability* | 2 | 2 | 2 | 2 | 2 | 2 |

*2: good, 1: moderate, 0: poor
**N.O.—No Odor; V.F.O.—very faint odor
ZT—zero time;
FTC—freeze thaw cycles test. In this test samples are stored in stressed conditions as follows: −10° C. follows by 40° C. This stress is repeated four times.

No polymer is needed for formulation. The results show that good quality foam having a good collapse time can be produced with one stabilizing emulsifier without polymer.

### Ex 1 Part C

### Microscopic Description of Formulations (72-74)

| | | | Range | |
|---|---|---|---|---|
| | | | of | |
| | Emulsion | Average of | droplet | Liquid |
| | (y/n) | droplet size | size | crystals |
| f-072 | Y | 4μ | 2-10μ | Very few |
| f-073 | Y | 4μ | 2-10μ | Very few |
| f-074 | Y | 4μ | 2-10μ | Very few |

Ex1 Part D—Picture 1-f-073

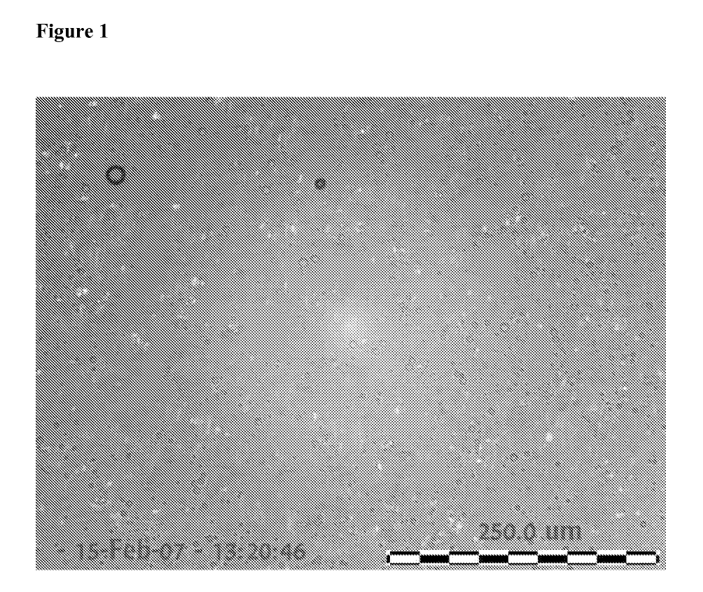

In FIG. 1, a silicon in polyethylene glycol emulsion can be observed.

### Example 2

### Silicones in Polyethylene Glycol 400 and 200 with Hydroxypropyl Cellulose at ZT

### Ex 2 Part A

### Formulations and Results—Silicones in Polyethylene Glycol 400 and 200 with Hydroxypropyl Cellulose at ZT

| | | f-010 | f-011 | f-012 | f-013 |
|---|---|---|---|---|---|
| Ingredients | Function | W/W % | W/W % | W/W % | W/W % |
| Methyl glucose sesqui | Emulsifier | 2.00 | 2.00 | 2.00 | 2.00 |
| stearate | | | | | |
| PEG-200 | Solvent | 42.00 | 65.00 | 65.00 | 65.00 |
| PEG-400 | Solvent | 42.00 | 17.50 | 19.50 | 21.50 |
| Polydimethylsiloxane | Silicone | 5.00 | 3.00 | 3.00 | 3.00 |
| Polymer | | | | | |
| Dow Corning(R) 345 | Silicone | — | | 1.50 | 1.50 |
| Fluid | | | | | |
| cyclopentasiloxane | | | 1.50 | | |
| DC245 | | | | | |
| Bis-PEG-18 Methyl | Silicone | 1.00 | 2.00 | 2.00 | — |
| Ether Dimethyl Silane | | | | | |
| Hydroxypropyl | Polymer | 2.00 | 2.00 | 2.00 | 2.00 |
| cellulose | | | | | |
| Steareth 2 | Emulsifier | 2.00 | 1.00 | 1.00 | 1.00 |
| Cetearyl alcohol | Emulsifier | — | 2.00 | — | — |
| (and)cetearyl | | | | | |
| glucoside | | | | | |
| Stearyl alcohol | Foam | 4.00 | 4.00 | 4.00 | 4.00 |
| | Adjuvant | | | | |
| Total | | 100 | 100 | 100 | 100 |
| Propellant*** | | 8 | 8 | 8 | 8 |

Results

| | | | | |
|---|---|---|---|---|
| Foam quality | Good | Good | Good | Good |
| Odor** | N.O. | N.O. | N.O. | N.O. |
| Color | White | White | White | White |
| Hardness (g) | N.M. | 11.55 | 19.42 | 9.65 |
| Density (g/mL) | 0.113 | 0.107 | 0.098 | 0.1 |
| Collapse (sec) | N.M. | 60 | 50 | 200 |
| Centrifuge 3k (% of creaming) | 10 | N.M. | N.M. | N.M. |
| Centrifuge 10k (% of creaming) | 20 | N.M. | N.M. | N.M. |
| Shakability* | 1 | 0 | 0 | 0 |

*2: good, 1: moderate, 0: poor
**N.O.—No Odor; V.F.O.—very faint odor
***Propellant is mix of propane, butane and isobutane

This formulation can be made without Cetearyl alcohol (and) cetearyl glucoside.(e.g. f-012)

Apparently, foam quality is not modified by varying the ratio of PEG 200 and PEG 400. (f010-13).

### Ex 2 Part B

### Formulations and Results—Silicones in Polyethylene Glycol 4000 and 2000 with Hydroxypropyl Cellulose at ZT

| | | f-014 | f-015 | F-044 | f-048 |
|---|---|---|---|---|---|
| Ingredients | Function | W/W % | W/W % | W/W % | W/W % |
| Methyl glucose sesqui | Emulsifier | 2.00 | 2.00 | — | — |
| stearate | | | | | |
| PEG-200 | Solvent | 54.50 | 20.50 | 46.00 | 44.00 |
| PEG-400 | Solvent | 32.00 | 65 | 45.00 | 44.00 |
| Polydimethylsiloxane | Silicone | 3.00 | 5.00 | 3.00 | 3.00 |
| Polymer | | | | | |
| Dow Corning(R) 345 | Silicone | 1.50 | 1.50 | 1.50 | 1.50 |
| Fluid | | | | | |
| Hydroxypropyl | Polymer | 2.00 | | 3.00 | 3.00 |
| cellulose | | | | | |
| Steareth 2 | Emulsifier | 1.00 | 2.00 | 1.5 | 4.50 |
| Stearyl alcohol | Foam | 4.00 | 4.00 | — | — |
| | Adjuvant | | | | |
| Total | | 100.00 | 100.00 | 100.00 | 100.00 |
| Propellant*** | | 8 | 8 | 8 | 8 |

Results

| | | | | |
|---|---|---|---|---|
| Foam quality | Good | Good | Good | Good |
| Odor** | N.O | N.O | N.O | N.O |
| Color | White | White | White | White |
| Hardness (g) | 21.39 | 8.52 | N.M. | N.M. |
| Density (g/mL) | 0.1 | 0.133 | 0.084 | 0.094 |
| Collapse (sec) | 50 | 50 | N.D | 70 |
| Centrifuge 3k (% of creaming) | N.M. | N.M. | 45 | stable |
| Centrifuge 10k (% of creaming) | N.M. | N.M. | 45 | stable |
| Shakability* | 0 | 1 | 1 | 2 |

*2: good, 1: moderate, 0: poor
**N.O.—No Odor; V.F.O.—very faint odor
***Propellant is mix of propane, butane and isobutane

Apparently, foam quality is not modified by varying the ratio of PEG 200 and PEG 400. Hardness may be reduced by omission of poymer.

### Ex 2 Part C

### Formulations and Results—Silicones in Polyethylene Glycol 400 and 200 at ZT

| | | f-049 | 059 |
|---|---|---|---|
| Ingredients | Function | W/W % | W/W % |
| Methyl glucose | Emulsifier | — | 3.00 |
| sesqui stearate | | | |
| PEG-200 | Solvent | 44.00 | 44.00 |
| PEG-400 | Solvent | 44.00 | 44.00 |
| Polydimethylsiloxane | Silicone | 3.00 | 3.00 |
| Polymer | | | |
| Dow Corning(R) | Silicone | 1.50 | 1.50 |
| 345 Fluid | | | |
| Steareth 2 | Emulsifier | 4.50 | 4.50 |
| Cetearyl alcohol | Emulsifier | 3.00 | — |
| (and)cetearyl glucoside | | | |
| Total | | 100 | 100 |
| Propellant*** | | 8 | 8 |

Results

Time point

| Parameter | ZT | ZT |
|---|---|---|
| Foam quality | Good | Good |
| Odor** | N.O. | N.O. |
| Color | White | White |
| Density (g/mL) | 0.103 | N.D |
| Centrifuge 3k (% of creaming) | 10 | 25 |
| Centrifuge 10k (% of creaming) | 5 | 25 |
| Shakability* | 2 | 2 |

*2: good, 1: moderate, 0: poor
**N.O.—No Odor; V.F.O.—very faint odor
***Propellant is mix of propane, butane and isobutane

Part C indicates that cetearyl alcohol (and) cetearyl glucoside (Montanov 68) (f-049) and Methyl glucose sesqui stearate are replaceable (f-059)

### Example 3

### Polyethylene Glycol Formulations with Aluminium Starch Octenylsuccinate (ASOS)

Formulations with PEGs were prepared with ASOS. Different emulsifiers were used and can be used in various concentrations.

### Ex 3 Part A

### PEG Formulatios with Different Silicones and ASOS

| | | f-075 | f-076 | f-077 |
|---|---|---|---|---|
| Ingredients | Function | W/W % | W/W % | W/W % |
| PEG-200 | Solvent | 40.00 | 40.00 | 40.00 |
| PEG-400 | Solvent | 39.50 | 39.50 | 39.50 |
| Aluminum starch | Polymer | 3 | 3 | 3 |
| octenylsuccinate | | | | |
| (ASOS) | | | | |
| Stearic acid | Co- | 9 | 9 | 9 |
| | emulsifying | | | |
| Polydimethylsiloxane | Silicone | — | 5 | — |
| Polymer | | | | |
| Dow Corning(R) 345 | Silicone | 5 | — | — |
| Fluid | | | | |
| Cetyl dimethicone | Silicone | — | — | 5 |
| Steareth 2 | Emulsifier | 3.5 | 3.5 | 3.5 |
| Total | | 100 | 100 | 100 |
| Propellant**** | | 8 | 8 | 8 |

****Propellant is mix of propane, butane and isobutane

### Ex 3 Part B

### Results with PEG with Different Silicones and ASOS

| | f-075 | | f-076 | | f-077 | |
|---|---|---|---|---|---|---|
| | Time point | | | | | |
| Parameter | ZT | FTC | ZT | FTC | ZT | FTC |
| Foam quality | Good | FG | Good | Good | Good | Good |
| Odor** | N.O. | N.O. | V.F.O | V.F.O | N.O. | N.O. |
| Color | White | White | White | White | White | White |
| Hardness (g) | 18.40 | N.M | 87.62 | 66.38 | 41.3 | 32.06 |
| Density (g/mL) | 0.148 | N.M | 0.103 | 0.123 | 0.085 | 0.070 |
| Collapse (sec) | >300 | N.M | 210. | 80 | >300 | 270 |
| ~Viscosity 10 RPM | >52700 | N.M | >52700 | N.M | >52700 | N.M |
| (cP) | | | | | | |
| Viscosity 1 RPM | 192279 | N.M | 194998 | N.M | 185400 | N.M |
| (cP) | | | | | | |
| Shakability* | 1 | | 2 | 2 | 2 | 2 |

| | | | |
|---|---|---|---|
| Centrifuge | 20% of creaming | 20% of creaming | 30% of creaming |
| 3k | No sediment | No sediment | 10% of sediment |
| Centrifuge | 20% of creaming | 20% of creaming | 30% of creaming |
| 10k | 5% of sediment | 5% of sediment | 10% of sediment |

*2: good, 1: moderate, 0: poor
**N.O.—No Odor; V.F.O.—very faint odor

Three types of silicones compared. Two successfully passed through FTC. The foam quality of the Dow corning 345 formulation was reduced after FTC. ASOS may cause sedimentation.

### Ex 3 Part C

### Silicone in Polyethylene Glycol 400 and 200 formulations with ASOS at ZT

| | | F-020 | F-022 |
|---|---|---|---|
| Ingredients | Function | W/W % | W/W % |
| Methyl glucose | Emulsifiers | 2 | 2 |
| sesqui stearate | | | |
| PEG-200 | Solvent | 42.0 | 41.5 |
| PEG-400 | Solvent | 42.5 | 42 |
| Aluminum starch | Polymer | 3 | 3 |
| octenylsuccinate | | | |
| Stearic acid | Co-emulsifying | 3 | 5 |
| Polydimethyl- | Silicone | 3 | 3 |
| siloxane Polymer | | | |
| Dow Corning(R) | Silicone | 1.5 | 1.5 |
| 345 Fluid | | | |
| Steareth 2 | Emulsifier | 1 | 2 |
| Stearyl alcohol | Co-emulsifying | 2 | — |
| Total | | 100 | 100 |
| Propellant*** | | 8 | 8 |

Results

| | | |
|---|---|---|
| Foam quality | Good | Good |
| Odor** | N.O. | N.O. |
| Color | White | White |
| Hardness (g) | N.M. | 9.01 |
| Density (g/mL) | 0.112 | 0.139 |
| Collapse (sec) | 80 | 120 |
| Shakability* | 2 | 2 |
| Centrifuge 3k | 10% of creaming | 20% of creaming |
| | 5% of sediment | No sediment |
| Centrifuge 10k | 5% of creaming | 5% of creaming |
| | 5% of sediment | 5% of sediment |

*2: good, 1: moderate, 0: poor
**N.O.—No Odor; V.F.O.—very faint odor
***Propellant is mix of propane, butane and isobutene

Stearyl Alcohol can be excluded from the formula without reducing foam quality (f-022).

### Ex 3 Part D Silicone in Polyethylene Glycol 400 and 200 Formulations with ASOS at ZT

| | | F-023 | F-024 |
|---|---|---|---|
| Ingredients | Function | W/W % | W/W % |
| Methyl glucose sesqui | Emulsifiers | 3 | — |
| stearate | | | |
| PEG-200 | Solvent | 40 | 40 |
| PEG-400 | Solvent | 40 | 40 |
| Aluminum starch | Polymer | 3 | 3 |
| octenylsuccinate (ASOS) | | | |
| Stearic acid | Co-emulsifying | 6 | 9 |
| Polydimethylsiloxane | Silicone | 3 | 3 |
| Polymer | | | |
| Dow Corning(R) 345 Fluid | Silicone | 1.5 | 1.5 |
| Steareth 2 | Emulsifier | 3.5 | 3.5 |
| Total | | 100 | 100 |
| Propellant*** | | 8 | 8 |

| | Time point | | |
|---|---|---|---|
| Parameter | ZT | ZT | FTC |
| Foam quality | Good | Good | Good |
| Odor** | N.O. | N.O. | N.O. |
| Color | White | White | White |
| Hardness (g) | 17.7 | N.D | N.M |
| Density (g/mL) | 0.113 | 0.113 | 0.11 |
| Collapse (sec) | 120 | 160 | 280 |
| Centrifuge 3k (% of creaming) | 30 | 10 | N.M |
| Shakability* | 2 | 2 | 2 |

| | F-023 | F-024 |
|---|---|---|
| Centrifuge | 30% of creaming | 5% of creaming |
| 10k | 10% of sediment | 5% of sediment |

*2: good, 1: moderate, 0: poor
**N.O.—No Odor; V.F.O.—very faint odor
***Propellant is mix of propane, butane and isobutane

### Ex 3 Part E Microscopic Description of Formulation f-024 Silicone in Polyethylene Glycol 400 and 200 Formulation with ASOS at ZT

| | | Average of | Range of | |
|---|---|---|---|---|
| | Emulsion (y/n) | droplet size | droplet size | Liquid crystals |
| f-024 | Y | 4μ | 2-10μ | Very few |

### Ex 3 Part F Microscopic Picture 2—of Formulation f-024 Silicone in Polyethylene Glycol 400 and 200 Formulation with ASOS at ZT

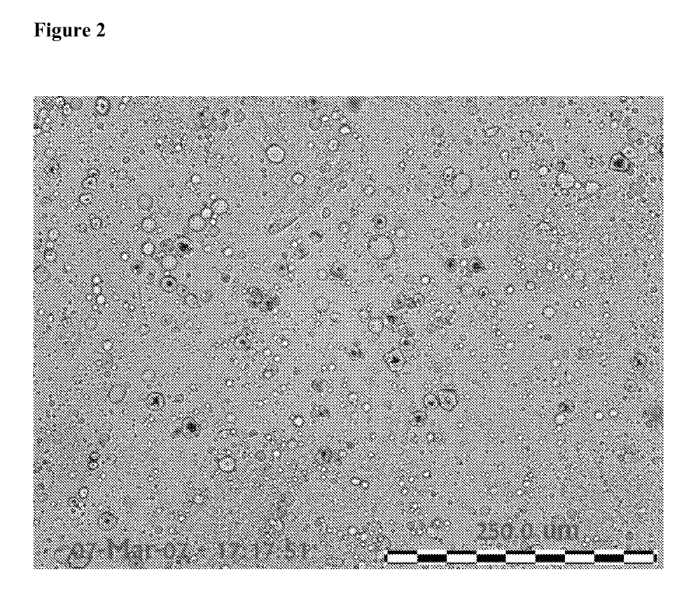

In FIG. 2, a silicon in polyethylene glycol emulsion can be observed.

### Ex 3 Part G

### Silicones in Vehicle Polyethylene Glycol 400 and 200 with ASOS at ZT with Polymer and Foam Adjuvant but No Emulsifier

| | | F-019 |
|---|---|---|
| Ingredients | Function | W/W % |
| PEG-200 | Solvent | 40.00 |
| PEG-400 | Solvent | 40.00 |
| Aluminum starch | Polymer | 3.00 |
| octenylsuccinate | | |
| Stearic acid | Foam | 9.00 |
| | Adjuvant | |
| Polydimethylsiloxane | Silicone | 3.00 |
| Polymer | | |
| Dow Corning(R) 345 | Silicone | 1.50 |
| Fluid | | |
| Bis-PEG-18 Methyl | Silicone | 3.50 |
| Ether Dimethyl Silane | | |
| Stearyl alcohol | Foam | 2 |
| | Adjuvant | |
| Total | | 100 |
| Propellant*** | | 8 |

Results

| | |
|---|---|
| Foam quality | FG |

***Propellant is mix of propane, butane and isobutane

Foam quality is apparently impaired with significant levels of silicone without the presense of an emulsifier.

### Example 4

### Silicones in Polyethylene Glycol 400 and 200 with Trolamine but no Polymer

### Ex 4 Part A

### Formulations of PEGS with Different Silicones and No Polymer

| | | f-078 | f-079 | f-080 |
|---|---|---|---|---|
| Ingredients | Function | W/W % | W/W % | W/W % |
| PEG-200 | Solvent | 42.00 | 42.00 | 42.00 |
| PEG-400 | Solvent | 42.00 | 42.00 | 42.00 |
| Trolamine | Stabilizing | 2 | 2 | 2 |
| Stearic acid | complex | 4 | 4 | 4 |
| Polydimethylsiloxane | Silicone | — | 5 | — |
| Polymer | | | | |
| Dow Corning(R) 345 | Silicone | 5 | — | — |
| Fluid | | | | |
| Cetyl dimethicone | Silicone | — | — | 5 |
| Steareth 2 | Emulsifier | 5 | 5 | 5 |
| Total | | 100 | 100 | 100 |
| Propellant*** | | 8 | 8 | 8 |

***Propellant is mix of propane, butane and isobutane

### Ex 4 Part B Results of PEGS with Different Silicones and No Polymer

| | f-078 | | f-079 | | f-080 | |
|---|---|---|---|---|---|---|
| | Time point | | | | | |
| Parameter | ZT | FTC | ZT | FTC | ZT | FTC |
| Foam quality | Good | Good | Good | Good | Good | Good |
| Odor** | V.F.O | N.O. | N.O. | V.F.O | N.O. | N.O. |
| Color | off- | off- | off- | off- | Off- | off- |
| | white | white | white | white | white | white |
| Hardness (g) | 23.71 | 23.33 | 14.92 | 17.78 | 74.88 | 57.88 |
| Density (g/mL) | 0.121 | 0.128 | 0.095 | 0.123 | 0.052 | 0.078 |
| Collapse (sec) | 160.00 | 170 | 240 | >300 | >300 | >300 |
| ~Viscosity 10 RPM | 34486 | N.M | >52700 | N.M | >52700 | N.M |
| (Cp) | | | | | | |
| Viscosity 1 RPM | 101098 | | 227911 | | 194998 | |
| (Cp) | | | | | | |
| Centrifuge 3k (% | 25 | | 25 | | 20 | |
| of creaming) | | | | | | |
| Centrifuge | 15 | | 15 | | 10 | |
| 10k ((% of | | | | | | |
| creaming) | | | | | | |
| Shakability* | 2 | 2 | 1 | 2 | 1 | 2 |

*2: good, 1: moderate, 0: poor
**N.O.—No Odor; V.F.O.—very faint odor

Three types of silicones were tested on one formula. Cetyl dimethicone did not affect foam quality and foam parameters and can be used in higher concentrations.

### Ex 4 Part C Formulations of Silicones in Polyethlene Glycol 400 and 200 with Trolamine

| | | F-041 | F-043 |
|---|---|---|---|
| Ingredients | Function | W/W % | W/W % |
| PEG-200 | Solvent | 42.5 | 42.5 |
| PEG-400 | Solvent | 42 | 42 |
| Trolamine | Stabilizing | 2 | 2 |
| Stearic acid | complex | 4 | — |
| Polydimethylsiloxane | Silicone | 3 | 3 |
| Polymer | | | |
| Dow Corning(R) 345 | Silicone | 1.5 | 1.5 |
| Fluid | | | |
| Steareth 2 | Emulsifier | 5 | 5 |
| Stearyl alcohol | Foam | — | 4 |
| | Adjuvant | | |
| Total | | 100 | 100 |
| Propellant*** | | 8 | 8 |

Results

| | Time point | | |
|---|---|---|---|
| Parameter | ZT | FTC | ZT |
| Foam quality | Good | Good | Good |
| Odor** | N.O. | N.O. | N.O. |
| Color | White | White | White |
| Hardness (g) | 28.51 | 22.45 | 52.03 |
| Density (g/mL) | 0.090 | 0.108 | 0.073 |
| Collapse (sec) | 220 | 200.00 | >300 |
| Centrifuge 3k (% of creaming) | 20 | N.M | N.M |
| Centrifuge 10k ((% of creaming) | 20 | | N.M |
| Shakability* | 1 | 2 | 2 |

*2: good, 1: moderate, 0: poor
**N.O.—No Odor; V.F.O.—very faint odor
***Propellant is mix of propane, butane and isobutane

Those formulations produced good quality foam without polymer addition. Stearyl alcohol can be replaced by of stearic acid.

### Ex 4 Part C Pictures of Formulation f-041 of Silicones in Polyethylene Glycol 400 and 200 with Trolamine

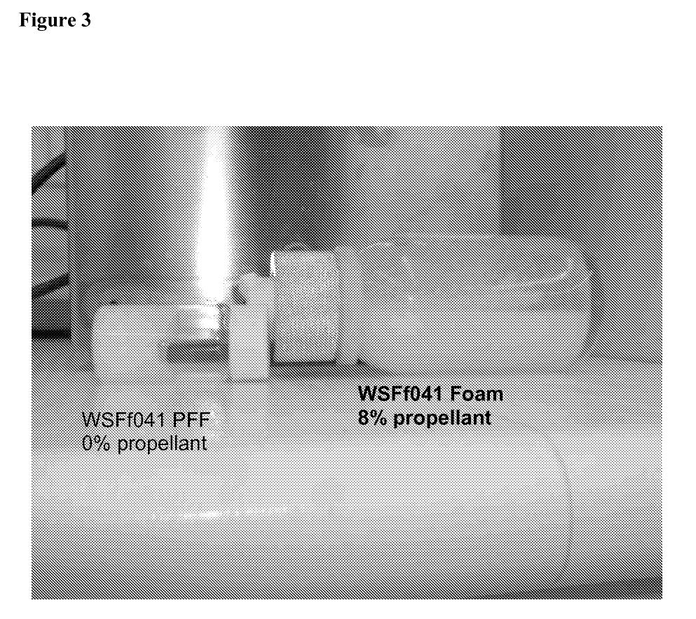

In FIG. 3, formulation f-041 is inverted horizontally before and after addition of the propellant. The formulation with propellant is presented in a special pressurized glass container. It will be noted that prior to propellant addition the formulation is solid and its inversion to the horizontal does not change its form so that its upper surface is vertical whilst unexpectedly after propellant addition the formulation is liquid and following inversion to the horizontal has flowed so that its upper surface is horizontal.

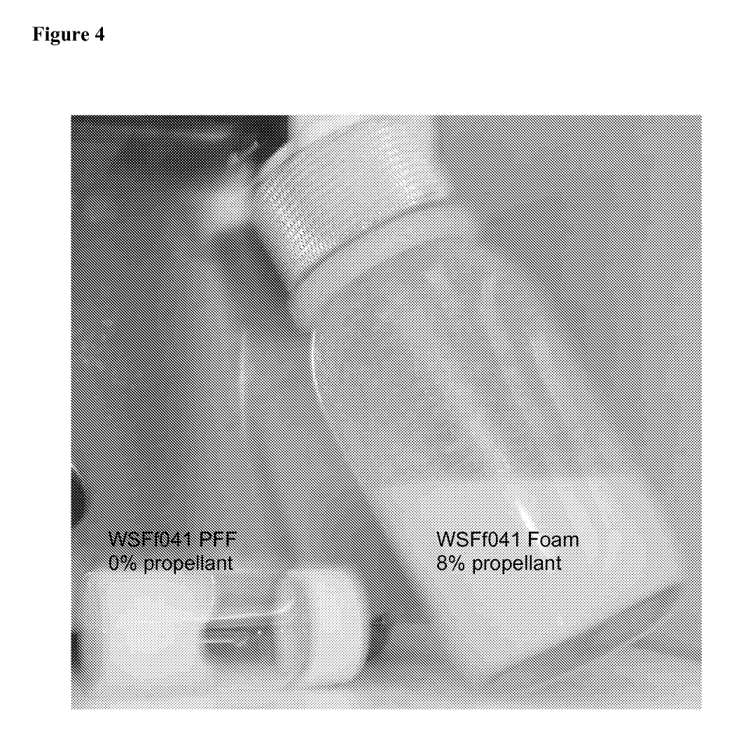

In FIG. 4, formulation f-041 is inverted at an angle after addition of the propellant. The formulation with propellant is presented in a special pressurized glass container. It will be noted that prior to propellant addition the formulation is solid and its inversion to the horizontal does not change its form so that its upper surface is vertical whilst unexpectedly after propellant addition the formulation is liquid and following inversion to an anglel has flowed so that its upper suface is horizontal.

Thus the “liquification” occurs following adding the propellant, which in turn will affect the viscosity substantially or radically. Thus in one or more embodiments the silicone emulsions are liquefied or further liquefied by the propellant.

### Section 2

### Propylene Glycol (“PG”) Platform

### Example 5

### Propylene Glycol Formulations with Hydroxypropyl Cellulose

### Ex 5 Part A Formulations of Silicones in PG vehicle and Hydroxypropyl Cellulose

| | | f-063 | f-064 | f-065 |
|---|---|---|---|---|
| Ingredients | Function | W/W % | W/W % | W/W % |
| Propylene glycol | Solvent | 84 | 84 | 84 |
| Methyl glucose | Emulsifier | 2 | 2 | 2 |
| sesqui stearate | | | | |
| Polydimethylsiloxane | Silicone | 6 | — | — |
| Polymer | | | | |
| Dow Corning(R) | Silicone | — | — | 6 |
| 345 Fluid | | | | |
| Cetyl dimethicone | Silicone | — | 6 | — |
| Hydroxypropyl cellulose | Polymer | 2 | 2 | 2 |
| Steareth 2 | Emulsifier | 2 | 2 | 2 |
| Stearyl alcohol | Foam | 4 | 4 | 4 |
| | Adjuvant | | | |
| Total | | 100 | 100 | 100 |
| Propellant*** | 8 | 8 | 8 | |

Results

| | Time point | | | | | |
|---|---|---|---|---|---|---|
| Parameter | ZT | FTC | ZT | FTC | ZT | FTC |
| Foam quality | Good | Good | Good | Good | Fairly | Good |
| | | | | | good | |
| Odor** | N.O. | V.F.O | N.O. | V.F.O | N.O. | N.O |
| Color | White | White | White | White | White | White |
| Hardness (g) | 25.08 | 14.51 | 101.4 | 69.6 | N.M | 11.69 |
| Density (g/mL) | 0.095 | 0.102 | 0.075 | 0.068 | N.M | 0.103 |
| Collapse (sec) | 130 | N.M. | >300 | N.M. | N.M | N.M. |
| Centrifuge 3k (% of creaming) | 90% | N.M. | 25% | N.M. | 40% | N.M. |
| Centrifuge 10k (% of creaming) | 40 | | 25 | | 25 | |
| Viscosity 10 RPM (cP) | 4772 | N.M. | 4761 | N.M. | 2848 | N.M. |
| Viscosity 1 RPM (cP) | N.M. | N.M. | 8639 | N.M. | N.M. | N.M. |
| Shakability* | 1 | 1 | 1 | 1 | 1 | 1 |

*2: good, 1: moderate, 0: poor
**N.O.—No Odor; V.F.O.—very faint odor
***Propellant is mix of propane, butane and isobutane

Three types of silicones were formulated. Cetyl dimethicone showed good quality and foam parameters. Polydimethylsiloxane Polymer shows reduced collapse time but good foam and Dow Corning® 345 Fluid foam quality is only fairly good and should be used in lower concentrations.

### Ex 5 Part B. Microscopic Description of Formulations (63-65) of Silicones in PG and Hydroxypropyl Cellulose

| | | | Range | |
|---|---|---|---|---|
| | | | of | |
| | Emulsion | Average of | droplet | Liquid |
| | (y/n) | droplet size | size | crystals |
| f-063 | Y | 4μ | 2-15μ | Y |
| f-064 | Y | 4μ | 2-15μ | Y |
| f-065 | Y | 4μ | 2-15μ | Y |

### Ex 5 Part C. Microscopic Picture of Formulation-f-063 of Silicones in PG and Hydroxypropyl Cellulose

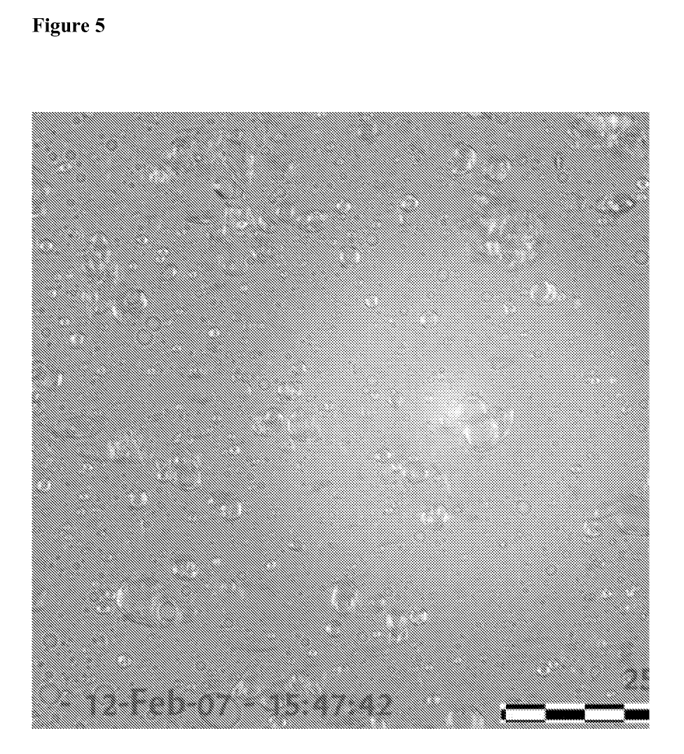

FIG. 5 illustrates a propylene glycol composition with hydroxypropyl cellulose and silicone.

### Ex 5 Part D. Silicones in Propylene Glycol Vehicle with Hydroxypropyl Cellulose

| | | f-002 | f-006 | f-008 |
|---|---|---|---|---|
| Ingredients | Function | W/W % | W/W % | W/W % |
| Propylene glycol | Solvent | 86.00 | 84.00 | 86.00 |
| Methyl glucose | Emulsifier | — | 2.00 | 2.00 |
| sesqui stearate | | | | |
| Polydimethylsiloxane | Silicone | 5.00 | 5.00 | 5.00 |
| Polymer | | | | |
| Bis-PEG-18 Methyl | Stabilizer | 1.00 | 1.00 | — |
| Ether Dimethyl Silane | | | | |
| Hydroxypropyl | Polymer | 1.00 | 2.00 | 2.00 |
| cellulose | | | | |
| Steareth 2 | Emulsifier | 1.00 | 2.00 | 1.00 |
| Cetearyl alcohol | Emulsifier | 2.00 | — | — |
| (and)cetearyl | | | | |
| glucoside | | | | |
| Stearyl alcohol | Foam | 4.00 | 4.00 | 4.00 |
| | Adjuvant | | | |
| Total | | 100 | 100 | 100 |
| Propellant*** | | 8 | 8 | 8 |

Results

Time point

| Parameter | ZT | ZT | FTC | ZT |
|---|---|---|---|---|
| Foam quality | Good | Good | Good | Good |
| Odor** | N.O. | N.O. | N.O. | N.O. |
| Color | White | White | White | White |
| Hardness (g) | N.M. | N.M. | 15.4 | N.M. |
| Density (g/mL) | N.M. | 0.095 | 0.1 | N.M. |
| Collapse (sec) | N.M. | 260 | 200 | N.M. |
| Shakability* | 2 | 1 | 1 | 1 |

*2: good, 1: moderate, 0: poor;
**N.O.—No Odor; V.F.O.—very faint odor
***Propellant is mix of propane, butane and isobutane

### Example 6

### Propylene Glycol Formulations with Aluminum Starch Octenylsuccinate (ASOS)

### Ex 6 Part A. Silicones in Propylene Glycol Vehicle and ASOS

| | | f-066 | f-067 | f-068 |
|---|---|---|---|---|
| Ingredients | Function | W/W % | W/W % | W/W % |
| Propylene glycol | Solvent | 72.00 | 72.00 | 72.00 |
| Methyl glucose | Emulsifier | 2.00 | 2.00 | 2.00 |
| sesqui stearate | | | | |
| Aluminum starch | Polymer | 4.00 | 4.00 | 4.00 |
| octenylsuccinate | | | | |
| Stearic acid | Foam | 9.00 | 9.00 | 9.00 |
| | Adjuvant | | | |
| Polydimethylsiloxane | Silicon | — | 5 | — |
| Polymer | | | | |
| Dow Corning(R) 345 | Silicon | 5 | — | — |
| Fluid | | | | |
| Cetyl dimethicone | Silicon | — | — | 5 |
| Steareth 2 | Emulsifier | 5.00 | 5.00 | 5.00 |
| Stearyl alcohol | Foam | 3.00 | 3.00 | 3.00 |
| | Adjuvant | | | |
| Total | | 100.00 | 100.00 | 100.00 |
| Propellant*** | | 8 | 8 | 8 |

***Propellant is mix of propane, butane and isobutane

### Ex 6 Part B Results of Silicones in Propylene Glycol and ASOS

| | Formulation number | | | | | |
|---|---|---|---|---|---|---|
| | f-066 | | f-067 | | f-068 | |
| | Time point | | | | | |
| Parameter | ZT | FTC | ZT | FTC | ZT | FTC |
| Foam quality | Good | Good | Good | Good | Good | Good |
| Odor** | V.F.O. | N.O. | N.O. | N.O. | N.O. | N.O. |
| Color | white | White | White | White | White | White |
| Hardness (g) | N.M. | N.M. | 13.34 | N.M. | 96.32 | 24.56 |
| Density (g/mL) | 0.295 | 0.393 | 0.138 | 0.140 | 0.073 | 0.060 |
| Collapse (sec) | 140.0 | N.M. | 70.00 | N.M. | >300 | N.M. |
| ~Viscosity 10 RPM | 48364 | N.M. | >52700 | N.M. | N.M. | N.M. |
| (cP) | | | | | | |
| Viscosity 1 RPM | 153087 | N.M. | 294017 | N.M. | 494934 | N.M. |
| (cP) | | | | | | |
| Shakability* | 1 | 2 | 2 | 2 | 2 | 2 |

| | | | |
|---|---|---|---|
| Centrifuge | 20% of creaming | 40% of creaming | 60% of creaming |
| 3k | 15% of sediment | 20% of sediment | 15% of sediment |
| Centrifuge | 20% of creaming | 40% of creaming | 50% creaming |
| 10k | 15% of sediment | 20% of sediment | 15% of sediment |

*2: good, 1: moderate, 0: poor
**N.O.—No Odor; V.F.O.—very faint odor

Aluminum starch octenylsuccinate is a Stabilizing polymer which also exerts improved skin feeling. ASOS may contribute to the sedimentation.

Three types of silicones were tested: Cetyl dimethicone had the higher viscosity and collapse time.

### Ex 6 Part C. Volatile and Non Volatile Silicone Combination in Propylene Glycol with ASOS

| | | f-045 | f-046 | f-047 |
|---|---|---|---|---|
| Ingredients | Function | W/W % | W/W % | W/W % |
| Propylene glycol | Solvent | 80.00 | 72.50 | 71.00 |
| Methyl glucose | Emulsifier | — | 2.00 | 2.00 |
| sesqui stearate | | | | |
| Alluminium starch | Polymer | 3.00 | 4.00 | 3.50 |
| octyl succinate | | | | |
| Stearic acid | Foam | 9.00 | 9.00 | 9.00 |
| | Adjuvant | | | |
| Polydimethylsiloxane | Silicon | 3.00 | 3.00 | 3.00 |
| Polymer | | | | |
| Dow Corning(R) | Silicon | 1.50 | 1.50 | 1.50 |
| 345 Fluid | | | | |
| Steareth 2 | Emulsifier | 3.50 | 5.00 | 5.00 |
| Myristyl alcohol | Emulsifier | — | — | 2.00 |
| Stearyl alcohol | Foam | — | 3.00 | 3.00 |
| | Adjuvant | | | |
| Total | | 100.00 | 100.00 | 100.00 |
| Propellant*** | | 8 | 8 | 8 |

Results

Time point

| Parameter | ZT | ZT | FTC | ZT |
|---|---|---|---|---|
| Foam quality | Fairy Good | Good | Good | Good |
| Odor** | N.O. | N.O. | N.O. | N.O. |
| Color | White | White | white | White |
| Hardness (g) | 6.74 | 17.11 | N.M | 31.52 |
| Density (g/mL) | 0.108 | 0.114 | 0.13 | 0.127 |
| Collapse (sec) | 40 | 120 | >300 | n/a |
| Shakability* | 2 | 1 | 2 | 2 |

| | f-045 | f-046 | f-047 |
|---|---|---|---|
| Centrifuge 3k | 30% of creaming | 25% of creaming | 42% of creaming |
| | 10% of sediment | 15% of sediment | 5% of sediment |
| Centrifuge 10k | 30% of creaming | 30% of creaming | 42% of creaming |
| | 10% of sediment | 20% of sediment | 5% of sediment |

*2: good, 1: moderate, 0: poor
**N.O.—No Odor; V.F.O.—very faint odor
***Propellant is mix of propane, butane and isobutane

Foam quality of formulation f-045 (fairly good) was improved by adding more stabilizing emulsifiers like methyl glucose sesqui stearate and stearyl alcohol (f-046).

### Example 7

### Silicones in Propylene Glycol with Carbopol

### Ex 7 Part A Formulations of Silicones in Propylene Glycol with Carbopol

| | | f052 | f053 |
|---|---|---|---|
| Ingredients | Function | W/W | W/W |
| Propylene glycol | Solvent | 83.50 | 81.50 |
| Carbopol 934P | Polymer | 1.00 | 1.00 |
| Stearic acid | Foam Adjuvant | 4.00 | 4.00 |
| Polydimethylsiloxane Polymer | Silicon | 3.00 | 3.00 |
| Dow Corning(R) 345 Fluid | Silicon | 1.50 | 1.50 |
| Steareth 2 | Emulsifier | 5.00 | 7.00 |
| Stearyl alcohol | Foam Adjuvant | 2.00 | 2.00 |
| Total | | 100 | 100 |
| Propellant*** | | 8 | 8 |

Results

| | Time point | | |
|---|---|---|---|
| Parameter | ZT | FTC | ZT |
| Foam quality | Good | Good | Good |
| Odor** | No | No | No Odor |
| | Odor | Odor | |
| Color | White | White | White |
| Hardness (g) | 32.82 | 47.69 | 37.14 |
| Density (g/mL) | 0.113 | 0.15 | 0.114 |
| Collapse (sec) | 100 | 150.00 | 63 |
| Centrifuge 3k (% of creaming) | stable | N.M | 10 |
| Centrifuge 10k (% of creaming) | stable | stable | |
| Shakability* | 2 | 2 | 2 |

*2: good, 1: moderate, 0: poor
**N.O.—No Odor; V.F.O.—very faint odor
***Propellant is mix of propane, butane and isobutene

The presence of carbopol appears to facilitate stability on challenge with centrifugation.

### Ex 7 Part B. Formulations of Silicones in Propylene Glycol with Carbopol

| | | f055 | f056 |
|---|---|---|---|
| Ingredients | Function | W/W | W/W |
| Propylene glycol | Solvent | 80.50 | 82.50 |
| Carbopol 934 P | Polymer | 1.00 | 1.00 |
| Stearic acid | Foam | 3.00 | 3.00 |
| | Adjuvant | | |
| Polydimethylsiloxane | Silicon | 3.00 | 3.00 |
| Polymer | | | |
| Dow Corning(R) 345 Fluid | Silicon | 1.50 | 1.50 |
| Steareth 2 | Emulsifier | 7.00 | 6.00 |
| Stearyl alcohol | Foam | 4.00 | 3.00 |
| | Adjuvant | | |
| Total | | 100.00 | 100.00 |
| Propellant*** | | 8 | 8 |

Results

| | | |
|---|---|---|
| Foam quality | Good | Good |
| Odor** | No | No |
| | Odor | Odor |
| Color | White | White |
| Density (g/mL) | 0.112 | 0.119 |
| Collapse (sec) | 65 | 93 |
| Centrifuge 3k (% of creaming) | N.M | stable |
| Centrifuge 10k (% of creaming) | N.M | 91 |
| Shakability* | Good | Good |

*2: good, 1: moderate, 0: poor
**N.O.—No Odor; V.F.O.—very faint odor
***Propellant is mix of propane, butane and isobutane

### Example 8

### Usability

Six healthy subjects were introduced to three kinds of silicone foam and one placebo foam (without silicone but otherwise with the same ingredients as one of the silicone foam examples).

The scoring was as follows:
1—No satisfaction
2—Little satisfaction
3—Satisfied
4—Wonderful

### Ex 8 Part A. Formulation F-024 with Volatile and Non Volatile Silicone and Polymer

| | | F-024 |
|---|---|---|
| Ingredients | Function | W/W % |
| PEG-200 | Solvent | 40 |
| PEG-400 | Solvent | 40 |
| Aluminum starch | Polymer | 3 |
| octenylsuccinate | | |
| Stearic acid | Co-emulsifying | 9 |
| Polydimethylsiloxane | Silicone | 3 |
| Polymer | | |
| Dow Corning(R) 345 | Silicone | 1.5 |
| Fluid | | |
| Steareth 2 | Emulsifier | 3.5 |
| Total | | 100 |
| Propellant | | 8 |

Total score is: 180

### Ex 8 Part B. Formulation F-024P without Silicone

(same formulation as previous F-024 but without silicone)

| | | F-024 |
|---|---|---|
| Ingredients | Function | W/W % |
| PEG-200 | Solvent | 44.5 |
| PEG-400 | Solvent | 40 |
| Aluminum starch | Polymer | 3 |
| octenylsuccinate | | |
| Stearic acid | Foam Adjuvant | 9 |
| Steareth 2 | Emulsifier | 3.5 |
| Total | | 100 |
| Propellant | | 8 |

Total score is: 178

### Ex 8 Part C. Formulation F-080 with Cetyl Dimethicone and No Polymer

The formulation is:

| | | f-080 |
|---|---|---|
| Ingredients | Function | W/W % |
| PEG-200 | Solvent | 42.00 |
| PEG-400 | Solvent | 42.00 |
| Trolamine | Stabilizing | 2 |
| Stearic acid | complex | 4 |
| Cetyl dimethicone | Silicone | 5 |
| Steareth 2 | Emulsifier | 5 |
| Total | | 100 |
| Propellant | | 8 |

Total score is: 184

### Ex 8 Part D. Volatile silicone and No Polymer F-078 Formulation

The formulation is:

| | | f-078 |
|---|---|---|
| Ingredients | Function | W/W % |
| PEG-200 | Solvent | 42.00 |
| PEG-400 | Solvent | 42.00 |
| Trolamine | Stabilizing | 2 |
| Stearic acid | complex | 4 |
| Dow Corning(R) 345 | Silicone | 5 |
| Fluid | | |
| Steareth 2 | Emulsifier | 5 |
| Total | | 100 |
| Propellant | | 8 |

Total score is: 171

### Ex 8 Part E. Summary Table of Mean Scoring of the Four Foams

Person ID

| Parameter | f-024P | f-024 | F-080 | F-078 |
|---|---|---|---|---|
| Dryness | 11 | 23 | 16 | 23 |
| Appearance | 17 | 16 | 19 | 19 |
| Smell | 24 | 24 | 23 | 22 |
| Fluidity | 24 | 24 | 24 | 23 |
| Ease of application | 21 | 22 | 23 | 21 |
| Absorbency | 20 | 17 | 19 | 15 |
| Shiny look | 18 | 15 | 17 | 12 |
| Stick | 23 | 18 | 22 | 19 |
| Comfort | 20 | 21 | 21 | 17 |

The placebo form is considered dry and the presense of silicone appears to reduce or alleviate the dry feeling of the waterless composition with two of the three silicone foams tested having an almost maximum score indicating the absence of a dry feeling.

### Example 9

### Reversibilty of Separation

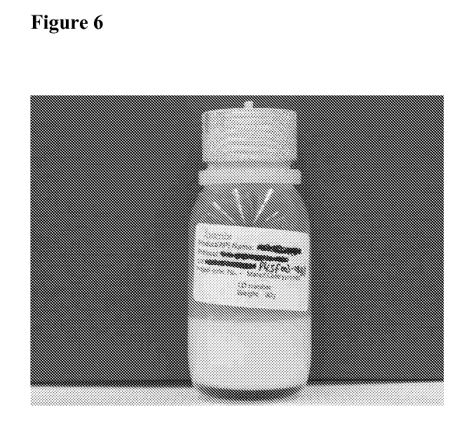

In FIG. 6, it can be seen that the silicone emulsion formulation f003 is homogenous 30 minutes after moderate shaking and remains so for a significant period thereafter. Thus, it can be appreciated that even though separation occurs in some cases when pentane is used to as a control to represent propellant, when submitting the formulation to centrifugation in order to simulate accelerated aging, the emulsion formulation with actual propellant easily reconstitutes on shaking. Thus, the separation is reversible and upon shaking with the actual propellant the emulsion readily reconstitutes and is homogenous. It therefore follows that in all the Examples herein where pentane was used and separation was observed then any separation that occurs with actual propellant should be readily reversible on shaking to form a homogenous emulsion.

1. Formulation Composition PWSF003-080311

| Material chemical | |
|---|---|
| name | % W/W |
| Propylene glycol | 68.00 |
| Steareth 2 | 4.00 |
| Stearyl alcohol | 3.00 |
| Dimethicone | 25.00 |
| Total | 100.00 |
| propellant (AP-70) | 8.00 |

2. Manufacturing Procedure:

### Step 1: Preparation of Waterless Phase

Heat Propylene glycol to 60 C and Add Steareth 2 and Stearyl alcohol while mixing to dissolution. Cool to 45-50 and Add Dimethicone. Cool to RT while mixing.

### Section 3

### (A) Examination of Influence of HLB and Surfactant Type (Solid vs Liquid) in Waterless PEG and PG Formulations with Minimal Ingredients

### Example 10

### Formulations with Solvent, Emulsifier and Silicone (without Polymer)

A-1) Solid Emulsifier with Low HLB-Formulations

| Ingredients | 166 | 171 | 176 |
|---|---|---|---|
| PEG 400 | 95.00 | 95.00 | 95.00 |
| Steareth 2 (4.9 HLB) | 3.00 | 3.00 | 3.00 |
| Dimethicone 200 5 cst/ | — | — | 2.00 |
| Cyclomethicone | — | 2.00 | — |
| Cetyl dimethicone | 2.00 | — | — |
| Total | 100.00 | 100.00 | 100.00 |
| Propellant (propane + | 8.00 | 8.00 | 8.00 |
| butane + isobutene) | | | |
| Foam Quality | E | E | E |
| Shakability | G | G | G |
| Emulsion | Stable | stable | stable |
| Centrifugation 3k | separation | separation | separation |

A-2) Solid Emulsifier with High HLB-Formulations

| Ingredients | 167 | 172 | 177 |
|---|---|---|---|
| PEG 400 | 95.00 | 95.00 | 95.00 |
| Steareth 21 (15.5 | 3.00 | 3.00 | 3.00 |
| HLB) | | | |
| Dimethicone 200 5 cst/ | — | — | 2.00 |
| Cyclomethicone | — | 2.00 | — |
| Cetyl dimethicone | 2.00 | — | — |
| Total | 100.00 | 100.00 | 100.00 |
| Propellant (propane + | 8.00 | 8.00 | 8.00 |
| butane + isobutene) | | | |
| Foam Quality | G | FG | FG |
| Shakability | Hardly | Hardly | Hardly |
| Emulsion | separation | stable | separation |
| Centrifugation 3k | separation | separation | separation |

Comment: In general terms low HLB formulations are preferred with solid emulsifiers. Without being bound by any theory, it may be the case here that the higher miscibility of low HLB steareth with the silicones accounts at least in part for the improved quality of the foam in A1 formulations compared with A2 formulations using high HLB steareth 21.

B-1) Liquid Emulsifier with Low HLB-Formulations

| Ingredients | 168 | 173 | 178 |
|---|---|---|---|
| PEG 400 | 95.00 | 95.00 | 95.00 |
| Sorbitan monooleate | 3.00 | 3.00 | 3.00 |
| (4.3 HLB) | | | |
| Dimethicone 200 5 cst/ | — | — | 2.00 |
| Cyclomethicone | — | 2.00 | — |
| Cetyl dimethicone | 2.00 | — | — |
| Total | 100.00 | 100.00 | 100.00 |
| Propellant (propane + | 8.00 | 8.00 | 8.00 |
| butane + isobutene) | | | |
| Foam Quality | VP | VP | VP |
| Shakability | Good | Good | Good |
| Emulsion | separation | separation | stable |
| Centrifugation 3k | separation | separation | separation |

B-2) Liquid Emulsifier with High ULB-Formulations

| Ingredients | 169 | 174 | 179 |
|---|---|---|---|
| PEG 400 | 95.00 | 95.00 | 95.00 |
| Polysorbate 80 | 3.00 | 3.00 | 3.00 |
| (15.0 HLB) | | | |
| Dimethicone 200 5 cst/ | — | — | 2.00 |
| Cyclomethicone | — | 2.00 | — |
| Cetyl dimethicone | 2.00 | — | — |
| Total | 100.00 | 100.00 | 100.00 |
| Propellant (propane + | 8.00 | 8.00 | 8.00 |
| butane + isobutene) | | | |
| Foam Quality | VP | VP | VP |
| Shakability | Good | Good | Good |
| Emulsion | stable | separation | stable |
| Centrifugation 3k | separation | separation | separation |

Comment: These formulations with liquid surfactants do not produce foams of any quality and it appears that solid emulsifiers should be used in the absence of a polymeric agent or other solid or gelling substance that can produce sufficient body or matrix in the foamable formulation that it can result in the expulsion of a breakable foam upon addition of propellant in a pressurized canister and release therefrom on actuation of the valve.

### Example 11

### Formulations with Polymer, Solvent, Emulsifier and Silicone

### (A) Where the Polymer is Kucel EF (Solid)

A-1) Solid Emulsifier with Low ULB and Klucel EF-Formulations

| Ingredients | 121 | 126 | 131 |
|---|---|---|---|
| PEG 400 | 92.00 | 92.00 | 92.00 |
| Steareth 2 (4.9 HLB) | 3.00 | 3.00 | 3.00 |
| Klucel EF | 3.00 | 3.00 | 3.00 |
| Dimethicone 200 5 cst/ | — | — | 2.00 |
| Cyclomethicone | — | 2.00 | — |
| Cetyl dimethicone | 2.00 | — | — |
| Total | 100.00 | 100.00 | 100.00 |
| Propellant (propane + | 8.00 | 8.00 | 8.00 |
| butane + isobutene) | | | |
| Foam Quality | E | E | E |
| Shakability | G | G | G |
| Emulsion | Stable | Stable | Stable |
| Centrifugation 3k | separation | separation | separation |

A-2) Solid Emulsifier with High HLB and Klucel EF-Formulations

| Ingredients | 122 | 127 | 132 |
|---|---|---|---|
| PEG 400 | 92.00 | 92.00 | 92.00 |
| Steareth 21 (15.5 HLB) | 3.00 | 3.00 | 3.00 |
| Klucel EF | 3.00 | 3.00 | 3.00 |
| Dimethicone 200 5 cst/ | — | — | 2.00 |
| Cyclomethicone | — | 2.00 | — |
| Cetyl dimethicone | 2.00 | — | — |
| Total | 100.00 | 100.00 | 100.00 |
| Propellant (propane + | 8.00 | 8.00 | 8.00 |
| butane + isobutene) | | | |
| Foam Quality | FG | FG | FG |
| Shakability | Hardly | No shaking | Hardly |
| Emulsion | Stable | Stable | Stable |
| Centrifugation 3k | separation | separation | separation |

Comment: In general terms low HLB formulations are preferred with solid emulsifiers in the presence of Klucel when compared to high HLB. Also the solid emulsifier is preferred to the liquid emulsifier.

A-3) Liquid Emulsifier with Low HLB and Klucel EF-Formulations

| Ingredients | 123 | 128 | 133 |
|---|---|---|---|
| PEG 400 | 92.00 | 92.00 | 92.00 |
| Sorbitan monooleate | 3.00 | 3.00 | 3.00 |
| (4.3 HLB) | | | |
| Klucel EF | 3.00 | 3.00 | 3.00 |
| Dimethicone 200 5 cst/ | — | — | 2.00 |
| Cyclomethicone | — | 2.00 | — |
| Cetyl dimethicone | 2.00 | — | — |
| Total | 100.00 | 100.00 | 100.00 |
| Propellant (propane + | 8.00 | 8.00 | 8.00 |
| butane + isobutene) | | | |
| Foam Quality | FG | FG | FG |
| Shakability | Good | Good | Good |
| Emulsion | Stable | Stable | Stable |
| Centrifugation 3k | separation | separation | separation |

A-4) Liquid Emulsifier with High HLB and Klucel EF-Formulations

| Ingredients | 124 | 129 | 134 |
|---|---|---|---|
| PEG 400 | 92.00 | 92.00 | 92.00 |
| Polysorbate 80 | 3.00 | 3.00 | 3.00 |
| (15.0 HLB) | | | |
| Klucel EF | 3.00 | 3.00 | 3.00 |
| Dimethicone 200 5 cst/ | — | — | 2.00 |
| Cyclomethicone | — | 2.00 | — |
| Cetyl di methicon | 2.00 | — | — |
| Total | 100.00 | 100.00 | 100.00 |
| Propellant (propane + | 8.00 | 8.00 | 8.00 |
| butane + isobutene) | | | |
| Foam Quality | FG | FG | FG |
| Shakability | Good | Good | Good |
| Emulsion | Stable | Stable | Stable |
| Centrifugation 3k | separation | separation | separation |

Comment: These formulations with liquid surfactants and klucel produced foams of fairly good quality. Klucel has a significant stabilizing effect. HLB does not appear to be a significant factor when klucel is the polymeric agent.

### (B) Where the Polymer is ASOS

B-1) Solid Emulsifier with Low HLB and ASOS-Formulations

| Ingredients | 152 | 157 | 162 |
|---|---|---|---|
| PEG 400 | 92.04 | 92.04 | 92.04 |
| Steareth 2 (4.9 HLB) | 2.8 | 2.8 | 2.8 |
| ASOS | 2.8 | 2.8 | 2.8 |
| Dimethicone 200 5 cst/ | — | — | 2.15 |
| Cyclomethicone | — | 2.15 | — |
| Cetyl dimethicone | 2.15 | — | — |
| Total | 100.00 | 100.00 | 100.00 |
| Propellant (propane + | 8.00 | 8.00 | 8.00 |
| butane + isobutene) | | | |
| Foam Quality | FG | FG | G |
| Shakability | Good | Good | Good |
| Emulsion | Separation | Separation | Stable |
| Centrifugation 3k | separation | separation | separation |

Comment: ASOS does not appear to have a stabilizing effect like Klucel and the similar formulation in Example 9 without ASOS resulted in a foam of better quality.

B-2) Liquid Emulsifier with High HLB and ASOS-Formulations

| Ingredients | 154 | 159 | 164 |
|---|---|---|---|
| PEG 400 | 92.00 | 92.00 | 92.00 |
| Polysorbate 80 | 3.00 | 3.00 | 3.00 |
| (15.0 HLB) | | | |
| ASOS | 3.00 | 3.00 | 3.00 |
| Dimethicone 200 5 cst/ | — | — | 2.00 |
| Cyclomethicone | — | 2.00 | — |
| Cetyl di methicon | 2.00 | — | — |
| Total | 100.00 | 100.00 | 100.00 |
| Propellant (propane + | 8.00 | 8.00 | 8.00 |
| butane + isobutene) | | | |
| Foam Quality | VP | VP | VP |
| Shakability | Good | Good | Good |
| Emulsion | Separation | Separation | separation |
| Centrifugation 3k | separation | separation | separation |

Comment: These formulations with liquid surfactants do not produce foams of any quality and it appears that solid emulsifiers should be used in the presense of ASOS.

### (C) Where the Polymer is Carbopol

C-1) Solid Emulsifier with High HLB and Carbopol-Formulations

| Ingredients | 137 | 142 | 147 |
|---|---|---|---|
| PEG 400 | 92.90 | 92.90 | 92.90 |
| Steareth 21 (15.5 HLB) | 2.80 | 2.80 | 2.80 |
| Carbopol | 1.93 | 1.93 | 1.93 |
| Dimethicone 200 5 cst/ | — | — | 2.15 |
| Cyclomethicone | — | 2.15 | — |
| Cetyl dimethicone | 2.15 | — | — |
| Total | 100.00 | 100.00 | 100.00 |
| Propellant (propane + butane + | 8.00 | 8.00 | 8.00 |
| isobutene) | | | |
| Foam Quality | G-FG | G | FG |
| Shakability | No shaking | No shaking | Moderate |
| Emulsion | Separation | Separation | Stable |
| Centrifugation 3k | Stable | Stable | Stable |

C-2) Liquid Emulsifier with High HLB and Carbopol-Formulations

| Ingredients | 139 | 144 | 149 |
|---|---|---|---|
| PEG 400 | 92.00 | 92.00 | 92.00 |
| Polysorbate 80 ((15.0 HLB) | 3.00 | 3.00 | 3.00 |
| Carbopol | 3.00 | 3.00 | 3.00 |
| Dimethicone 200 5 cst/ | — | — | 2.00 |
| Cyclomethicone | — | 2.00 | — |
| Cetyl dimethicone | 2.00 | — | — |
| Total | 100.00 | 100.00 | 100.00 |
| Propellant (propane + butane + | 8.00 | 8.00 | 8.00 |
| isobutene) | | | |
| Foam Quality | VP | VP | VP |
| Shakability | Good | Good | Good |
| Emulsion | Separation | Separation | separation |

Comment: These formulations with liquid surfactants do not produce foams of any quality and it appears that solid emulsifiers should be used in the presence of carbopol a polymeric agent.

### Section 3

### (B) Comparative Examination of the Results in Example 10 and in Example 11 on the Influence of HLB and Surfactant Type (Solid vs. Liquid) in Waterless PEG Formulations with Minimal Ingredients and with/without Gelling Agents

| | | | | | | | | Comments |
|---|---|---|---|---|---|---|---|---|
| | solid | | liquid | | | | | More Preferred > Less |
| | emulsifier | | emulsifier | | | | | preferred |
| | high | low | high | low | klucel EF | carbopol | ASOS | Same/Similar = Same/Similar |
| | HLB | HLB | HLB | HLB | (gelling | (gelling | (gelling | High refers to high HLB; |
| | 15.5 | 4.9 | 15 | 4.3 | agent) | agent) | agent) | Low refers to Low HLB |
| 1 | ✓ | ✓ | | | | | | FQ: Low > High |
| | | | | | | | | ES: Low > High |
| 2 | | | ✓ | ✓ | | | | FQ: High = Low |
| | | | | | | | | ES: High = Low |
| 3 | ✓ | | ✓ | | | | | FQ: High solid > high liquid |
| | | | | | | | | ES: High solid = high liquid |
| 4 | | ✓ | | ✓ | | | | FQ: low solid > low liquid |
| | | | | | | | | ES: low solid > low liquid |
| 5 | ✓ | ✓ | | | ✓ | | | FQ: Low > High |
| | | | | | | | | ES: Low = High |
| 6 | | | ✓ | ✓ | ✓ | | | FQ: High = Low |
| | | | | | | | | ES: High = Low |
| 7 | ✓ | | ✓ | | ✓ | | | FQ: High solid = high liquid |
| | | | | | | | | ES: High solid = high liquid |
| 8 | | ✓ | | ✓ | ✓ | | | FQ: low solid > low liquid |
| | | | | | | | | ES: low solid = low liquid |
| 9 | ✓ | | ✓ | | | ✓ | | FQ: High solid > high liquid |
| | | | | | | | | ES: High solid = high liquid |
| 10 | ✓ | | ✓ | | | | ✓ | FQ: High solid > high liquid |
| | | | | | | | | ES: High solid = high liquid |

FQ; Foam quality; ES: Emulsion stability

For example, looking at item 8 it can be seen that a solid surfactant with a low HLB resulted in foam of better quality than a corresponding formulation with a low HLB liquid surfactant. In general terms the combined quality and stability properties of the foam formulations comprising surfactant and klucel seen in items 6, 7, and 8 respectively were overall about the same or better than those seen in 2, 3, and 4 respectively. The additional presence of klucel primarily resulted in a significant improvement in and helped to stabilize the foam where the surfactant was liquid rather than solid. In other words the results for liquid surfactants with klucel seen in item 6 were better than in item 2; in item 7 better than in item, and in item 8 better than in item 4. However, when comparing formulations with and without klucel in items 4 and 1 respectively, the presence of klucel was not seen to be significant.

Note a) Items 1-4 are with surfactant alone; b) Items 5-8 are with surfactant plus klucel (a solid polymeric agent); c) With respect to the formulations with carbopol and with ASOS there are only partial comparative experiments; and d) although the results have been presented in terms of solid vs liquid and high vs low HLB it should not be discounted that the different chemical natures of the solid and liquid surfactants used may impact on the results such that other surfactant and polymer combinations may achieve different results. Moreover as the formulations become more sophisticated and complex the presence of other significant components like foam adjuvants and active agents must be taken into account.

### Section 4

### Comparison of Formulations with High (20%-25%) and Low (2%-5%) Levels of Silicones in PEG and in PG Vehicles

In the examples below it can be seen that it is possible to incorporate high levels (25%) of non volatile or volatile silicones in a PEG or PG based foamable waterless emulsion composition and produce foam of good quality. It may be—without being bound by any theory that the unique interactions in a waterless emulsion environment are such that silicone, which is known to act as a defoamer in oil/water emulsions does not substantially destroy the foam at least with the solid surfactants used herein or with solid surfactant/polymeric agent combinations described herein.(e.g. steareth surfactant plus klucel polymer). On the other hand we also see herein that lower levels of silicone 2%-5% can apparently destroy foam formation with for example a high HLB liquid emulsifier with or without ASOS or carbopol (See Examples 10 B1; 10 B2 and 11 C2 above)

### Example 12

### PEG Vehicle with Different Silicones (with polymer-carbopol)

### Ex 12A. Formulations

| | WSF147- | PWSF001- | PWSF005- |
|---|---|---|---|
| Ingredients | 080214 | 080214 | 080220 |
| PEG 400 | 93.00 | 69.00 | 68.00 |
| cetyl dimethicone | 2.00 | | |
| Dimethicone | | 20.00 | 25.00 |
| Steareth-2 | | 6.00 | 3.50 |
| Steareth 21 | 3.50 | | |
| Stearyl alcohol | | 4.00 | 3.00 |
| carbopol | 1.50 | 1.00 | 0.50 |
| Total: | 100.00 | 100.00 | 100.00 |
| Propellant is a mixture of | 8.00 | 8.00 | 8.00 |
| propane butane and isobutane | | | |
| (AP-70) | | | |

### Ex 12B. Results

| | WSF147- | PWSF001- | PWSF005- |
|---|---|---|---|
| | 080214 | 080214 | 080220 |
| PFF | | | |
| Cent. 3K (with pentane) | 80% creaming | stable | separation |
| Cent. 10K (with pentane) | 30% creaming | 30% creaming | separation |
| Cent. 3K (no pentane) | stable | stable | stable |
| Cent. 10K (no pentane) | stable | stable | stable |
| viscosity. (1 RPM) | 192598.90 | 772635.10 | |
| | | (0.5 RPM) | |
| viscosity. (10 RPM) | 36200.28 | n/a | |
| Foam | | | |
| Foam quality | G-E | block | G-E |
| Color | W | | W |
| Odor | No | | No |
| Shakability | moderate | | Moderate |
| Density | 0.135 | | |
| Hardness | 21.5 | | |
| Collapse time | 240/F | | |
| BUBBLE SIZE (μm) | 137 | | |
| BUBBLE SIZE | 0 | | |
| (above 500 μm) | | | |

Comments: Using 25% Dimethicone (or other silicone) PEG, carbopol and suitable surfactant, it is possible to achieve a stable emulsion (without pentane), that produces good to excellent foam. The vehicle with low silicone is resistant to centrifugation and does not separate. There is a balance between the objective to obtain a formulation resistant to centrifugation and one that forms a block and cannot be expelled from a canister. By reducing the surfactant and foam adjuvant levels the block is overcome but the formulation becomes susceptible to phase separation on addition of pentane. Nevertheless in the absence of pentane the formulations are stable.

### Example 13

### PEG Vehicle with Different Silicones (without polymer)

### Ex 13A. Formulations

| | WSFF081- | WSFF072- | PWSF002- |
|---|---|---|---|
| Ingredients | 080212 | 080212 | 080217 |
| PEG 400 | 87.50 | 43.50 | 68.00 |
| PEG 200 | | 44.00 | |
| cetyl dimethicone | 5.00 | | |
| Cyclomethicone | | 5.00 | |
| Dimethicone | | | 25.00 |
| Steareth-2 | 7.50 | 4.50 | 4.00 |
| Methyl glucose seasquist | | 3.00 | |
| stearate | | | |
| Stearyl alcohol | | | 3.00 |
| Total: | 100.00 | 100.00 | 100.00 |
| Propellent is a mixture of | 8.00 | 8.00 | 8.00 |
| propane butane and | | | |
| isobutane (AP-70) | | | |

### Ex 13A. Results

| | WSFF081- | WSFF072- | PWSF002- |
|---|---|---|---|
| | 080212 | 080212 | 080217 |
| PFF | | | |
| Cent. 3K (with pentane) | separation | 15% creaming | separation |
| Cent. 10K (with pentane) | separation | 15% creaming | separation |
| Cent. 3K (no pentane) | stable | stable | stable |
| Cent. 10K (no pentane) | stable | stable | stable |
| viscosity. (1 RPM) | 274021.53 | 79822.97 | 226991.56 |
| viscosity. (10 RPM) | n/a | n/a | n/a |
| Foam | | | |
| Foam quality | G-E | G-E | G-E |
| Color | W | W | W |
| Odor | No | No | No |
| Shakability | Good | Good | moderate |
| Density | 0.077 | 0.147 | 0.087 |
| Hardness | 62.56 | 24.06 | 69.3 |
| Collapse time | >300/G | 240/F | >300/G |
| BUBBLE SIZE (μm) | 61 | 87 | 63 |
| BUBBLE SIZE | 0 | 0 | 0 |
| (above 500 μm) | | | |

Comments: All the formulations produce stable emulsions prior to pentane addition. The high level of silicone may contribute to the separation. Where the silicone is volatile it may at low levels—without being bound by any particular theory—perhaps help the pentane to mix in the formulation. All the formulations produced good quality foam and collapse time having small average bubble size.

### Example 13B

### Formulation

| Ingredients | PWSF004-080218 | PWSF006-080221 |
|---|---|---|
| PEG 400 | 68.00 | 63.00 |
| Cyclomethicone | 25.00 | |
| Dimethicone | | 25.00 |
| Steareth-2 | 2.90 | 7.00 |
| Stearyl alcohol | 3.00 | 5.00 |
| Total: | 100.00 | 100.00 |
| Propellent (AP-70) | 8.00 | 8.00 |

| | PWSF004-080218 | PWSF006-080221 |
|---|---|---|
| PFF | | |
| Cent. 3K (with pentane) | separation | 80% creaming |
| Cent. 10K (with pentane) | separation | separation |
| Cent. 3K (no pentane) | stable | stable |
| Cent. 10K (no pentane) | stable | stable |
| viscosity. (1 RPM) | 98219.04 | |
| viscosity. (10 RPM) | 46981.98 | |
| Foam | | |
| Foam quality | G-E | G-E |
| Color | W | W |
| Odor | No | No |
| Shakability | Moderate | Moderate |
| Density | 0.513 | |
| Hardness | 39.81 | |
| Collapse time | 180/F | |
| BUBBLE SIZE (μm) | 182 | |
| BUBBLE SIZE | 0 | |
| (above 500 μm) | | |

Comments: while using 25% Dimethicone (or other silicone), PEG and suitable surfactant, it is possible to achieve stable emulsion (without pentane), that produces good to excellent foam.

### Example 14

### Propylene Glycol Vehicle with Different Silicones (with and without polymer-carbopol)

### Ex 14A. Formulations

| | WSFF052-080213 | PWSF003-080217 |
|---|---|---|
| Propylene glycol | 83.50 | 68.00 |
| Cyclomethicone | 1.50 | |
| Dimethicone | 3.00 | 25.00 |
| Steareth-2 | 5.00 | 4.00 |
| Stearic acid | 4.00 | |
| Stearyl alcohol | 2.00 | 3.00 |
| carbopol | 1.00 | |
| Total: | 100.00 | 100.00 |
| Propellent is a mixture of | 8.00 | 8.00 |
| propane butane and isobutane | | |
| (AP-70) | | |

### Ex 14B. Results

| | WSFF052-080213 | PWSF003-080217 |
|---|---|---|
| PFF | | |
| Cent. 3K (with pentane) | 10% creaming | separation |
| Cent. 10K (with pentane) | 10% creaming | separation |
| Cent. 3K (no pentane) | stable | stable |
| Cent. 10K (no pentane) | stable | stable |
| viscosity. (1 RPM) | 137410.68 | 121893.99 |
| viscosity. (10 RPM) | n/a | 32968.97 |
| Foam | | |
| Foam quality | G-E | G-E |
| Color | W | W |
| Odor | No | No |
| Shakability | Good | Good |
| Density | 0.088 | 0.072 |
| Hardness | 54.59 | 54.54 |
| Collapse time | >300/FG | >300/G |
| BUBBLE SIZE (μm) | 66 | 108 |
| BUBBLE SIZE | 0 | 0 |
| (above 500 μm) | | |

Comments: While using high (e.g. 25% Dimethicone) silicone, propylene glycol suitable surfactant and with/without carbopol, it's possible to achieve stable emulsion (without pentane), that produces good to excellent foam with a good collapse time and small bubble size. When the silicone is low the formulation shows some resistance to creaming in the presence of pentane.

### Section 5

### Prophetic Examples

### Example 15

### Prophetic

a) Foamable Polyols Compositions, Containing a Steroid Drug

The following steroids can be included in carriers, compositions and foams: betamethasone valerate 0.12%, clobetasol propionate 0.05%, betamethasone dipropionate 0.05%, fluocinolone acetonide 0.025%, hydrocortisone acetate 0.5% and hydrocortisone butyrate 0.1%.

b) Foamable Polyols Compositions, Containing a Vitamin and a Steroid Drug

Additionally, one or more of the following vitamins can be included in the carriers, compositions and foams: vitamin C (ascorbic acid) between 0.1 and 5% say, 0.1% 1%, 2% 3%, 4%, or 5%; vitamin C (magnesium ascorbyl phosphate) 3%, retinol 1%, retinoic acid 0.1%, niacinamide 2% and tocopherol 1% and Vitamin K, between 0.1 and 2% say, 0.1% or 1% or 2%.

### Example 16

### Prophetic Foamable Vitamin Compositions with an Additional Therapeutic Agent

Foamable vitamin compositions at either say 1%, 2%, 3%, 4%, or 5%, by weight of composition are made up with an active agent and added to any of the compositions illustrated in Examples 1-14 wherein the percentage amount of one or both solvents is reduced by an approximately equivalent amount by weight in the composition.

### Example 17

### Prophetic Foamable Therapeutic Agent Compositions

More particularly exemplary concentrations of additional therapeutic agents in foamable compositions are set out in Table 1 below. Each active agent is added into, for example, any of the carriers listed in any of Examples 1-14 above in a therapeutically effective concentration and amount. The methodology of addition is well known to those of the art. The composition is adjusted in each case so that it is made up to 100% w/w as appropriate by solvent.

TABLE 1
Exemplary Concentrations of Examples of Active Agents

| Additional | | |
|---|---|---|
| therapeutic | Exemplary | |
| agent | Concentration | Exemplary Use |
| Hydrocortisone | 1% | Steroid responsive inflammation and |
| acetate | | |
| Betamethasone | 0.1% | psoriasis or atopic dermatitis |
| valerate | | |
| Clobetasol | 0.05% | |
| propionate | | |
| Acyclovir | 5% | Viral infection, herpes |
| Ciclopirox | 1% | Fungal infection, seborrhea, |
| | | dandruff, |
| Clindamycin | 2% | Bacterial infection, acne, rosacea, |
| Azelaic acid | 15% | Acne, rosacea, |
| | | pigmentation disorder and various |
| | | dermatoses |
| Metronidazol | 0.25%-2% | Rosacea, bacterial infections and |
| | | parasite infestations |
| Diclofenac | 1% | Osteoarthritis, joint pain |
| Tacrolimus | 0.2% | Atopic dermatitis, eczema and |
| | | inflammation |
| Benzoyl peroxide | 1%-10% | Acne |
| Alpha-hydroxy | 1%-20% | Aging, wrinkles |
| acids | | |
| Salicylic acid | 1%-10% | Acne |
| Hydroquinone | 1%-10% | Pigmentation disorders |
| Caffeine | 1%-10% | Anti Cellulite |
| Coenzyme Q 10 | 0.1%-10% | Aging, pigmentation |
| Clotrimazole | 1% | Fungal infection |
| Lidocaine base | 2% | Local anaesthetic |
| Terbinafine HCL | 1% | Fungal infection |
| Gentamycin | 0.1% | Bacterial skin infections, burns or |
| | | ulcers |
| Dexpanthenol | 5% | Wounds, ulcers, minor skin |
| | | infections |
| Urea | 5-10% | Emollient and keratolytic |
| | | Atopic dermatitis, eczema, |
| | | ichthyosis and hyperkeratotic skin |
| | | disorders |
| Ammonium | 12%-17.5% | Dry scaly conditions of the skin |
| lactate | | including ichthyosis |
| Povidone-iodine | 10% | Antimicrobial - antiseptic |
| Calcitriol | ~0.005% | Psoriasis |
| Calcipotriol | ~0.005% | Psoriasis |

The above examples represent different drug classes and it is to be understood that other drugs belonging to each of the classes represented above may be included and used in the compositions in a safe and effective amount.

### Example 18

### Prophetic Foamable Compositions Comprising Microposnges

A microsponge is added into, for example, any of the carriers listed in any of Examples 1-13 above. The microsponges are loaded with active agents in a therapeutically effective concentration and amount and the microsponges are incorporated into one of the said carriers. The methodology of addition is well known to those of the art. The composition is adjusted in each case so that it is made up to 100% w/w as appropriate by solvent. Care should be taken in selecting and preparing the formulation such that the microsponges are distributed substantially homogenously and so that any aggregation of microsponges is minimized such that they do not block the caniter valve and thereby prevent release of foam.

### Example 18A

### Prophetic Hydrophilic Solvent Foamable Formulation Comprising Microsponges Loaded with Active Agent

| Ingredients | % w/w | % w/w |
|---|---|---|
| Drug Microsponge ® | 10 | 10 |
| Propylene glycol | To 100 | |
| PEG 400 | | To 100 |
| Silicone | 1-25 | 1-25 |
| Steareth-2 | 1-5 | 1-5 |
| carbopol | 0.50 | 0.50 |
| Propellant | 8.00 | 8.00 |

This prophetic formulation can be adapted for a high range of hydrophilic solvent content of upto about 95%.

The amount of microsponges may be varied from about 1% to about 25% of the formulation by increasing or decreasing the amount of the hydrophilic solvent.

Any active agent suitable for loading in microsponges may be use. Non-limiting examples are benzyl peroxide, tretinoin, hydroquinone and the like or any of the active agents described in Examples 14, 15 and 16 above.

In an embodiment the microsponges are loaded with one or more vitamins or with one or more flavonoids or combinations thereof.

The liquefied or gas propellant can be added at a concentration of about 3% to about 35%, for example in a ratio of carrier composition to propellant of at least about 100:3, or about 100:3 to about 100:35.

### SECTION 6

### Compostions with Various Active Agents

### Example 19

### Hydrophilic PEG 200/400 Compositions with a Silicone Combination, ASOS and bmv or mupirocin as Active Agents

| Ingredients | % w/w | % w/w |
|---|---|---|
| PEG 400 | 40.38 | 38.50 |
| PEG 200 | 39.50 | 39.50 |
| DIMETHICONE | 3.00 | 3.00 |
| Cyclomethicone | 1.50 | 1.50 |
| ASOS | 3.00 | 3.00 |
| Stearic acid | 9.00 | 9.00 |
| Steareth-2 | 3.50 | 3.50 |
| Betamethasone 17 valerate | 0.12 | |
| micronized | | |
| Mupirocin | | 2.00 |
| Total | 100.00 | 100.00 |

RESULTS/APPEARANCE

| | | |
|---|---|---|
| QUALITY | Good | Good |
| COLOR | White | White |
| ODOR | No Odor | No Odor |
| SHAKABILITY | Yes | Yes |

Comments: formulations based on a polyethylene glycol mixture, polymeric agent surfactant, and a silicone produced good (G) white (W) No odor (N.O.) and shakable foams. The propellant can be added at a concentration of about 3% to about 25% or more, for example in a ratio of carrier composition to propellant of at least about 100:3 or about 100:3 to about 100:35.

### Example 20

### Hydrophilic PEG 200 Composition with a Volatile Silicone, ASOS and Ascorbic Acid

### Ex 20 Part A Formulation

| | | % |
|---|---|---|
| Ingredient | Function | w/w |
| PEG 200 (Polyethylene Glycol 200) | Solvent | 76.00 |
| Alluminum Starch Octynylsuccinate | Polymer | 4.00 |
| CETEARYL ALCOHOL | Emulsifier | 2.00 |
| (and)CETEARYL GLUCOSIDE | | |
| Cyclomethicone (Dow Corning 345 | Silicone | 2.00 |
| Fluid) | | |
| Stearic Acid | Foam | 4.00 |
| | Adjuvant | |
| Steareth-2 ((Brij 72) | Emulsifier | 2.00 |
| Stearyl Alcohol | Foam | 2.00 |
| | Adjuvant | |
| Vitamin C (Ascorbic acid) | API | 8.00 |
| Control: | | 100.00 |
| Propellant | | 8 |
| mixture of propane, butane and isobutane | | |

### Ex 20 Part B—Results

| | WAS-014 | |
|---|---|---|
| | Time point | |
| Parameter | ZT | FTC* |
| Foam quality | Good | Good |
| Odor** | N.O. | N.O. |
| Color | Off white | Off white |
| Hardness (g) | 100.81 | 93.020 |
| Density (g/mL) | 0.088 | 0.065 |
| Collapse (sec) | >300. | >300. |
| Microscopic Examination | No crystals were | No crystals were |
| (compared to placebo) | determined that were not | determined that |
| | present in placebo | were not present |
| | formulations | in placebo |
| | | formulations |

*FTC - Samples are stored in stressed conditions as follows: −10° C. follows by 40° C. This stress is repeated four times.
**NO = No Odor;

What is claimed is:

1. A waterless carrier, composition or foam formulation comprising:
(a) a silicone;
(b) about 25% to about 98% of a solvent selected from the group consisting of (1) a propylene glycol or derivative and (2) a polyethylene glycol (PEG) or derivative or mixtures thereof;
(c) 0% to about 48% of at least one secondary solvent;
(d) about 0.05% to about 20% of an Accommodating Agent or Complex;
(e) optionally about 0.01% to about 5% by weight of at least one polymeric agent; and
wherein the formulation is a silicone in glycol emulsion; and
wherein the Accomodating Agent or Complex is selected from one or more of the group consisting of
a. at least one surface-active agent at a concentration of about 0.1% to less than about 15% by weight;
b. at least one polymeric agent at a concentration of about 0.1% to about 5% by weight, selected from the group consisting of a bioadhesive agent, a gelling agent, a film forming agent and a phase change agent;
c. at least one foam adjuvant at a concentration of about 0.1% to about 5% by weight selected from the group consisting of a fatty alcohol, a fatty acid and a hydroxyl fatty acid; and
d. at least one Stabilizing agent at a concentration of about 0.1% to about 5% by weight; and
wherein at least one of the formulation components is solid, semi solid or waxy.

2. The formulation of claim 1 wherein the formulation has at least partial resistance to creaming when subjected to centrifugation at 3000 rpm for 10 minutes or following one freeze-thaw cycle.

3. A waterless foamable hydrophilic carrier formulation, comprising:
(a) a silicone;
(b) about 25% to about 98% of a primary waterless solvent selected from the group consisting of (1) a propylene glycol or derivative, (2) a polyethylene glycol (PEG) or derivative, and mixtures thereof;
(c) 0% to about 48% of at least one secondary waterless solvent;
(d) about 0.05% to about 20% of an Accommodating Agent or Complex;
(e) about 0.01% to about 5% by weight of at least one polymeric agent when the primary waterless solvent is a propylene glycol; and
(f) a liquefied or compressed gas propellant at a concentration of about 3% to about 25% by weight of the total composition;
wherein the formulation is a hygroscopic emulsion; and
wherein the Accomodating Agent or Complex is selected from one or more of the group consisting of
a. at least one surface-active agent at a concentration of about 0.1% to less than about 15% by weight;
b. at least one polymeric agent at a concentration of about 0.1% to about 5% by weight, wherein the at least one polymeric agent is selected from the group consisting of a bioadhesive agent, a gelling agent, a film forming agent and a phase change agent;
c. at least one foam adjuvant at a concentration of about 0.1% to about 5% by weight; selected from the group consisting of a fatty alcohol, a fatty acid and a hydroxyl fatty acid, and
d. at least one Stabilizing agent at a concentration of about 0.1% to about 5% by weight; and
wherein at least one of the formulation components is solid, semi solid or waxy; and
wherein the composition is shakable or flowable; and
wherein the composition is stored in an aerosol container and upon release expands to form a breakable foam.

4. The formulation of claim 3 further comprising an effective amount of an active agent and optionally a modulating agent.

5. The formulation of claim 3 wherein the surface active agent is selected from one of the groups consisting of
a. a solid, semi solid or waxy surfactant;
b. a combination of two or more surface active agents, wherein at least one of the two or more surface active agents is solid, semi solid or waxy; and
c. a surfactant capable of forming liquid crystals.

6. The formulation of claim 3 wherein the surface active agent is selected from one of the groups consisting of
a. polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkylyl ether, polyoxyethylene cetyl ether, polyoxyethylene palmityl ether, polyethylene oxide hexadecyl ether, polyethylene glycol cetyl ether, brij 38, brij 52, brij 56 and brij W1, a sucrose ester, a partial ester of sorbitol, sorbitan monolaurate, sorbitan monolaurate a monoglyceride, a diglyceride, isoceteth-20, staereth 2 and a mono, di or tri fatty acid sucrose ester;
b. laureth-4, glyceryl stearate, PEG-100 stearate, ceteareth-6, stearyl alcohol, myrj 52, steareth-2, steareth 21, poyglyceryl 10 laurate, POE (2) cetyl ether, cetearyl glucoside, cetyryl alcohol, methyl glucose sesquistearate, span 60, sucrose stearic acid esters, sorbitan stearate, sucrose cocoate, Peg 40 stearate and isostearath 20;
c. a polymeric emulsifier, particularly Permulen (TR1 or TR2); liquid crystal systems, particularly Arlatone (2121), Stepan (Mild RM1), Nikomulese (41) and Montanov (68);
d. a combination of at least two surfactants selected from the group consisting of steareth-2 and steareth-21; glyceryl stearate and PEG-100 stearate; ceteareth-6 and stearyl alcohol; cetearyl glucoside and cetyryl alcohol; sorbitan stearate and sucrose cocoate; and polysorbate 80 and PEG-40 stearate; seareth 2 and methyl glucose sesqui stearate; and steareth 2 and cetearyl glucoside and cetearyl alcohol; and
e. a combination of at least two surfactants selected from the group consisting of combinations of polyoxyethylene alkyl ethers, particularly Brij 59/Brij 10; Brij 52/Brij 10; Steareth 2/Steareth 20; Steareth 2/Steareth 21 (Brij 72/BRIJ 721); Myrj 52/Myrj 59; combinations of sucrose esters, particularly Surphope 1816/Surphope 1807; combinations of sorbitan esters, particularly Span 20/Span 80; Span 20/Span 60; combinations of sucrose esters and sorbitan esters, particularly Surphope 1811 and Span 60; and combinations of liquid polysorbate detergents and PEG compounds, particularly Twin 80/PEG-40 stearate/methyl glucose sequistearate.

7. The formulation of claim 3 wherein the polymeric agent is selected from the groups consisting of
a. locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, an amine-bearing polymer, chitosan, alginic acid, hyaluronic acid, a chemically modified starch, a carboxyvinyl polymer, polyvinylpyrrolidone, polyvinyl alcohol, a polyacrylic acid polymer, a polymethacrylic acid polymer, polyvinyl acetate, a polyvinyl chloride polymer, a polyvinylidene chloride polymer, methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, a cationic cellulose PEG 1000, PEG 4000, PEG 6000 and PEG 8000;
b. hydroxypropylcellulose, aluminum starch octenylsuccinate and a carbomer; and
c. Carbopol® 934, Carbopol® 940, Carbopol® 941, Carbopol® 980 and Carbopol® 981.

8. The formulation of claim 3 wherein the silicone is selected from one or more of the groups consisting of
a) an unmodified silicone;
b) a liquid silicone;
c) a cyclic silicone;
d) a linear silicone,
e) a branched silicone;
f) a low molecular weight silicone;
g) a high molecular weight silicone;
h) a volatile, a-non volatile, a partially volatile silicone or combinations of two or more thereof; and
i) at least one non-volatile silicone and at least one volatile or partially volatile silicone, wherein, on application to a site, the at least one volatile or partially volatile silicone evaporates and the at least one non-volatile silicone remains at the site of application.

9. The formulation of claim 3, wherein the silicone is selected from the group consisting of dimethicone, cetyl dimethicone, cyclomethicone, cyclodimethicone, simethicone, polydimethylsiloxane polymer, cyclopentasiloxane DC245, Dow Corning® 345 Fluid, and bis-PEG-18 methyl ether dimethyl silane and mixtures thereof.

10. The formulation of claim 3, wherein the primary waterless is a polyethylene glycol or a derivative thereof and less than 15% of the PEG or derivative thereof is a solid.

11. The formulation of claim 10, wherein less than 5% of the polyethylene glycol or derivative is a solid.

12. The formulation of claim 6, wherein the surface active agent further comprises an ionic surfactant, selected from the group consisting of a cationic surfactant, a zwitterionic surfactant, an amphoteric surfactant and an ampholytic surfactant.

13. The formulation of claim 3 further comprising a hygroscopic substance selected from the group consisting of
a) one or more polyethylene glycols (PEGs);
b) one or more surfactants comprising PEG;
c) one or more polyols;
d) one or more monosaccharides, disaccharides, oligosaccharides and sugar alcohols in an amount to provide hygroscopic properties; and
e) honey.

14. The formulation of claim 3 further comprising at least one co-solvent selected from one of the groups consisting of
(a) a polyol selected from one of the groups consisting of (i) a diol, a triol and a saccharide, wherein the triol is selected from the group consisting of glycerin, butane-1,2,3-triol, butane-1,2,4-triol and hexane-1,2,6-triol, and wherein the diol is selected from the group consisting of propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol and dibutylene glycol; and (ii) at least one diol and at least one triol, wherein the ratio between the diol and triol is between 9:1 and 1:1; and
(b) dimethyl isosorbide, tetrahydrofurfuryl alcohol polyethyleneglycol, ether, DMSO, a pyrrolidone, N-Methyl-2-pyrrolidone, 1-Methyl-2-pyrrolidinone, ethyl proxitol, dimethylacetamide, a PEG-type surfactant, an alpha hydroxy acid, lactic acid and glycolic acid or an alkyl alcohol.

15. The formulation of claim 3 wherein the ratio between the primary waterless solvent and the secondary waterless solvent is between about 9:1 and about 1:1.

16. The formulation of claim 3 wherein when the primary solvent is propylene glycol, the Accomodating Agent or Complex comprises a combination of a surface active agent and a polymeric agent.

17. The formulation of claim 3 further comprising a hydrophobic solvent.

18. The formulation of claim 3 further comprising up to about 5% water.

19. The formulation of claim 3 further comprising an additional component selected from the group consisting of an anti-perspirant, an anti-static agent, a buffering agent, a bulking agent, a chelating agent, a colorant, a conditioner, a deodorant, a diluent, a dye, an emollient, fragrance, a humectant, an occlusive agent, a penetration enhancer, a perfuming agent, a permeation enhancer, a pH-adjusting agent, a preservative, a skin penetration enhancer, a sunscreen, a sun blocking agent, and a sunless tanning agent.

20. The formulation of claim 3, wherein the composition ingredients are pretreated to reduce, remove or eliminate any residual or associated or absorbed water.

21. The formulation of claim 4, wherein the active agent is selected from the group consisting of antiinfective, antifungal, antiviral, anesthesic analgesic, corticosterois, non steroid anti inflammatory, retinoids, lubricating agents anti warts, antiproliferative, vasoactive, keratolytic, insectiside and repellants, dicarboxylic acids and esters; calcium channel blockers, cholinergic, N-oxide doners, photodynamic, anti acne, anti wrinkle, antioxidants, self tanning active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers.

22. The formulation of claim 4 wherein the active agent is selected from the group consisting of: acyclovir, azelaic acid, benzoyl peroxide, betamethasone 17 valerate micronized, caffeine, calcipotriol, calcipotriol hydrate, calcitriol, ciclopiroxolamine, diclofenac sodium, ketoconazole, miconazole nitrate, minoxidil, mupirocin, nifedipine regular, permethrin bpc (cis:trans 25:75), piroxicam, salicylic acid and terbinafine hcl.

23. The formulation of claim 4 wherein the active agent is unstable in the presence of water or is susceptible to oxidation.

24. A waterless foamable hydrophilic carrier formulation, comprising a precursor and a liquefied or compressed gas propellant, the precursor comprising:
(a) a silicone, wherein the silicone is selected from the group consisting of dimethicone, cetyl dimethicone, cyclomethicone, cyclodimethicone, simethicone, polydimethylsiloxane polymer, cyclopentasiloxane DC245, Dow Corning® 345 Fluid, and bis-PEG-18 methyl ether dimethyl silane and mixtures thereof.
(b) about 25% to about 98% of a primary waterless solvent selected from the group consisting of (1) a propylene glycol or derivative, (2) a polyethylene glycol (PEG) or derivative, and mixtures thereof;
(c) 0% to about 48% of at least one secondary waterless solvent
(d) about 0.05% to about 20% of an Accommodating Agent or Complex;
(e) about 0.01% to about 5% by weight of at least one polymeric agent when the primary waterless solvent is a propylene glycol;
(f) a therapeutically effective amount of an active agent; and
(g) 0% to about 1% of a modulating agent;
wherein the ratio of the precursor to the liquefied or compressed gas propellant is about 100:3 to about 100:25 by weight; and
wherein the formulation is a hygroscopic emulsion; and
wherein the Accomodating Agent or Complex comprises at least one surface-active agent at a concentration of about 0.1% to less than about 15% by weight; at least one polymeric agent at a concentration of about 0.1% to about 5% by weight, wherein the at least one polymeric agent is selected from a bioadhesive agent, a gelling agent, a film forming agent and a phase change agent; and at least one foam adjuvant at a concentration of about 0% to about 5% by weight; selected from the group consisting of a fatty alcohol, a fatty acid and a hydroxyl fatty acid;
wherein at least one component of the formulation components is solid, semi solid or waxy;
wherein the formulation has some or partial resistance to creaming when subjected to centrifugation at 3000 rpm for 10 min or to FTC for at least one cycle;
wherein the composition is shakable or flowable; and
wherein the composition is stored in an aerosol container and upon release expands to form a breakable foam.

25. The formulation of claim 4, wherein the primary waterless solvent comprises about 70% to about 96.5% of a polyethylene glycol (PEG) or derivative or mixtures thereof.

26. The formulation of claim 4, wherein the primary waterless solvent comprising about 70% to about 96.5% of a propylene glycol or derivative.

27. The formulation of claim 25 wherein the formulation has at least partial resistance to creaming when subjected to centrifugation at 3000 rpm for 10 minutes or following one freeze-thaw cycle.

28. The formulation of claim 26 wherein the formulation has at least partial resistance to creaming when subjected to centrifugation at 3000 rpm for 10 minutes or following one freeze-thaw cycle.

29. A method of treating, alleviating or preventing a dermatological, cosmetic or mucosal disorder, comprising administering topically to a subject having said disorder a therapeutically effective amount of the formulation according to claim 4.

30. A hygroscopic silicone in glycol emulsion composition comprising a polyethylene glycol or derivatives and mixtures thereof or comprising a propylene glycol or derivatives thereof at a sufficient concentration alone as a component in the composition or with one or more other hygroscopic substances to provide
(a) at least one hygroscopic substance at a sufficient concentration to provide an Aw value of the hygroscopic therapeutic containing composition of less than 0.9; and
(b) a therapeutic agent thereof or combinations thereof.

31. The formulation of claim 30, wherein the Aw value is selected from the group consisting of a) in the range of about 0.8 and about 0.9; b) in the range of about 0.7 and about 0.8; and c) in the range of about less than about 0.7.

32. The formulation of claim 4 having an average droplet size of less than 15 microns.

33. The formulation of claim 4 having an average bubble size of less than 200 microns.

34. The formulation of claim 3 having a reduced sensation of dryness when applied topically to the skin in comparison to a similar formulation without silicone.

35. The formulation of claim 4 further comprising about 1% to about 10% microsponges containing an effective amount of at least one active agent.

36. The formulation of claim 1, wherein the carrier is essentially solid or gel like.

37. A method of liquefying a carrier, composition or foam formulation of claim 1 comprising
a) preparing and adding the carrier, composition or foam formulation to a container capable of being sealed and withstanding pressure of a liquefied or compressed gas propellant at a concentration of about 3% to about 25% by weight of the total composition;
b) allowing the composition to form a wax solid or gel;
c) adding a liquefied or compressed gas propellant at a concentration of about 3% to about 25% by weight of the total composition to the sealed container;
d) shaking or agitating the container.

38. The method of claim 37, wherein the ratio of propellant to the solid or gel is in the range of about or less than 1:4 to about or less than 1:15

39. A kit comprising a dual chamber device or dual dispenser head, a first canister comprising a first foamable composition according to claim 4 comprising a first active pharmaceutical ingredient and a second canister comprising a second foamable composition according to claim 4 comprising a second active pharmaceutical ingredient, wherein each canister is connectable to the said device or head.

40. The kit of claim 39, wherein the first active pharmaceutical ingredient is a steroid and the second active pharmaceutical ingredient is a vitamin D derivative.
